SEQUENCE LISTING (1387 sequences)

SEQ ID NO: 1 - DNA, 24 bp, Artificial Sequence
  Synthetic
aaattgtgaaagattgtttgtgta

SEQ ID NO: 2 - DNA, 24 bp, Artificial Sequence
  Synthetic
gttagagttaattgtatttgatga

SEQ ID NO: 3 - DNA, 24 bp, Artificial Sequence
  Synthetic
atgttaaagtaagtgttgaaatgt

SEQ ID NO: 4 - DNA, 24 bp, Artificial Sequence
  Synthetic
tgatgttagaagtatattgtgaat

SEQ ID NO: 5 - DNA, 24 bp, Artificial Sequence
  Synthetic
tttgtgtagaatatgtgttgttaa

SEQ ID NO: 6 - DNA, 24 bp, Artificial Sequence
  Synthetic
ataagtgtaagtgaaataagaaga

SEQ ID NO: 7 - DNA, 24 bp, Artificial Sequence
  Synthetic
aagagtatttgttgtgagttaaat

SEQ ID NO: 8 - DNA, 24 bp, Artificial Sequence
  Synthetic
gtgtttatgttatatgtgaagttt

SEQ ID NO: 9 - DNA, 24 bp, Artificial Sequence
  Synthetic
aaagagaatagaatatgtgtaagt

SEQ ID NO: 10 - DNA, 24 bp, Artificial Sequence
  Synthetic
tatgaaagagtgagataatgttta

SEQ ID NO: 11 - DNA, 24 bp, Artificial Sequence
  Synthetic
atgagaaatatgttagaatgtgat

SEQ ID NO: 12 - DNA, 24 bp, Artificial Sequence
  Synthetic
ttagttgttgatgtttagtagttt

SEQ ID NO: 13 - DNA, 24 bp, Artificial Sequence
  Synthetic
gtaaagagtataagtttgatgata

SEQ ID NO: 14 - DNA, 24 bp, Artificial Sequence
  Synthetic
aaagtaagaatgatgtaataagtg

SEQ ID NO: 15 - DNA, 24 bp, Artificial Sequence
  Synthetic
gtagaaatagtttattgatgattg

SEQ ID NO: 16 - DNA, 24 bp, Artificial Sequence
  Synthetic
tgtaagtgaaatagtgagttattt

SEQ ID NO: 17 - DNA, 24 bp, Artificial Sequence
  Synthetic
aaatagatgatataagtgagaatg

SEQ ID NO: 18 - DNA, 24 bp, Artificial Sequence
  Synthetic
ataagttataagtgttatgtgagt

SEQ ID NO: 19 - DNA, 24 bp, Artificial Sequence
  Synthetic
tatagataaagagatgatttgttg

SEQ ID NO: 20 - DNA, 24 bp, Artificial Sequence
  Synthetic
agagttgagaatgtatagtattat

SEQ ID NO: 21 - DNA, 24 bp, Artificial Sequence
  Synthetic
aagtagtttgtaagaatgattgta

SEQ ID NO: 22 - DNA, 24 bp, Artificial Sequence
  Synthetic
ttatgaaattgagtgaagattgat

SEQ ID NO: 23 - DNA, 24 bp, Artificial Sequence
  Synthetic
gtatatgtaaattgttatgttgag

SEQ ID NO: 24 - DNA, 24 bp, Artificial Sequence
  Synthetic
gaattgtataaagtattagatgtg

SEQ ID NO: 25 - DNA, 24 bp, Artificial Sequence
  Synthetic
tagatgagattaagtgttatttga

SEQ ID NO: 26 - DNA, 24 bp, Artificial Sequence
  Synthetic
gttaagtttgtttatgtatagaag

SEQ ID NO: 27 - DNA, 24 bp, Artificial Sequence
  Synthetic
gagtattagtaaagtgatatgata

SEQ ID NO: 28 - DNA, 24 bp, Artificial Sequence
  Synthetic
gtgaatgatttagtaaatgattga

SEQ ID NO: 29 - DNA, 24 bp, Artificial Sequence
  Synthetic
gattgaagttatagaaatgattag

SEQ ID NO: 30 - DNA, 24 bp, Artificial Sequence
  Synthetic
agtgataaatgttagttgaattgt

SEQ ID NO: 31 - DNA, 24 bp, Artificial Sequence
  Synthetic
tatatagtaaatgtttgtgtgttg

SEQ ID NO: 32 - DNA, 24 bp, Artificial Sequence
  Synthetic
ttaagtgttagttatttgttgtag

SEQ ID NO: 33 - DNA, 24 bp, Artificial Sequence
  Synthetic
gtagtaatatgaagtgagaatata

SEQ ID NO: 34 - DNA, 24 bp, Artificial Sequence
  Synthetic
tagtgtatagaatgtagatttagt

SEQ ID NO: 35 - DNA, 24 bp, Artificial Sequence
  Synthetic
ttgtagattagatgtgtttgtaaa

SEQ ID NO: 36 - DNA, 24 bp, Artificial Sequence
  Synthetic
tagtatagagtagagatgatattt

SEQ ID NO: 37 - DNA, 24 bp, Artificial Sequence
  Synthetic
attgtgaaagaaagagaagaaatt

SEQ ID NO: 38 - DNA, 24 bp, Artificial Sequence
  Synthetic
tgtgagaattaagattaagaatgt

SEQ ID NO: 39 - DNA, 24 bp, Artificial Sequence
  Synthetic
atattagttaagaaagaagagttg

SEQ ID NO: 40 - DNA, 24 bp, Artificial Sequence
  Synthetic
ttgtagttgagaaatatgtagttt

SEQ ID NO: 41 - DNA, 24 bp, Artificial Sequence
  Synthetic
tagagttgttaaagagtgtaaata

SEQ ID NO: 42 - DNA, 24 bp, Artificial Sequence
  Synthetic
gttatgatgtgtataagtaatatg

SEQ ID NO: 43 - DNA, 24 bp, Artificial Sequence
  Synthetic
tttgttagaatgagaagatttatg

SEQ ID NO: 44 - DNA, 24 bp, Artificial Sequence
  Synthetic
agtatagtttaaagaagtagtaga

SEQ ID NO: 45 - DNA, 24 bp, Artificial Sequence
  Synthetic
gtgagatatagatttagaaagtaa

SEQ ID NO: 46 - DNA, 24 bp, Artificial Sequence
  Synthetic
ttgtttatagtgaagtgaatagta

SEQ ID NO: 47 - DNA, 24 bp, Artificial Sequence
  Synthetic
aagtaagtagtaatagtgtgttaa

SEQ ID NO: 48 - DNA, 24 bp, Artificial Sequence
  Synthetic
atttgtgagttatgaaagataaga

SEQ ID NO: 49 - DNA, 24 bp, Artificial Sequence
  Synthetic
gaaagtagagaataaagataagaa

SEQ ID NO: 50 - DNA, 24 bp, Artificial Sequence
  Synthetic
atttaagattgttaagagtagaag

SEQ ID NO: 51 - DNA, 24 bp, Artificial Sequence
  Synthetic
gtttaaagattgtaagaatgtgta

SEQ ID NO: 52 - DNA, 24 bp, Artificial Sequence
  Synthetic
tttgtgaagatgaagtatttgtat

SEQ ID NO: 53 - DNA, 24 bp, Artificial Sequence
  Synthetic
tgtgtttagaatttagtatgtgta

SEQ ID NO: 54 - DNA, 24 bp, Artificial Sequence
  Synthetic
gataatgattatagaaagtgtttg

SEQ ID NO: 55 - DNA, 24 bp, Artificial Sequence
  Synthetic
gttatttgtaagttaagatagtag

SEQ ID NO: 56 - DNA, 24 bp, Artificial Sequence
  Synthetic
agtttattgaaagagtttgaatag

SEQ ID NO: 57 - DNA, 24 bp, Artificial Sequence
  Synthetic
ttgtgtttattgtgtagtttaaag

SEQ ID NO: 58 - DNA, 24 bp, Artificial Sequence
  Synthetic
attgtgagaagatatgaaagttat

SEQ ID NO: 59 - DNA, 24 bp, Artificial Sequence
  Synthetic
tgagaatgtaaagaatgtttattg

SEQ ID NO: 60 - DNA, 24 bp, Artificial Sequence
  Synthetic
atgtgaaagttatgatgttaattg

SEQ ID NO: 61 - DNA, 24 bp, Artificial Sequence
  Synthetic
gtttagtattagttgttaagattg

SEQ ID NO: 62 - DNA, 24 bp, Artificial Sequence
  Synthetic
gattgatatttgaatgtttgtttg

SEQ ID NO: 63 - DNA, 24 bp, Artificial Sequence
  Synthetic
tgaattgaaagtgtaatgttgtat

SEQ ID NO: 64 - DNA, 24 bp, Artificial Sequence
  Synthetic
gattgtattgttgagaatagaata

SEQ ID NO: 65 - DNA, 24 bp, Artificial Sequence
  Synthetic
aaatttgagatttgtgatagagta

SEQ ID NO: 66 - DNA, 24 bp, Artificial Sequence
  Synthetic
gtaattagatttgtttgttgttgt

SEQ ID NO: 67 - DNA, 24 bp, Artificial Sequence
  Synthetic
gtttgtattgttagtgaatatagt

SEQ ID NO: 68 - DNA, 24 bp, Artificial Sequence
  Synthetic
atgtagtagtagatgtttatgaat

SEQ ID NO: 69 - DNA, 24 bp, Artificial Sequence
  Synthetic
tgtttaaagatgattgaagaaatg

SEQ ID NO: 70 - DNA, 24 bp, Artificial Sequence
  Synthetic
tgtgataatgatgttatttgtgta

SEQ ID NO: 71 - DNA, 24 bp, Artificial Sequence
  Synthetic
atagttgtgagaatttgtaattag

SEQ ID NO: 72 - DNA, 24 bp, Artificial Sequence
  Synthetic
atagatgtaagagaaattgtgaaa

SEQ ID NO: 73 - DNA, 24 bp, Artificial Sequence
  Synthetic
agattaagagaagttaatagagta

SEQ ID NO: 74 - DNA, 24 bp, Artificial Sequence
  Synthetic
gaagtaaattgtgaatgaaagaaa

SEQ ID NO: 75 - DNA, 24 bp, Artificial Sequence
  Synthetic
aatgtaagaaagaagattgttgta

SEQ ID NO: 76 - DNA, 24 bp, Artificial Sequence
  Synthetic
tttgatttatgtgttatgttgagt

SEQ ID NO: 77 - DNA, 24 bp, Artificial Sequence
  Synthetic
gtattgagaaatttgaagaatgaa

SEQ ID NO: 78 - DNA, 24 bp, Artificial Sequence
  Synthetic
gaattgtatgaaatgaattgtaag

SEQ ID NO: 79 - DNA, 24 bp, Artificial Sequence
  Synthetic
tattgtagaagtaaagttagaagt

SEQ ID NO: 80 - DNA, 24 bp, Artificial Sequence
  Synthetic
tttatgtaatgataagtgtagttg

SEQ ID NO: 81 - DNA, 24 bp, Artificial Sequence
  Synthetic
atatagttgaaattgtgatagtgt

SEQ ID NO: 82 - DNA, 24 bp, Artificial Sequence
  Synthetic
ataagaaattagagagttgtaaag

SEQ ID NO: 83 - DNA, 24 bp, Artificial Sequence
  Synthetic
gaattgtgaaatgtgattgatata

SEQ ID NO: 84 - DNA, 24 bp, Artificial Sequence
  Synthetic
aaataagtagtttaatgagagaag

SEQ ID NO: 85 - DNA, 24 bp, Artificial Sequence
  Synthetic
gattaaagaagtaagtgaatgttt

SEQ ID NO: 86 - DNA, 24 bp, Artificial Sequence
  Synthetic
tatgtgtgttgtttagtgttatta

SEQ ID NO: 87 - DNA, 24 bp, Artificial Sequence
  Synthetic
gagttatatgtagttagagttata

SEQ ID NO: 88 - DNA, 24 bp, Artificial Sequence
  Synthetic
gaaagaaagaagtgttaagttaaa

SEQ ID NO: 89 - DNA, 24 bp, Artificial Sequence
  Synthetic
tagtattagtaagtatgtgattgt

SEQ ID NO: 90 - DNA, 24 bp, Artificial Sequence
  Synthetic
ttgtgtgattgaatattgtgaaat

SEQ ID NO: 91 - DNA, 24 bp, Artificial Sequence
  Synthetic
atgtgaaagagttaagtgattaaa

SEQ ID NO: 92 - DNA, 24 bp, Artificial Sequence
  Synthetic
gattgaatgattgagatatgtaaa

SEQ ID NO: 93 - DNA, 24 bp, Artificial Sequence
  Synthetic
aagatgatagttaagtgtaagtta

SEQ ID NO: 94 - DNA, 24 bp, Artificial Sequence
  Synthetic
tagttgttattgagaatttagaag

SEQ ID NO: 95 - DNA, 24 bp, Artificial Sequence
  Synthetic
tttatagtgaattatgagtgaaag

SEQ ID NO: 96 - DNA, 24 bp, Artificial Sequence
  Synthetic
gatagatttagaatgaattaagtg

SEQ ID NO: 97 - DNA, 24 bp, Artificial Sequence
  Synthetic
tttgaagaagagatttgaaattga

SEQ ID NO: 98 - DNA, 24 bp, Artificial Sequence
  Synthetic
atgaataagagttgataaatgtga

SEQ ID NO: 99 - DNA, 24 bp, Artificial Sequence
  Synthetic
tgtttatgtagtgtagattgaatt

SEQ ID NO: 100 - DNA, 24 bp, Artificial Sequence
  Synthetic
tttaagtgagttatagaagtagta

SEQ ID NO: 101 - DNA, 24 bp, Artificial Sequence
  Synthetic
gatttatgtgtttgaagttaagat

SEQ ID NO: 102 - DNA, 24 bp, Artificial Sequence
  Synthetic
tagttagagaaagtgataaagtta

SEQ ID NO: 103 - DNA, 24 bp, Artificial Sequence
  Synthetic
gtaatgataatgaagtgtatatag

SEQ ID NO: 104 - DNA, 24 bp, Artificial Sequence
  Synthetic
aatgaagtgttagtatagatagta

SEQ ID NO: 105 - DNA, 24 bp, Artificial Sequence
  Synthetic
taaattgagtttgtttgattgtag

SEQ ID NO: 106 - DNA, 24 bp, Artificial Sequence
  Synthetic
taatgaagaataagtatgagtgtt

SEQ ID NO: 107 - DNA, 24 bp, Artificial Sequence
  Synthetic
aaatgtaatagtgttgttagttag

SEQ ID NO: 108 - DNA, 24 bp, Artificial Sequence
  Synthetic
agagttagtgaaatgttgttaaat

SEQ ID NO: 109 - DNA, 24 bp, Artificial Sequence
  Synthetic
gaaatagaaatgtattgtttgtga

SEQ ID NO: 110 - DNA, 24 bp, Artificial Sequence
  Synthetic
agttataagtttgtgagaattaag

SEQ ID NO: 111 - DNA, 24 bp, Artificial Sequence
  Synthetic
gagtttatagttagaatatgttgt

SEQ ID NO: 112 - DNA, 24 bp, Artificial Sequence
  Synthetic
agagttattagaagaagatttaag

SEQ ID NO: 113 - DNA, 24 bp, Artificial Sequence
  Synthetic
gagttaatgaaataagtatttgtg

SEQ ID NO: 114 - DNA, 24 bp, Artificial Sequence
  Synthetic
atgatgaatagttgaagtatatag

SEQ ID NO: 115 - DNA, 24 bp, Artificial Sequence
  Synthetic
atagatatgagatgaaagttagta

SEQ ID NO: 116 - DNA, 24 bp, Artificial Sequence
  Synthetic
tatgtaaagaaagtgaaagaagaa

SEQ ID NO: 117 - DNA, 24 bp, Artificial Sequence
  Synthetic
tgaatgtagaaatgaatgttgaaa

SEQ ID NO: 118 - DNA, 24 bp, Artificial Sequence
  Synthetic
aattgaatagtgtgtgagtttaat

SEQ ID NO: 119 - DNA, 24 bp, Artificial Sequence
  Synthetic
agatattgtttgattaatgaagag

SEQ ID NO: 120 - DNA, 24 bp, Artificial Sequence
  Synthetic
aaagttgtaaagttgaagataaag

SEQ ID NO: 121 - DNA, 24 bp, Artificial Sequence
  Synthetic
gttaagagattatgagatgtatta

SEQ ID NO: 122 - DNA, 24 bp, Artificial Sequence
  Synthetic
agaagatataagaagattgaattg

SEQ ID NO: 123 - DNA, 24 bp, Artificial Sequence
  Synthetic
gtagaaatttgaattgatgtgaaa

SEQ ID NO: 124 - DNA, 24 bp, Artificial Sequence
  Synthetic
aagagtagattgataagtatatga

SEQ ID NO: 125 - DNA, 24 bp, Artificial Sequence
  Synthetic
tgatatagtagtgaagaaataagt

SEQ ID NO: 126 - DNA, 24 bp, Artificial Sequence
  Synthetic
agataatgatgagaaatgaagata

SEQ ID NO: 127 - DNA, 24 bp, Artificial Sequence
  Synthetic
atgtgaaagtatttgtgatatagt

SEQ ID NO: 128 - DNA, 24 bp, Artificial Sequence
  Synthetic
aataagagaattgatatgaagatg

SEQ ID NO: 129 - DNA, 24 bp, Artificial Sequence
  Synthetic
taagtgtatttagtagaatgaagt

SEQ ID NO: 130 - DNA, 24 bp, Artificial Sequence
  Synthetic
tatgttagatttgttgagattgat

SEQ ID NO: 131 - DNA, 24 bp, Artificial Sequence
  Synthetic
agtttgtatgaagagatagtattt

SEQ ID NO: 132 - DNA, 24 bp, Artificial Sequence
  Synthetic
gagaaatgttatgtatttagtagt

SEQ ID NO: 133 - DNA, 24 bp, Artificial Sequence
  Synthetic
tatgtgagaatgtgtttgatttaa

SEQ ID NO: 134 - DNA, 24 bp, Artificial Sequence
  Synthetic
gtatgtttgtttatagaatgtatg

SEQ ID NO: 135 - DNA, 24 bp, Artificial Sequence
  Synthetic
gagtatatagaagaaagaaatttg

SEQ ID NO: 136 - DNA, 24 bp, Artificial Sequence
  Synthetic
atgagtgaagtaaatgtagttatt

SEQ ID NO: 137 - DNA, 24 bp, Artificial Sequence
  Synthetic
ttaagaagtgagttattgtgatat

SEQ ID NO: 138 - DNA, 24 bp, Artificial Sequence
  Synthetic
atgaaatgagaatattgttgtttg

SEQ ID NO: 139 - DNA, 24 bp, Artificial Sequence
  Synthetic
gattaatgattatgtgaattgatg

SEQ ID NO: 140 - DNA, 24 bp, Artificial Sequence
  Synthetic
gaaatgttaaagatatgaaagtag

SEQ ID NO: 141 - DNA, 24 bp, Artificial Sequence
  Synthetic
tattgttgatttgatattagtgtg

SEQ ID NO: 142 - DNA, 24 bp, Artificial Sequence
  Synthetic
tttatgtttgtgtatgtaagtagt

SEQ ID NO: 143 - DNA, 24 bp, Artificial Sequence
  Synthetic
aattgaaagaattgtgtgaattga

SEQ ID NO: 144 - DNA, 24 bp, Artificial Sequence
  Synthetic
tgagtttgaatttgtttgagtaat

SEQ ID NO: 145 - DNA, 24 bp, Artificial Sequence
  Synthetic
gatgtataatgatgtgtgtaaatt

SEQ ID NO: 146 - DNA, 24 bp, Artificial Sequence
  Synthetic
atgtgagagaagaatttgtttatt

SEQ ID NO: 147 - DNA, 24 bp, Artificial Sequence
  Synthetic
gtgataaagtattgttgatagaaa

SEQ ID NO: 148 - DNA, 24 bp, Artificial Sequence
  Synthetic
gaagtagaatagaaagttaataga

SEQ ID NO: 149 - DNA, 24 bp, Artificial Sequence
  Synthetic
ttgtgtagttaagagttgtttaat

SEQ ID NO: 150 - DNA, 24 bp, Artificial Sequence
  Synthetic
tagtagtaagttgttagaatagtt

SEQ ID NO: 151 - DNA, 24 bp, Artificial Sequence
  Synthetic
aatttgaagtataatgaatgtgtg

SEQ ID NO: 152 - DNA, 24 bp, Artificial Sequence
  Synthetic
tagaaattgtagtatttgagagaa

SEQ ID NO: 153 - DNA, 24 bp, Artificial Sequence
  Synthetic
tgtatatgttaatgagatgttgta

SEQ ID NO: 154 - DNA, 24 bp, Artificial Sequence
  Synthetic
tatttgataagagaatgaagaagt

SEQ ID NO: 155 - DNA, 24 bp, Artificial Sequence
  Synthetic
ttgaatagtgtaatgaatatgatg

SEQ ID NO: 156 - DNA, 24 bp, Artificial Sequence
  Synthetic
gtagtttgtgaatagaattagttt

SEQ ID NO: 157 - DNA, 24 bp, Artificial Sequence
  Synthetic
aaagatgattgtaatttgtgtgaa

SEQ ID NO: 158 - DNA, 24 bp, Artificial Sequence
  Synthetic
gaagattgttgagttaatagataa

SEQ ID NO: 159 - DNA, 24 bp, Artificial Sequence
  Synthetic
agattatgtagtgatgtaaatgtt

SEQ ID NO: 160 - DNA, 24 bp, Artificial Sequence
  Synthetic
gaatttagatgtagatatgaatgt

SEQ ID NO: 161 - DNA, 24 bp, Artificial Sequence
  Synthetic
gatagaagtgtattaagtaagtta

SEQ ID NO: 162 - DNA, 24 bp, Artificial Sequence
  Synthetic
tatgaattatgagaagaatagagt

SEQ ID NO: 163 - DNA, 24 bp, Artificial Sequence
  Synthetic
tttgttatgaagtgatttgtttgt

SEQ ID NO: 164 - DNA, 24 bp, Artificial Sequence
  Synthetic
gtaaagattgtgttatatgaaatg

SEQ ID NO: 165 - DNA, 24 bp, Artificial Sequence
  Synthetic
ttgtgatagtagttagatatttgt

SEQ ID NO: 166 - DNA, 24 bp, Artificial Sequence
  Synthetic
gaattaagataaagaagagaagta

SEQ ID NO: 167 - DNA, 24 bp, Artificial Sequence
  Synthetic
gattgtagaatgaatttgtagtat

SEQ ID NO: 168 - DNA, 24 bp, Artificial Sequence
  Synthetic
aaataagagagagaatgatttagt

SEQ ID NO: 169 - DNA, 24 bp, Artificial Sequence
  Synthetic
aattatgtgaatagattgttgaag

SEQ ID NO: 170 - DNA, 24 bp, Artificial Sequence
  Synthetic
ttaagatttatgtgatagtagagt

SEQ ID NO: 171 - DNA, 24 bp, Artificial Sequence
  Synthetic
ttaaagatagtgtttgttgtgtta

SEQ ID NO: 172 - DNA, 24 bp, Artificial Sequence
  Synthetic
tattgatttatgaagagtatagtg

SEQ ID NO: 173 - DNA, 24 bp, Artificial Sequence
  Synthetic
aaatttgatgagtagtttaagaga

SEQ ID NO: 174 - DNA, 24 bp, Artificial Sequence
  Synthetic
ataaagttgtttgatgtttgaatg

SEQ ID NO: 175 - DNA, 24 bp, Artificial Sequence
  Synthetic
gattgtgatgaataatgttattga

SEQ ID NO: 176 - DNA, 24 bp, Artificial Sequence
  Synthetic
gatgaagaaatatgatatgaatag

SEQ ID NO: 177 - DNA, 24 bp, Artificial Sequence
  Synthetic
ttaaagttattgaagtgaagttga

SEQ ID NO: 178 - DNA, 24 bp, Artificial Sequence
  Synthetic
ttgtaagaaatagagatttgtgtt

SEQ ID NO: 179 - DNA, 24 bp, Artificial Sequence
  Synthetic
gagattgagtttaagtattagatt

SEQ ID NO: 180 - DNA, 24 bp, Artificial Sequence
  Synthetic
agtgataatagaatgataaatgtg

SEQ ID NO: 181 - DNA, 24 bp, Artificial Sequence
  Synthetic
gataatagtgaatttgagttgtat

SEQ ID NO: 182 - DNA, 24 bp, Artificial Sequence
  Synthetic
agatatttgtagtagaaagtatgt

SEQ ID NO: 183 - DNA, 24 bp, Artificial Sequence
  Synthetic
gttatgaatgttgaatttgaatgt

SEQ ID NO: 184 - DNA, 24 bp, Artificial Sequence
  Synthetic
atgaaagatttagttgtgagatat

SEQ ID NO: 185 - DNA, 24 bp, Artificial Sequence
  Synthetic
aaatagagaagttatgatgtgata

SEQ ID NO: 186 - DNA, 24 bp, Artificial Sequence
  Synthetic
ttagtgagaaatgtttaatgtgat

SEQ ID NO: 187 - DNA, 24 bp, Artificial Sequence
  Synthetic
tgaagaatatgtgaaattagtttg

SEQ ID NO: 188 - DNA, 24 bp, Artificial Sequence
  Synthetic
gtttgatagtttaatgagtattga

SEQ ID NO: 189 - DNA, 24 bp, Artificial Sequence
  Synthetic
gttgtaagtaatgataaagtatga

SEQ ID NO: 190 - DNA, 24 bp, Artificial Sequence
  Synthetic
taagagtagtaattgttgtttaga

SEQ ID NO: 191 - DNA, 24 bp, Artificial Sequence
  Synthetic
tttgagagagtatgtatgattatt

SEQ ID NO: 192 - DNA, 24 bp, Artificial Sequence
  Synthetic
attgattgtgaattagatagaaga

SEQ ID NO: 193 - DNA, 24 bp, Artificial Sequence
  Synthetic
gattagtatttagtagtaatagag

SEQ ID NO: 194 - DNA, 24 bp, Artificial Sequence
  Synthetic
tatgtattagagatattgaaagtg

SEQ ID NO: 195 - DNA, 24 bp, Artificial Sequence
  Synthetic
tatgtgaaagtaatgataaatgag

SEQ ID NO: 196 - DNA, 24 bp, Artificial Sequence
  Synthetic
gtaattagtaatgatttgaatgag

SEQ ID NO: 197 - DNA, 24 bp, Artificial Sequence
  Synthetic
gtttattgtaaagatgtaagtgaa

SEQ ID NO: 198 - DNA, 24 bp, Artificial Sequence
  Synthetic
tagtagaattgttgttaaagaatg

SEQ ID NO: 199 - DNA, 24 bp, Artificial Sequence
  Synthetic
tattgttagttatgtagtgtgtaa

SEQ ID NO: 200 - DNA, 24 bp, Artificial Sequence
  Synthetic
gagtgaaagttatatgaaagtata

SEQ ID NO: 201 - DNA, 24 bp, Artificial Sequence
  Synthetic
atatagaagttgatgagtttatga

SEQ ID NO: 202 - DNA, 24 bp, Artificial Sequence
  Synthetic
tttagaagtaagaataagtgagta

SEQ ID NO: 203 - DNA, 24 bp, Artificial Sequence
  Synthetic
tgtgtataagatatttgtaagaag

SEQ ID NO: 204 - DNA, 24 bp, Artificial Sequence
  Synthetic
tagaagagttgtattgttataagt

SEQ ID NO: 205 - DNA, 24 bp, Artificial Sequence
  Synthetic
gtgttattagtttaagttagagta

SEQ ID NO: 206 - DNA, 24 bp, Artificial Sequence
  Synthetic
aatatagtgatgtgaaattgaatg

SEQ ID NO: 207 - DNA, 24 bp, Artificial Sequence
  Synthetic
ttagagaatagagtgattatagtt

SEQ ID NO: 208 - DNA, 24 bp, Artificial Sequence
  Synthetic
gaagtgagttaatgatttgtaaat

SEQ ID NO: 209 - DNA, 24 bp, Artificial Sequence
  Synthetic
aatgtaaagtaaagaaagtgatga

SEQ ID NO: 210 - DNA, 24 bp, Artificial Sequence
  Synthetic
gttagttatgatgaatattgtgta

SEQ ID NO: 211 - DNA, 24 bp, Artificial Sequence
  Synthetic
aaatgagttagagtagaattatgt

SEQ ID NO: 212 - DNA, 24 bp, Artificial Sequence
  Synthetic
gatatagaagattagttagtgata

SEQ ID NO: 213 - DNA, 24 bp, Artificial Sequence
  Synthetic
atagtttgttgagatttatgagta

SEQ ID NO: 214 - DNA, 24 bp, Artificial Sequence
  Synthetic
tagaatagttagtagtaagagtat

SEQ ID NO: 215 - DNA, 24 bp, Artificial Sequence
  Synthetic
gaatttgtattgtgaagtttagta

SEQ ID NO: 216 - DNA, 24 bp, Artificial Sequence
  Synthetic
gtagtaagaagagaattagattaa

SEQ ID NO: 217 - DNA, 24 bp, Artificial Sequence
  Synthetic
aatgtgttatgtatgtaaatagtg

SEQ ID NO: 218 - DNA, 24 bp, Artificial Sequence
  Synthetic
gaattagttagagtaaattgtttg

SEQ ID NO: 219 - DNA, 24 bp, Artificial Sequence
  Synthetic
gaaattgaagatagtaagaaatga

SEQ ID NO: 220 - DNA, 24 bp, Artificial Sequence
  Synthetic
gtgtattatgtgatttatgataga

SEQ ID NO: 221 - DNA, 24 bp, Artificial Sequence
  Synthetic
tattatgagaaagttgaatagtag

SEQ ID NO: 222 - DNA, 24 bp, Artificial Sequence
  Synthetic
tatgtattgtattgagtagatgaa

SEQ ID NO: 223 - DNA, 24 bp, Artificial Sequence
  Synthetic
gtgattgaatagtagattgtttaa

SEQ ID NO: 224 - DNA, 24 bp, Artificial Sequence
  Synthetic
agtaagttgtttgattgaaatttg

SEQ ID NO: 225 - DNA, 24 bp, Artificial Sequence
  Synthetic
gaagtttgatttaagtttaagaag

SEQ ID NO: 226 - DNA, 24 bp, Artificial Sequence
  Synthetic
gagaagataaatgatattgttatg

SEQ ID NO: 227 - DNA, 24 bp, Artificial Sequence
  Synthetic
atgatgagttgttaatagttagtt

SEQ ID NO: 228 - DNA, 24 bp, Artificial Sequence
  Synthetic
tatgatatttgaagagtgttaaga

SEQ ID NO: 229 - DNA, 24 bp, Artificial Sequence
  Synthetic
gagatgattaaagtgatttatgaa

SEQ ID NO: 230 - DNA, 24 bp, Artificial Sequence
  Synthetic
atagttaagagtgatgagaataaa

SEQ ID NO: 231 - DNA, 24 bp, Artificial Sequence
  Synthetic
tttattgttagataaagagttgag

SEQ ID NO: 232 - DNA, 24 bp, Artificial Sequence
  Synthetic
agaatattgatagttgaagttgaa

SEQ ID NO: 233 - DNA, 24 bp, Artificial Sequence
  Synthetic
tagtgtaaagtgtagattgtaaat

SEQ ID NO: 234 - DNA, 24 bp, Artificial Sequence
  Synthetic
agtagtgatatgatttgaatattg

SEQ ID NO: 235 - DNA, 24 bp, Artificial Sequence
  Synthetic
tgtattgaattagaatagtgagaa

SEQ ID NO: 236 - DNA, 24 bp, Artificial Sequence
  Synthetic
tgatatgagatagaagtttaatgt

SEQ ID NO: 237 - DNA, 24 bp, Artificial Sequence
  Synthetic
gaagaagtaagtataaagtaaatg

SEQ ID NO: 238 - DNA, 24 bp, Artificial Sequence
  Synthetic
tttaagtgtgataagaaagataga

SEQ ID NO: 239 - DNA, 24 bp, Artificial Sequence
  Synthetic
tattgttgaatgtgtttaaagaga

SEQ ID NO: 240 - DNA, 24 bp, Artificial Sequence
  Synthetic
gaataatgatgagatgattattga

SEQ ID NO: 241 - DNA, 24 bp, Artificial Sequence
  Synthetic
tagagaaagagagaattgtattaa

SEQ ID NO: 242 - DNA, 24 bp, Artificial Sequence
  Synthetic
atgtataatgagatatgtttgtga

SEQ ID NO: 243 - DNA, 24 bp, Artificial Sequence
  Synthetic
aatagataagattgattgtgtttg

SEQ ID NO: 244 - DNA, 24 bp, Artificial Sequence
  Synthetic
tttgatgataatagaagagaatga

SEQ ID NO: 245 - DNA, 24 bp, Artificial Sequence
  Synthetic
agatgaataagttgtgaatgttta

SEQ ID NO: 246 - DNA, 24 bp, Artificial Sequence
  Synthetic
agatgaaagaaagtgtagaatatt

SEQ ID NO: 247 - DNA, 24 bp, Artificial Sequence
  Synthetic
tgttaaatgtatgtagtaattgag

SEQ ID NO: 248 - DNA, 24 bp, Artificial Sequence
  Synthetic
tagtagtgtgaagttatttgttat

SEQ ID NO: 249 - DNA, 24 bp, Artificial Sequence
  Synthetic
agtgaatgtttgtaaagagtttaa

SEQ ID NO: 250 - DNA, 24 bp, Artificial Sequence
  Synthetic
gataaatgagaattgagtaattgt

SEQ ID NO: 251 - DNA, 24 bp, Artificial Sequence
  Synthetic
tgatgagaaattgtttaagtgttt

SEQ ID NO: 252 - DNA, 24 bp, Artificial Sequence
  Synthetic
aaataagtagtgtgagtaatagta

SEQ ID NO: 253 - DNA, 24 bp, Artificial Sequence
  Synthetic
tatgaaatatgtgatagtaagaga

SEQ ID NO: 254 - DNA, 24 bp, Artificial Sequence
  Synthetic
attgtaagagtgattatagatgat

SEQ ID NO: 255 - DNA, 24 bp, Artificial Sequence
  Synthetic
agagtaagaatgaaagagataata

SEQ ID NO: 256 - DNA, 24 bp, Artificial Sequence
  Synthetic
taagtaagtagatgttaaagagat

SEQ ID NO: 257 - DNA, 24 bp, Artificial Sequence
  Synthetic
aaatagaaagaattgtagagtagt

SEQ ID NO: 258 - DNA, 24 bp, Artificial Sequence
  Synthetic
atagatttaagtgaagagagttat

SEQ ID NO: 259 - DNA, 24 bp, Artificial Sequence
  Synthetic
gaatgtttgtaaatgtatagatag

SEQ ID NO: 260 - DNA, 24 bp, Artificial Sequence
  Synthetic
aaatagaatgagtagtgaaatatg

SEQ ID NO: 261 - DNA, 24 bp, Artificial Sequence
  Synthetic
ttgaattatgtagagaaagtaaag

SEQ ID NO: 262 - DNA, 24 bp, Artificial Sequence
  Synthetic
tagtaaattgagagtagttgaatt

SEQ ID NO: 263 - DNA, 24 bp, Artificial Sequence
  Synthetic
tgtaaagtgtttatagtgtgtaat

SEQ ID NO: 264 - DNA, 24 bp, Artificial Sequence
  Synthetic
atatgatttgagatgagaatgtaa

SEQ ID NO: 265 - DNA, 24 bp, Artificial Sequence
  Synthetic
aatattgatatgtgttgtgaagta

SEQ ID NO: 266 - DNA, 24 bp, Artificial Sequence
  Synthetic
agtgagattatgagtattgattta

SEQ ID NO: 267 - DNA, 24 bp, Artificial Sequence
  Synthetic
ttgtatttagatagtgagattatg

SEQ ID NO: 268 - DNA, 24 bp, Artificial Sequence
  Synthetic
atagaaatgaaagatagatagaag

SEQ ID NO: 269 - DNA, 24 bp, Artificial Sequence
  Synthetic
gattgtatatgtaaagtagtttag

SEQ ID NO: 270 - DNA, 24 bp, Artificial Sequence
  Synthetic
tatgaatgttattgtgtgttgatt

SEQ ID NO: 271 - DNA, 24 bp, Artificial Sequence
  Synthetic
gatattagtagagtaagtatattg

SEQ ID NO: 272 - DNA, 24 bp, Artificial Sequence
  Synthetic
tgagatgaatttgtgttatgatat

SEQ ID NO: 273 - DNA, 24 bp, Artificial Sequence
  Synthetic
tatgaatgaagtaaagagatgtaa

SEQ ID NO: 274 - DNA, 24 bp, Artificial Sequence
  Synthetic
gagtgaatttgttgtaatttgttt

SEQ ID NO: 275 - DNA, 24 bp, Artificial Sequence
  Synthetic
agaaattgtagagttaattgtgta

SEQ ID NO: 276 - DNA, 24 bp, Artificial Sequence
  Synthetic
gtgttaatgaaagttgtgaataat

SEQ ID NO: 277 - DNA, 24 bp, Artificial Sequence
  Synthetic
tgtgatttgttaagaagattaatg

SEQ ID NO: 278 - DNA, 24 bp, Artificial Sequence
  Synthetic
agtagtattgtaaagtataaagag

SEQ ID NO: 279 - DNA, 24 bp, Artificial Sequence
  Synthetic
tgattgttgtatagttattgtgta

SEQ ID NO: 280 - DNA, 24 bp, Artificial Sequence
  Synthetic
gattgtagtttaatgttaagaatg

SEQ ID NO: 281 - DNA, 24 bp, Artificial Sequence
  Synthetic
atgaaataagaaattgagtagaga

SEQ ID NO: 282 - DNA, 24 bp, Artificial Sequence
  Synthetic
tatgatgatatttgttgtatgtgt

SEQ ID NO: 283 - DNA, 24 bp, Artificial Sequence
  Synthetic
tttagagtttgattagtatgtttg

SEQ ID NO: 284 - DNA, 24 bp, Artificial Sequence
  Synthetic
aataagagattgtgatgagaaata

SEQ ID NO: 285 - DNA, 24 bp, Artificial Sequence
  Synthetic
aatgaatagaatagagaatgtaga

SEQ ID NO: 286 - DNA, 24 bp, Artificial Sequence
  Synthetic
gtagtagtaatttgaatgtttgaa

SEQ ID NO: 287 - DNA, 24 bp, Artificial Sequence
  Synthetic
agtgagtaattgattgattgttaa

SEQ ID NO: 288 - DNA, 24 bp, Artificial Sequence
  Synthetic
gaataatgtttagtgtgtttgaaa

SEQ ID NO: 289 - DNA, 24 bp, Artificial Sequence
  Synthetic
atatgaaagtagagaaagtgttat

SEQ ID NO: 290 - DNA, 24 bp, Artificial Sequence
  Synthetic
tgagttattgtatttagtttgaag

SEQ ID NO: 291 - DNA, 24 bp, Artificial Sequence
  Synthetic
tagttgagtttaaagttgaaagaa

SEQ ID NO: 292 - DNA, 24 bp, Artificial Sequence
  Synthetic
taaagagtgatgtaaatagaagtt

SEQ ID NO: 293 - DNA, 24 bp, Artificial Sequence
  Synthetic
tgtagtgtttagagtaagttatta

SEQ ID NO: 294 - DNA, 24 bp, Artificial Sequence
  Synthetic
agagattaatgtgttgaaagatta

SEQ ID NO: 295 - DNA, 24 bp, Artificial Sequence
  Synthetic
gtaataagttgtgaaagaagatta

SEQ ID NO: 296 - DNA, 24 bp, Artificial Sequence
  Synthetic
gagatgttatagataatgaaagaa

SEQ ID NO: 297 - DNA, 24 bp, Artificial Sequence
  Synthetic
tttagttgattgttgaatagagta

SEQ ID NO: 298 - DNA, 24 bp, Artificial Sequence
  Synthetic
attattgaaagtagatgttagatg

SEQ ID NO: 299 - DNA, 24 bp, Artificial Sequence
  Synthetic
tttatgtgtgattgagtgtttaat

SEQ ID NO: 300 - DNA, 24 bp, Artificial Sequence
  Synthetic
tatttagttagatagatagagagt

SEQ ID NO: 301 - DNA, 24 bp, Artificial Sequence
  Synthetic
atgtgtttatgtgaaagatttgta

SEQ ID NO: 302 - DNA, 24 bp, Artificial Sequence
  Synthetic
atagtaattagaagagaagaatgt

SEQ ID NO: 303 - DNA, 24 bp, Artificial Sequence
  Synthetic
tatgagtgattagaattgtatttg

SEQ ID NO: 304 - DNA, 24 bp, Artificial Sequence
  Synthetic
ttaatgtattgtttaaagagtgtg

SEQ ID NO: 305 - DNA, 24 bp, Artificial Sequence
  Synthetic
atagagaattaagaattgtttgag

SEQ ID NO: 306 - DNA, 24 bp, Artificial Sequence
  Synthetic
gttataagtagaaatgtatagaag

SEQ ID NO: 307 - DNA, 24 bp, Artificial Sequence
  Synthetic
agtaattagtttgaaatgtgtagt

SEQ ID NO: 308 - DNA, 24 bp, Artificial Sequence
  Synthetic
gaaagattatgattgtaaagtgat

SEQ ID NO: 309 - DNA, 24 bp, Artificial Sequence
  Synthetic
gtaagattagaagttaatgaagaa

SEQ ID NO: 310 - DNA, 24 bp, Artificial Sequence
  Synthetic
gagaatgttgaataagaagtaatt

SEQ ID NO: 311 - DNA, 24 bp, Artificial Sequence
  Synthetic
ttaagagtgtttgaatagtgttta

SEQ ID NO: 312 - DNA, 24 bp, Artificial Sequence
  Synthetic
ataaagaaagagtatgagattatg

SEQ ID NO: 313 - DNA, 24 bp, Artificial Sequence
  Synthetic
agttattgattgaagatgagaaat

SEQ ID NO: 314 - DNA, 24 bp, Artificial Sequence
  Synthetic
gtttgtgtttgtataagttgttaa

SEQ ID NO: 315 - DNA, 24 bp, Artificial Sequence
  Synthetic
ttgtatgtgagtttagattaatga

SEQ ID NO: 316 - DNA, 24 bp, Artificial Sequence
  Synthetic
tagttaaagtatagttgtttgagt

SEQ ID NO: 317 - DNA, 24 bp, Artificial Sequence
  Synthetic
aaatttgtgttgagatttgtatag

SEQ ID NO: 318 - DNA, 24 bp, Artificial Sequence
  Synthetic
tattagtgttatgataaagagaag

SEQ ID NO: 319 - DNA, 24 bp, Artificial Sequence
  Synthetic
tataagaagtaatttgagaagagt

SEQ ID NO: 320 - DNA, 24 bp, Artificial Sequence
  Synthetic
taagttgagatgtttgtttgataa

SEQ ID NO: 321 - DNA, 24 bp, Artificial Sequence
  Synthetic
gtgtagatttatgaattgagtaat

SEQ ID NO: 322 - DNA, 24 bp, Artificial Sequence
  Synthetic
tatagagaagtgtttagttgtata

SEQ ID NO: 323 - DNA, 24 bp, Artificial Sequence
  Synthetic
ataaagaagaatagttgttgtgta

SEQ ID NO: 324 - DNA, 24 bp, Artificial Sequence
  Synthetic
agattgaaatagattagaaagttg

SEQ ID NO: 325 - DNA, 24 bp, Artificial Sequence
  Synthetic
gttgttataagaaatagtttgttg

SEQ ID NO: 326 - DNA, 24 bp, Artificial Sequence
  Synthetic
agaaatagagtaagagtgtttaaa

SEQ ID NO: 327 - DNA, 24 bp, Artificial Sequence
  Synthetic
agagatagtagtaaatagttattg

SEQ ID NO: 328 - DNA, 24 bp, Artificial Sequence
  Synthetic
aaatgattgtgtaagttatgtatg

SEQ ID NO: 329 - DNA, 24 bp, Artificial Sequence
  Synthetic
aagaagtaagagagaaatttgaat

SEQ ID NO: 330 - DNA, 24 bp, Artificial Sequence
  Synthetic
gtgtgtatttagttgataattgat

SEQ ID NO: 331 - DNA, 24 bp, Artificial Sequence
  Synthetic
attgttgttgttgagaaatgtatt

SEQ ID NO: 332 - DNA, 24 bp, Artificial Sequence
  Synthetic
agataagttaaagtaaagagaatg

SEQ ID NO: 333 - DNA, 24 bp, Artificial Sequence
  Synthetic
tagttgaagttagtttaagtgtta

SEQ ID NO: 334 - DNA, 24 bp, Artificial Sequence
  Synthetic
agtaagaatgtaatatgatgatag

SEQ ID NO: 335 - DNA, 24 bp, Artificial Sequence
  Synthetic
atgagattgaaagatttatgaatg

SEQ ID NO: 336 - DNA, 24 bp, Artificial Sequence
  Synthetic
tgattgaattagagagaatgtata

SEQ ID NO: 337 - DNA, 24 bp, Artificial Sequence
  Synthetic
agttagtaagagaatatagtgaat

SEQ ID NO: 338 - DNA, 24 bp, Artificial Sequence
  Synthetic
attaagattgtatagttagtgatg

SEQ ID NO: 339 - DNA, 24 bp, Artificial Sequence
  Synthetic
gagataaagaattgaaatagaaga

SEQ ID NO: 340 - DNA, 24 bp, Artificial Sequence
  Synthetic
agagtaaatgttaagaaagaagtt

SEQ ID NO: 341 - DNA, 24 bp, Artificial Sequence
  Synthetic
aaagtttgttatgtgtgaagaatt

SEQ ID NO: 342 - DNA, 24 bp, Artificial Sequence
  Synthetic
attgtgtttaagaaatatgatgag

SEQ ID NO: 343 - DNA, 24 bp, Artificial Sequence
  Synthetic
tattgaaatgagatgtatgtagtt

SEQ ID NO: 344 - DNA, 24 bp, Artificial Sequence
  Synthetic
atttgtgtgatgtttgaaatatga

SEQ ID NO: 345 - DNA, 24 bp, Artificial Sequence
  Synthetic
taagataatagtgagagaaattga

SEQ ID NO: 346 - DNA, 24 bp, Artificial Sequence
  Synthetic
atttatgattagtgtaagtgttgt

SEQ ID NO: 347 - DNA, 24 bp, Artificial Sequence
  Synthetic
gattaagaataaagtgtgaagaat

SEQ ID NO: 348 - DNA, 24 bp, Artificial Sequence
  Synthetic
gtaattgatgaagagttagtttat

SEQ ID NO: 349 - DNA, 24 bp, Artificial Sequence
  Synthetic
tgtgttatgttataagaagtgata

SEQ ID NO: 350 - DNA, 24 bp, Artificial Sequence
  Synthetic
agagaaattgaatttagaaatgtg

SEQ ID NO: 351 - DNA, 24 bp, Artificial Sequence
  Synthetic
ttattgaatgtgagaaagtatttg

SEQ ID NO: 352 - DNA, 24 bp, Artificial Sequence
  Synthetic
tgttaatgagaagataatgatagt

SEQ ID NO: 353 - DNA, 24 bp, Artificial Sequence
  Synthetic
gaaagtatttgttgattattgttg

SEQ ID NO: 354 - DNA, 24 bp, Artificial Sequence
  Synthetic
tagtttatgtagttaattgttgag

SEQ ID NO: 355 - DNA, 24 bp, Artificial Sequence
  Synthetic
gttgaaagatagtttgatatgtat

SEQ ID NO: 356 - DNA, 24 bp, Artificial Sequence
  Synthetic
ttagaagatagattattgagaaag

SEQ ID NO: 357 - DNA, 24 bp, Artificial Sequence
  Synthetic
aataatgttgtgaaatagatgtga

SEQ ID NO: 358 - DNA, 24 bp, Artificial Sequence
  Synthetic
agtaagaaagtttagtttagttag

SEQ ID NO: 359 - DNA, 24 bp, Artificial Sequence
  Synthetic
tagtttaatgagatgtttgatatg

SEQ ID NO: 360 - DNA, 24 bp, Artificial Sequence
  Synthetic
ttaaagatgttaaagaatgagtga

SEQ ID NO: 361 - DNA, 24 bp, Artificial Sequence
  Synthetic
aaagtgtgtatatgttagaaagta

SEQ ID NO: 362 - DNA, 24 bp, Artificial Sequence
  Synthetic
attaagttatgtgtttatgtgttg

SEQ ID NO: 363 - DNA, 24 bp, Artificial Sequence
  Synthetic
tttgaagaagtgtttgtattatgt

SEQ ID NO: 364 - DNA, 24 bp, Artificial Sequence
  Synthetic
tgttaagaagtttagttaaagttg

SEQ ID NO: 365 - DNA, 24 bp, Artificial Sequence
  Synthetic
tttaagtataagattgtgtgagat

SEQ ID NO: 366 - DNA, 24 bp, Artificial Sequence
  Synthetic
agatatttgatagatagaagaaag

SEQ ID NO: 367 - DNA, 24 bp, Artificial Sequence
  Synthetic
atttagagttgtaagaagatattg

SEQ ID NO: 368 - DNA, 24 bp, Artificial Sequence
  Synthetic
gagaaattgtaattgttagagtat

SEQ ID NO: 369 - DNA, 24 bp, Artificial Sequence
  Synthetic
gaagtatatgttaagatgtaatag

SEQ ID NO: 370 - DNA, 24 bp, Artificial Sequence
  Synthetic
aatattgaagatgtagtgagttat

SEQ ID NO: 371 - DNA, 24 bp, Artificial Sequence
  Synthetic
gagtttagaaatgataaagaattg

SEQ ID NO: 372 - DNA, 24 bp, Artificial Sequence
  Synthetic
taagaaatgagttatatgttgaga

SEQ ID NO: 373 - DNA, 24 bp, Artificial Sequence
  Synthetic
ttgatataagaagttgtgataagt

SEQ ID NO: 374 - DNA, 24 bp, Artificial Sequence
  Synthetic
aagtgtttaatgtaagagaatgaa

SEQ ID NO: 375 - DNA, 24 bp, Artificial Sequence
  Synthetic
gttgtgagaattagaaatagtata

SEQ ID NO: 376 - DNA, 24 bp, Artificial Sequence
  Synthetic
tttagtttgatgtgtttatgagat

SEQ ID NO: 377 - DNA, 24 bp, Artificial Sequence
  Synthetic
gtaattgaaagtatgagtagtaat

SEQ ID NO: 378 - DNA, 24 bp, Artificial Sequence
  Synthetic
tagttgaataagattgagagaaat

SEQ ID NO: 379 - DNA, 24 bp, Artificial Sequence
  Synthetic
ttaagtgaagtgttgtttattgaa

SEQ ID NO: 380 - DNA, 24 bp, Artificial Sequence
  Synthetic
attgatttgttgaaataagtgttg

SEQ ID NO: 381 - DNA, 24 bp, Artificial Sequence
  Synthetic
tgaattgttgataagttatgaaga

SEQ ID NO: 382 - DNA, 24 bp, Artificial Sequence
  Synthetic
gtttgttattgagtaagttgaatt

SEQ ID NO: 383 - DNA, 24 bp, Artificial Sequence
  Synthetic
tgatttagtatgtattagagttga

SEQ ID NO: 384 - DNA, 24 bp, Artificial Sequence
  Synthetic
taaatagagatgagaataagaaag

SEQ ID NO: 385 - DNA, 24 bp, Artificial Sequence
  Synthetic
agaatgttatatgtagagaaattg

SEQ ID NO: 386 - DNA, 24 bp, Artificial Sequence
  Synthetic
atttatgtagttgagagtgataaa

SEQ ID NO: 387 - DNA, 24 bp, Artificial Sequence
  Synthetic
gtaaagatagtttgagtaatttga

SEQ ID NO: 388 - DNA, 24 bp, Artificial Sequence
  Synthetic
gaaatagtataatgttaagtgaga

SEQ ID NO: 389 - DNA, 24 bp, Artificial Sequence
  Synthetic
attgtatattgtgttgaagaaagt

SEQ ID NO: 390 - DNA, 24 bp, Artificial Sequence
  Synthetic
gagttaagtgtaaatgaaatgtaa

SEQ ID NO: 391 - DNA, 24 bp, Artificial Sequence
  Synthetic
atagattgtgtgaaagaaagaatt

SEQ ID NO: 392 - DNA, 24 bp, Artificial Sequence
  Synthetic
ttaatagaagtttgtagtatgatg

SEQ ID NO: 393 - DNA, 24 bp, Artificial Sequence
  Synthetic
ttgtatgtgagaataaagtttagt

SEQ ID NO: 394 - DNA, 24 bp, Artificial Sequence
  Synthetic
gtgattagatatgatgatatgaat

SEQ ID NO: 395 - DNA, 24 bp, Artificial Sequence
  Synthetic
tgaagaagaatttagatttgtaag

SEQ ID NO: 396 - DNA, 24 bp, Artificial Sequence
  Synthetic
tgtatgattattgattagtgtgtt

SEQ ID NO: 397 - DNA, 24 bp, Artificial Sequence
  Synthetic
tgtgaaagagaatgatagatattt

SEQ ID NO: 398 - DNA, 24 bp, Artificial Sequence
  Synthetic
aattgaaatgagtgtgtttaagaa

SEQ ID NO: 399 - DNA, 24 bp, Artificial Sequence
  Synthetic
attatagagttagtttagaatgag

SEQ ID NO: 400 - DNA, 24 bp, Artificial Sequence
  Synthetic
aaagatagaaattgagtgtatgat

SEQ ID NO: 401 - DNA, 24 bp, Artificial Sequence
  Synthetic
gtagtttgttaatgttgtataatg

SEQ ID NO: 402 - DNA, 24 bp, Artificial Sequence
  Synthetic
agagatattagaatgtaagaatag

SEQ ID NO: 403 - DNA, 24 bp, Artificial Sequence
  Synthetic
agaagtttgaaatatgatagaatg

SEQ ID NO: 404 - DNA, 24 bp, Artificial Sequence
  Synthetic
tagaatgtaaagtttagtatagag

SEQ ID NO: 405 - DNA, 24 bp, Artificial Sequence
  Synthetic
agtagatgtatgttaatgtgaata

SEQ ID NO: 406 - DNA, 24 bp, Artificial Sequence
  Synthetic
tgaaagtgaaatatgaaatgttgt

SEQ ID NO: 407 - DNA, 24 bp, Artificial Sequence
  Synthetic
atagtatattgagtttgtatgaag

SEQ ID NO: 408 - DNA, 24 bp, Artificial Sequence
  Synthetic
gaagaaatgtttgtagaataagta

SEQ ID NO: 409 - DNA, 24 bp, Artificial Sequence
  Synthetic
aatgagtattgaagaaatgtatag

SEQ ID NO: 410 - DNA, 24 bp, Artificial Sequence
  Synthetic
gtgatagaatttgtgtttaatgaa

SEQ ID NO: 411 - DNA, 24 bp, Artificial Sequence
  Synthetic
tgtagtatgaagaataatgaaatg

SEQ ID NO: 412 - DNA, 24 bp, Artificial Sequence
  Synthetic
atagaagttaatgataattgtgtg

SEQ ID NO: 413 - DNA, 24 bp, Artificial Sequence
  Synthetic
gtgattgtaagtaagtaaagataa

SEQ ID NO: 414 - DNA, 24 bp, Artificial Sequence
  Synthetic
tatgtagtttgtgttatttgaaga

SEQ ID NO: 415 - DNA, 24 bp, Artificial Sequence
  Synthetic
tgagtaagtttgtatgtttaagta

SEQ ID NO: 416 - DNA, 24 bp, Artificial Sequence
  Synthetic
taaatgtatgagtgtgtaaagaaa

SEQ ID NO: 417 - DNA, 24 bp, Artificial Sequence
  Synthetic
gtaagagtattgaaattagtaaga

SEQ ID NO: 418 - DNA, 24 bp, Artificial Sequence
  Synthetic
gttgagtgtaaagattattgataa

SEQ ID NO: 419 - DNA, 24 bp, Artificial Sequence
  Synthetic
agtatgagttattagataaagtga

SEQ ID NO: 420 - DNA, 24 bp, Artificial Sequence
  Synthetic
atttgttatagagttgtgttgtat

SEQ ID NO: 421 - DNA, 24 bp, Artificial Sequence
  Synthetic
taattagtagtgtgttgaaatttg

SEQ ID NO: 422 - DNA, 24 bp, Artificial Sequence
  Synthetic
tgtattgagattgttattgtattg

SEQ ID NO: 423 - DNA, 24 bp, Artificial Sequence
  Synthetic
gttattagaagagataattgagtt

SEQ ID NO: 424 - DNA, 24 bp, Artificial Sequence
  Synthetic
ttgagttgtgattaagtagtatat

SEQ ID NO: 425 - DNA, 24 bp, Artificial Sequence
  Synthetic
gatagtataatgattgaagtaatg

SEQ ID NO: 426 - DNA, 24 bp, Artificial Sequence
  Synthetic
gtgaaagatatttgagagataaat

SEQ ID NO: 427 - DNA, 24 bp, Artificial Sequence
  Synthetic
agttatgatttgaagaaattgttg

SEQ ID NO: 428 - DNA, 24 bp, Artificial Sequence
  Synthetic
gtaagtatttgaatttgatgagtt

SEQ ID NO: 429 - DNA, 24 bp, Artificial Sequence
  Synthetic
taatagtgttataagtgaaagagt

SEQ ID NO: 430 - DNA, 24 bp, Artificial Sequence
  Synthetic
aaatgaattgatgtgtatatgaag

SEQ ID NO: 431 - DNA, 24 bp, Artificial Sequence
  Synthetic
agaaagtgagttgttaagtattta

SEQ ID NO: 432 - DNA, 24 bp, Artificial Sequence
  Synthetic
tttatgtgtgaattgtgtatatag

SEQ ID NO: 433 - DNA, 24 bp, Artificial Sequence
  Synthetic
gtaatatgatagaaatgtaaagag

SEQ ID NO: 434 - DNA, 24 bp, Artificial Sequence
  Synthetic
gagaattgtttaaagatagttgta

SEQ ID NO: 435 - DNA, 24 bp, Artificial Sequence
  Synthetic
gaatttgttaagaatgagtttgat

SEQ ID NO: 436 - DNA, 24 bp, Artificial Sequence
  Synthetic
atagtgatgattaaagagaatttg

SEQ ID NO: 437 - DNA, 24 bp, Artificial Sequence
  Synthetic
atagatgtttagttgagattattg

SEQ ID NO: 438 - DNA, 24 bp, Artificial Sequence
  Synthetic
aagagtgtaaatagaaagtgatat

SEQ ID NO: 439 - DNA, 24 bp, Artificial Sequence
  Synthetic
tgtgtattgattgttgagataaat

SEQ ID NO: 440 - DNA, 24 bp, Artificial Sequence
  Synthetic
tagtatagtgagaaagagttaaat

SEQ ID NO: 441 - DNA, 24 bp, Artificial Sequence
  Synthetic
aaagataagaaagagatgatgttt

SEQ ID NO: 442 - DNA, 24 bp, Artificial Sequence
  Synthetic
gaagttattgaaatagagaagtat

SEQ ID NO: 443 - DNA, 24 bp, Artificial Sequence
  Synthetic
atgtatgtatagaaagagtaaatg

SEQ ID NO: 444 - DNA, 24 bp, Artificial Sequence
  Synthetic
gatgtttgtaaagattgaaattga

SEQ ID NO: 445 - DNA, 24 bp, Artificial Sequence
  Synthetic
aatttagagagtatttgtgttgta

SEQ ID NO: 446 - DNA, 24 bp, Artificial Sequence
  Synthetic
aatttgtttgaaagaaagtaagtg

SEQ ID NO: 447 - DNA, 24 bp, Artificial Sequence
  Synthetic
aaagagtagtgttattgttagata

SEQ ID NO: 448 - DNA, 24 bp, Artificial Sequence
  Synthetic
gtatgttgtatatgttgttgatat

SEQ ID NO: 449 - DNA, 24 bp, Artificial Sequence
  Synthetic
gtagaatttgttgagtatttgtaa

SEQ ID NO: 450 - DNA, 24 bp, Artificial Sequence
  Synthetic
atgaatttagttagtgtaagaaag

SEQ ID NO: 451 - DNA, 24 bp, Artificial Sequence
  Synthetic
atgataagaaatgttgatgaagta

SEQ ID NO: 452 - DNA, 24 bp, Artificial Sequence
  Synthetic
ttgatgatgaagataatgtagata

SEQ ID NO: 453 - DNA, 24 bp, Artificial Sequence
  Synthetic
agatgatatgatatagattagatg

SEQ ID NO: 454 - DNA, 24 bp, Artificial Sequence
  Synthetic
ttgaaagttagaaagatagatgtt

SEQ ID NO: 455 - DNA, 24 bp, Artificial Sequence
  Synthetic
gtttaatgttagttagaaagtaag

SEQ ID NO: 456 - DNA, 24 bp, Artificial Sequence
  Synthetic
gagatttaagtttgaagtgaaata

SEQ ID NO: 457 - DNA, 24 bp, Artificial Sequence
  Synthetic
tttgttagtagttgttataagaga

SEQ ID NO: 458 - DNA, 24 bp, Artificial Sequence
  Synthetic
tatgagaatagtttgttagtgaat

SEQ ID NO: 459 - DNA, 24 bp, Artificial Sequence
  Synthetic
ttgaaagtttaaagaagagataag

SEQ ID NO: 460 - DNA, 24 bp, Artificial Sequence
  Synthetic
aagtgagttgaaatgaaatatgtt

SEQ ID NO: 461 - DNA, 24 bp, Artificial Sequence
  Synthetic
gttagaaatgaaatgagtagttat

SEQ ID NO: 462 - DNA, 24 bp, Artificial Sequence
  Synthetic
taagtattgtatttgtgtgtgtat

SEQ ID NO: 463 - DNA, 24 bp, Artificial Sequence
  Synthetic
tgtattagtaaagaagagagaata

SEQ ID NO: 464 - DNA, 24 bp, Artificial Sequence
  Synthetic
gagaagagaaataagttgaaataa

SEQ ID NO: 465 - DNA, 24 bp, Artificial Sequence
  Synthetic
gtaaagtagaaatagaattgagtt

SEQ ID NO: 466 - DNA, 24 bp, Artificial Sequence
  Synthetic
gtgtgttatttgtttgtaaagtat

SEQ ID NO: 467 - DNA, 24 bp, Artificial Sequence
  Synthetic
tttgatgtatgaatatagtatgag

SEQ ID NO: 468 - DNA, 24 bp, Artificial Sequence
  Synthetic
aagattgtgtgaatagttgaaatt

SEQ ID NO: 469 - DNA, 24 bp, Artificial Sequence
  Synthetic
tataaagtttgaagatgagtgata

SEQ ID NO: 470 - DNA, 24 bp, Artificial Sequence
  Synthetic
agataaagagatttaagatgtatg

SEQ ID NO: 471 - DNA, 24 bp, Artificial Sequence
  Synthetic
gaagaattaagttgagaattaaga

SEQ ID NO: 472 - DNA, 24 bp, Artificial Sequence
  Synthetic
tagagaaatttgataaagaaagag

SEQ ID NO: 473 - DNA, 24 bp, Artificial Sequence
  Synthetic
aaagtttatgaagttattgagtag

SEQ ID NO: 474 - DNA, 24 bp, Artificial Sequence
  Synthetic
aaatagtgtaagtaaagagatgat

SEQ ID NO: 475 - DNA, 24 bp, Artificial Sequence
  Synthetic
tatgatgatttagttataagagtg

SEQ ID NO: 476 - DNA, 24 bp, Artificial Sequence
  Synthetic
tagataaatgttatgatgagtaag

SEQ ID NO: 477 - DNA, 24 bp, Artificial Sequence
  Synthetic
agattgattgtgatgatttgtata

SEQ ID NO: 478 - DNA, 24 bp, Artificial Sequence
  Synthetic
ttaagaagaattgtatatgagagt

SEQ ID NO: 479 - DNA, 24 bp, Artificial Sequence
  Synthetic
gtagaatgtttagagttgaatata

SEQ ID NO: 480 - DNA, 24 bp, Artificial Sequence
  Synthetic
gagaaatagtaagaagtaaataga

SEQ ID NO: 481 - DNA, 24 bp, Artificial Sequence
  Synthetic
attgaagttgttatgtgaagattt

SEQ ID NO: 482 - DNA, 24 bp, Artificial Sequence
  Synthetic
taaatgttgtgtagagtaattaga

SEQ ID NO: 483 - DNA, 24 bp, Artificial Sequence
  Synthetic
aaataagagtttgagaagttgttt

SEQ ID NO: 484 - DNA, 24 bp, Artificial Sequence
  Synthetic
agttgtaataagaagtgatttaag

SEQ ID NO: 485 - DNA, 24 bp, Artificial Sequence
  Synthetic
gttagaatgtatatagagttagat

SEQ ID NO: 486 - DNA, 24 bp, Artificial Sequence
  Synthetic
ttgatattgaaagagaaagttatg

SEQ ID NO: 487 - DNA, 24 bp, Artificial Sequence
  Synthetic
ttaaagagagaaatgtttgattag

SEQ ID NO: 488 - DNA, 24 bp, Artificial Sequence
  Synthetic
tgtgaatttgagtattagtaagaa

SEQ ID NO: 489 - DNA, 24 bp, Artificial Sequence
  Synthetic
taatttgaatgtgaaagttgttag

SEQ ID NO: 490 - DNA, 24 bp, Artificial Sequence
  Synthetic
atgtgtttgaaagatgatgattta

SEQ ID NO: 491 - DNA, 24 bp, Artificial Sequence
  Synthetic
aagttatgttgatattgagtgaaa

SEQ ID NO: 492 - DNA, 24 bp, Artificial Sequence
  Synthetic
tagataaagaagatagagatttag

SEQ ID NO: 493 - DNA, 24 bp, Artificial Sequence
  Synthetic
gatgaatgtagatatatgtaatga

SEQ ID NO: 494 - DNA, 24 bp, Artificial Sequence
  Synthetic
gaagaatagtttatgtaaatgatg

SEQ ID NO: 495 - DNA, 24 bp, Artificial Sequence
  Synthetic
gtagtatatagttaaagatgagtt

SEQ ID NO: 496 - DNA, 24 bp, Artificial Sequence
  Synthetic
gttatttgtgtatgattatgattg

SEQ ID NO: 497 - DNA, 24 bp, Artificial Sequence
  Synthetic
agagattagaaattgagagaatta

SEQ ID NO: 498 - DNA, 24 bp, Artificial Sequence
  Synthetic
gtatgatagagtttatagtgataa

SEQ ID NO: 499 - DNA, 24 bp, Artificial Sequence
  Synthetic
gttagaaagaatgaaattgaagta

SEQ ID NO: 500 - DNA, 24 bp, Artificial Sequence
  Synthetic
aagaatgagaatatagagatgaat

SEQ ID NO: 501 - DNA, 24 bp, Artificial Sequence
  Synthetic
aaagagaatagtgtttaagaagat

SEQ ID NO: 502 - DNA, 24 bp, Artificial Sequence
  Synthetic
gatgtgttattgatagaaattaga

SEQ ID NO: 503 - DNA, 24 bp, Artificial Sequence
  Synthetic
tagagttatagagatattgtatga

SEQ ID NO: 504 - DNA, 24 bp, Artificial Sequence
  Synthetic
gagagttgaataagttaaagatat

SEQ ID NO: 505 - DNA, 24 bp, Artificial Sequence
  Synthetic
agatatgaaatagattgttagaga

SEQ ID NO: 506 - DNA, 24 bp, Artificial Sequence
  Synthetic
gagtgaatagaaagatatgttaat

SEQ ID NO: 507 - DNA, 24 bp, Artificial Sequence
  Synthetic
aaagagatattgaagagaataaag

SEQ ID NO: 508 - DNA, 24 bp, Artificial Sequence
  Synthetic
gttatagaataagttgtaaagtgt

SEQ ID NO: 509 - DNA, 24 bp, Artificial Sequence
  Synthetic
tgatagtatgataatgtgtttatg

SEQ ID NO: 510 - DNA, 24 bp, Artificial Sequence
  Synthetic
tttgttgttaagtatgtgatttag

SEQ ID NO: 511 - DNA, 24 bp, Artificial Sequence
  Synthetic
taaagtgttgtgttaaagattaag

SEQ ID NO: 512 - DNA, 24 bp, Artificial Sequence
  Synthetic
tgtgtttgattgattaatgttatg

SEQ ID NO: 513 - DNA, 24 bp, Artificial Sequence
  Synthetic
attaatgaatgagtgttgtaatgt

SEQ ID NO: 514 - DNA, 24 bp, Artificial Sequence
  Synthetic
tagatgtttgtgagtttgatatta

SEQ ID NO: 515 - DNA, 24 bp, Artificial Sequence
  Synthetic
gaatgaatagtaatagatgatttg

SEQ ID NO: 516 - DNA, 24 bp, Artificial Sequence
  Synthetic
aatagtgtgttgttatatgattag

SEQ ID NO: 517 - DNA, 24 bp, Artificial Sequence
  Synthetic
tagattagaagatgttgtgtatta

SEQ ID NO: 518 - DNA, 24 bp, Artificial Sequence
  Synthetic
aatgtgtgtgttaaatgaatttgt

SEQ ID NO: 519 - DNA, 24 bp, Artificial Sequence
  Synthetic
gaattaagtatatgagtgtagaaa

SEQ ID NO: 520 - DNA, 24 bp, Artificial Sequence
  Synthetic
ttattgtgtgtaagtagtgtaaat

SEQ ID NO: 521 - DNA, 24 bp, Artificial Sequence
  Synthetic
gtagtaaagagaattgtttagtat

SEQ ID NO: 522 - DNA, 24 bp, Artificial Sequence
  Synthetic
aagtttgtaagaagtagttgaata

SEQ ID NO: 523 - DNA, 24 bp, Artificial Sequence
  Synthetic
agttatagtatagtagtatagaga

SEQ ID NO: 524 - DNA, 24 bp, Artificial Sequence
  Synthetic
gaaagaaatgtgtatagtttaatg

SEQ ID NO: 525 - DNA, 24 bp, Artificial Sequence
  Synthetic
ttgtgagtaatgaatgatgtatta

SEQ ID NO: 526 - DNA, 24 bp, Artificial Sequence
  Synthetic
gtagagttgtaaatagagaataaa

SEQ ID NO: 527 - DNA, 24 bp, Artificial Sequence
  Synthetic
attaatgtagattgtaagagatag

SEQ ID NO: 528 - DNA, 24 bp, Artificial Sequence
  Synthetic
ttagtgtgtttgtagatagaatta

SEQ ID NO: 529 - DNA, 24 bp, Artificial Sequence
  Synthetic
agagagtttgtgtatatgtataaa

SEQ ID NO: 530 - DNA, 24 bp, Artificial Sequence
  Synthetic
ttaagtttagtgagatttgttaag

SEQ ID NO: 531 - DNA, 24 bp, Artificial Sequence
  Synthetic
atgaagtttattgaatagtagtga

SEQ ID NO: 532 - DNA, 24 bp, Artificial Sequence
  Synthetic
atatttgtgttgtatgtttgtgaa

SEQ ID NO: 533 - DNA, 24 bp, Artificial Sequence
  Synthetic
aaagtgtttatagaagatttgatg

SEQ ID NO: 534 - DNA, 24 bp, Artificial Sequence
  Synthetic
aagagatatgatttgttagttgta

SEQ ID NO: 535 - DNA, 24 bp, Artificial Sequence
  Synthetic
aagaagaaatgagtgataatgtaa

SEQ ID NO: 536 - DNA, 24 bp, Artificial Sequence
  Synthetic
tagtgtttgatatgttaagaagtt

SEQ ID NO: 537 - DNA, 24 bp, Artificial Sequence
  Synthetic
gtagaaagtgatagattagtaata

SEQ ID NO: 538 - DNA, 24 bp, Artificial Sequence
  Synthetic
gataaatgttaagttagtatgatg

SEQ ID NO: 539 - DNA, 24 bp, Artificial Sequence
  Synthetic
agattagaagaattgtttagaatg

SEQ ID NO: 540 - DNA, 24 bp, Artificial Sequence
  Synthetic
atatttgagaagtgtgaaatgaat

SEQ ID NO: 541 - DNA, 24 bp, Artificial Sequence
  Synthetic
tgagtaaatagtttatgagtagta

SEQ ID NO: 542 - DNA, 24 bp, Artificial Sequence
  Synthetic
ttagagagtagataaagatttgat

SEQ ID NO: 543 - DNA, 24 bp, Artificial Sequence
  Synthetic
attgtttaagttgttgataagatg

SEQ ID NO: 544 - DNA, 24 bp, Artificial Sequence
  Synthetic
gttgtaaagttaaagtgtgaattt

SEQ ID NO: 545 - DNA, 24 bp, Artificial Sequence
  Synthetic
atagattgtgtgtttgttatagta

SEQ ID NO: 546 - DNA, 24 bp, Artificial Sequence
  Synthetic
gtaagttattgagaatgataatag

SEQ ID NO: 547 - DNA, 24 bp, Artificial Sequence
  Synthetic
tagattagttgataagtgtgtaat

SEQ ID NO: 548 - DNA, 24 bp, Artificial Sequence
  Synthetic
aaatgtaaatgaagagtgtttgtt

SEQ ID NO: 549 - DNA, 24 bp, Artificial Sequence
  Synthetic
gatagaagaaatgtatatagtgat

SEQ ID NO: 550 - DNA, 24 bp, Artificial Sequence
  Synthetic
tatagagtgtatgttatgataaag

SEQ ID NO: 551 - DNA, 24 bp, Artificial Sequence
  Synthetic
tatgaagtgataagatgaagaatt

SEQ ID NO: 552 - DNA, 24 bp, Artificial Sequence
  Synthetic
tgttgagaatagtaagagaattta

SEQ ID NO: 553 - DNA, 24 bp, Artificial Sequence
  Synthetic
tagataatgtgaagtaataagtga

SEQ ID NO: 554 - DNA, 24 bp, Artificial Sequence
  Synthetic
gtattatgatgatagtagtaagta

SEQ ID NO: 555 - DNA, 24 bp, Artificial Sequence
  Synthetic
agatatgatttagtattgaatgtg

SEQ ID NO: 556 - DNA, 24 bp, Artificial Sequence
  Synthetic
aattaagtttgtagagtgatttga

SEQ ID NO: 557 - DNA, 24 bp, Artificial Sequence
  Synthetic
aagaaatagatgtagtaagatgtt

SEQ ID NO: 558 - DNA, 24 bp, Artificial Sequence
  Synthetic
ttgagaagttgttgtaataagaat

SEQ ID NO: 559 - DNA, 24 bp, Artificial Sequence
  Synthetic
agtgtgaaatagtgaaagtttaaa

SEQ ID NO: 560 - DNA, 24 bp, Artificial Sequence
  Synthetic
tttatgtagtagatttatgtgaag

SEQ ID NO: 561 - DNA, 24 bp, Artificial Sequence
  Synthetic
attaatgagaaattagtgtgttag

SEQ ID NO: 562 - DNA, 24 bp, Artificial Sequence
  Synthetic
atgttaatagtgatagtaaagtga

SEQ ID NO: 563 - DNA, 24 bp, Artificial Sequence
  Synthetic
tatgttgataaatgattatgagtg

SEQ ID NO: 564 - DNA, 24 bp, Artificial Sequence
  Synthetic
ttattagagttgtgtgtgatatat

SEQ ID NO: 565 - DNA, 24 bp, Artificial Sequence
  Synthetic
tgttgttatgattgagttagaata

SEQ ID NO: 566 - DNA, 24 bp, Artificial Sequence
  Synthetic
aatttgagttaagaagaagtgtaa

SEQ ID NO: 567 - DNA, 24 bp, Artificial Sequence
  Synthetic
aaagataaagttaagtgtttgtag

SEQ ID NO: 568 - DNA, 24 bp, Artificial Sequence
  Synthetic
tgttgagatgatattgtataagtt

SEQ ID NO: 569 - DNA, 24 bp, Artificial Sequence
  Synthetic
taaatagtgaatgagttatagagt

SEQ ID NO: 570 - DNA, 24 bp, Artificial Sequence
  Synthetic
atagatgttatgatagttagttag

SEQ ID NO: 571 - DNA, 24 bp, Artificial Sequence
  Synthetic
gttaagtgaagatatgtattgtta

SEQ ID NO: 572 - DNA, 24 bp, Artificial Sequence
  Synthetic
taagaaagtaaagtttgtagatgt

SEQ ID NO: 573 - DNA, 24 bp, Artificial Sequence
  Synthetic
aagagaaagtttgattgaataaag

SEQ ID NO: 574 - DNA, 24 bp, Artificial Sequence
  Synthetic
atattagatgtgagttatatgtgt

SEQ ID NO: 575 - DNA, 24 bp, Artificial Sequence
  Synthetic
agtttgagtttagtattgtgaata

SEQ ID NO: 576 - DNA, 24 bp, Artificial Sequence
  Synthetic
atgttaaatgagagattgtgtata

SEQ ID NO: 577 - DNA, 24 bp, Artificial Sequence
  Synthetic
taaatgttgtgattattgtgagat

SEQ ID NO: 578 - DNA, 24 bp, Artificial Sequence
  Synthetic
taagaattgaagtaagagttattg

SEQ ID NO: 579 - DNA, 24 bp, Artificial Sequence
  Synthetic
agagatagaattaagtttgttgat

SEQ ID NO: 580 - DNA, 24 bp, Artificial Sequence
  Synthetic
gaagaatgttaagaaatatgtaag

SEQ ID NO: 581 - DNA, 24 bp, Artificial Sequence
  Synthetic
tatttgtgattaagaagttgagaa

SEQ ID NO: 582 - DNA, 24 bp, Artificial Sequence
  Synthetic
agttagaatttgtgtagtagaatt

SEQ ID NO: 583 - DNA, 24 bp, Artificial Sequence
  Synthetic
aagtttattgttgatgttgtattg

SEQ ID NO: 584 - DNA, 24 bp, Artificial Sequence
  Synthetic
gaatgagtttaagagtttatagta

SEQ ID NO: 585 - DNA, 24 bp, Artificial Sequence
  Synthetic
agtgaagattgtatgtagtataaa

SEQ ID NO: 586 - DNA, 24 bp, Artificial Sequence
  Synthetic
agttgaaatgagtattaagtaatg

SEQ ID NO: 587 - DNA, 24 bp, Artificial Sequence
  Synthetic
atgtgttatttgagatgagtaatt

SEQ ID NO: 588 - DNA, 24 bp, Artificial Sequence
  Synthetic
aaatagtgttgttgaagttgttat

SEQ ID NO: 589 - DNA, 24 bp, Artificial Sequence
  Synthetic
gtagagaaagatatatgtagttta

SEQ ID NO: 590 - DNA, 24 bp, Artificial Sequence
  Synthetic
gagagtatttgatgaatgattata

SEQ ID NO: 591 - DNA, 24 bp, Artificial Sequence
  Synthetic
gagtataagtttagtgtatattga

SEQ ID NO: 592 - DNA, 24 bp, Artificial Sequence
  Synthetic
ataatgtgattattgattgagaga

SEQ ID NO: 593 - DNA, 24 bp, Artificial Sequence
  Synthetic
ttagttgttatgtgagagtaataa

SEQ ID NO: 594 - DNA, 24 bp, Artificial Sequence
  Synthetic
aaatgagtatattgaattgtgatg

SEQ ID NO: 595 - DNA, 24 bp, Artificial Sequence
  Synthetic
aattagaagtaagtagagtttaag

SEQ ID NO: 596 - DNA, 24 bp, Artificial Sequence
  Synthetic
tgtaagtttaaagtaagaaatgtg

SEQ ID NO: 597 - DNA, 24 bp, Artificial Sequence
  Synthetic
gaaatgataagttgatataagaag

SEQ ID NO: 598 - DNA, 24 bp, Artificial Sequence
  Synthetic
aatgagtagtttgtatttgagttt

SEQ ID NO: 599 - DNA, 24 bp, Artificial Sequence
  Synthetic
agtgaatgtaagattatgtatttg

SEQ ID NO: 600 - DNA, 24 bp, Artificial Sequence
  Synthetic
gtaattgaattgaaagataagtgt

SEQ ID NO: 601 - DNA, 24 bp, Artificial Sequence
  Synthetic
tatgtttaagtagtgaaatagagt

SEQ ID NO: 602 - DNA, 24 bp, Artificial Sequence
  Synthetic
gtattgaaattgaattagaagtag

SEQ ID NO: 603 - DNA, 24 bp, Artificial Sequence
  Synthetic
aatatgtaatgtagttgaaagtga

SEQ ID NO: 604 - DNA, 24 bp, Artificial Sequence
  Synthetic
tgaatattgagaattatgagagtt

SEQ ID NO: 605 - DNA, 24 bp, Artificial Sequence
  Synthetic
tagtgtaaatgatgaagaaagtat

SEQ ID NO: 606 - DNA, 24 bp, Artificial Sequence
  Synthetic
gtatgtgtaaagaaatttgatgta

SEQ ID NO: 607 - DNA, 24 bp, Artificial Sequence
  Synthetic
aattgtttgaaagtttgttgagaa

SEQ ID NO: 608 - DNA, 24 bp, Artificial Sequence
  Synthetic
aattgtttgagtagtattagtagt

SEQ ID NO: 609 - DNA, 24 bp, Artificial Sequence
  Synthetic
taattgagtttgaataagagagtt

SEQ ID NO: 610 - DNA, 24 bp, Artificial Sequence
  Synthetic
tgttgattgtaagtgtttattgtt

SEQ ID NO: 611 - DNA, 24 bp, Artificial Sequence
  Synthetic
gaaatttgtgagtatgtatttgaa

SEQ ID NO: 612 - DNA, 24 bp, Artificial Sequence
  Synthetic
taagaatgaatgtgaagtgaatat

SEQ ID NO: 613 - DNA, 24 bp, Artificial Sequence
  Synthetic
taatgtgaagtttgtgaaagatat

SEQ ID NO: 614 - DNA, 24 bp, Artificial Sequence
  Synthetic
ttgtatatgaaagtaagaagaagt

SEQ ID NO: 615 - DNA, 24 bp, Artificial Sequence
  Synthetic
tagagagaagaagaaataagaata

SEQ ID NO: 616 - DNA, 24 bp, Artificial Sequence
  Synthetic
atttgaaatgttaatgagagagat

SEQ ID NO: 617 - DNA, 24 bp, Artificial Sequence
  Synthetic
ttgtgtgtatatagtattagaatg

SEQ ID NO: 618 - DNA, 24 bp, Artificial Sequence
  Synthetic
attgttagtattgatgtgaagtta

SEQ ID NO: 619 - DNA, 24 bp, Artificial Sequence
  Synthetic
tgtttgtatttgaatgaaatgaag

SEQ ID NO: 620 - DNA, 24 bp, Artificial Sequence
  Synthetic
tgttagattgtgttaaatgtagtt

SEQ ID NO: 621 - DNA, 24 bp, Artificial Sequence
  Synthetic
tatagagtattgtatagagagaaa

SEQ ID NO: 622 - DNA, 24 bp, Artificial Sequence
  Synthetic
aaatagtaagaatgtagttgttga

SEQ ID NO: 623 - DNA, 24 bp, Artificial Sequence
  Synthetic
tgagtgtgatttatgattaagtta

SEQ ID NO: 624 - DNA, 24 bp, Artificial Sequence
  Synthetic
agaatttgttgtagtgttatgatt

SEQ ID NO: 625 - DNA, 24 bp, Artificial Sequence
  Synthetic
gattgaagaaagaaatagtttgaa

SEQ ID NO: 626 - DNA, 24 bp, Artificial Sequence
  Synthetic
gataatagagaatagtagagttaa

SEQ ID NO: 627 - DNA, 24 bp, Artificial Sequence
  Synthetic
gattgaaatttgtagttatagtga

SEQ ID NO: 628 - DNA, 24 bp, Artificial Sequence
  Synthetic
gatttaagaagatgaataatgtag

SEQ ID NO: 629 - DNA, 24 bp, Artificial Sequence
  Synthetic
tttgagagaaagtagaataagata

SEQ ID NO: 630 - DNA, 24 bp, Artificial Sequence
  Synthetic
gattaagagtaaatgagtataaga

SEQ ID NO: 631 - DNA, 24 bp, Artificial Sequence
  Synthetic
tttgatagaattgaaatttgagag

SEQ ID NO: 632 - DNA, 24 bp, Artificial Sequence
  Synthetic
tgaagaagagtgttataagattta

SEQ ID NO: 633 - DNA, 24 bp, Artificial Sequence
  Synthetic
gtgaaatgatttagagtaataagt

SEQ ID NO: 634 - DNA, 24 bp, Artificial Sequence
  Synthetic
aaataagaatagagagagaaagtt

SEQ ID NO: 635 - DNA, 24 bp, Artificial Sequence
  Synthetic
gttgtaaagtaatagagaaattag

SEQ ID NO: 636 - DNA, 24 bp, Artificial Sequence
  Synthetic
agtgatttagattatgtgatgatt

SEQ ID NO: 637 - DNA, 24 bp, Artificial Sequence
  Synthetic
agagtatagtttagatttatgtag

SEQ ID NO: 638 - DNA, 24 bp, Artificial Sequence
  Synthetic
atgattagatagtgaaattgttag

SEQ ID NO: 639 - DNA, 24 bp, Artificial Sequence
  Synthetic
atgaaatgtattagtttagagttg

SEQ ID NO: 640 - DNA, 24 bp, Artificial Sequence
  Synthetic
atattgagtgagagttattgttaa

SEQ ID NO: 641 - DNA, 24 bp, Artificial Sequence
  Synthetic
agatgtgtattgaattaagaagtt

SEQ ID NO: 642 - DNA, 24 bp, Artificial Sequence
  Synthetic
taatgtgttgatagaatagagata

SEQ ID NO: 643 - DNA, 24 bp, Artificial Sequence
  Synthetic
aaattagttgaaagtatgagaaag

SEQ ID NO: 644 - DNA, 24 bp, Artificial Sequence
  Synthetic
tttagagttgaagaaatgttaatg

SEQ ID NO: 645 - DNA, 24 bp, Artificial Sequence
  Synthetic
gattgttgattattgatgaatttg

SEQ ID NO: 646 - DNA, 24 bp, Artificial Sequence
  Synthetic
tgttgttgttgaattgaagaatta

SEQ ID NO: 647 - DNA, 24 bp, Artificial Sequence
  Synthetic
attaagtaagaattgagagtttga

SEQ ID NO: 648 - DNA, 24 bp, Artificial Sequence
  Synthetic
gtatgttgtaatgtattaagaaag

SEQ ID NO: 649 - DNA, 24 bp, Artificial Sequence
  Synthetic
tagttgtgatttatgtaatgattg

SEQ ID NO: 650 - DNA, 24 bp, Artificial Sequence
  Synthetic
tgataatgaaagtttatagagaga

SEQ ID NO: 651 - DNA, 24 bp, Artificial Sequence
  Synthetic
gtaagattgtttgtatgataagat

SEQ ID NO: 652 - DNA, 24 bp, Artificial Sequence
  Synthetic
ttgaattaagagtaagatgtttag

SEQ ID NO: 653 - DNA, 24 bp, Artificial Sequence
  Synthetic
aagtgtttgtttagagtaaagata

SEQ ID NO: 654 - DNA, 24 bp, Artificial Sequence
  Synthetic
agagagataaagtatagaagttaa

SEQ ID NO: 655 - DNA, 24 bp, Artificial Sequence
  Synthetic
attatgaatagttagaaagagagt

SEQ ID NO: 656 - DNA, 24 bp, Artificial Sequence
  Synthetic
ttgttgatattagagaatgtgttt

SEQ ID NO: 657 - DNA, 24 bp, Artificial Sequence
  Synthetic
tttattgagagtttgttatttgtg

SEQ ID NO: 658 - DNA, 24 bp, Artificial Sequence
  Synthetic
agtgttaagaagttgattattgat

SEQ ID NO: 659 - DNA, 24 bp, Artificial Sequence
  Synthetic
gagaaatgattgaatgttgataat

SEQ ID NO: 660 - DNA, 24 bp, Artificial Sequence
  Synthetic
gataagtattagtatgagtgtaat

SEQ ID NO: 661 - DNA, 24 bp, Artificial Sequence
  Synthetic
tttgatttaagagtgttgaatgta

SEQ ID NO: 662 - DNA, 24 bp, Artificial Sequence
  Synthetic
aagttagtaaatagagtagaaaga

SEQ ID NO: 663 - DNA, 24 bp, Artificial Sequence
  Synthetic
gtaaagtatgaatatgtgaaatgt

SEQ ID NO: 664 - DNA, 24 bp, Artificial Sequence
  Synthetic
taataagtgtgttgtgaatgtaat

SEQ ID NO: 665 - DNA, 24 bp, Artificial Sequence
  Synthetic
aaagatttagagtagaaagagaat

SEQ ID NO: 666 - DNA, 24 bp, Artificial Sequence
  Synthetic
ttagtttgagttgaaatagtaaag

SEQ ID NO: 667 - DNA, 24 bp, Artificial Sequence
  Synthetic
taatagtatgagtaagattgaaag

SEQ ID NO: 668 - DNA, 24 bp, Artificial Sequence
  Synthetic
gaagattagattgatgttagttaa

SEQ ID NO: 669 - DNA, 24 bp, Artificial Sequence
  Synthetic
taaagagagaagttagtaatagaa

SEQ ID NO: 670 - DNA, 24 bp, Artificial Sequence
  Synthetic
taagtatgagaaatgatgtgttat

SEQ ID NO: 671 - DNA, 24 bp, Artificial Sequence
  Synthetic
gagtttgtttgttagttattgata

SEQ ID NO: 672 - DNA, 24 bp, Artificial Sequence
  Synthetic
aagtaaagaaatgttaagagtagt

SEQ ID NO: 673 - DNA, 24 bp, Artificial Sequence
  Synthetic
atgagaattgttgttgaaatgtaa

SEQ ID NO: 674 - DNA, 24 bp, Artificial Sequence
  Synthetic
ttagattagagtagtagaagaata

SEQ ID NO: 675 - DNA, 24 bp, Artificial Sequence
  Synthetic
tagtgatgaagaagttagaaatta

SEQ ID NO: 676 - DNA, 24 bp, Artificial Sequence
  Synthetic
taatgtagtaatgtgatgataagt

SEQ ID NO: 677 - DNA, 24 bp, Artificial Sequence
  Synthetic
ttgagaaagaataagtagtgtaaa

SEQ ID NO: 678 - DNA, 24 bp, Artificial Sequence
  Synthetic
taatgagtgagattatagattgtt

SEQ ID NO: 679 - DNA, 24 bp, Artificial Sequence
  Synthetic
gtataagaaatgtgtgtttgatta

SEQ ID NO: 680 - DNA, 24 bp, Artificial Sequence
  Synthetic
gtgaatgtgttaatgaagatatat

SEQ ID NO: 681 - DNA, 24 bp, Artificial Sequence
  Synthetic
gaaagttattagtagttaaagatg

SEQ ID NO: 682 - DNA, 24 bp, Artificial Sequence
  Synthetic
tagaattgtgtttgataagtgata

SEQ ID NO: 683 - DNA, 24 bp, Artificial Sequence
  Synthetic
tgatttagattgagagttaaatga

SEQ ID NO: 684 - DNA, 24 bp, Artificial Sequence
  Synthetic
attattgagtttgaatgttgatag

SEQ ID NO: 685 - DNA, 24 bp, Artificial Sequence
  Synthetic
atagtagttatgtttgatttagtg

SEQ ID NO: 686 - DNA, 24 bp, Artificial Sequence
  Synthetic
atagaagaagaataaagttagaga

SEQ ID NO: 687 - DNA, 24 bp, Artificial Sequence
  Synthetic
gatgttgaaagtaatgaatttgta

SEQ ID NO: 688 - DNA, 24 bp, Artificial Sequence
  Synthetic
gagattgatagtagaaatgataaa

SEQ ID NO: 689 - DNA, 24 bp, Artificial Sequence
  Synthetic
tgagagaataaagtatgaatttga

SEQ ID NO: 690 - DNA, 24 bp, Artificial Sequence
  Synthetic
tataaagatgatgtgaattagtag

SEQ ID NO: 691 - DNA, 24 bp, Artificial Sequence
  Synthetic
ttatgtaagaatgtttgagagaaa

SEQ ID NO: 692 - DNA, 24 bp, Artificial Sequence
  Synthetic
agtaaatgatgaatgatatgatga

SEQ ID NO: 693 - DNA, 24 bp, Artificial Sequence
  Synthetic
gaaatttgtgttaaagttgaatga

SEQ ID NO: 694 - DNA, 24 bp, Artificial Sequence
  Synthetic
gatgaatgattgtgtttaagtata

SEQ ID NO: 695 - DNA, 24 bp, Artificial Sequence
  Synthetic
gaaataagtgagagttaatgaaat

SEQ ID NO: 696 - DNA, 24 bp, Artificial Sequence
  Synthetic
tgttgaaatagttattagtttgtg

SEQ ID NO: 697 - DNA, 24 bp, Artificial Sequence
  Synthetic
tttgagagtatattgatatgagaa

SEQ ID NO: 698 - DNA, 24 bp, Artificial Sequence
  Synthetic
attgtgtgtaaagtaagatttaag

SEQ ID NO: 699 - DNA, 24 bp, Artificial Sequence
  Synthetic
tatagtttgaagtgtgatgtattt

SEQ ID NO: 700 - DNA, 24 bp, Artificial Sequence
  Synthetic
gtgaagttatagtgtataaagaat

SEQ ID NO: 701 - DNA, 24 bp, Artificial Sequence
  Synthetic
gtatgttgaatagtaaatagattg

SEQ ID NO: 702 - DNA, 24 bp, Artificial Sequence
  Synthetic
ttagaaagtgtgatttgtgtattt

SEQ ID NO: 703 - DNA, 24 bp, Artificial Sequence
  Synthetic
tttagtaatatgtaagagatgtga

SEQ ID NO: 704 - DNA, 24 bp, Artificial Sequence
  Synthetic
agtatgtatagatgatgtttgttt

SEQ ID NO: 705 - DNA, 24 bp, Artificial Sequence
  Synthetic
atttaagtaaagtgtagagataag

SEQ ID NO: 706 - DNA, 24 bp, Artificial Sequence
  Synthetic
atttgtgttgaattgtaaagtgaa

SEQ ID NO: 707 - DNA, 24 bp, Artificial Sequence
  Synthetic
atgttattagattgtgatgaatga

SEQ ID NO: 708 - DNA, 24 bp, Artificial Sequence
  Synthetic
tagtagtagaatatgaaattagag

SEQ ID NO: 709 - DNA, 24 bp, Artificial Sequence
  Synthetic
tttaatgagaagagttagagtata

SEQ ID NO: 710 - DNA, 24 bp, Artificial Sequence
  Synthetic
aaagtttagtagagtgtatgtaaa

SEQ ID NO: 711 - DNA, 24 bp, Artificial Sequence
  Synthetic
atatatgatagtagagtagattag

SEQ ID NO: 712 - DNA, 24 bp, Artificial Sequence
  Synthetic
tgagaagttaattgtatagattga

SEQ ID NO: 713 - DNA, 24 bp, Artificial Sequence
  Synthetic
tatagagatgttatatgaagttgt

SEQ ID NO: 714 - DNA, 24 bp, Artificial Sequence
  Synthetic
aaatttgttaagttgttgttgttg

SEQ ID NO: 715 - DNA, 24 bp, Artificial Sequence
  Synthetic
ttgttgaagatgaaagtagaatta

SEQ ID NO: 716 - DNA, 24 bp, Artificial Sequence
  Synthetic
aagagataagtagtgtttatgttt

SEQ ID NO: 717 - DNA, 24 bp, Artificial Sequence
  Synthetic
aataagaagaagtgaaagattgat

SEQ ID NO: 718 - DNA, 24 bp, Artificial Sequence
  Synthetic
taagttaaagttgatgattgatag

SEQ ID NO: 719 - DNA, 24 bp, Artificial Sequence
  Synthetic
atataagataagagtgtaagtgat

SEQ ID NO: 720 - DNA, 24 bp, Artificial Sequence
  Synthetic
gttaaatgttgttgtttaagtgat

SEQ ID NO: 721 - DNA, 24 bp, Artificial Sequence
  Synthetic
gagttaagttattagttaagaagt

SEQ ID NO: 722 - DNA, 24 bp, Artificial Sequence
  Synthetic
tattagagtttgagaataagtagt

SEQ ID NO: 723 - DNA, 24 bp, Artificial Sequence
  Synthetic
taatgttgttatgtgttagatgtt

SEQ ID NO: 724 - DNA, 24 bp, Artificial Sequence
  Synthetic
gaaagttgatagaatgtaatgttt

SEQ ID NO: 725 - DNA, 24 bp, Artificial Sequence
  Synthetic
tgatagatgaattgattgattagt

SEQ ID NO: 726 - DNA, 24 bp, Artificial Sequence
  Synthetic
atgatagagtaaagaataagttgt

SEQ ID NO: 727 - DNA, 24 bp, Artificial Sequence
  Synthetic
agtaagtgttagatagtattgaat

SEQ ID NO: 728 - DNA, 24 bp, Artificial Sequence
  Synthetic
atgtagattaaagtagtgtatgtt

SEQ ID NO: 729 - DNA, 24 bp, Artificial Sequence
  Synthetic
ttattgataatgagagagttaaag

SEQ ID NO: 730 - DNA, 24 bp, Artificial Sequence
  Synthetic
atttgttatgataaatgtgtagtg

SEQ ID NO: 731 - DNA, 24 bp, Artificial Sequence
  Synthetic
ttgaagaaataagagtaataagag

SEQ ID NO: 732 - DNA, 24 bp, Artificial Sequence
  Synthetic
tgtgtaataagtagtaagattaga

SEQ ID NO: 733 - DNA, 24 bp, Artificial Sequence
  Synthetic
atgaaagttagagtttatgataag

SEQ ID NO: 734 - DNA, 24 bp, Artificial Sequence
  Synthetic
attagttaagagagtttgtagatt

SEQ ID NO: 735 - DNA, 24 bp, Artificial Sequence
  Synthetic
tgtagtattgtatgattaaagtgt

SEQ ID NO: 736 - DNA, 24 bp, Artificial Sequence
  Synthetic
agttgataaagaagaagagtatat

SEQ ID NO: 737 - DNA, 24 bp, Artificial Sequence
  Synthetic
gtaatgagataaagagagataatt

SEQ ID NO: 738 - DNA, 24 bp, Artificial Sequence
  Synthetic
tgtgttgaagataaagtttatgat

SEQ ID NO: 739 - DNA, 24 bp, Artificial Sequence
  Synthetic
aagaagagtagttagaattgatta

SEQ ID NO: 740 - DNA, 24 bp, Artificial Sequence
  Synthetic
gaatgaagatgaagtttgttaata

SEQ ID NO: 741 - DNA, 24 bp, Artificial Sequence
  Synthetic
aaattgttgagataagatagtgat

SEQ ID NO: 742 - DNA, 24 bp, Artificial Sequence
  Synthetic
tgattgtttaatgatgtgtgatta

SEQ ID NO: 743 - DNA, 24 bp, Artificial Sequence
  Synthetic
atgaagtattgttgagtgatttaa

SEQ ID NO: 744 - DNA, 24 bp, Artificial Sequence
  Synthetic
gtgtaaatgtttgagatgtatatt

SEQ ID NO: 745 - DNA, 24 bp, Artificial Sequence
  Synthetic
aattgatgagtttaaagagttgat

SEQ ID NO: 746 - DNA, 24 bp, Artificial Sequence
  Synthetic
tttgtgtaatatgattgagagttt

SEQ ID NO: 747 - DNA, 24 bp, Artificial Sequence
  Synthetic
gtagtagatgattaagaagataaa

SEQ ID NO: 748 - DNA, 24 bp, Artificial Sequence
  Synthetic
tttaatgtgaaatttgttgtgagt

SEQ ID NO: 749 - DNA, 24 bp, Artificial Sequence
  Synthetic
gtaaagaattagataaagagtgat

SEQ ID NO: 750 - DNA, 24 bp, Artificial Sequence
  Synthetic
aatagttaagtttaagagttgtgt

SEQ ID NO: 751 - DNA, 24 bp, Artificial Sequence
  Synthetic
gtgtgatgtttatagatttgttat

SEQ ID NO: 752 - DNA, 24 bp, Artificial Sequence
  Synthetic
gtatagtgtgattagatttgtaaa

SEQ ID NO: 753 - DNA, 24 bp, Artificial Sequence
  Synthetic
gttgtaagaaagatatgtaagaaa

SEQ ID NO: 754 - DNA, 24 bp, Artificial Sequence
  Synthetic
atattagattgtaaagagagtgaa

SEQ ID NO: 755 - DNA, 24 bp, Artificial Sequence
  Synthetic
gagtgatattgaaattagattgta

SEQ ID NO: 756 - DNA, 24 bp, Artificial Sequence
  Synthetic
taagaagttaaagaagagagttta

SEQ ID NO: 757 - DNA, 24 bp, Artificial Sequence
  Synthetic
gatgttagataaagtttaagtagt

SEQ ID NO: 758 - DNA, 24 bp, Artificial Sequence
  Synthetic
gtgattgtatgagaaatgttaaat

SEQ ID NO: 759 - DNA, 24 bp, Artificial Sequence
  Synthetic
tgattattgtaagaaagattgaga

SEQ ID NO: 760 - DNA, 24 bp, Artificial Sequence
  Synthetic
aagaattgtgtaagtttatgagta

SEQ ID NO: 761 - DNA, 24 bp, Artificial Sequence
  Synthetic
ttgtatttagaagatttgtagatg

SEQ ID NO: 762 - DNA, 24 bp, Artificial Sequence
  Synthetic
tatatgtttgtgtaagaagaaatg

SEQ ID NO: 763 - DNA, 24 bp, Artificial Sequence
  Synthetic
gataatgtgtgaatttgtgaataa

SEQ ID NO: 764 - DNA, 24 bp, Artificial Sequence
  Synthetic
ttagaaatgtgagatttaagagtt

SEQ ID NO: 765 - DNA, 24 bp, Artificial Sequence
  Synthetic
agtgtagaatttgtatttagttgt

SEQ ID NO: 766 - DNA, 24 bp, Artificial Sequence
  Synthetic
tagttaagatagagtaaatgatag

SEQ ID NO: 767 - DNA, 24 bp, Artificial Sequence
  Synthetic
gaagtgatattgtaaattgataag

SEQ ID NO: 768 - DNA, 24 bp, Artificial Sequence
  Synthetic
gtaattgtgttagatttaagaagt

SEQ ID NO: 769 - DNA, 24 bp, Artificial Sequence
  Synthetic
tgatatttgtgaattgatagtatg

SEQ ID NO: 770 - DNA, 24 bp, Artificial Sequence
  Synthetic
aagtaaagagatatagttaagttg

SEQ ID NO: 771 - DNA, 24 bp, Artificial Sequence
  Synthetic
attagttaagttatttgtgagtga

SEQ ID NO: 772 - DNA, 24 bp, Artificial Sequence
  Synthetic
agatgaagtagtttatgaattaga

SEQ ID NO: 773 - DNA, 24 bp, Artificial Sequence
  Synthetic
tgagttagttaagtgatagttaaa

SEQ ID NO: 774 - DNA, 24 bp, Artificial Sequence
  Synthetic
ttattgtagatttagagaagatga

SEQ ID NO: 775 - DNA, 24 bp, Artificial Sequence
  Synthetic
tatttgtgtttgttgattagatag

SEQ ID NO: 776 - DNA, 24 bp, Artificial Sequence
  Synthetic
gtataatgtgtgtgaaagttataa

SEQ ID NO: 777 - DNA, 24 bp, Artificial Sequence
  Synthetic
tatatgttgagtataaagagagaa

SEQ ID NO: 778 - DNA, 24 bp, Artificial Sequence
  Synthetic
ttagttagtttaaagattgtgagt

SEQ ID NO: 779 - DNA, 24 bp, Artificial Sequence
  Synthetic
tttagaataagtgatgtgatgaaa

SEQ ID NO: 780 - DNA, 24 bp, Artificial Sequence
  Synthetic
agagtaatgtgtaaatagttagat

SEQ ID NO: 781 - DNA, 24 bp, Artificial Sequence
  Synthetic
tgtgataaagagaaattagttgtt

SEQ ID NO: 782 - DNA, 24 bp, Artificial Sequence
  Synthetic
gaatttagtgaatgtttgagatta

SEQ ID NO: 783 - DNA, 24 bp, Artificial Sequence
  Synthetic
tgtgatgtgtaagtatatgaaatt

SEQ ID NO: 784 - DNA, 24 bp, Artificial Sequence
  Synthetic
ttgtgaatgattaatgaatagaag

SEQ ID NO: 785 - DNA, 24 bp, Artificial Sequence
  Synthetic
aatgttgtttagattgagaaagtt

SEQ ID NO: 786 - DNA, 24 bp, Artificial Sequence
  Synthetic
agattgtgttagtattagtataag

SEQ ID NO: 787 - DNA, 24 bp, Artificial Sequence
  Synthetic
ttgatgtattagaaagtttatgtg

SEQ ID NO: 788 - DNA, 24 bp, Artificial Sequence
  Synthetic
tatgattgtgtgttagagaattta

SEQ ID NO: 789 - DNA, 24 bp, Artificial Sequence
  Synthetic
tagtgtagatatttgatagttatg

SEQ ID NO: 790 - DNA, 24 bp, Artificial Sequence
  Synthetic
agtttaatgtgtttagttgttatg

SEQ ID NO: 791 - DNA, 24 bp, Artificial Sequence
  Synthetic
tgtgtaaagtagaaagtaaagatt

SEQ ID NO: 792 - DNA, 24 bp, Artificial Sequence
  Synthetic
gttatgatatagtgagttgttatt

SEQ ID NO: 793 - DNA, 24 bp, Artificial Sequence
  Synthetic
tttgattgaatgttaatagtgtgt

SEQ ID NO: 794 - DNA, 24 bp, Artificial Sequence
  Synthetic
agagtattagtagttattgtaagt

SEQ ID NO: 795 - DNA, 24 bp, Artificial Sequence
  Synthetic
taagtagaaagaagaagatatttg

SEQ ID NO: 796 - DNA, 24 bp, Artificial Sequence
  Synthetic
agaaagagaattatgtaatgaaag

SEQ ID NO: 797 - DNA, 24 bp, Artificial Sequence
  Synthetic
ttagatttgttagtgtgatttaag

SEQ ID NO: 798 - DNA, 24 bp, Artificial Sequence
  Synthetic
gatgattaagatatagagatagtt

SEQ ID NO: 799 - DNA, 24 bp, Artificial Sequence
  Synthetic
atatttgagtgattaagagtaatg

SEQ ID NO: 800 - DNA, 24 bp, Artificial Sequence
  Synthetic
tgtattgtgagttaagtataagtt

SEQ ID NO: 801 - DNA, 24 bp, Artificial Sequence
  Synthetic
aatttagtagaaagtgttgtgttt

SEQ ID NO: 802 - DNA, 24 bp, Artificial Sequence
  Synthetic
gttagaagattaagttgaataatg

SEQ ID NO: 803 - DNA, 24 bp, Artificial Sequence
  Synthetic
taaagtatgtgagatgatttatgt

SEQ ID NO: 804 - DNA, 24 bp, Artificial Sequence
  Synthetic
tgaaatgattaaagatgaagatga

SEQ ID NO: 805 - DNA, 24 bp, Artificial Sequence
  Synthetic
ttattagatgttgagtgtttgttt

SEQ ID NO: 806 - DNA, 24 bp, Artificial Sequence
  Synthetic
tagtgtttaaagagtagtatatga

SEQ ID NO: 807 - DNA, 24 bp, Artificial Sequence
  Synthetic
agttataagtaaatgatgttgatg

SEQ ID NO: 808 - DNA, 24 bp, Artificial Sequence
  Synthetic
ttaagagagaaataagtgtattgt

SEQ ID NO: 809 - DNA, 24 bp, Artificial Sequence
  Synthetic
gatattgaaatgtgtaaatgatga

SEQ ID NO: 810 - DNA, 24 bp, Artificial Sequence
  Synthetic
atgatgaattaagaaagaaagaga

SEQ ID NO: 811 - DNA, 24 bp, Artificial Sequence
  Synthetic
gaatagtttgatttgtgtttgtta

SEQ ID NO: 812 - DNA, 24 bp, Artificial Sequence
  Synthetic
agttgtttagatttgatttgtaag

SEQ ID NO: 813 - DNA, 24 bp, Artificial Sequence
  Synthetic
gtatgagatttgatataagattag

SEQ ID NO: 814 - DNA, 24 bp, Artificial Sequence
  Synthetic
tttatagtgagtatagtgatgatt

SEQ ID NO: 815 - DNA, 24 bp, Artificial Sequence
  Synthetic
tatatgtgaagatataagtgtttg

SEQ ID NO: 816 - DNA, 24 bp, Artificial Sequence
  Synthetic
attgatagatgatagtaattgagt

SEQ ID NO: 817 - DNA, 24 bp, Artificial Sequence
  Synthetic
tgatagatgtgaagaatttgattt

SEQ ID NO: 818 - DNA, 24 bp, Artificial Sequence
  Synthetic
gaagatattgaaagaatttgatgt

SEQ ID NO: 819 - DNA, 24 bp, Artificial Sequence
  Synthetic
gatgtttagtgtagatatagattt

SEQ ID NO: 820 - DNA, 24 bp, Artificial Sequence
  Synthetic
gaatattgagttataagtagtagt

SEQ ID NO: 821 - DNA, 24 bp, Artificial Sequence
  Synthetic
agtgagtaagtaatagaaagattt

SEQ ID NO: 822 - DNA, 24 bp, Artificial Sequence
  Synthetic
gtagaataagtaatttgtgagata

SEQ ID NO: 823 - DNA, 24 bp, Artificial Sequence
  Synthetic
gagttatttgagatttagatgttt

SEQ ID NO: 824 - DNA, 24 bp, Artificial Sequence
  Synthetic
gaaatgatgattgaatttagagat

SEQ ID NO: 825 - DNA, 24 bp, Artificial Sequence
  Synthetic
aaatagtgtgagaatagttaagta

SEQ ID NO: 826 - DNA, 24 bp, Artificial Sequence
  Synthetic
atgtgttaagttgtagaagaataa

SEQ ID NO: 827 - DNA, 24 bp, Artificial Sequence
  Synthetic
ataatgagttaatagtgtaagaag

SEQ ID NO: 828 - DNA, 24 bp, Artificial Sequence
  Synthetic
ataagagatgtttaagttagaaag

SEQ ID NO: 829 - DNA, 24 bp, Artificial Sequence
  Synthetic
tgttagtgttagaaatatgaaaga

SEQ ID NO: 830 - DNA, 24 bp, Artificial Sequence
  Synthetic
tttagaagattgttagataagttg

SEQ ID NO: 831 - DNA, 24 bp, Artificial Sequence
  Synthetic
gtgtaatgtataagatagttaagt

SEQ ID NO: 832 - DNA, 24 bp, Artificial Sequence
  Synthetic
tattagagagaaattgtagagatt

SEQ ID NO: 833 - DNA, 24 bp, Artificial Sequence
  Synthetic
tagtgagataaagtaaagtttatg

SEQ ID NO: 834 - DNA, 24 bp, Artificial Sequence
  Synthetic
ttgtgaaagttaagtaagttagtt

SEQ ID NO: 835 - DNA, 24 bp, Artificial Sequence
  Synthetic
aaagtgtaagttgaagaatattga

SEQ ID NO: 836 - DNA, 24 bp, Artificial Sequence
  Synthetic
gaatagagtgttatttgaaataga

SEQ ID NO: 837 - DNA, 24 bp, Artificial Sequence
  Synthetic
tataagagagagataagtaataag

SEQ ID NO: 838 - DNA, 24 bp, Artificial Sequence
  Synthetic
tgagtgaaattgatagagtaaatt

SEQ ID NO: 839 - DNA, 24 bp, Artificial Sequence
  Synthetic
gatgaataagtttaagtgagaaat

SEQ ID NO: 840 - DNA, 24 bp, Artificial Sequence
  Synthetic
gtgtgatatgtttattgattaagt

SEQ ID NO: 841 - DNA, 24 bp, Artificial Sequence
  Synthetic
taaagtgagtgtaaatgataatga

SEQ ID NO: 842 - DNA, 24 bp, Artificial Sequence
  Synthetic
gtagagtttgatttgaaagaatat

SEQ ID NO: 843 - DNA, 24 bp, Artificial Sequence
  Synthetic
gaatattgttatgtttgttatgag

SEQ ID NO: 844 - DNA, 24 bp, Artificial Sequence
  Synthetic
gtgtaataagatgtattgttgttt

SEQ ID NO: 845 - DNA, 24 bp, Artificial Sequence
  Synthetic
taaattgattgtgagttgaagaat

SEQ ID NO: 846 - DNA, 24 bp, Artificial Sequence
  Synthetic
tgagatagttatagttaagtttag

SEQ ID NO: 847 - DNA, 24 bp, Artificial Sequence
  Synthetic
agtttgttaagattatgtagaaag

SEQ ID NO: 848 - DNA, 24 bp, Artificial Sequence
  Synthetic
gaatgtgtagaataagagattaaa

SEQ ID NO: 849 - DNA, 24 bp, Artificial Sequence
  Synthetic
gtattatgaaagaagttgttgttt

SEQ ID NO: 850 - DNA, 24 bp, Artificial Sequence
  Synthetic
gtgttatagaagttaaatgttaag

SEQ ID NO: 851 - DNA, 24 bp, Artificial Sequence
  Synthetic
ttaagagtagtgaatatgatagta

SEQ ID NO: 852 - DNA, 24 bp, Artificial Sequence
  Synthetic
aatgttataagatgagagtttagt

SEQ ID NO: 853 - DNA, 24 bp, Artificial Sequence
  Synthetic
atataagatttgatgtagtgtagt

SEQ ID NO: 854 - DNA, 24 bp, Artificial Sequence
  Synthetic
tatgtttgttgttgttaagtttga

SEQ ID NO: 855 - DNA, 24 bp, Artificial Sequence
  Synthetic
gatagtttagtatagaagataaag

SEQ ID NO: 856 - DNA, 24 bp, Artificial Sequence
  Synthetic
gttgaatatagagatagtaaatag

SEQ ID NO: 857 - DNA, 24 bp, Artificial Sequence
  Synthetic
agagaagatttagtaagaatgata

SEQ ID NO: 858 - DNA, 24 bp, Artificial Sequence
  Synthetic
tgaatgagaaagatattgagtatt

SEQ ID NO: 859 - DNA, 24 bp, Artificial Sequence
  Synthetic
tgaagattatagtagttgtataga

SEQ ID NO: 860 - DNA, 24 bp, Artificial Sequence
  Synthetic
gattagtagtattgaagattatgt

SEQ ID NO: 861 - DNA, 24 bp, Artificial Sequence
  Synthetic
tgaaatgtgtatttgtatgtttag

SEQ ID NO: 862 - DNA, 24 bp, Artificial Sequence
  Synthetic
attaaagttgatatgaaagaagtg

SEQ ID NO: 863 - DNA, 24 bp, Artificial Sequence
  Synthetic
aatgtagagattgtagtgaatatt

SEQ ID NO: 864 - DNA, 24 bp, Artificial Sequence
  Synthetic
ttatttgttgagtgtaaatgtgat

SEQ ID NO: 865 - DNA, 24 bp, Artificial Sequence
  Synthetic
atgtaattgtgaataatgtatgtg

SEQ ID NO: 866 - DNA, 24 bp, Artificial Sequence
  Synthetic
gatttgtatagagattagtaagta

SEQ ID NO: 867 - DNA, 24 bp, Artificial Sequence
  Synthetic
aatattgttgtttagagaaagaag

SEQ ID NO: 868 - DNA, 24 bp, Artificial Sequence
  Synthetic
atgatgatgtatttgtaaagagta

SEQ ID NO: 869 - DNA, 24 bp, Artificial Sequence
  Synthetic
aatgtatttgtgtgattgtgtaaa

SEQ ID NO: 870 - DNA, 24 bp, Artificial Sequence
  Synthetic
agtgttatgaagaatagtaagaat

SEQ ID NO: 871 - DNA, 24 bp, Artificial Sequence
  Synthetic
gttatgtagagatgaaagaaatta

SEQ ID NO: 872 - DNA, 24 bp, Artificial Sequence
  Synthetic
gtttgtattagataaatgagttgt

SEQ ID NO: 873 - DNA, 24 bp, Artificial Sequence
  Synthetic
tgatttatgagattaagagaaaga

SEQ ID NO: 874 - DNA, 24 bp, Artificial Sequence
  Synthetic
tttgtgtgttattgtaattgagat

SEQ ID NO: 875 - DNA, 24 bp, Artificial Sequence
  Synthetic
gatgtgtgatatgattaaagaaat

SEQ ID NO: 876 - DNA, 24 bp, Artificial Sequence
  Synthetic
agattatagatttgtagagaaagt

SEQ ID NO: 877 - DNA, 24 bp, Artificial Sequence
  Synthetic
gaagagtatgtaatagtattgtat

SEQ ID NO: 878 - DNA, 24 bp, Artificial Sequence
  Synthetic
tttgtaatgttgttgagtttaaga

SEQ ID NO: 879 - DNA, 24 bp, Artificial Sequence
  Synthetic
agtaaatagtagtatgaataagag

SEQ ID NO: 880 - DNA, 24 bp, Artificial Sequence
  Synthetic
gaatgttgaattgaaatatgagtt

SEQ ID NO: 881 - DNA, 24 bp, Artificial Sequence
  Synthetic
agtagttaattgatagtaagtttg

SEQ ID NO: 882 - DNA, 24 bp, Artificial Sequence
  Synthetic
agtgtaaagaaatgaatgaataag

SEQ ID NO: 883 - DNA, 24 bp, Artificial Sequence
  Synthetic
tgttagatatttgtgaaatgtgaa

SEQ ID NO: 884 - DNA, 24 bp, Artificial Sequence
  Synthetic
tgtatgttgagtttgaattgttat

SEQ ID NO: 885 - DNA, 24 bp, Artificial Sequence
  Synthetic
tgagtgaattagttatgttgttat

SEQ ID NO: 886 - DNA, 24 bp, Artificial Sequence
  Synthetic
gaagaaagaaatgagaaagattat

SEQ ID NO: 887 - DNA, 24 bp, Artificial Sequence
  Synthetic
ttaagtaagttgtgttgatattag

SEQ ID NO: 888 - DNA, 24 bp, Artificial Sequence
  Synthetic
atgatgtgtttgatttgaattgaa

SEQ ID NO: 889 - DNA, 24 bp, Artificial Sequence
  Synthetic
aagtaagtgaaattgttgtttgaa

SEQ ID NO: 890 - DNA, 24 bp, Artificial Sequence
  Synthetic
atgaagtgtaaagtttgaaagaaa

SEQ ID NO: 891 - DNA, 24 bp, Artificial Sequence
  Synthetic
agagagtaagataattgtatagta

SEQ ID NO: 892 - DNA, 24 bp, Artificial Sequence
  Synthetic
tttatgagatagatgaaataagtg

SEQ ID NO: 893 - DNA, 24 bp, Artificial Sequence
  Synthetic
agaaattagtagtaatgatttgtg

SEQ ID NO: 894 - DNA, 24 bp, Artificial Sequence
  Synthetic
gatttgagattgaatgagaatata

SEQ ID NO: 895 - DNA, 24 bp, Artificial Sequence
  Synthetic
gattagaaagatgaataaagatga

SEQ ID NO: 896 - DNA, 24 bp, Artificial Sequence
  Synthetic
tagatagaaagtatatgttgtagt

SEQ ID NO: 897 - DNA, 24 bp, Artificial Sequence
  Synthetic
gaagatagtaaagtaaagtaagtt

SEQ ID NO: 898 - DNA, 24 bp, Artificial Sequence
  Synthetic
aaatgtgtgtttagtagttgtaaa

SEQ ID NO: 899 - DNA, 24 bp, Artificial Sequence
  Synthetic
ttgttgaagtaagagatgaataaa

SEQ ID NO: 900 - DNA, 24 bp, Artificial Sequence
  Synthetic
tatttgagagaaagaaagagttta

SEQ ID NO: 901 - DNA, 24 bp, Artificial Sequence
  Synthetic
tatttagtgatgaatttgtgatgt

SEQ ID NO: 902 - DNA, 24 bp, Artificial Sequence
  Synthetic
ttatagtgatgatgataagttgat

SEQ ID NO: 903 - DNA, 24 bp, Artificial Sequence
  Synthetic
taaagataattgtagaaagtagtg

SEQ ID NO: 904 - DNA, 24 bp, Artificial Sequence
  Synthetic
gtttagtattgatattgtgtgtaa

SEQ ID NO: 905 - DNA, 24 bp, Artificial Sequence
  Synthetic
gtgttgtgaataagattgaaatat

SEQ ID NO: 906 - DNA, 24 bp, Artificial Sequence
  Synthetic
aaagaaagtataaagtgagataga

SEQ ID NO: 907 - DNA, 24 bp, Artificial Sequence
  Synthetic
tatttgtaagaagtgtagatattg

SEQ ID NO: 908 - DNA, 24 bp, Artificial Sequence
  Synthetic
tagaagatgaaattgtgatttgtt

SEQ ID NO: 909 - DNA, 24 bp, Artificial Sequence
  Synthetic
ataatagtaagtgaatgatgagat

SEQ ID NO: 910 - DNA, 24 bp, Artificial Sequence
  Synthetic
aatgtgaataagataaagtgtgta

SEQ ID NO: 911 - DNA, 24 bp, Artificial Sequence
  Synthetic
attgaagataaagatgttgtttag

SEQ ID NO: 912 - DNA, 24 bp, Artificial Sequence
  Synthetic
tgaaatagaagtgagattatagta

SEQ ID NO: 913 - DNA, 24 bp, Artificial Sequence
  Synthetic
agttattgtgaaagagtttatgat

SEQ ID NO: 914 - DNA, 24 bp, Artificial Sequence
  Synthetic
aaatagtagtgatagagaagattt

SEQ ID NO: 915 - DNA, 24 bp, Artificial Sequence
  Synthetic
agtgtatgaagtgtaataagatta

SEQ ID NO: 916 - DNA, 24 bp, Artificial Sequence
  Synthetic
tgattaagattgtgtagtgttata

SEQ ID NO: 917 - DNA, 24 bp, Artificial Sequence
  Synthetic
agtttatgatatttgtagatgagt

SEQ ID NO: 918 - DNA, 24 bp, Artificial Sequence
  Synthetic
tatgtgtatgaagattatagttag

SEQ ID NO: 919 - DNA, 24 bp, Artificial Sequence
  Synthetic
gaaattgttgtatagagtgatata

SEQ ID NO: 920 - DNA, 24 bp, Artificial Sequence
  Synthetic
tagaaatagtttaagtatagtgtg

SEQ ID NO: 921 - DNA, 24 bp, Artificial Sequence
  Synthetic
tgatttagatgtttattgtgagaa

SEQ ID NO: 922 - DNA, 24 bp, Artificial Sequence
  Synthetic
aagttgatatttgttgttagatga

SEQ ID NO: 923 - DNA, 24 bp, Artificial Sequence
  Synthetic
tgatgtgataatgagaataaagaa

SEQ ID NO: 924 - DNA, 24 bp, Artificial Sequence
  Synthetic
aaagtttagtttgtattagtagag

SEQ ID NO: 925 - DNA, 24 bp, Artificial Sequence
  Synthetic
agtttgatgtgatagtaaatagaa

SEQ ID NO: 926 - DNA, 24 bp, Artificial Sequence
  Synthetic
aagtgttattgaatgtgatgttat

SEQ ID NO: 927 - DNA, 24 bp, Artificial Sequence
  Synthetic
aaattgaagtgtgataatgtttgt

SEQ ID NO: 928 - DNA, 24 bp, Artificial Sequence
  Synthetic
gtttagtgattaaagatagattag

SEQ ID NO: 929 - DNA, 24 bp, Artificial Sequence
  Synthetic
ataagtgtataagagaagtgttaa

SEQ ID NO: 930 - DNA, 24 bp, Artificial Sequence
  Synthetic
atgaatttgtttgtgatgaagtta

SEQ ID NO: 931 - DNA, 24 bp, Artificial Sequence
  Synthetic
aaagaattgagaaatgaaagttag

SEQ ID NO: 932 - DNA, 24 bp, Artificial Sequence
  Synthetic
agtgtaagagtataaagtatttga

SEQ ID NO: 933 - DNA, 24 bp, Artificial Sequence
  Synthetic
gaattaagattgttatatgtgagt

SEQ ID NO: 934 - DNA, 24 bp, Artificial Sequence
  Synthetic
tatgaaagtgttgtttaagtaaga

SEQ ID NO: 935 - DNA, 24 bp, Artificial Sequence
  Synthetic
taaagtaaatgttatgtgagagaa

SEQ ID NO: 936 - DNA, 24 bp, Artificial Sequence
  Synthetic
aaagatattgattgagatagagtt

SEQ ID NO: 937 - DNA, 24 bp, Artificial Sequence
  Synthetic
aagtgatatgaatatgtgagaaat

SEQ ID NO: 938 - DNA, 24 bp, Artificial Sequence
  Synthetic
aaatagagtttgttaatgtaagtg

SEQ ID NO: 939 - DNA, 24 bp, Artificial Sequence
  Synthetic
gatttagatgagttaagaatttag

SEQ ID NO: 940 - DNA, 24 bp, Artificial Sequence
  Synthetic
ttgtaaatgagtgtgaatattgta

SEQ ID NO: 941 - DNA, 24 bp, Artificial Sequence
  Synthetic
agtagtgtatttgagataatagaa

SEQ ID NO: 942 - DNA, 24 bp, Artificial Sequence
  Synthetic
tgagttaaagagttgttgatattt

SEQ ID NO: 943 - DNA, 24 bp, Artificial Sequence
  Synthetic
aaagagtgtattagaaatagtttg

SEQ ID NO: 944 - DNA, 24 bp, Artificial Sequence
  Synthetic
gtttagttatttgatgagataatg

SEQ ID NO: 945 - DNA, 24 bp, Artificial Sequence
  Synthetic
aagtgtaaatgaataaagagttgt

SEQ ID NO: 946 - DNA, 24 bp, Artificial Sequence
  Synthetic
aataaagtgagtagaagtgtaatt

SEQ ID NO: 947 - DNA, 24 bp, Artificial Sequence
  Synthetic
tattgagtttgtgtaaagaagata

SEQ ID NO: 948 - DNA, 24 bp, Artificial Sequence
  Synthetic
tttatagttgttgtgttgaaagtt

SEQ ID NO: 949 - DNA, 24 bp, Artificial Sequence
  Synthetic
atgaaatatgattgtgtttgttgt

SEQ ID NO: 950 - DNA, 24 bp, Artificial Sequence
  Synthetic
aaagagatgtaaagtgagttatta

SEQ ID NO: 951 - DNA, 24 bp, Artificial Sequence
  Synthetic
ttgaagaaagttagatgatgaatt

SEQ ID NO: 952 - DNA, 24 bp, Artificial Sequence
  Synthetic
atgttatttgtttagtttgtgtga

SEQ ID NO: 953 - DNA, 24 bp, Artificial Sequence
  Synthetic
aaatatgaatttgaagagaagtga

SEQ ID NO: 954 - DNA, 24 bp, Artificial Sequence
  Synthetic
gattagatatagaatattgaagag

SEQ ID NO: 955 - DNA, 24 bp, Artificial Sequence
  Synthetic
ttagaataagagaaatgtatgtgt

SEQ ID NO: 956 - DNA, 24 bp, Artificial Sequence
  Synthetic
tttatgaaagagaagtgtattatg

SEQ ID NO: 957 - DNA, 24 bp, Artificial Sequence
  Synthetic
gtaagtattaagtgtgatttagta

SEQ ID NO: 958 - DNA, 24 bp, Artificial Sequence
  Synthetic
ataaagagaagtaaagagtaaagt

SEQ ID NO: 959 - DNA, 24 bp, Artificial Sequence
  Synthetic
attgttaattgaagtgtatgaaag

SEQ ID NO: 960 - DNA, 24 bp, Artificial Sequence
  Synthetic
tatatagttgagttgagtaagatt

SEQ ID NO: 961 - DNA, 24 bp, Artificial Sequence
  Synthetic
tagatgagatatatgaaagatagt

SEQ ID NO: 962 - DNA, 24 bp, Artificial Sequence
  Synthetic
ataagaagatgatttgtgtaaatg

SEQ ID NO: 963 - DNA, 24 bp, Artificial Sequence
  Synthetic
ttagtaataagaaagatgaagaga

SEQ ID NO: 964 - DNA, 24 bp, Artificial Sequence
  Synthetic
gatttgtgagtaaagtaaatagaa

SEQ ID NO: 965 - DNA, 24 bp, Artificial Sequence
  Synthetic
aaatagatgtagaatttgtgtgtt

SEQ ID NO: 966 - DNA, 24 bp, Artificial Sequence
  Synthetic
gaaattagtgtttgtgtgtattat

SEQ ID NO: 967 - DNA, 24 bp, Artificial Sequence
  Synthetic
atttgagtatgatagaagattgtt

SEQ ID NO: 968 - DNA, 24 bp, Artificial Sequence
  Synthetic
atagagttgaagtatgtaaagttt

SEQ ID NO: 969 - DNA, 24 bp, Artificial Sequence
  Synthetic
taatttgtgaatgttgttattgtg

SEQ ID NO: 970 - DNA, 24 bp, Artificial Sequence
  Synthetic
ttagtttatgagagtgagatttaa

SEQ ID NO: 971 - DNA, 24 bp, Artificial Sequence
  Synthetic
gttgttagagtgtttatgaaattt

SEQ ID NO: 972 - DNA, 24 bp, Artificial Sequence
  Synthetic
tttattgtgatgtgaaataagaga

SEQ ID NO: 973 - DNA, 24 bp, Artificial Sequence
  Synthetic
gtaagtaatatgatagtgattaag

SEQ ID NO: 974 - DNA, 24 bp, Artificial Sequence
  Synthetic
tgagatgatgtatatgtagtaata

SEQ ID NO: 975 - DNA, 24 bp, Artificial Sequence
  Synthetic
aattgagaaagagataaatgatag

SEQ ID NO: 976 - DNA, 24 bp, Artificial Sequence
  Synthetic
tttgaagtgatgttagaatgttta

SEQ ID NO: 977 - DNA, 24 bp, Artificial Sequence
  Synthetic
agttgttgtgtaattgttagtaaa

SEQ ID NO: 978 - DNA, 24 bp, Artificial Sequence
  Synthetic
atagtgagaagtgataagatattt

SEQ ID NO: 979 - DNA, 24 bp, Artificial Sequence
  Synthetic
gtgtgataagtaattgagttaaat

SEQ ID NO: 980 - DNA, 24 bp, Artificial Sequence
  Synthetic
tagttattgtttgtgaatttgaga

SEQ ID NO: 981 - DNA, 24 bp, Artificial Sequence
  Synthetic
atagttgaatagtaatttgaagag

SEQ ID NO: 982 - DNA, 24 bp, Artificial Sequence
  Synthetic
atgtttgtgtttgaatagagaata

SEQ ID NO: 983 - DNA, 24 bp, Artificial Sequence
  Synthetic
tgataaagatatgagagattgtaa

SEQ ID NO: 984 - DNA, 24 bp, Artificial Sequence
  Synthetic
taaagatgagatgttgttaaagtt

SEQ ID NO: 985 - DNA, 24 bp, Artificial Sequence
  Synthetic
aagtgaaatttgtaagaattagtg

SEQ ID NO: 986 - DNA, 24 bp, Artificial Sequence
  Synthetic
gaaatgagagttattgatagttta

SEQ ID NO: 987 - DNA, 24 bp, Artificial Sequence
  Synthetic
tttgtaaatgagatatagtgttag

SEQ ID NO: 988 - DNA, 24 bp, Artificial Sequence
  Synthetic
gttaattgtgatatttgattagtg

SEQ ID NO: 989 - DNA, 24 bp, Artificial Sequence
  Synthetic
agagtgttgataaagatgtttata

SEQ ID NO: 990 - DNA, 24 bp, Artificial Sequence
  Synthetic
aattgtgagaaattgataagaaga

SEQ ID NO: 991 - DNA, 24 bp, Artificial Sequence
  Synthetic
ttaaagagaattgagaagagaaat

SEQ ID NO: 992 - DNA, 24 bp, Artificial Sequence
  Synthetic
ttgttagaagaattgaatgtatgt

SEQ ID NO: 993 - DNA, 24 bp, Artificial Sequence
  Synthetic
agttaagatatgtgtgatgtttaa

SEQ ID NO: 994 - DNA, 24 bp, Artificial Sequence
  Synthetic
tgagttatgttgtaatagaaattg

SEQ ID NO: 995 - DNA, 24 bp, Artificial Sequence
  Synthetic
ttagataagtttagagattgagaa

SEQ ID NO: 996 - DNA, 24 bp, Artificial Sequence
  Synthetic
atgagtaataagagtatttgaagt

SEQ ID NO: 997 - DNA, 24 bp, Artificial Sequence
  Synthetic
tgtttaagtgtaatgatttgttag

SEQ ID NO: 998 - DNA, 24 bp, Artificial Sequence
  Synthetic
ttgaagaagattgttattgttgaa

SEQ ID NO: 999 - DNA, 24 bp, Artificial Sequence
  Synthetic
tatagaaagattaaagagtgaatg

SEQ ID NO: 1000 - DNA, 24 bp, Artificial Sequence
  Synthetic
taaattgttagaaatttgagtgtg

SEQ ID NO: 1001 - DNA, 24 bp, Artificial Sequence
  Synthetic
attgttagtgtgttattgattatg

SEQ ID NO: 1002 - DNA, 24 bp, Artificial Sequence
  Synthetic
gagaattatgtgtgaatatagaaa

SEQ ID NO: 1003 - DNA, 24 bp, Artificial Sequence
  Synthetic
ttgattgataaagtaaagagtgta

SEQ ID NO: 1004 - DNA, 24 bp, Artificial Sequence
  Synthetic
gtgtgtaaattgaatatgttaatg

SEQ ID NO: 1005 - DNA, 24 bp, Artificial Sequence
  Synthetic
aaagtaaagaaagaagtttgaaag

SEQ ID NO: 1006 - DNA, 24 bp, Artificial Sequence
  Synthetic
tttagttgaagaatagaaagaaag

SEQ ID NO: 1007 - DNA, 24 bp, Artificial Sequence
  Synthetic
gtgtaataagagtgaatagtaatt

SEQ ID NO: 1008 - DNA, 24 bp, Artificial Sequence
  Synthetic
tattgaaataagagagatttgtga

SEQ ID NO: 1009 - DNA, 24 bp, Artificial Sequence
  Synthetic
atgagaaagaagaagttaagattt

SEQ ID NO: 1010 - DNA, 24 bp, Artificial Sequence
  Synthetic
aagagtgagtatattgttaaagaa

SEQ ID NO: 1011 - DNA, 24 bp, Artificial Sequence
  Synthetic
tttgtaaagtgatgatgtaagata

SEQ ID NO: 1012 - DNA, 24 bp, Artificial Sequence
  Synthetic
gatgttatgtgatgaaatatgtat

SEQ ID NO: 1013 - DNA, 24 bp, Artificial Sequence
  Synthetic
gtagaataaagtgttaaagtgtta

SEQ ID NO: 1014 - DNA, 24 bp, Artificial Sequence
  Synthetic
aaagagtatgtgtgtatgatattt

SEQ ID NO: 1015 - DNA, 24 bp, Artificial Sequence
  Synthetic
aaagataagagttagtaaattgtg

SEQ ID NO: 1016 - DNA, 24 bp, Artificial Sequence
  Synthetic
aagaattagagaataagtgtgata

SEQ ID NO: 1017 - DNA, 24 bp, Artificial Sequence
  Synthetic
gataagaaagtgaaatgtaaattg

SEQ ID NO: 1018 - DNA, 24 bp, Artificial Sequence
  Synthetic
gatgaaagatgtttaaagtttgtt

SEQ ID NO: 1019 - DNA, 24 bp, Artificial Sequence
  Synthetic
agtgtaagtaataagtttgagaaa

SEQ ID NO: 1020 - DNA, 24 bp, Artificial Sequence
  Synthetic
gttgagaattagaatttgataaag

SEQ ID NO: 1021 - DNA, 24 bp, Artificial Sequence
  Synthetic
ttaagaaatttgtatgtgttgttg

SEQ ID NO: 1022 - DNA, 24 bp, Artificial Sequence
  Synthetic
agaagatttagatgaaatgagttt

SEQ ID NO: 1023 - DNA, 24 bp, Artificial Sequence
  Synthetic
taagtttgagataaagatgatatg

SEQ ID NO: 1024 - DNA, 24 bp, Artificial Sequence
  Synthetic
tgagatagtttgtaatatgtttgt

SEQ ID NO: 1025 - DNA, 24 bp, Artificial Sequence
  Synthetic
agtttgaaattgtaagtttgatga

SEQ ID NO: 1026 - DNA, 24 bp, Artificial Sequence
  Synthetic
tagaattgattaatgatgagtagt

SEQ ID NO: 1027 - DNA, 24 bp, Artificial Sequence
  Synthetic
agagatttgtaataagtattgaag

SEQ ID NO: 1028 - DNA, 24 bp, Artificial Sequence
  Synthetic
ataatgatgtaatgtaagtagtgt

SEQ ID NO: 1029 - DNA, 24 bp, Artificial Sequence
  Synthetic
tgaaatttgatgagagatatgtta

SEQ ID NO: 1030 - DNA, 24 bp, Artificial Sequence
  Synthetic
tgtgtaaagtatagtttatgttag

SEQ ID NO: 1031 - DNA, 24 bp, Artificial Sequence
  Synthetic
tgaataagtgaaatagaatgaatg

SEQ ID NO: 1032 - DNA, 24 bp, Artificial Sequence
  Synthetic
aaagaaagattgtaataagtagag

SEQ ID NO: 1033 - DNA, 24 bp, Artificial Sequence
  Synthetic
aatgaaatagtgttaaatgagtgt

SEQ ID NO: 1034 - DNA, 24 bp, Artificial Sequence
  Synthetic
gtagataaagatgtgaattatgat

SEQ ID NO: 1035 - DNA, 24 bp, Artificial Sequence
  Synthetic
gatagtatatgtgtgtatttgttt

SEQ ID NO: 1036 - DNA, 24 bp, Artificial Sequence
  Synthetic
atgtttgtagaaatgtttgaagat

SEQ ID NO: 1037 - DNA, 24 bp, Artificial Sequence
  Synthetic
aaatttgtagagagaaatttgttg

SEQ ID NO: 1038 - DNA, 24 bp, Artificial Sequence
  Synthetic
tagaataagattagtaagtgtaga

SEQ ID NO: 1039 - DNA, 24 bp, Artificial Sequence
  Synthetic
tgatttagagaaatatgagtagaa

SEQ ID NO: 1040 - DNA, 24 bp, Artificial Sequence
  Synthetic
aatagagtatgttgtttatgagaa

SEQ ID NO: 1041 - DNA, 24 bp, Artificial Sequence
  Synthetic
gatgatgaagagtttattgtaaat

SEQ ID NO: 1042 - DNA, 24 bp, Artificial Sequence
  Synthetic
aagtaaagaagaagaaatgtgtta

SEQ ID NO: 1043 - DNA, 24 bp, Artificial Sequence
  Synthetic
ttgaagaattaagtgtttagtgta

SEQ ID NO: 1044 - DNA, 24 bp, Artificial Sequence
  Synthetic
agaaagaatgttgatttatgatgt

SEQ ID NO: 1045 - DNA, 24 bp, Artificial Sequence
  Synthetic
gattaaagagatgttgattgaaat

SEQ ID NO: 1046 - DNA, 24 bp, Artificial Sequence
  Synthetic
aatgataattgttgagagagtaat

SEQ ID NO: 1047 - DNA, 24 bp, Artificial Sequence
  Synthetic
gtttgttgaaagtgtaaagtatat

SEQ ID NO: 1048 - DNA, 24 bp, Artificial Sequence
  Synthetic
tgagttatatgagaaagtgtaatt

SEQ ID NO: 1049 - DNA, 24 bp, Artificial Sequence
  Synthetic
ttgtgagaaagaagtatatagaat

SEQ ID NO: 1050 - DNA, 24 bp, Artificial Sequence
  Synthetic
gtaagtttagagttatagagttta

SEQ ID NO: 1051 - DNA, 24 bp, Artificial Sequence
  Synthetic
gatagatagataagttaattgaag

SEQ ID NO: 1052 - DNA, 24 bp, Artificial Sequence
  Synthetic
agagatgattgtttatgtattatg

SEQ ID NO: 1053 - DNA, 24 bp, Artificial Sequence
  Synthetic
aaagttaagaaattgtagtgatag

SEQ ID NO: 1054 - DNA, 24 bp, Artificial Sequence
  Synthetic
tttgatattgtttgtgagtgtata

SEQ ID NO: 1055 - DNA, 24 bp, Artificial Sequence
  Synthetic
atttgtagaaagttgttatgagtt

SEQ ID NO: 1056 - DNA, 24 bp, Artificial Sequence
  Synthetic
gatttgagtaagtttatagatgaa

SEQ ID NO: 1057 - DNA, 24 bp, Artificial Sequence
  Synthetic
aagataaagtgagttgatttagat

SEQ ID NO: 1058 - DNA, 24 bp, Artificial Sequence
  Synthetic
gatattgtaagatatgttgtaaag

SEQ ID NO: 1059 - DNA, 24 bp, Artificial Sequence
  Synthetic
gtaagagtgtattgtaagttaatt

SEQ ID NO: 1060 - DNA, 24 bp, Artificial Sequence
  Synthetic
gtgtgattagtaatgaagtattta

SEQ ID NO: 1061 - DNA, 24 bp, Artificial Sequence
  Synthetic
gtaagaaagattaagtgttagtaa

SEQ ID NO: 1062 - DNA, 24 bp, Artificial Sequence
  Synthetic
agtagaaagttgaaattgattatg

SEQ ID NO: 1063 - DNA, 24 bp, Artificial Sequence
  Synthetic
taagagaagttgagtaatgtattt

SEQ ID NO: 1064 - DNA, 24 bp, Artificial Sequence
  Synthetic
gttaagaaatagtagataagtgaa

SEQ ID NO: 1065 - DNA, 24 bp, Artificial Sequence
  Synthetic
taagtaaattgaaagtgtatagtg

SEQ ID NO: 1066 - DNA, 24 bp, Artificial Sequence
  Synthetic
aagatgtatgtttattgttgtgta

SEQ ID NO: 1067 - DNA, 24 bp, Artificial Sequence
  Synthetic
atttagaatatagtgaagagatag

SEQ ID NO: 1068 - DNA, 24 bp, Artificial Sequence
  Synthetic
gttatgaaagagtatgtgttaaat

SEQ ID NO: 1069 - DNA, 24 bp, Artificial Sequence
  Synthetic
tattatgtgaagaagaatgattag

SEQ ID NO: 1070 - DNA, 24 bp, Artificial Sequence
  Synthetic
taataagttgaagagaattgttgt

SEQ ID NO: 1071 - DNA, 24 bp, Artificial Sequence
  Synthetic
tgatgtttgatgtaattgttaaag

SEQ ID NO: 1072 - DNA, 24 bp, Artificial Sequence
  Synthetic
gtgaaagatttgagtttgtataat

SEQ ID NO: 1073 - DNA, 24 bp, Artificial Sequence
  Synthetic
agagaatatagattgagatttgtt

SEQ ID NO: 1074 - DNA, 24 bp, Artificial Sequence
  Synthetic
tttgagatgtgatgataaagttaa

SEQ ID NO: 1075 - DNA, 24 bp, Artificial Sequence
  Synthetic
gttgtaaattgtagtaaagaagta

SEQ ID NO: 1076 - DNA, 24 bp, Artificial Sequence
  Synthetic
gtgttatgatgttgtttgtattat

SEQ ID NO: 1077 - DNA, 24 bp, Artificial Sequence
  Synthetic
attattgtgtagatgtattaagag

SEQ ID NO: 1078 - DNA, 24 bp, Artificial Sequence
  Synthetic
gttagaaagatttagaagttagtt

SEQ ID NO: 1079 - DNA, 24 bp, Artificial Sequence
  Synthetic
ttgtgtattaagagagtgaaatat

SEQ ID NO: 1080 - DNA, 24 bp, Artificial Sequence
  Synthetic
gtttaagatagaaagagtgattta

SEQ ID NO: 1081 - DNA, 24 bp, Artificial Sequence
  Synthetic
aatgagaaatagatagttattgtg

SEQ ID NO: 1082 - DNA, 24 bp, Artificial Sequence
  Synthetic
tgaattgaataagaatttgttgtg

SEQ ID NO: 1083 - DNA, 24 bp, Artificial Sequence
  Synthetic
aataagattgaattagtgagtaag

SEQ ID NO: 1084 - DNA, 24 bp, Artificial Sequence
  Synthetic
aatgtttgagagatttagtaaaga

SEQ ID NO: 1085 - DNA, 24 bp, Artificial Sequence
  Synthetic
agtttagaatagaaatgtgtttga

SEQ ID NO: 1086 - DNA, 24 bp, Artificial Sequence
  Synthetic
tataagtaagtgttaagatttgag

SEQ ID NO: 1087 - DNA, 24 bp, Artificial Sequence
  Synthetic
gtagtgaataagttagtgttaata

SEQ ID NO: 1088 - DNA, 24 bp, Artificial Sequence
  Synthetic
aagtgtgttaaagtaaatgtagat

SEQ ID NO: 1089 - DNA, 24 bp, Artificial Sequence
  Synthetic
agagatgtttatgttgtgaattaa

SEQ ID NO: 1090 - DNA, 24 bp, Artificial Sequence
  Synthetic
agttgaatattgatgataagaaga

SEQ ID NO: 1091 - DNA, 24 bp, Artificial Sequence
  Synthetic
tgaatgtgagatgtttagaataat

SEQ ID NO: 1092 - DNA, 24 bp, Artificial Sequence
  Synthetic
aataatgatgtaagtttgagtttg

SEQ ID NO: 1093 - DNA, 24 bp, Artificial Sequence
  Synthetic
aaagagtgaatagaaataagagaa

SEQ ID NO: 1094 - DNA, 24 bp, Artificial Sequence
  Synthetic
aataaagttattgagagagtttag

SEQ ID NO: 1095 - DNA, 24 bp, Artificial Sequence
  Synthetic
agtagtgttgtagtttagtatata

SEQ ID NO: 1096 - DNA, 24 bp, Artificial Sequence
  Synthetic
gtaagaatgtattagatatttgtg

SEQ ID NO: 1097 - DNA, 24 bp, Artificial Sequence
  Synthetic
gataaatgtttgataaagtagttg

SEQ ID NO: 1098 - DNA, 24 bp, Artificial Sequence
  Synthetic
atagtatgtatgtgtgaagattta

SEQ ID NO: 1099 - DNA, 24 bp, Artificial Sequence
  Synthetic
atgaatgtagagtgattagtttaa

SEQ ID NO: 1100 - DNA, 24 bp, Artificial Sequence
  Synthetic
gtagtatttagtgatgtaagaata

SEQ ID NO: 1101 - DNA, 24 bp, Artificial Sequence
  Synthetic
agaattgtattgaagaagaatatg

SEQ ID NO: 1102 - DNA, 24 bp, Artificial Sequence
  Synthetic
tttatagaattgagagaagttaag

SEQ ID NO: 1103 - DNA, 24 bp, Artificial Sequence
  Synthetic
aaagtagtagagatttgagaatta

SEQ ID NO: 1104 - DNA, 24 bp, Artificial Sequence
  Synthetic
tttaaagaaagtattgtaagagtg

SEQ ID NO: 1105 - DNA, 24 bp, Artificial Sequence
  Synthetic
aaattgagaaagtgaatgaagttt

SEQ ID NO: 1106 - DNA, 24 bp, Artificial Sequence
  Synthetic
aagaaataagtatgatagtagtag

SEQ ID NO: 1107 - DNA, 24 bp, Artificial Sequence
  Synthetic
atttgaattgtattgtagtttgtg

SEQ ID NO: 1108 - DNA, 24 bp, Artificial Sequence
  Synthetic
aagagaataatgtagagatataag

SEQ ID NO: 1109 - DNA, 24 bp, Artificial Sequence
  Synthetic
tgtgtaatagttgttaatgagtaa

SEQ ID NO: 1110 - DNA, 24 bp, Artificial Sequence
  Synthetic
tatagttgtagtttagatgaatgt

SEQ ID NO: 1111 - DNA, 24 bp, Artificial Sequence
  Synthetic
attgtgttagaatgatgttaatag

SEQ ID NO: 1112 - DNA, 24 bp, Artificial Sequence
  Synthetic
gtttgtatagtatttgattgatgt

SEQ ID NO: 1113 - DNA, 24 bp, Artificial Sequence
  Synthetic
agagtaaagtatgagttatgaata

SEQ ID NO: 1114 - DNA, 24 bp, Artificial Sequence
  Synthetic
gaaagtttaagtgatgtatattgt

SEQ ID NO: 1115 - DNA, 24 bp, Artificial Sequence
  Synthetic
ttaaatgataaagagtagtgaagt

SEQ ID NO: 1116 - DNA, 24 bp, Artificial Sequence
  Synthetic
ttaaatgtgtgagaagatgaataa

SEQ ID NO: 1117 - DNA, 24 bp, Artificial Sequence
  Synthetic
atttgtataaagtgaagaagagaa

SEQ ID NO: 1118 - DNA, 24 bp, Artificial Sequence
  Synthetic
tgattagtatttgtgaagagattt

SEQ ID NO: 1119 - DNA, 24 bp, Artificial Sequence
  Synthetic
tttgaatgaaattgatgatagatg

SEQ ID NO: 1120 - DNA, 24 bp, Artificial Sequence
  Synthetic
agagtaagattaagaataagaaag

SEQ ID NO: 1121 - DNA, 24 bp, Artificial Sequence
  Synthetic
attgaattgagaagtgaagtaaat

SEQ ID NO: 1122 - DNA, 24 bp, Artificial Sequence
  Synthetic
tttagagaagtattgtttgaaaga

SEQ ID NO: 1123 - DNA, 24 bp, Artificial Sequence
  Synthetic
taaagtgaaagatttgaaatgatg

SEQ ID NO: 1124 - DNA, 24 bp, Artificial Sequence
  Synthetic
gaaagttagagaaatgtagaaatt

SEQ ID NO: 1125 - DNA, 24 bp, Artificial Sequence
  Synthetic
gtgaataatgaagaagttatgtta

SEQ ID NO: 1126 - DNA, 24 bp, Artificial Sequence
  Synthetic
ttgtgaataaagtagatgtgttat

SEQ ID NO: 1127 - DNA, 24 bp, Artificial Sequence
  Synthetic
ttatatgatatgagtttgtgttga

SEQ ID NO: 1128 - DNA, 24 bp, Artificial Sequence
  Synthetic
ttgatttgtgtgagtattagttat

SEQ ID NO: 1129 - DNA, 24 bp, Artificial Sequence
  Synthetic
aaagtgattaagttagtttgagat

SEQ ID NO: 1130 - DNA, 24 bp, Artificial Sequence
  Synthetic
ttgtatttgtataatgttgaagag

SEQ ID NO: 1131 - DNA, 24 bp, Artificial Sequence
  Synthetic
gtttgaaattagtgtgagaaatat

SEQ ID NO: 1132 - DNA, 24 bp, Artificial Sequence
  Synthetic
aatgttgagattgataatgttgaa

SEQ ID NO: 1133 - DNA, 24 bp, Artificial Sequence
  Synthetic
tagtagtagtattgttgtaataag

SEQ ID NO: 1134 - DNA, 24 bp, Artificial Sequence
  Synthetic
gttgtaatttgagtgttagttatt

SEQ ID NO: 1135 - DNA, 24 bp, Artificial Sequence
  Synthetic
tgaatatgatagttagtaattgtg

SEQ ID NO: 1136 - DNA, 24 bp, Artificial Sequence
  Synthetic
tgatagtatgtttgtgattaaaga

SEQ ID NO: 1137 - DNA, 24 bp, Artificial Sequence
  Synthetic
gatgtataaagagtatgttataag

SEQ ID NO: 1138 - DNA, 24 bp, Artificial Sequence
  Synthetic
agtgagatttagaagatgttatta

SEQ ID NO: 1139 - DNA, 24 bp, Artificial Sequence
  Synthetic
atgagaatttgttaaagagaaagt

SEQ ID NO: 1140 - DNA, 24 bp, Artificial Sequence
  Synthetic
aaagaattagtatgatagatgaga

SEQ ID NO: 1141 - DNA, 24 bp, Artificial Sequence
  Synthetic
tagagttgtatagtttatagttga

SEQ ID NO: 1142 - DNA, 24 bp, Artificial Sequence
  Synthetic
gtagaatgattgtttagaagattt

SEQ ID NO: 1143 - DNA, 24 bp, Artificial Sequence
  Synthetic
gtttatgtttgagaagagttattt

SEQ ID NO: 1144 - DNA, 24 bp, Artificial Sequence
  Synthetic
tagaagtttgaaagttattgattg

SEQ ID NO: 1145 - DNA, 24 bp, Artificial Sequence
  Synthetic
gatgaagagtatttgttatatgta

SEQ ID NO: 1146 - DNA, 24 bp, Artificial Sequence
  Synthetic
gatgaatatagtaagtattgagta

SEQ ID NO: 1147 - DNA, 24 bp, Artificial Sequence
  Synthetic
tagtgatgaaatttgagatagata

SEQ ID NO: 1148 - DNA, 24 bp, Artificial Sequence
  Synthetic
gaaagaaattgaagagtttgatat

SEQ ID NO: 1149 - DNA, 24 bp, Artificial Sequence
  Synthetic
atttgagtatttgtgtattgaatg

SEQ ID NO: 1150 - DNA, 24 bp, Artificial Sequence
  Synthetic
atgagttgaaatttgaagtattgt

SEQ ID NO: 1151 - DNA, 24 bp, Artificial Sequence
  Synthetic
ttaatagtgagagagtatatgtaa

SEQ ID NO: 1152 - DNA, 24 bp, Artificial Sequence
  Synthetic
attaagagagtgagtaaatgtaaa

SEQ ID NO: 1153 - DNA, 24 bp, Artificial Sequence
  Synthetic
aagaatagatgagattagaaatag

SEQ ID NO: 1154 - DNA, 24 bp, Artificial Sequence
  Synthetic
agtttaaagagttagaattgaaag

SEQ ID NO: 1155 - DNA, 24 bp, Artificial Sequence
  Synthetic
gtaagatttgttgaataaagaaga

SEQ ID NO: 1156 - DNA, 24 bp, Artificial Sequence
  Synthetic
agagaaagaagttaaagtgatatt

SEQ ID NO: 1157 - DNA, 24 bp, Artificial Sequence
  Synthetic
taatagagaagagatgtatgaata

SEQ ID NO: 1158 - DNA, 24 bp, Artificial Sequence
  Synthetic
ttattagtgataagtgaagtttag

SEQ ID NO: 1159 - DNA, 24 bp, Artificial Sequence
  Synthetic
ataatgtaaagatgagtttatgag

SEQ ID NO: 1160 - DNA, 24 bp, Artificial Sequence
  Synthetic
ttgatttgagagttgataagattt

SEQ ID NO: 1161 - DNA, 24 bp, Artificial Sequence
  Synthetic
atgattattgtgtgtagaattaga

SEQ ID NO: 1162 - DNA, 24 bp, Artificial Sequence
  Synthetic
tataaagatatagtagatgatgtg

SEQ ID NO: 1163 - DNA, 24 bp, Artificial Sequence
  Synthetic
tttagttgagatgaagttattaga

SEQ ID NO: 1164 - DNA, 24 bp, Artificial Sequence
  Synthetic
attgaattgatatagtgtaaagtg

SEQ ID NO: 1165 - DNA, 24 bp, Artificial Sequence
  Synthetic
gaagaaagattattgtattgagtt

SEQ ID NO: 1166 - DNA, 24 bp, Artificial Sequence
  Synthetic
attgagtgtagtgatttagaaata

SEQ ID NO: 1167 - DNA, 24 bp, Artificial Sequence
  Synthetic
aataaagtgtttaagagtagagta

SEQ ID NO: 1168 - DNA, 24 bp, Artificial Sequence
  Synthetic
gtagagataattgatgtgtaattt

SEQ ID NO: 1169 - DNA, 19 bp, Artificial Sequence
  Synthetic
tgatcgtagctacgccgcg

SEQ ID NO: 1170 - DNA, 16 bp, Artificial Sequence
  Synthetic
cgtacgattgcaacgt

SEQ ID NO: 1171-1172 - skipped (2 entries)

SEQ ID NO: 1173 - DNA, 24 bp, Artificial Sequence
  Synthetic
gatttgtattgattgagattaaag

SEQ ID NO: 1174 - DNA, 24 bp, Artificial Sequence
  Synthetic
tgattgtagtatgtattgataaag

SEQ ID NO: 1175 - DNA, 24 bp, Artificial Sequence
  Synthetic
gattgtaagatttgataaagtgta

SEQ ID NO: 1176 - DNA, 24 bp, Artificial Sequence
  Synthetic
gatttgaagattattggtaatgta

SEQ ID NO: 1177 - DNA, 24 bp, Artificial Sequence
  Synthetic
gattgattattgtgatttgaattg

SEQ ID NO: 1178 - DNA, 24 bp, Artificial Sequence
  Synthetic
gatttgattgtaaaagattgttga

SEQ ID NO: 1179 - DNA, 24 bp, Artificial Sequence
  Synthetic
attggtaaattggtaaatgaattg

SEQ ID NO: 1180 - DNA, 24 bp, Artificial Sequence
  Synthetic
attggatttgataaaggtaaatga

SEQ ID NO: 1181 - DNA, 24 bp, Artificial Sequence
  Synthetic
gtaagtaatgaatgtaaaaggatt

SEQ ID NO: 1182 - DNA, 24 bp, Artificial Sequence
  Synthetic
gattgattgattgattgatttgat

SEQ ID NO: 1183 - DNA, 24 bp, Artificial Sequence
  Synthetic
tgatgattaaagaaagtgattgat

SEQ ID NO: 1184 - DNA, 24 bp, Artificial Sequence
  Synthetic
aaaggatttgattgataaagtgat

SEQ ID NO: 1185 - DNA, 24 bp, Artificial Sequence
  Synthetic
tgtagatttgtatgtatgtatgat

SEQ ID NO: 1186 - DNA, 24 bp, Artificial Sequence
  Synthetic
gatttgataaagaaaggattgatt

SEQ ID NO: 1187 - DNA, 24 bp, Artificial Sequence
  Synthetic
gattaaagtgattgatgatttgta

SEQ ID NO: 1188 - DNA, 24 bp, Artificial Sequence
  Synthetic
aaagaaagaaagaaagaaagtgta

SEQ ID NO: 1189 - DNA, 24 bp, Artificial Sequence
  Synthetic
tgtaaaaggattgatttgtatgta

SEQ ID NO: 1190 - DNA, 24 bp, Artificial Sequence
  Synthetic
aaagtgtagattgattaaagaaag

SEQ ID NO: 1191 - DNA, 24 bp, Artificial Sequence
  Synthetic
aaagttgattgattgaaaaggtat

SEQ ID NO: 1192 - DNA, 24 bp, Artificial Sequence
  Synthetic
ttgattgagattgattttgagtat

SEQ ID NO: 1193 - DNA, 24 bp, Artificial Sequence
  Synthetic
tgaattgatgaatgaatgaagtat

SEQ ID NO: 1194 - DNA, 24 bp, Artificial Sequence
  Synthetic
gtaatgaagtatgtatgtaagtaa

SEQ ID NO: 1195 - DNA, 24 bp, Artificial Sequence
  Synthetic
tgatgatttgaatgaagattgatt

SEQ ID NO: 1196 - DNA, 24 bp, Artificial Sequence
  Synthetic
tgataaagtgataaaggattaaag

SEQ ID NO: 1197 - DNA, 24 bp, Artificial Sequence
  Synthetic
tgatttgagtatttgagattttga

SEQ ID NO: 1198 - DNA, 24 bp, Artificial Sequence
  Synthetic
tgtagtaagattgattaaaggtaa

SEQ ID NO: 1199 - DNA, 24 bp, Artificial Sequence
  Synthetic
gtataaaggattgattttgaaaag

SEQ ID NO: 1200 - DNA, 24 bp, Artificial Sequence
  Synthetic
gtatttgagtaagtaattgattga

SEQ ID NO: 1201 - DNA, 24 bp, Artificial Sequence
  Synthetic
gtaaaaagttgagtattgaaaaag

SEQ ID NO: 1202 - DNA, 24 bp, Artificial Sequence
  Synthetic
gatttgataaaggatttgtattga

SEQ ID NO: 1203 - DNA, 24 bp, Artificial Sequence
  Synthetic
gattgtattgaagtattgtaaaag

SEQ ID NO: 1204 - DNA, 24 bp, Artificial Sequence
  Synthetic
tgatgattttgatgaaaaagttga

SEQ ID NO: 1205 - DNA, 24 bp, Artificial Sequence
  Synthetic
tgatttgagattaaagaaaggatt

SEQ ID NO: 1206 - DNA, 24 bp, Artificial Sequence
  Synthetic
tgattgaattgagtaaaaaggatt

SEQ ID NO: 1207 - DNA, 24 bp, Artificial Sequence
  Synthetic
aaagtgtaaaaggatttgatgtat

SEQ ID NO: 1208 - DNA, 24 bp, Artificial Sequence
  Synthetic
aaaggtatttgagatttgattgaa

SEQ ID NO: 1209 - DNA, 24 bp, Artificial Sequence
  Synthetic
aaagttgagatttgaatgattgaa

SEQ ID NO: 1210 - DNA, 24 bp, Artificial Sequence
  Synthetic
tgtattgaaaaggtatgatttgaa

SEQ ID NO: 1211 - DNA, 24 bp, Artificial Sequence
  Synthetic
gtattgtattgaaaaggtaattga

SEQ ID NO: 1212 - DNA, 24 bp, Artificial Sequence
  Synthetic
ttgagtaatgataaagtgaagatt

SEQ ID NO: 1213 - DNA, 24 bp, Artificial Sequence
  Synthetic
tgaagatttgaagtaattgaaaag

SEQ ID NO: 1214 - DNA, 24 bp, Artificial Sequence
  Synthetic
tgaaaaagtgtagattttgagtaa

SEQ ID NO: 1215 - DNA, 24 bp, Artificial Sequence
  Synthetic
tgtatgaatgaagatttgattgta

SEQ ID NO: 1216 - DNA, 24 bp, Artificial Sequence
  Synthetic
aaagttgagtattgatttgaaaag

SEQ ID NO: 1217 - DNA, 24 bp, Artificial Sequence
  Synthetic
gatttgtagatttgtattgagatt

SEQ ID NO: 1218 - DNA, 24 bp, Artificial Sequence
  Synthetic
aaagaaaggatttgtagtaagatt

SEQ ID NO: 1219 - DNA, 24 bp, Artificial Sequence
  Synthetic
gtaaaaagaaaggtataaaggtaa

SEQ ID NO: 1220 - DNA, 24 bp, Artificial Sequence
  Synthetic
gattaaagttgattgaaaagtgaa

SEQ ID NO: 1221 - DNA, 24 bp, Artificial Sequence
  Synthetic
tgaaaaaggtaattgatgtatgaa

SEQ ID NO: 1222 - DNA, 24 bp, Artificial Sequence
  Synthetic
aaaggattaaagtgaagtaattga

SEQ ID NO: 1223 - DNA, 24 bp, Artificial Sequence
  Synthetic
atgaattggtatgtatatgaatga

SEQ ID NO: 1224 - DNA, 24 bp, Artificial Sequence
  Synthetic
tgaaatgaatgaatgatgaaattg

SEQ ID NO: 1225 - DNA, 24 bp, Artificial Sequence
  Synthetic
attgattgtgaatgaaatgaattg

SEQ ID NO: 1226 - DNA, 24 bp, Artificial Sequence
  Synthetic
attgaaagatgaaaagatgaaaag

SEQ ID NO: 1227 - DNA, 24 bp, Artificial Sequence
  Synthetic
attgttgaaaagtgtaatgattga

SEQ ID NO: 1228 - DNA, 24 bp, Artificial Sequence
  Synthetic
atgatgtaatgaaaagattgtgta

SEQ ID NO: 1229 - DNA, 24 bp, Artificial Sequence
  Synthetic
aaagattgaaagatgatgtaattg

SEQ ID NO: 1230 - DNA, 24 bp, Artificial Sequence
  Synthetic
attgatgagtatattgtgtagtaa

SEQ ID NO: 1231 - DNA, 24 bp, Artificial Sequence
  Synthetic
aaagattgtgtaattgatgatgaa

SEQ ID NO: 1232 - DNA, 24 bp, Artificial Sequence
  Synthetic
aaaggtatattgtgtaatgagtaa

SEQ ID NO: 1233 - DNA, 24 bp, Artificial Sequence
  Synthetic
tgtaatgagtattgtaattgaaag

SEQ ID NO: 1234 - DNA, 24 bp, Artificial Sequence
  Synthetic
gtataaagaaagattggtaaatga

SEQ ID NO: 1235 - DNA, 24 bp, Artificial Sequence
  Synthetic
ttgagtaattgaattgtgaaatga

SEQ ID NO: 1236 - DNA, 24 bp, Artificial Sequence
  Synthetic
tgtattgaatgaattgttgatgta

SEQ ID NO: 1237 - DNA, 24 bp, Artificial Sequence
  Synthetic
tgtaattggtaaatgagtaaaaag

SEQ ID NO: 1238 - DNA, 24 bp, Artificial Sequence
  Synthetic
tgaatgaaattgatgagtataaag

SEQ ID NO: 1239 - DNA, 24 bp, Artificial Sequence
  Synthetic
gtaagtaaattgaaagattgatga

SEQ ID NO: 1240 - DNA, 24 bp, Artificial Sequence
  Synthetic
gtaaatgatgatattggtatattg

SEQ ID NO: 1241 - DNA, 24 bp, Artificial Sequence
  Synthetic
attgttgatgattgattgaaatga

SEQ ID NO: 1242 - DNA, 24 bp, Artificial Sequence
  Synthetic
attgtgaagtataaagatgattga

SEQ ID NO: 1243 - DNA, 24 bp, Artificial Sequence
  Synthetic
atgaaaagttgagtaaattgtgat

SEQ ID NO: 1244 - DNA, 24 bp, Artificial Sequence
  Synthetic
atgaattgaaagtgattgaaaaag

SEQ ID NO: 1245 - DNA, 24 bp, Artificial Sequence
  Synthetic
gtaaattgatgaaaagttgatgat

SEQ ID NO: 1246 - DNA, 24 bp, Artificial Sequence
  Synthetic
aaagtgatgtatatgagtaaattg

SEQ ID NO: 1247 - DNA, 24 bp, Artificial Sequence
  Synthetic
gtaatgataaagatgatgatattg

SEQ ID NO: 1248 - DNA, 24 bp, Artificial Sequence
  Synthetic
ttgaaaagattggtaatgatatga

SEQ ID NO: 1249 - DNA, 24 bp, Artificial Sequence
  Synthetic
aaagtgaaaaagattgattgatga

SEQ ID NO: 1250 - DNA, 24 bp, Artificial Sequence
  Synthetic
attgatgagattgattattgtgta

SEQ ID NO: 1251 - DNA, 24 bp, Artificial Sequence
  Synthetic
atgagattattggatttgtagatt

SEQ ID NO: 1252 - DNA, 24 bp, Artificial Sequence
  Synthetic
tgaagattatgaattggtaagatt

SEQ ID NO: 1253 - DNA, 24 bp, Artificial Sequence
  Synthetic
attggattatgagattatgattga

SEQ ID NO: 1254 - DNA, 24 bp, Artificial Sequence
  Synthetic
attgttgaattggattaaagatga

SEQ ID NO: 1255 - DNA, 24 bp, Artificial Sequence
  Synthetic
aaagatgagtaagtaaattggatt

SEQ ID NO: 1256 - DNA, 24 bp, Artificial Sequence
  Synthetic
aaaggtaagattattgatgaaaag

SEQ ID NO: 1257 - DNA, 24 bp, Artificial Sequence
  Synthetic
attgatgagattaaagttgaattg

SEQ ID NO: 1258 - DNA, 24 bp, Artificial Sequence
  Synthetic
gattattggattatgaaaaggatt

SEQ ID NO: 1259 - DNA, 24 bp, Artificial Sequence
  Synthetic
gatttgtaattgttgagtaaatga

SEQ ID NO: 1260 - DNA, 24 bp, Artificial Sequence
  Synthetic
aaagaaagattgttgagattatga

SEQ ID NO: 1261 - DNA, 24 bp, Artificial Sequence
  Synthetic
gtataaaggattttgaattgatga

SEQ ID NO: 1262 - DNA, 24 bp, Artificial Sequence
  Synthetic
ttgagattgtaaatgaattgttga

SEQ ID NO: 1263 - DNA, 24 bp, Artificial Sequence
  Synthetic
gtatattgattgtgtaatgaaaag

SEQ ID NO: 1264 - DNA, 24 bp, Artificial Sequence
  Synthetic
tgatatgaattggattattggtat

SEQ ID NO: 1265 - DNA, 24 bp, Artificial Sequence
  Synthetic
atgaatgatgaatgatgattattg

SEQ ID NO: 1266 - DNA, 24 bp, Artificial Sequence
  Synthetic
atgaattgattggattgtaatgat

SEQ ID NO: 1267 - DNA, 24 bp, Artificial Sequence
  Synthetic
gattgtaattgagtaaattgatga

SEQ ID NO: 1268 - DNA, 24 bp, Artificial Sequence
  Synthetic
gattattggattaaaggtaaatga

SEQ ID NO: 1269 - DNA, 24 bp, Artificial Sequence
  Synthetic
attgttgaattgatgagatttgat

SEQ ID NO: 1270 - DNA, 24 bp, Artificial Sequence
  Synthetic
gattatgagtaaattgattgtgat

SEQ ID NO: 1271 - DNA, 24 bp, Artificial Sequence
  Synthetic
gattattgttgatgaatgatattg

SEQ ID NO: 1272 - DNA, 24 bp, Artificial Sequence
  Synthetic
tgtaaaagattgaaaggtatgatt

SEQ ID NO: 1273 - DNA, 24 bp, Artificial Sequence
  Synthetic
gtatttagatgagtttgttagatt

SEQ ID NO: 1274 - DNA, 24 bp, Artificial Sequence
  Synthetic
tgaagttatgtaatagaaagtgat

SEQ ID NO: 1275 - DNA, 24 bp, Artificial Sequence
  Synthetic
gtatgtattgtatgtagttaattg

SEQ ID NO: 1276 - DNA, 24 bp, Artificial Sequence
  Synthetic
tgatatagatagttagatagatag

SEQ ID NO: 1277 - DNA, 24 bp, Artificial Sequence
  Synthetic
atgatgatgtattgtagttatgaa

SEQ ID NO: 1278 - DNA, 24 bp, Artificial Sequence
  Synthetic
ttagtgaatgtattagttgatgta

SEQ ID NO: 1279 - DNA, 24 bp, Artificial Sequence
  Synthetic
gttagttagattattgttagttag

SEQ ID NO: 1280 - DNA, 24 bp, Artificial Sequence
  Synthetic
gttaattgtgtagtttgttattga

SEQ ID NO: 1281 - DNA, 24 bp, Artificial Sequence
  Synthetic
gttatgaaatagtgatattgttag

SEQ ID NO: 1282 - DNA, 24 bp, Artificial Sequence
  Synthetic
attgttagaaagtgtagattaaag

SEQ ID NO: 1283 - DNA, 24 bp, Artificial Sequence
  Synthetic
atgagtatgttattagtgtatgta

SEQ ID NO: 1284 - DNA, 24 bp, Artificial Sequence
  Synthetic
tgtaatagtgaagttagattgtat

SEQ ID NO: 1285 - DNA, 24 bp, Artificial Sequence
  Synthetic
attgatagatgattagttagttga

SEQ ID NO: 1286 - DNA, 24 bp, Artificial Sequence
  Synthetic
atgagtttgtttatgagattaaag

SEQ ID NO: 1287 - DNA, 24 bp, Artificial Sequence
  Synthetic
tgatgtttgattatgatgtagtat

SEQ ID NO: 1288 - DNA, 24 bp, Artificial Sequence
  Synthetic
atgagttagttatgaattagatga

SEQ ID NO: 1289 - DNA, 24 bp, Artificial Sequence
  Synthetic
attgttagtgatgttagtaattag

SEQ ID NO: 1290 - DNA, 24 bp, Artificial Sequence
  Synthetic
tgatgtaagtattgatgttagttt

SEQ ID NO: 1291 - DNA, 24 bp, Artificial Sequence
  Synthetic
gattgtaaatagaaagtgaagtaa

SEQ ID NO: 1292 - DNA, 24 bp, Artificial Sequence
  Synthetic
attgtgtatgaagtattgtatgat

SEQ ID NO: 1293 - DNA, 24 bp, Artificial Sequence
  Synthetic
atagtgatgttatgaagattgtta

SEQ ID NO: 1294 - DNA, 24 bp, Artificial Sequence
  Synthetic
ttagatgaattgtgaagtatttag

SEQ ID NO: 1295 - DNA, 24 bp, Artificial Sequence
  Synthetic
gtaagttatgattgatgttatgaa

SEQ ID NO: 1296 - DNA, 24 bp, Artificial Sequence
  Synthetic
gtattgatgtttaaagtgtaatag

SEQ ID NO: 1297 - DNA, 24 bp, Artificial Sequence
  Synthetic
gattgtaagtaagattgtatattg

SEQ ID NO: 1298 - DNA, 24 bp, Artificial Sequence
  Synthetic
gtttgtatttagatgaatagaaag

SEQ ID NO: 1299 - DNA, 24 bp, Artificial Sequence
  Synthetic
gtttgatttgtaatagtgattgta

SEQ ID NO: 1300 - DNA, 24 bp, Artificial Sequence
  Synthetic
tgtatgtagtatttagaaagatga

SEQ ID NO: 1301 - DNA, 24 bp, Artificial Sequence
  Synthetic
atgaattgtgataaagaaagttag

SEQ ID NO: 1302 - DNA, 24 bp, Artificial Sequence
  Synthetic
ttagtgtagtaagtttaaagtgta

SEQ ID NO: 1303 - DNA, 24 bp, Artificial Sequence
  Synthetic
gtatgattgtttgtaattagtgat

SEQ ID NO: 1304 - DNA, 24 bp, Artificial Sequence
  Synthetic
gtttaaagttagttgagttagtat

SEQ ID NO: 1305 - DNA, 24 bp, Artificial Sequence
  Synthetic
atagtgtatgtagattatgagatt

SEQ ID NO: 1306 - DNA, 24 bp, Artificial Sequence
  Synthetic
ttgaatgattagttgagtatgatt

SEQ ID NO: 1307 - DNA, 24 bp, Artificial Sequence
  Synthetic
gtatgtaagttagtatgatttgaa

SEQ ID NO: 1308 - DNA, 24 bp, Artificial Sequence
  Synthetic
tgtagtatattgttgaattgtgat

SEQ ID NO: 1309 - DNA, 24 bp, Artificial Sequence
  Synthetic
atagtgattgtatgtatgataaag

SEQ ID NO: 1310 - DNA, 24 bp, Artificial Sequence
  Synthetic
ttagtgattgatgtatattgaaag

SEQ ID NO: 1311 - DNA, 24 bp, Artificial Sequence
  Synthetic
gtaagattatgagttatgatgtaa

SEQ ID NO: 1312 - DNA, 24 bp, Artificial Sequence
  Synthetic
gttatgaaattgttagtgtagatt

SEQ ID NO: 1313 - DNA, 24 bp, Artificial Sequence
  Synthetic
gttagatttgtagtttaaagatag

SEQ ID NO: 1314 - DNA, 24 bp, Artificial Sequence
  Synthetic
ttagtgattgaaatgatgtagatt

SEQ ID NO: 1315 - DNA, 24 bp, Artificial Sequence
  Synthetic
aaagtgtagttattagttagttag

SEQ ID NO: 1316 - DNA, 24 bp, Artificial Sequence
  Synthetic
aaagaaagtgtatgatgttattag

SEQ ID NO: 1317 - DNA, 24 bp, Artificial Sequence
  Synthetic
gattgtatattgtgtatgatgatt

SEQ ID NO: 1318 - DNA, 24 bp, Artificial Sequence
  Synthetic
ttgagattgttatgatatgagtat

SEQ ID NO: 1319 - DNA, 24 bp, Artificial Sequence
  Synthetic
atgagtatgattgttatgatgttt

SEQ ID NO: 1320 - DNA, 24 bp, Artificial Sequence
  Synthetic
tgatttagtgaaattgtgtattag

SEQ ID NO: 1321 - DNA, 24 bp, Artificial Sequence
  Synthetic
tgaatgtatgtagtatgtttgtta

SEQ ID NO: 1322 - DNA, 24 bp, Artificial Sequence
  Synthetic
gttagtattgatgattatgagtta

SEQ ID NO: 1323 - DNA, 24 bp, Artificial Sequence
  Synthetic
gtatattgtgatttagttgagatt

SEQ ID NO: 1324 - DNA, 24 bp, Artificial Sequence
  Synthetic
gttagtttaaagttgagattgttt

SEQ ID NO: 1325 - DNA, 24 bp, Artificial Sequence
  Synthetic
gtatattgttagatgagatttgta

SEQ ID NO: 1326 - DNA, 24 bp, Artificial Sequence
  Synthetic
tgatgtatgttagtttatgaatga

SEQ ID NO: 1327 - DNA, 24 bp, Artificial Sequence
  Synthetic
tgtagtatgtaatgtagtatttga

SEQ ID NO: 1328 - DNA, 24 bp, Artificial Sequence
  Synthetic
atgagttatgtattgagttagtat

SEQ ID NO: 1329 - DNA, 24 bp, Artificial Sequence
  Synthetic
tgtatgatgattatagttgagtaa

SEQ ID NO: 1330 - DNA, 24 bp, Artificial Sequence
  Synthetic
attgatgaatgagtttgtataaag

SEQ ID NO: 1331 - DNA, 24 bp, Artificial Sequence
  Synthetic
ttgagtttatgattagaaagaaag

SEQ ID NO: 1332 - DNA, 24 bp, Artificial Sequence
  Synthetic
tgatattgatgagttagtattgaa

SEQ ID NO: 1333 - DNA, 24 bp, Artificial Sequence
  Synthetic
atagaaagtgaaatgagtatgtta

SEQ ID NO: 1334 - DNA, 24 bp, Artificial Sequence
  Synthetic
ttgatgtagatttgatgtatatag

SEQ ID NO: 1335 - DNA, 24 bp, Artificial Sequence
  Synthetic
ttgagattatagtgtagtttatag

SEQ ID NO: 1336 - DNA, 24 bp, Artificial Sequence
  Synthetic
tgatgttagattgtttgattattg

SEQ ID NO: 1337 - DNA, 24 bp, Artificial Sequence
  Synthetic
tgtattagatagtgatttgaatga

SEQ ID NO: 1338 - DNA, 24 bp, Artificial Sequence
  Synthetic
gattatgatgaatgtagtatgtaa

SEQ ID NO: 1339 - DNA, 24 bp, Artificial Sequence
  Synthetic
tgaatgattgatatgaatagtgta

SEQ ID NO: 1340 - DNA, 24 bp, Artificial Sequence
  Synthetic
gtaatgatttagtgtattgagttt

SEQ ID NO: 1341 - DNA, 24 bp, Artificial Sequence
  Synthetic
tgtagtaatgatttgatgataaag

SEQ ID NO: 1342 - DNA, 24 bp, Artificial Sequence
  Synthetic
tgaagattgttattagtgatattg

SEQ ID NO: 1343 - DNA, 24 bp, Artificial Sequence
  Synthetic
gtatttgaatgatgtaatagtgta

SEQ ID NO: 1344 - DNA, 24 bp, Artificial Sequence
  Synthetic
gtatatgatgtattagattgaaag

SEQ ID NO: 1345 - DNA, 24 bp, Artificial Sequence
  Synthetic
aaagttagattgaaagtgataaag

SEQ ID NO: 1346 - DNA, 24 bp, Artificial Sequence
  Synthetic
gtaagatgttgatatagaagatta

SEQ ID NO: 1347 - DNA, 24 bp, Artificial Sequence
  Synthetic
taatatgagatgaaagtgaattag

SEQ ID NO: 1348 - DNA, 24 bp, Artificial Sequence
  Synthetic
ttagtgaagaagtatagtttattg

SEQ ID NO: 1349 - DNA, 24 bp, Artificial Sequence
  Synthetic
gtagttgagaagatagtaattaat

SEQ ID NO: 1350 - DNA, 24 bp, Artificial Sequence
  Synthetic
atgagatgatatttgagaagtaat

SEQ ID NO: 1351 - DNA, 24 bp, Artificial Sequence
  Synthetic
gatgtgaagaagatgaatatatat

SEQ ID NO: 1352 - DNA, 24 bp, Artificial Sequence
  Synthetic
aaagtatagtaagatgtatagtag

SEQ ID NO: 1353 - DNA, 24 bp, Artificial Sequence
  Synthetic
gaagtaatatgagtagttgaatat

SEQ ID NO: 1354 - DNA, 24 bp, Artificial Sequence
  Synthetic
ttgataatgtttgtttgtttgtag

SEQ ID NO: 1355 - DNA, 24 bp, Artificial Sequence
  Synthetic
tgaagaagaaagtataatgatgaa

SEQ ID NO: 1356 - DNA, 24 bp, Artificial Sequence
  Synthetic
gtagattagtttgaagtgaataat

SEQ ID NO: 1357 - DNA, 24 bp, Artificial Sequence
  Synthetic
tatagtagtgaagatgatatatga

SEQ ID NO: 1358 - DNA, 24 bp, Artificial Sequence
  Synthetic
tataatgagttgttagatatgttg

SEQ ID NO: 1359 - DNA, 24 bp, Artificial Sequence
  Synthetic
gttgtgaaattagatgtgaaatat

SEQ ID NO: 1360 - DNA, 24 bp, Artificial Sequence
  Synthetic
taatgttgtgaataatgtagaaag

SEQ ID NO: 1361 - DNA, 24 bp, Artificial Sequence
  Synthetic
gtttatagtgaaatatgaagatag

SEQ ID NO: 1362 - DNA, 24 bp, Artificial Sequence
  Synthetic
attatgaagtaagttaatgagaag

SEQ ID NO: 1363 - DNA, 24 bp, Artificial Sequence
  Synthetic
gatgaaagtaatgtttattgtgaa

SEQ ID NO: 1364 - DNA, 24 bp, Artificial Sequence
  Synthetic
attattgagatgtgaagtttgttt

SEQ ID NO: 1365 - DNA, 24 bp, Artificial Sequence
  Synthetic
tgtagaagatgagatgtataatta

SEQ ID NO: 1366 - DNA, 24 bp, Artificial Sequence
  Synthetic
taatttgagttgtgtatatagtag

SEQ ID NO: 1367 - DNA, 24 bp, Artificial Sequence
  Synthetic
tgatattagtaagaagttgaatag

SEQ ID NO: 1368 - DNA, 24 bp, Artificial Sequence
  Synthetic
gttagttattgagaagtgtatata

SEQ ID NO: 1369 - DNA, 24 bp, Artificial Sequence
  Synthetic
gtagtaatgttaatgaattagtag

SEQ ID NO: 1370 - DNA, 24 bp, Artificial Sequence
  Synthetic
gtttgtttgatgtgattgaataat

SEQ ID NO: 1371 - DNA, 24 bp, Artificial Sequence
  Synthetic
gtaagtagtaatttgaatatgtag

SEQ ID NO: 1372 - DNA, 24 bp, Artificial Sequence
  Synthetic
gtttgaagatatgtttgaagtata

SEQ ID NO: 1373 - DNA, 24 bp, Artificial Sequence
  Synthetic
atgataattgaagatgtaatgttg

SEQ ID NO: 1374 - DNA, 24 bp, Artificial Sequence
  Synthetic
gtagatagtatagttgtaatgtta

SEQ ID NO: 1375 - DNA, 24 bp, Artificial Sequence
  Synthetic
gatgtgaatgtaatatgtttatag

SEQ ID NO: 1376 - DNA, 24 bp, Artificial Sequence
  Synthetic
tgaaattagtttgtaagatgtgta

SEQ ID NO: 1377 - DNA, 24 bp, Artificial Sequence
  Synthetic
tgtagtataaagtatatgaagtag

SEQ ID NO: 1378 - DNA, 24 bp, Artificial Sequence
  Synthetic
atatgttgttgagttgatagtata

SEQ ID NO: 1379 - DNA, 24 bp, Artificial Sequence
  Synthetic
attattgagtagaaagatagaaag

SEQ ID NO: 1380 - DNA, 24 bp, Artificial Sequence
  Synthetic
gttgttgaatattgaatatagttg

SEQ ID NO: 1381 - DNA, 24 bp, Artificial Sequence
  Synthetic
atgagaagttagtaatgtaaatag

SEQ ID NO: 1382 - DNA, 24 bp, Artificial Sequence
  Synthetic
tgaaatgagaagattaatgagttt

SEQ ID NO: 1383 - DNA, 24 bp, Artificial Sequence
  Synthetic
atgtttagtgaaaagttagtattg

SEQ ID NO: 1384 - DNA, 24 bp, Artificial Sequence
  Synthetic
atgttagtgaatagtatagtattg

SEQ ID NO: 1385 - DNA, 22 bp, Artificial Sequence
  Synthetic
atgttagtgaaagttagtattg

SEQ ID NO: 1386 - DNA, 10 bp, Artificial Sequence
  Synthetic
gatttgtatt

SEQ ID NO: 1387 - DNA, 10 bp, Artificial Sequence
  Synthetic
attgataaag

(19) United States
(12) Patent Application Publication
(10) Pub. No.: US 2008/0131875 A1
(43) Pub. Date: Jun. 5, 2008

(54) Multiplex assays

(75) Inventor: Jeff G. Hall, Waunakee, WI (US)

Correspondence Address: Casimir Jones, S.C., 440 Science Drive, Suite 203, Madison, WI, 53711, US

(73) Assignee: Third Wave Technologies, Inc., Madison, WI (US)

(21) Appl. No.: 11/449,463

(22) Filed: Jun. 7, 2006

(51) Int. Cl. C12Q 1/68; C07H 21/04

(52) U.S. Cl. 435/6; 536/23.1

(57) ABSTRACT

The present invention relates to compositions and methods for the detection and characterization of nucleic acid molecules. More particularly, the present invention relates to methods and compositions employing non cross-hybridizing and minimally cross-hybridizing tags on the 5′ ends of invasive cleavage probes.

### FIELD OF THE INVENTION

This invention relates to the use of families of oligonucleotides tags, for example, in the sorting of molecules, identification of target nucleic acid molecules or for analyzing the presence of a mutation or polymorphism at a locus of each target nucleic acid molecule.

### BACKGROUND

With the completion of the nucleic acid sequencing of the human genome, the demand for fast, reliable, cost-effective and user-friendly tests for genomics research and related drug design efforts has greatly increased. A number of institutions are actively mining the available genetic sequence information to identify correlations between genes, gene expression and phenotypes (e.g., disease states, metabolic responses, and the like). These analyses include an attempt to characterize the effect of gene mutations and genetic and gene expression heterogeneity in individuals and populations. Often, it is desirable to look at many different loci and alleles in parallel, generally in a single reaction.

Working in a highly parallel hybridization environment requiring specific hybridization imposes very rigorous selection criteria for the design of families of oligonucleotides that are to be used. The success of these approaches is dependent on the specific hybridization of a probe and its complement. Problems arise as the family of nucleic acid molecules cross-hybridize or hybridize incorrectly to the target sequences. While it is common to obtain incorrect hybridization resulting in false positives or an inability to form hybrids resulting in false negatives, the frequency of such results must be minimized. In order to achieve this goal certain thermodynamic properties of forming nucleic acid hybrids must be considered.

Design of families of oligonucleotide sequences that can be used in multiplexed hybridization reactions includes consideration for the thermodynamic properties of oligonucleotides and duplex formation that will reduce or eliminate cross hybridization behavior within the designed oligonucleotide set.

In the INVADER Assay and other 5′ nuclease assays, one system of multiplexing involved the use of different 5′ arms or “flaps” for different alleles or loci. The use of different flaps is one way of detecting many different sequences in a single “multiplex” reaction. Thus, it is desirable to have a large number of “flap” molecules incorporated into the INVADER Assay, with the “flap” sequences selected such that each flap is highly selective for its own complement sequence.

### SUMMARY OF THE INVENTION

The present invention relates to the use of minimally cross-hybridizing oligonucleotide sequences in the INVADER Assay. The incorporation of these sequences into one of the two oligonucleotides that forms an invasive cleavage structure with a target nucleic acid, and subsequent structure-dependent cleavage of the oligonucleotide comprising the minimally cross-hybridizing sequence provides a way of using the INVADER Assay in massively parallel analysis of multiple genes, e.g., in a gene microarray. The present invention provides, for example, oligonucleotide probes for cleavage in INVADER assays, wherein the oligonucleotide probes comprise an a 5′ portion a minimally cross-hybridizing nucleic acid tag, such that at least a portion of the tag is released when the probe is cleaved.

In some embodiments, the present invention comprises a composition comprising a cleavage structure, said cleavage structure comprising:
a) a target nucleic acid having a first region and a second region, wherein said second region is located adjacent to and downstream of said first region;
b) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and is selected from the group consisting of tag identifiers 1-210
   wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is a base in which:
   (i)
      (a)
         W=one of A, T/U, G, and C,
         X=one of A, T/U, G, and C,
         Y=one of A, T/U, G, and C,
         and each of W, X and Y is selected so as to be different from all of the others of W, X and Y,
      (b) an unselected said base of (i)(a) can be substituted any number of times for any one of W, X and Y, or
   (ii)
      (a)
         W=G or C,
         X=A or T/U,
         Y=A or T/U,
         and X≠Y, and
      (b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
   (II) for any phantom sequence generated from any pair of first and second sequences of the set L₁ and L₂ in length, respectively, by selection from the first and second sequences of identical bases in identical sequence with each other:
      (i) any consecutive sequence of bases in the phantom sequence which is identical to a consecutive sequence of bases in each of the first and second sequences from which it is generated is less than ((¾×L)−1) bases in length;
      (ii) the phantom sequence, if greater than or equal to (⅚×L) in length, contains at least three insertions/deletions or mismatches when compared to the first and second sequences from which it is generated; and
      (iii) the phantom sequence is not greater than or equal to ( 11/12)×L) in length;
      where L=L₁, or if L₁≠L₂, where L is the greater of L₁ and L₂; and
         wherein any base present may be substituted by an analogue thereof; and
c) a second nucleic acid molecule comprising a 3′ portion and a 5′ portion, wherein said 5′ portion is completely complementary to said second region of said target nucleic acid.

In preferred embodiments, the tag identifiers 1-210 are selected from SEQ ID NOS: 1173-1382.

In some embodiments, the composition further comprises a 5′ nuclease. In preferred embodiments, the 5′ nuclease is a FEN-1 nuclease. In particularly preferred embodiments, the FEN-1 nuclease is a thermostable FEN-1 nuclease.

In some embodiments, the present invention provides a method for detecting the presence of a target nucleic acid molecule in a sample, comprising:
a) incubating a sample with a thermostable 5′ nuclease under conditions wherein a cleavage structure is formed, said cleavage structure comprising:
   i) a target nucleic acid having a first region and a second region, wherein said second region is located adjacent to and downstream of said first region;
ii) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and is selected from the group consisting of tag identifiers 1-210
   wherein:
(A) each of 1 to 22 is a 4mer selected from the group of 4mers consisting of WWWW, WWWX, WWWY, WWXW, WWXX, WWXY, WWYW, WWYX, WWYY, WXWW; WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is a base in which:
   (i) (a) W=one of A, T/U, G, and C,
      X=one of A, T/U, G, and C,
      Y=one of A, T/U, G, and C,
      and each of W, X and Y is selected so as to be different from all of the others of W, X and Y,
      (b) an unselected said base of (i)(a) can be substituted any number of times for any one of W, X and Y, or
   (ii) (a) W=G or C,
      X=A or T/U,
      Y=A or T/U,
      and X≠Y, and
      (b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F)
   (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
   (II) for any phantom sequence generated from any pair of first and second sequences of the set L₁ and L₂ in length, respectively, by selection from the first and second sequences of identical bases in identical sequence with each other:
      (i) any consecutive sequence of bases in the phantom sequence which is identical to a consecutive sequence of bases in each of the first and second sequences from which it is generated is less than ((¾×L)−1) bases in length;
      (ii) the phantom sequence, if greater than or equal to (⅚×L) in length, contains at least three insertions/deletions or mismatches when compared to the first and second sequences from which it is generated; and
      (iii) the phantom sequence is not greater than or equal to ( 11/12)×L) in length;
      where L=L₁, or if L₁≠L₂, where L is the greater of L₁ and L₂; and
         wherein any base present may be substituted by an analogue thereof; and
iii) a second nucleic acid molecule comprising a 3′ portion and a 5′ portion, wherein said 5′ portion is completely complementary to said second region of said target nucleic acid;

wherein said thermostable 5′ nuclease lacks synthesis activity, and wherein at least a portion of said first nucleic acid molecule is annealed to said first region of said target nucleic acid, and wherein at least a portion of said second nucleic acid molecule is annealed to said second region of said target nucleic acid;
b) cleaving said cleavage structure with said thermostable 5′ nuclease so as to generate non-target cleavage product; and
c) detecting the cleavage of said cleavage structure.

In some embodiments, said non-target cleavage product comprises the 5′ portion of said first nucleic acid molecule, and detecting the cleavage of the cleavage structure comprises detecting annealing of the non-target cleavage product to a third nucleic acid molecule, wherein the third nucleic acid molecule comprises a nucleic acid sequence complementary to the tag identifier selected in step (a)(iv).

In some preferred embodiments, the tag identifiers 1-210 are selected from SEQ ID NOS: 1173-1382.

In some embodiments, the target nucleic acid comprises an amplified nucleic acid. In some preferred embodiments, the amplified nucleic acid is produced using a polymerase chain reaction.

In some embodiments, the detecting of the cleavage of said cleavage structure comprises detection of fluorescence. In preferred embodiments, the detecting of the cleavage of said cleavage structure comprises detection of fluorescence energy transfer. In some embodiments, the detecting of the cleavage of said cleavage structure comprises detection of radioactivity, luminescence, phosphorescence, fluorescence polarization, and/or charge.

In some embodiments, the target nucleic acid comprises DNA and in some embodiments the target nucleic acid comprises RNA.

In some embodiments, the 3′ portion of the second nucleic acid molecule comprises a 3′ terminal nucleotide not complementary to said target nucleic acid. In other embodiments, the 3′ portion of the second nucleic acid molecule comprises a 3′ terminal nucleotide complementary to said target nucleic acid.

In some embodiments, the 3′ portion of the second nucleic acid molecule consists of a single nucleotide. In some embodiments, the single nucleotide is not complementary to said target nucleic acid, while in other embodiments, the single nucleotide is complementary to said target nucleic acid.

In some embodiments, the 3′ terminal nucleotide of the second nucleic acid molecule comprises a naturally occurring nucleotide, while in other embodiments, the 3′ terminal nucleotide comprises a nucleotide analog.

In some embodiments, the 3′ portion of the second nucleic acid molecule is completely complementary to the target nucleic acid.

The present invention provides a composition comprising a cleavage structure, said cleavage structure comprising:

i) a target nucleic acid having a first region and a second region, wherein said second region is located adjacent to and downstream of said first region;

ii) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and that is selected from the group consisting of tag identifiers 211-1378, wherein
each of 1 to 3 is a nucleotide base selected to be different from the others of 1 to 3, with the proviso that up to three nucleotide bases of each sequence can be substituted with any nucleotide base provided that for any pair of sequences of the set:
   M1≦16, M2≦13, M3≦20, M4≦16, and M5≦19, where:
      M1 is the maximum number of matches for any alignment in which there are no internal indels;
      M2 is the maximum length of a block of matches for any alignment;
      M3 is the maximum number of matches for any alignment having a maximum score;
      M4 is the maximum sum of the lengths of the longest two blocks of matches for any alignment of maximum score; and
      M5 is the maximum sum of the lengths of all the blocks of matches having a length of at least 3, for any alignment of maximum score; wherein
      the score of an alignment is determined according to the equation (A×m)−(B×mm)−(C×(o−g+eg))−(D×eg)), wherein:
         for each of (i) to (iv):
         (i) m=6, mm=6, og=0 and eg=6,
         (ii) m=6, mm=6, og=5 and eg=1,
         (iii) m=6, mm=2, og=5 and eg=1, and
         (iv) m=6, mm=6, og=6 and eg=0,
      A is the total number of matched pairs of bases in the alignment;
      B is the total number of internal mismatched pairs in the alignment;
      C is the total number of internal gaps in the alignment; and
      D is the total number of internal indels in the alignment minus the total number of internal gaps in the alignment; and
   wherein the maximum score is determined separately for each of (i), (ii), (iii) and (iv); and
iii) a second nucleic acid molecule comprising a 3′ portion and a 5′ portion, wherein said 5′ portion is completely complementary to said second region of said target nucleic acid.

In preferred embodiments, the tag identifiers 211-1378 are selected from SEQ ID NOS: 1-1172.

In some embodiments, the composition further comprises a 5′ nuclease. In preferred embodiments, the 5′ nuclease is a FEN-1 nuclease. In particularly preferred embodiments, the FEN-1 nuclease is a thermostable FEN-1 nuclease.

The present invention provides a method for detecting the presence of a target nucleic acid molecule in a sample, comprising:

a) incubating a sample with a thermostable 5′ nuclease under conditions wherein a cleavage structure is formed, said cleavage structure comprising:
i) a target nucleic acid having a first region and a second region, wherein said second region is located adjacent to and downstream of said first region;
ii) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and that is selected from the group consisting of tag identifiers 211-1378, wherein
each of 1 to 3 is a nucleotide base selected to be different from the others of 1 to 3, with the proviso that up to three nucleotide bases of each sequence can be substituted with any nucleotide base provided that for any pair of sequences of the set:
   M1≦16, M2≦13, M3≦20, M4≦16, and M5≦19, where:
      M1 is the maximum number of matches for any alignment in which there are no internal indels;
      M2 is the maximum length of a block of matches for any alignment;
      M3 is the maximum number of matches for any alignment having a maximum score;
      M4 is the maximum sum of the lengths of the longest two blocks of matches for any alignment of maximum score; and
      M5 is the maximum sum of the lengths of all the blocks of matches having a length of at least 3, for any alignment of maximum score; wherein
      the score of an alignment is determined according to the equation (A×m)−(B×mm)−(C×(o−g+eg))−(D×eg)), wherein:
         for each of (i) to (iv):
         (i) m=6, mm=6, og=0 and eg=6,
         (ii) m=6, mm=6, og=5 and eg=1,
         (iii) m=6, mm=2, og=5 and eg=1, and
         (iv) m=6, mm=6, og=6 and eg=0,
      A is the total number of matched pairs of bases in the alignment;
      B is the total number of internal mismatched pairs in the alignment;
      C is the total number of internal gaps in the alignment; and
      D is the total number of internal indels in the alignment minus the total number of internal gaps in the alignment; and
   wherein the maximum score is determined separately for each of (i), (ii), (iii) and (iv); and
iii) a second nucleic acid molecule comprising a 3′ portion and a 5′ portion, wherein said 5′ portion is completely complementary to said second region of said target nucleic acid;

wherein said thermostable 5′ nuclease lacks synthesis activity, and wherein at least a portion of said first nucleic acid molecule is annealed to said first region of said target nucleic acid, and wherein at least a portion of said second nucleic acid molecule is annealed to said second region of said target nucleic acid;

b) cleaving said cleavage structure with said thermostable 5′ nuclease so as to generate non-target cleavage product; and

c) detecting the cleavage of said cleavage structure.

In some embodiments, the non-target cleavage product comprises the 5′ portion of the first nucleic acid molecule, and wherein the detecting the cleavage of the cleavage structure comprises detecting annealing of the non-target cleavage product to a third nucleic acid molecule, wherein the third nucleic acid molecule comprises a nucleic acid sequence complementary to the tag identifier selected in step (a) (iv). In preferred embodiments, the tag identifiers 211-1378 are selected from the group consisting of SEQ ID NOS: 1-1172.

In some embodiments, the target nucleic acid comprises an amplified nucleic acid. In some preferred embodiments, the amplified nucleic acid is produced using a polymerase chain reaction.

In some embodiments, the detecting of the cleavage of said cleavage structure comprises detection of fluorescence. In preferred embodiments, the detecting of the cleavage of said cleavage structure comprises detection of fluorescence energy transfer. In some embodiments, the detecting of the cleavage of said cleavage structure comprises detection of radioactivity, luminescence, phosphorescence, fluorescence polarization, and/or charge.

In some embodiments, the target nucleic acid comprises DNA and in some embodiments the target nucleic acid comprises RNA.

In some embodiments, the 3′ portion of the second nucleic acid molecule comprises a 3′ terminal nucleotide not complementary to said target nucleic acid. In other embodiments, the 3′ portion of the second nucleic acid molecule comprises a 3′ terminal nucleotide complementary to said target nucleic acid.

In some embodiments, the 3′ portion of the second nucleic acid molecule consists of a single nucleotide. In some embodiments, the single nucleotide is not complementary to said target nucleic acid, while in other embodiments, the single nucleotide is complementary to said target nucleic acid.

In some embodiments, the 3′ terminal nucleotide of the second nucleic acid molecule comprises a naturally occurring nucleotide, while in other embodiments, the 3′ terminal nucleotide comprises a nucleotide analog.

In some embodiments, the 3′ portion of the second nucleic acid molecule is completely complementary to the target nucleic acid.

Embodiments of the invention are described in this summary, and in the Detailed Description of the Invention, below, which is incorporated here by reference. Although the invention has been described in connection with specific preferred embodiments, it should be understood that the invention as claimed should not be unduly limited to such specific embodiments.

### DESCRIPTION OF THE DRAWINGS

FIG. 1 shows a schematic diagram of one embodiment of an INVADER Assay configured using a 5′ tag on an oligonucleotide probe.

FIG. 2 shows a schematic diagram of one embodiment of an INVADER Assay configured using a 5′ tag on an oligonucleotide probe, wherein the non-cross hybridizing 5′ tag is used in a secondary cleavage reaction. In the embodiment shown, “D” and “Q” on the secondary cleavage structure represent a fluorescent dye and a quenching moiety, respectively.

FIG. 3 shows a schematic diagram of one embodiment of an INVADER Assay configured using a first 5′ tag on an oligonucleotide probe, wherein the non-cross hybridizing first 5′ tag portion of the cleaved first oligonucleotide is used in a secondary cleavage reaction, wherein the second cleavage structure comprises a second non-cross hybridizing 5′ tag. One or both of the first and second cleavage structures may comprise a 5′ tag.

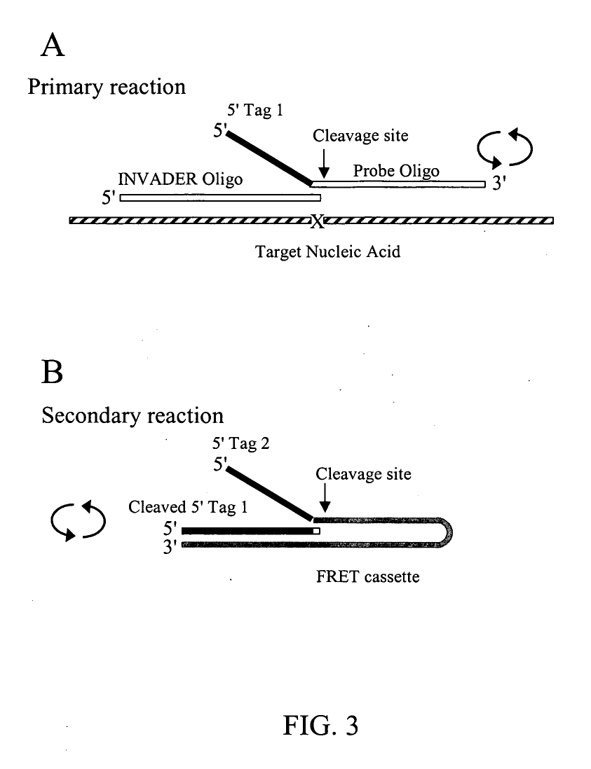

FIG. 4 shows a schematic diagram of one embodiment of an INVADER Assay configured using a 5′ tag on an oligonucleotide probe, wherein the non-cross hybridizing 5′ tag portion of the cleaved first oligonucleotide hybridizes to surface-bound oligonucleotide (e.g., in an oligonucleotide array).

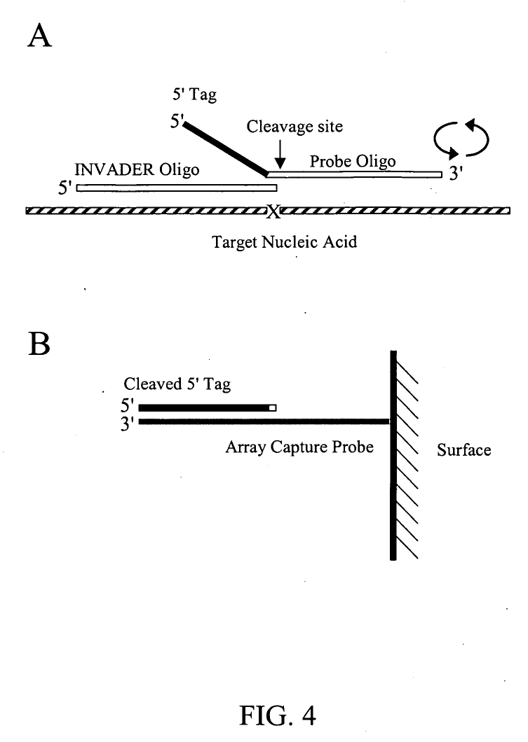

### DEFINITIONS

To facilitate an understanding of the present invention, a number of terms and phrases are defined below:

As used herein, the terms “subject” and “patient” refer to any organisms including plants, microorganisms and animals (e.g., mammals such as dogs, cats, livestock, and humans).

As used herein, the term “INVADER assay reagents” refers to one or more reagents for detecting target sequences, said reagents comprising oligonucleotides capable of forming an invasive cleavage structure in the presence of the target sequence. In some embodiments, the INVADER assay reagents further comprise an agent for detecting the presence of an invasive cleavage structure (e.g., a cleavage agent). In some embodiments, the oligonucleotides comprise first and second oligonucleotides, said first oligonucleotide comprising a portion complementary to a first region of the target nucleic acid and said second oligonucleotide comprising a 3′ portion and a 5′ portion, said 5′ portion complementary to a second region of the target nucleic acid downstream of and contiguous to the first region. In some embodiments, the 3′ portion of the second oligonucleotide comprises a 3′ terminal nucleotide not complementary to the target nucleic acid. In preferred embodiments, the 3′ portion of the second oligonucleotide consists of a single nucleotide not complementary to the target nucleic acid. In some embodiments, the 3′ portion of the second oligonucleotide comprises a moiety that is not a nucleotide. In preferred embodiments, the 3′ portion of the second oligonucleotide comprises an aromatic ring moiety that is not a nucleotide. In some embodiments, the first oligonucleotide further comprises a 5′ portion comprising a tag sequence. In preferred embodiments, the tag sequence is a non-cross-hybridizing tag as described herein.

In some embodiments, INVADER assay reagents are configured to detect a target nucleic acid sequence comprising first and second non-contiguous single-stranded regions separated by an intervening region comprising a double-stranded region. In preferred embodiments, the INVADER assay reagents comprise a bridging oligonucleotide capable of binding to said first and second non-contiguous single-stranded regions of a target nucleic acid sequence. In particularly preferred embodiments, either or both of said first or said second oligonucleotides of said INVADER assay reagents are bridging oligonucleotides. See, e.g., U.S. Pat. No. 6,709,815, which is incorporated herein by reference.

In some embodiments, the INVADER assay reagents further comprise a solid support. For example, in some embodiments, the one or more oligonucleotides of the assay reagents (e.g., first and/or second oligonucleotide, whether bridging or non-bridging) is attached to said solid support. In some embodiments, the INVADER assay reagents further comprise a buffer solution. In some preferred embodiments, the buffer solution comprises a source of divalent cations (e.g., Mn²⁺ and/or Mg²⁺ ions). Individual ingredients (e.g., oligonucleotides, enzymes, buffers, target nucleic acids) that collectively make up INVADER assay reagents are termed “INVADER assay reagent components.”

In some embodiments, the INVADER assay reagents further comprise a third oligonucleotide complementary to a third region of the target nucleic acid upstream of the first region of the first target nucleic acid. In yet other embodiments, the INVADER assay reagents further comprise a target nucleic acid. In some embodiments, the INVADER assay reagents further comprise a second target nucleic acid. In yet other embodiments, the INVADER assay reagents further comprise a third oligonucleotide comprising a 5′ portion complementary to a first region of the second target nucleic acid. In some specific embodiments, the 3′ portion of the third oligonucleotide is covalently linked to the second target nucleic acid. In other specific embodiments, the second target nucleic acid further comprises a 5′ portion, wherein the 5′ portion of the second target nucleic acid is the third oligonucleotide. In some embodiments, the third oligonucleotide further comprises a 5′ terminal portion comprising a tag sequence. In preferred embodiments, the tag sequence is a non-cross-hybridizing tag as described herein. In still other embodiments, the INVADER assay reagents further comprise an arrestor molecule (e.g., arrestor oligonucleotide).

In some preferred embodiments, the INVADER assay reagents further comprise reagents for detecting a nucleic acid cleavage product. In some embodiments, one or more oligonucleotides in the INVADER assay reagents comprise a label. In some preferred embodiments, said first oligonucleotide comprises a label. In other preferred embodiments, said third oligonucleotide comprises a label. In particularly preferred embodiments, the reagents comprise a first and/or a third oligonucleotide labeled with moieties that produce a fluorescence resonance energy transfer (FRET) effect.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In preferred embodiments, predispensed assay reagent components are predispensed and are provided in a reaction vessel (including but not limited to a reaction tube or a well, as in, e.g., a microtiter plate, or in a microfluidic card or chip). In certain preferred embodiments, the INVADER assay reagents are provided in microfluidic devices such as those described in U.S. Pat. Nos. 6,627,159; 6,720,187; 6,734,401; and 6,814,935, as well as U.S. Pat. Pub. 2002/0064885, all of which are herein incorporated by reference. In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In some embodiments, the present invention provides INVADER assay reagent kits comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, software (e.g., for collecting and analyzing data), inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; mass tags; and fluorescent dyes alone or in combination with moieties that can suppress (“quench”) or shift emission spectra by fluorescence resonance energy transfer (FRET). FRET is a distance-dependent interaction between the electronic excited states of two molecules (e.g., two dye molecules, or a dye molecule and a non-fluorescing quencher molecule) in which excitation is transferred from a donor molecule to an acceptor molecule without emission of a photon. (Stryer et al., 1978, Ann. Rev. Biochem., 47:819; Selvin, 1995, Methods Enzymol., 246:300, each incorporated herein by reference). As used herein, the term “donor” refers to a fluorophore that absorbs at a first wavelength and emits at a second, longer wavelength. The term “acceptor” refers to a moiety such as a fluorophore, chromophore, or quencher that has an absorption spectrum that overlaps the donor's emission spectrum, and that is able to absorb some or most of the emitted energy from the donor when it is near the donor group (typically between 1-100 nm). If the acceptor is a fluorophore, it generally then re-emits at a third, still longer wavelength; if it is a chromophore or quencher, it then releases the energy absorbed from the donor without emitting a photon. In some embodiments, changes in detectable emission from a donor dye (e.g. when an acceptor moiety is near or distant) are detected. In some embodiments, changes in detectable emission from an acceptor dye are detected. In preferred embodiments, the emission spectrum of the acceptor dye is distinct from the emission spectrum of the donor dye such that emissions from the dyes can be differentiated (e.g., spectrally resolved) from each other.

In some embodiments, a donor dye is used in combination with multiple acceptor moieties. In a preferred embodiment, a donor dye is used in combination with a non-fluorescing quencher and with an acceptor dye, such that when the donor dye is close to the quencher, its excitation is transferred to the quencher rather than the acceptor dye, and when the quencher is removed (e.g., by cleavage of a probe), donor dye excitation is transferred to an acceptor dye. In particularly preferred embodiments, emission from the acceptor dye is detected. See, e.g., Tyagi, et al., Nature Biotechnology 18:1191 (2000), which is incorporated herein by reference. Labels may provide signals detectable by fluorescence (e.g., simple fluorescence, FRET, time-resolved fluorescence, fluorescence polarization, etc.), radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

In some embodiments a label comprises a particle for detection. In preferred embodiments, the particle is a phosphor particle. In particularly preferred embodiments, the phosphor particle is an up-converting phosphor particle (see, e.g., Ostermayer, F. W. Preparation and properties of infrared-to-visible conversion phosphors. Metall. Trans. 752, 747-755 [1971]). In some embodiments, rare earth-doped ceramic particles are used as phosphor particles. Phosphor particles may be detected by any suitable method, including but not limited to up-converting phosphor technology (UPT), in which up-converting phosphors transfer low energy infrared (IR) radiation to high-energy visible light. While the present invention is not limited to any particular mechanism, in some embodiments the UPT up-converts infrared light to visible light by multi-photon absorption and subsequent emission of dopant-dependant phosphorescence. See, e.g., U.S. Pat. No. 6,399,397, Issued Jun. 4, 2002 to Zarling, et al.; van De Rijke, et al., Nature Biotechnol. 19(3):273-6 [2001]; Corstjens, et al., IEE Proc. Nanobiotechnol. 152(2):64 [2005], each incorporated by reference herein in its entirety.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “homology” and “homologous” refers to a degree of identity. There may be partial homology or complete homology. A partially homologous sequence is one that is less than 100% identical to another sequence. In the context of this invention, pairs of sequences are compared with each other based on the amount of “homology” between the sequences. By way of example, two sequences are said to have a 50% “maximum homology” with each other if, when the two sequences are aligned side-by-side with each other so as to obtain the (absolute) maximum number of identically paired bases, the number of identically paired bases is 50% of the total number of bases in one of the sequences. (If the sequences being compared are of different lengths, then it would be of the total number of bases in the shorter of the two sequences.)

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of Tₘ.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant” or “polymorphic” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof. In some embodiments, oligonucleotides that form invasive cleavage structures are generated in a reaction (e.g., by extension of a primer in an enzymatic extension reaction).

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, as in a purified restriction digest or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “cleavage structure” as used herein, refers to a structure that is formed by the interaction of at least one probe oligonucleotide and a target nucleic acid, forming a structure comprising a duplex, the resulting structure being cleavable by a cleavage means, including but not limited to an enzyme. The cleavage structure is a substrate for specific cleavage by the cleavage means in contrast to a nucleic acid molecule that is a substrate for non-specific cleavage by agents such as phosphodiesterases which cleave nucleic acid molecules without regard to secondary structure (i.e., no formation of a duplexed structure is required).

The term “cleavage means” or “cleavage agent” as used herein refers to any means that is capable of cleaving a cleavage structure, including but not limited to enzymes. “Structure-specific nucleases” or “structure-specific enzymes” are enzymes that recognize specific secondary structures in a nucleic molecule and cleave these structures. The cleavage means of the invention cleave a nucleic acid molecule in response to the formation of cleavage structures; it is not necessary that the cleavage means cleave the cleavage structure at any particular location within the cleavage structure.

The cleavage means may include nuclease activity provided from a variety of sources including the CLEAVASE enzymes, the FEN-1 endonucleases (including RAD2 and XPG proteins), Taq DNA polymerase and E. coli DNA polymerase I. The cleavage means may include enzymes having 5′ nuclease activity (e.g., Taq DNA polymerase (DNAP), E. coli DNA polymerase I). The cleavage means may also include modified DNA polymerases having 5′ nuclease activity but lacking synthetic activity. Examples of cleavage means suitable for use in the method and kits of the present invention are provided in U.S. Pat. Nos. 5,614,402; 5,795,763; 5,843,669; 6,090,606; PCT Appln. Nos WO 98/23774; WO 02/070755A2; WO0190337A2; and WO03073067, each of which is herein incorporated by reference it its entirety.

The term “thermostable” when used in reference to an enzyme, such as a 5′ nuclease, indicates that the enzyme is functional or active (i.e., can perform catalysis) at an elevated temperature, i.e., at about 55° C. or higher.

The term “cleavage products” as used herein, refers to products generated by the reaction of a cleavage means with a cleavage structure (i.e., the treatment of a cleavage structure with a cleavage means).

The term “target nucleic acid,” when used in reference to an invasive cleavage reaction, refers to a nucleic acid molecule containing a sequence that has at least partial complementarity with at least a probe oligonucleotide and may also have at least partial complementarity with an INVADER oligonucleotide. The target nucleic acid may comprise single- or double-stranded DNA or RNA.

The term “non-target cleavage product” refers to a product of a cleavage reaction that is not derived from the target nucleic acid. As discussed above, in the methods of the present invention, cleavage of the cleavage structure generally occurs within the probe oligonucleotide. The fragments of the probe oligonucleotide generated by this target nucleic acid-dependent cleavage are “non-target cleavage products.”

The term “probe oligonucleotide,” when used in reference to an invasive cleavage reaction, refers to an oligonucleotide that interacts with a target nucleic acid to form a cleavage structure in the presence or absence of an INVADER oligonucleotide. When annealed to the target nucleic acid, the probe oligonucleotide and target form a cleavage structure and cleavage occurs within the probe oligonucleotide.

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide-whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide.

The term “cassette,” when used in reference to an invasive cleavage reaction, as used herein refers to an oligonucleotide or combination of oligonucleotides configured to generate a detectable signal in response to cleavage of a probe oligonucleotide in an INVADER assay. In preferred embodiments, the cassette hybridizes to a non-target cleavage product (e.g., a minimally cross-hybridizing 5′ tag) from cleavage of the probe oligonucleotide to form a second invasive cleavage structure, such that the cassette can then be cleaved.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label. In some embodiments, the cassette comprises a 5′ tag of the present invention. In particularly preferred embodiments, cassette comprises labeled moieties that produce a fluorescence resonance energy transfer (FRET) effect.

As used herein, the phrase “non-amplified oligonucleotide detection assay” refers to a detection assay configured to detect the presence or absence of a particular polymorphism (e.g., SNP, repeat sequence, etc.) in a target sequence (e.g. genomic DNA) that has not been amplified (e.g. by PCR), without creating copies of the target sequence. A “non-amplified oligonucleotide detection assay” may, for example, amplify a signal used to indicate the presence or absence of a particular polymorphism in a target sequence, so long as the target sequence is not copied.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs comprise modified forms of deoxyribonucleotides as well as ribonucleotides.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

An oligonucleotide is said to be present in “excess” relative to another oligonucleotide (or target nucleic acid sequence) if that oligonucleotide is present at a higher molar concentration that the other oligonucleotide (or target nucleic acid sequence). When an oligonucleotide such as a probe oligonucleotide is present in a cleavage reaction in excess relative to the concentration of the complementary target nucleic acid sequence, the reaction may be used to indicate the amount of the target nucleic acid present. Typically, when present in excess, the probe oligonucleotide will be present at least a 100-fold molar excess; typically at least 1 pmole of each probe oligonucleotide would be used when the target nucleic acid sequence was present at about 10 fmoles or less.

The term “nucleic acid sequence” as used herein refers to an oligonucleotide, nucleotide or polynucleotide, and fragments or portions thereof, and to DNA or RNA of genomic or synthetic origin that may be single or double stranded, and represent the sense or antisense strand. Similarly, “amino acid sequence” as used herein refers to peptide or protein sequence.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

As used herein, the term “non-cross-hybridization” refers to the absence of hybridization between two nucleic acids that are not perfect complements of each other.

As used herein, the term “cross-hybridization” refers to the hydrogen bonding of a single-stranded nucleic acid sequence that is partially but not entirely complementary to a single-stranded substrate.

As used herein, the term “minimal cross-hybridization” refers to low-level cross hybridization such that any cross hybridization detectable is of little or no consequence (e.g., in an experiment or assay).

As used herein, the term “tag” refers to an oligonucleotide or a portion of an oligonucleotide that comprising a non-cross-hybridizing or minimally cross-hybridizing sequence. In preferred embodiments, a tag sequence is not the same as, or complementary to, the portion of target nucleic acid recognized by (e.g., complementary to) a target-specific portion of a tag-containing oligonucleotide.

As used herein, the term “block sequence” refers to a symbolic representation of a sequence of blocks. In its most general form a block sequence is a representative sequence in which no particular value, mathematical variable, or other designation is assigned to each block of the sequence.

As used herein, the term “incidence matrix” refers to the well-defined term in the field of Discrete Mathematics. However, an incidence matrix cannot be defined without first defining a “graph”. In the method described herein, a subset of general graphs called simple graphs is used. Members of this subcategory are further defined as follows.

A simple graph G is a pair (V, E) where V represents the set of vertices of the simple graph and E is a set of un-oriented edges of the simple graph. An edge is defined as a 2-component combination of members of the set of vertices. In other words, in a simple graph G there are some pairs of vertices that are connected by an edge. In this application, a graph is based on nucleic acid sequences generated using sequence templates and vertices represent DNA sequences and edges represent a relative property of any pair of sequences.

The incidence matrix is a mathematical object that allows one to describe any given graph. For the subset of simple graphs used herein, the simple graph G=(V,E), and for a pre-selected and fixed ordering of vertices, V={v₁,v₂, . . . vₙ}, elements of the incidence matrix A(G)=[aᵢⱼ] are defined by the following rules:

(1) a_{i j}=1 for any pair of vertices {vᵢ,vⱼ} that is a member of the set of edges; and

(2) aᵢⱼ=0 for any pair of vertices {vᵢ,vⱼ}that is not a member of the set of edges.

This is an exact unequivocal definition of the incidence matrix. In effect, one selects the indices: 1, 2, . . . n of the vertices and then forms an (n×n) square matrix with elements aᵢⱼ=1 if the vertices vᵢ and vⱼ are connected by an edge and aᵢⱼ=0 if the vertices vᵢ and vⱼ are not connected by an edge.

To define the term “class property” as used herein, the term “complete simple graph” or “clique” must first be defined. The complete simple graph is required because all sequences that result from the method described herein should collectively share the relative property of any pair of sequences defining an edge of graph G, for example not violating the threshold rule that is, do not have a “maximum simple homology” greater than a predetermined amount, whatever pair of the sequences are chosen from the final set. It is possible that additional “local” rules, based on known or empirically determined behavior of particular nucleotides, or nucleotide sequences, are applied to sequence pairs in addition to the basic threshold rule.

In the language of a simple graph, G=(V, E), this means in the final graph there should be no pair of vertices (no sequence pair) not connected by an edge (because an edge means that the sequences represented by vᵢ and vⱼ do not violate the threshold rule).

Because the incidence matrix of any simple graph can be generated by the above definition of its elements, the consequence of defining a simple complete graph is that the corresponding incidence matrix for a simple complete graph will have all off-diagonal elements equal to 1 and all diagonal elements equal to 0. This is because if one aligns a sequence with itself, the threshold rule is of course violated, and all other sequences are connected by an edge.

For any simple graph, there might be a complete subgraph. First, the definition of a subgraph of a graph is as follows. The subgraph Gs=(Vs, Es) of a simple graph G=(V, E) is a simple graph that contains the subsets of vertices Vs of the set V of vertices and inclusion of the set Vs into the set V is immersion (a mathematical term). This means that one generates a subgraph Gs=(Vs,Es) of a simple graph G in two steps. First select some vertices Vs from G. Then select those edges Es from G that connect the chosen vertices and do not select edges that connect selected with non selected vertices.

We desire a subgraph of G that is a complete simple graph. By using this property of the complete simple graph generated from the simple graph G of all sequences generated by the template based algorithm, the pairwise property of any pair of the sequences (violating/non-violating the threshold rule) is converted into the property of all members of the set, termed “the class property”.

By selecting a subgraph of a simple graph G that is a complete simple graph, this assures that, up to the tests involving the local rules described herein, there are no pairs of sequences in the resulting set that violate the threshold rule, also described above, independent of which pair of sequences in the set are chosen. This feature is called the “desired class property”.

The present invention thus includes reducing the potential for non cross-hybridization behavior by taking into account local homologies of the sequences and appears to have greater rigor than known approaches. For example, the method described herein involves the sliding of one sequence relative to the other sequence in order to form a sequence alignment that would accommodate insertions or deletions. (Kane et al., Nucleic Acids Res.; 28, 4552-4557: 2000).

### DETAILED DESCRIPTION OF THE INVENTION

The present invention relates to the use of non and minimally cross-hybridizing oligonucleotide sequences for use in the INVADER Assay. The incorporation of these sequences into one of the two oligonucleotides that forms an invasive cleavage structure with a target nucleic acid, and subsequent structure-dependent cleavage of the oligonucleotide comprising the minimally cross-hybridizing sequence provides a way of using the INVADER Assay in massively parallel analysis of multiple genes, e.g., in a gene microarray.

Invasive cleavage assays, or INVADER assays comprise forming a nucleic acid cleavage structure that is dependent upon the presence of a target nucleic acid and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced. Such embodiments have been termed the INVADER assay (Third Wave Technologies) and are described in U.S. Pat. Nos. 5,846,717, 5,985,557, 5,994,069, 6,001,567, and 6,090,543, WO 97/27214, WO 98/42873, Lyamichev et al., Nat. Biotech., 17:292 (1999), Hall et al., PNAS, USA, 97:8272 (2000), each of which is herein incorporated by reference in their entirety for all purposes). The INVADER assay detects hybridization of probes to a target by enzymatic cleavage of specific structures by structure specific enzymes.

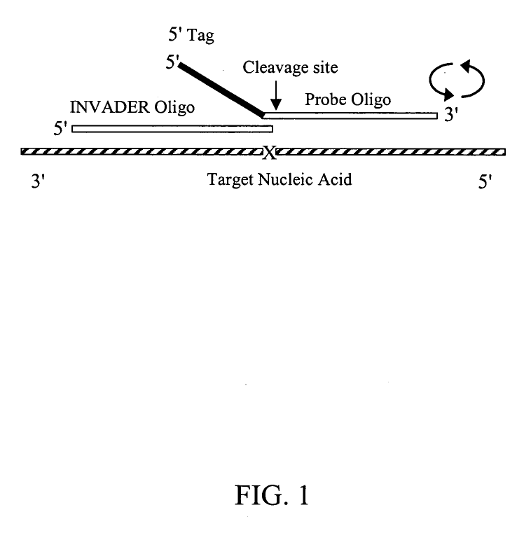

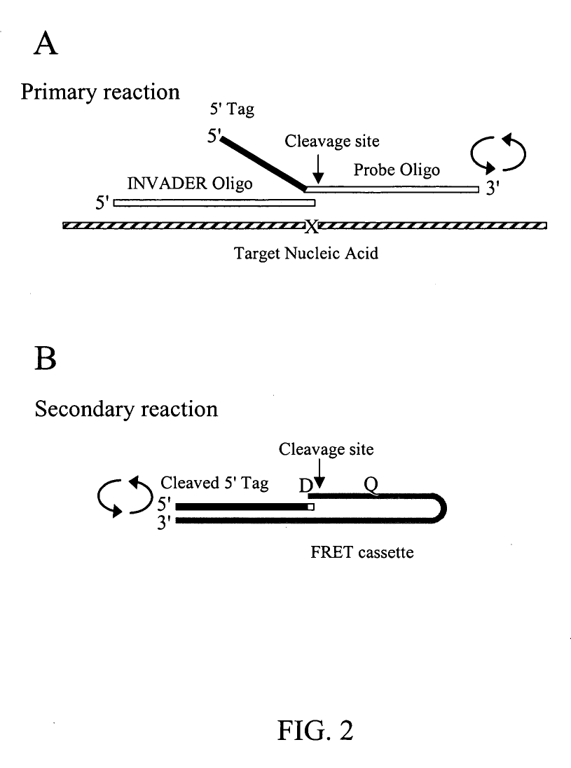

The INVADER assay detects specific DNA and RNA sequences by using structure-specific enzymes (e.g. FEN endonucleases) to cleave a complex formed by the hybridization of overlapping oligonucleotide probes (See, e.g. FIG. 1). Elevated temperature and an excess of one of the probes enable multiple probes to be cleaved for each target sequence present without temperature cycling. In some embodiments, these cleaved probes then direct cleavage of a second labeled probe (see, e.g., FIG. 2). The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified, as well as amplified, RNA and DNA including genomic DNA. In the embodiments shown schematically in FIG. 2, the INVADER assay uses two cascading steps (a primary and a secondary reaction) both to generate and then to amplify the target-specific signal. In the primary reaction (FIG. 2, panel A), the primary probe and the INVADER oligonucleotide hybridize in tandem to the target nucleic acid to form an overlapping structure. An unpaired “flap” (e.g., comprising a non-cross hybridizing tag) is included on the 5′ end of the primary probe. A structure-specific enzyme (e.g. the CLEAVASE enzyme, Third Wave Technologies) recognizes the overlap and cleaves off the unpaired flap, releasing it as a target-specific product. In embodiments of the present invention, the flap comprises a non-cross hybridizing tag. In embodiments comprising a secondary reaction, this cleaved product serves as an INVADER oligonucleotide on the fluorescence resonance energy transfer (FRET) cassette to again create the structure recognized by the structure specific enzyme (FIG. 2, panel B). When the two dyes on a single FRET cassette are separated by cleavage (indicated by the arrow in FIG. 2), a detectable fluorescent signal above background fluorescence is produced. Consequently, cleavage of this second structure results in an increase in fluorescence, indicating the presence of the target allele. In preferred embodiments, FRET probes having different labels (e.g. resolvable by difference in emission or excitation wavelengths, or resolvable by time-resolved fluorescence detection) are provided for each allele or locus to be detected, such that the different alleles or loci can be detected in a single reaction. In such embodiments, the primary probe sets and the different FRET probes may be combined in a single assay, allowing comparison of the signals from each allele or locus in the same sample. In some embodiments the cassette comprises two oligonucleotides, e.g., a secondary probe oligonucleotide comprising the FRET labels, and a secondary target nucleic acid to which the cleaved 5′ tag and the secondary probe anneal to form the second cleavage structure.

If the primary probe oligonucleotide and the target nucleotide sequence do not match perfectly at the cleavage site, the overlapped structure does not form and cleavage is suppressed. The structure specific enzyme (e.g., CLEAVASE VIII enzyme, Third Wave Technologies) used cleaves the overlapped structure more efficiently (e.g., at least 340-fold) than the non-overlapping structure, allowing excellent discrimination of the alleles.

In the INVADER assays, the probes turn can over without temperature cycling to produce many signals per target (i.e., linear signal amplification). Similarly, each target-specific product can enable the cleavage of many FRET probes. The primary INVADER assay reaction is directed against the target DNA (or RNA) being detected. The target DNA is the limiting component in the first invasive cleavage, since the INVADER and primary probe are supplied in molar excess. In the second invasive cleavage, it is the released flap that is limiting. When these two cleavage reactions are performed sequentially, the fluorescence signal from the composite reaction accumulates linearly with respect to the target DNA amount.

In certain embodiments, the INVADER assay, or other nucleotide detection assays, are performed with accessible site designed oligonucleotides and/or bridging oligonucleotides. Such methods, procedures and compositions are described in U.S. Pat. No. 6,194,149, WO9850403, and WO0198537, all of which are specifically incorporated by reference in their entireties.

In certain embodiments, the target nucleic acid sequences are amplified prior to detection (e.g., such that amplified products are generated). See, for example, co-pending application Ser. Nos. 10/356,861 and 10/967,711, each of which is incorporated by reference herein in its entirety for all purposes. In some embodiments, the target nucleic acid comprises genomic DNA. In other embodiments, the target nucleic acid comprises synthetic DNA or RNA. In some preferred embodiments, synthetic DNA within a sample is created using a purified polymerase. In some preferred embodiments, the creation of synthetic DNA using a purified polymerase occurs in the same reaction mixture as the INVADER assay. In some preferred embodiments, creation of synthetic DNA using a purified polymerase comprises the use of PCR. In other preferred embodiments, creation of synthetic DNA using a purified DNA polymerase, suitable for use with the methods of the present invention, comprises use of rolling circle amplification, (e.g., as in U.S. Pat. Nos. 6,210,884, 6,183,960 and 6,235,502, herein incorporated by reference in their entireties). In other preferred embodiments, creation of synthetic DNA comprises copying genomic DNA by priming from a plurality of sites on a genomic DNA sample. In some embodiments, priming from a plurality of sites on a genomic DNA sample comprises using short (e.g., fewer than about 8 nucleotides) oligonucleotide primers. In other embodiments, priming from a plurality of sites on a genomic DNA comprises extension of 3′ ends in nicked, double-stranded genomic DNA (i.e., where a 3′ hydroxyl group has been made available for extension by breakage or cleavage of one strand of a double stranded region of DNA). Some examples of making synthetic DNA using a purified polymerase on nicked genomic DNAs, suitable for use with the methods and compositions of the present invention, are provided in U.S. Pat. No. 6,117,634, issued Sep. 12, 2000, and U.S. Pat. No. 6,197,557, issued Mar. 6, 2001, and in PCT application WO 98/39485, each incorporated by reference herein in their entireties for all purposes.

In other embodiments, synthetic DNA suitable for use with the methods and compositions of the present invention is made using a purified polymerase on multiply-primed genomic DNA, as provided, e.g., in U.S. Pat. Nos. 6,291,187, and 6,323,009, and in PCT applications WO 01/88190 and WO 02/00934, each herein incorporated by reference in their entireties for all purposes. In these embodiments, amplification of DNA such as genomic DNA is accomplished using a DNA polymerase, such as the highly processive Φ 29 polymerase (as described, e.g., in U.S. Pat. Nos. 5,198,543 and 5,001,050, each herein incorporated by reference in their entireties for all purposes) in combination with exonuclease-resistant random primers, such as hexamers.

The present invention further provides assays in which the target nucleic acid is reused or recycled during multiple rounds of hybridization with oligonucleotide probes and cleavage of the probes without the need to use temperature cycling (e.g., for periodic denaturation of target nucleic acid strands) or nucleic acid synthesis (e.g., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (The kinetics of oligonucleotide replacement. Luis P. Reynaldo, Alexander V. Vologodskii, Bruce P. Neri and Victor I. Lyamichev. J. Mol. Biol. 97: 511-520 (2000)), multiple probes can hybridize to the same target, allowing multiple cleavages, and the generation of multiple cleavage products.

The present invention provides INVADER assays using probes comprising non- and minimally cross-hybridizing tags.

A family of 210 sequences has been described to have optimal hybridization properties for use in nucleic acid detection assays. The sequence set of 210 oligonucleotides was characterized in hybridization assays, demonstrating the ability of family members to correctly hybridize to their complementary sequences with an absence of cross hybridization. (See U.S. Patent Publication No. 2005/0186573 to Janeczko, incorporated by reference herein in its entirety). These are the sequences having SEQ ID NOs:1173 to 1382 of Table I.

A family of complements is obtained from a set of oligonucleotides based on a family of oligonucleotides such as those of Table I. For illustrative purposes, providing a family of complements based on the oligonucleotides of Table I will be described.

Firstly, the groups of sequences based on the oligonucleotides of Table I can be represented as follows:

TABLE IA
Numeric sequences corresponding to word patterns of a set
of oligonucleotides Tag Identifier Numeric Pattern

| | |
|---|---|
| Tag | |
| Identifier | Numeric Pattern |

| | | | | | | |
|---|---|---|---|---|---|---|
| 1 | 1 | 4 | 6 | 6 | 1 | 3 |
| 2 | 2 | 4 | 5 | 5 | 2 | 3 |
| 3 | 1 | 8 | 1 | 2 | 3 | 4 |
| 4 | 1 | 7 | 1 | 9 | 8 | 4 |
| 5 | 1 | 1 | 9 | 2 | 6 | 9 |
| 6 | 1 | 2 | 4 | 3 | 9 | 6 |
| 7 | 9 | 8 | 9 | 8 | 10 | 9 |
| 8 | 9 | 1 | 2 | 3 | 8 | 10 |
| 9 | 8 | 8 | 7 | 4 | 3 | 1 |
| 10 | 1 | 1 | 1 | 1 | 1 | 2 |
| 11 | 2 | 1 | 3 | 3 | 2 | 2 |
| 12 | 3 | 1 | 2 | 2 | 3 | 2 |
| 13 | 4 | 1 | 4 | 4 | 4 | 2 |
| 14 | 1 | 2 | 3 | 3 | 1 | 1 |
| 15 | 1 | 3 | 2 | 2 | 1 | 4 |
| 16 | 3 | 3 | 3 | 3 | 3 | 4 |
| 17 | 4 | 3 | 1 | 1 | 4 | 4 |
| 18 | 3 | 4 | 1 | 1 | 3 | 3 |
| 19 | 3 | 6 | 6 | 6 | 3 | 5 |
| 20 | 6 | 6 | 1 | 1 | 6 | 5 |
| 21 | 7 | 6 | 7 | 7 | 7 | 5 |
| 22 | 8 | 7 | 5 | 5 | 8 | 8 |
| 23 | 2 | 1 | 7 | 7 | 1 | 1 |
| 24 | 2 | 3 | 2 | 3 | 1 | 3 |
| 25 | 2 | 6 | 5 | 6 | 1 | 6 |
| 26 | 4 | 8 | 1 | 1 | 3 | 8 |
| 27 | 5 | 3 | 1 | 1 | 6 | 3 |
| 28 | 5 | 6 | 8 | 8 | 6 | 6 |
| 29 | 8 | 3 | 6 | 5 | 7 | 3 |
| 30 | 1 | 2 | 3 | 1 | 4 | 6 |
| 31 | 1 | 5 | 7 | 5 | 4 | 3 |
| 32 | 2 | 1 | 6 | 7 | 3 | 6 |
| 33 | 2 | 6 | 1 | 3 | 3 | 1 |
| 34 | 2 | 7 | 6 | 8 | 3 | 1 |
| 35 | 3 | 4 | 3 | 1 | 2 | 5 |
| 36 | 3 | 5 | 6 | 1 | 2 | 7 |
| 37 | 3 | 6 | 1 | 7 | 2 | 7 |
| 38 | 4 | 6 | 3 | 5 | 1 | 7 |
| 39 | 5 | 4 | 6 | 3 | 8 | 6 |
| 40 | 6 | 8 | 2 | 3 | 7 | 1 |
| 41 | 7 | 1 | 7 | 8 | 6 | 3 |
| 42 | 7 | 3 | 4 | 1 | 6 | 8 |
| 43 | 4 | 7 | 7 | 1 | 2 | 4 |
| 44 | 3 | 6 | 5 | 2 | 6 | 3 |
| 45 | 1 | 4 | 1 | 4 | 6 | 1 |
| 46 | 3 | 3 | 1 | 4 | 8 | 1 |
| 47 | 8 | 3 | 3 | 5 | 3 | 8 |
| 48 | 1 | 3 | 6 | 6 | 3 | 7 |
| 49 | 7 | 3 | 8 | 6 | 4 | 7 |
| 50 | 3 | 1 | 3 | 7 | 8 | 6 |
| 51 | 10 | 9 | 5 | 5 | 10 | 10 |
| 52 | 7 | 10 | 10 | 10 | 7 | 9 |
| 53 | 9 | 9 | 7 | 7 | 10 | 9 |
| 54 | 9 | 3 | 10 | 3 | 10 | 3 |
| 55 | 9 | 6 | 3 | 4 | 10 | 6 |
| 56 | 10 | 4 | 10 | 3 | 9 | 4 |
| 57 | 3 | 9 | 3 | 10 | 4 | 9 |
| 58 | 9 | 10 | 5 | 9 | 4 | 8 |
| 59 | 3 | 9 | 4 | 9 | 10 | 7 |
| 60 | 3 | 5 | 9 | 4 | 10 | 8 |
| 61 | 4 | 10 | 5 | 4 | 9 | 3 |
| 62 | 5 | 3 | 3 | 9 | 8 | 10 |
| 63 | 6 | 8 | 6 | 9 | 7 | 10 |
| 64 | 4 | 6 | 10 | 9 | 6 | 4 |
| 65 | 4 | 9 | 8 | 10 | 8 | 3 |
| 66 | 7 | 7 | 9 | 10 | 5 | 3 |
| 67 | 8 | 8 | 9 | 3 | 9 | 10 |
| 68 | 8 | 10 | 2 | 9 | 5 | 9 |
| 69 | 9 | 6 | 2 | 2 | 7 | 10 |
| 70 | 9 | 7 | 5 | 3 | 10 | 6 |
| 71 | 10 | 3 | 6 | 8 | 9 | 2 |
| 72 | 10 | 9 | 3 | 2 | 7 | 3 |
| 73 | 8 | 9 | 10 | 3 | 6 | 2 |
| 74 | 3 | 2 | 5 | 10 | 8 | 9 |
| 75 | 8 | 2 | 3 | 10 | 2 | 9 |
| 76 | 6 | 3 | 9 | 8 | 2 | 10 |
| 77 | 3 | 7 | 3 | 9 | 9 | 10 |
| 78 | 9 | 10 | 1 | 1 | 9 | 4 |
| 79 | 10 | 1 | 9 | 1 | 4 | 1 |
| 80 | 7 | 1 | 10 | 9 | 8 | 1 |
| 81 | 9 | 1 | 10 | 1 | 10 | 6 |
| 82 | 9 | 6 | 9 | 1 | 3 | 10 |
| 83 | 3 | 10 | 8 | 8 | 9 | 1 |
| 84 | 3 | 8 | 1 | 9 | 10 | 3 |
| 85 | 9 | 10 | 1 | 3 | 6 | 9 |
| 86 | 1 | 9 | 1 | 10 | 3 | 1 |
| 87 | 1 | 4 | 9 | 6 | 8 | 10 |
| 88 | 3 | 3 | 9 | 6 | 1 | 10 |
| 89 | 5 | 3 | 1 | 6 | 9 | 10 |
| 90 | 6 | 1 | 8 | 10 | 9 | 6 |
| 91 | 5 | 9 | 9 | 4 | 10 | 3 |
| 92 | 2 | 10 | 9 | 1 | 9 | 5 |
| 93 | 10 | 10 | 7 | 2 | 1 | 9 |
| 94 | 10 | 9 | 9 | 1 | 8 | 2 |
| 95 | 1 | 8 | 6 | 8 | 9 | 10 |
| 96 | 1 | 9 | 1 | 3 | 8 | 10 |
| 97 | 9 | 6 | 9 | 10 | 1 | 2 |
| 98 | 1 | 10 | 8 | 9 | 9 | 2 |
| 99 | 1 | 9 | 6 | 7 | 2 | 9 |
| 100 | 4 | 3 | 9 | 3 | 5 | 1 |
| 101 | 5 | 11 | 10 | 14 | 12 | 1 |
| 102 | 7 | 12 | 4 | 13 | 3 | 2 |
| 103 | 5 | 5 | 4 | 4 | 12 | 9 |
| 104 | 2 | 13 | 13 | 11 | 13 | 13 |
| 105 | 10 | 2 | 5 | 4 | 12 | 7 |
| 106 | 11 | 7 | 4 | 11 | 6 | 4 |
| 107 | 12 | 12 | 1 | 9 | 11 | 11 |
| 108 | 12 | 9 | 4 | 14 | 12 | 6 |
| 109 | 12 | 7 | 13 | 2 | 9 | 11 |
| 110 | 9 | 11 | 3 | 4 | 1 | 3 |
| 111 | 10 | 5 | 12 | 11 | 4 | 4 |
| 112 | 4 | 13 | 7 | 12 | 1 | 5 |
| 113 | 9 | 13 | 10 | 11 | 11 | 6 |
| 114 | 10 | 14 | 14 | 10 | 1 | 3 |
| 115 | 2 | 14 | 1 | 10 | 4 | 5 |
| 116 | 10 | 12 | 12 | 7 | 11 | 10 |
| 117 | 9 | 11 | 2 | 12 | 8 | 11 |
| 118 | 2 | 8 | 5 | 2 | 12 | 14 |
| 119 | 1 | 8 | 13 | 3 | 7 | 8 |
| 120 | 9 | 4 | 7 | 5 | 4 | 2 |
| 121 | 13 | 2 | 12 | 7 | 1 | 12 |
| 122 | 11 | 10 | 9 | 7 | 5 | 11 |
| 123 | 8 | 12 | 2 | 2 | 12 | 7 |
| 124 | 5 | 2 | 14 | 3 | 4 | 13 |
| 125 | 1 | 8 | 8 | 1 | 5 | 9 |
| 126 | 14 | 5 | 11 | 10 | 13 | 3 |
| 127 | 14 | 1 | 4 | 13 | 2 | 4 |
| 128 | 4 | 4 | 5 | 11 | 3 | 10 |
| 129 | 10 | 9 | 2 | 3 | 3 | 11 |
| 130 | 11 | 4 | 8 | 14 | 3 | 4 |
| 131 | 5 | 1 | 14 | 8 | 11 | 2 |
| 132 | 14 | 3 | 11 | 6 | 12 | 5 |
| 133 | 13 | 4 | 4 | 1 | 10 | 1 |
| 134 | 6 | 10 | 11 | 6 | 5 | 1 |
| 135 | 5 | 8 | 12 | 5 | 1 | 7 |
| 136 | 4 | 5 | 9 | 6 | 9 | 2 |
| 137 | 13 | 2 | 4 | 4 | 2 | 3 |
| 138 | 11 | 2 | 2 | 5 | 9 | 3 |
| 139 | 8 | 1 | 10 | 12 | 2 | 8 |
| 140 | 12 | 7 | 9 | 11 | 4 | 1 |
| 141 | 12 | 1 | 4 | 14 | 3 | 13 |
| 142 | 11 | 2 | 7 | 10 | 4 | 1 |
| 143 | 3 | 4 | 12 | 11 | 11 | 11 |
| 144 | 3 | 3 | 4 | 2 | 12 | 11 |
| 145 | 1 | 5 | 9 | 4 | 2 | 1 |
| 146 | 6 | 1 | 12 | 2 | 10 | 5 |
| 147 | 10 | 5 | 1 | 12 | 2 | 14 |
| 148 | 2 | 11 | 7 | 9 | 4 | 11 |
| 149 | 7 | 4 | 4 | 5 | 14 | 12 |
| 150 | 12 | 5 | 2 | 1 | 10 | 12 |
| 151 | 5 | 9 | 2 | 11 | 6 | 1 |
| 152 | 12 | 14 | 3 | 6 | 1 | 14 |
| 153 | 5 | 9 | 11 | 10 | 1 | 4 |
| 154 | 2 | 5 | 12 | 14 | 10 | 10 |
| 155 | 4 | 5 | 8 | 4 | 5 | 6 |
| 156 | 10 | 12 | 4 | 6 | 12 | 5 |
| 157 | 4 | 2 | 1 | 13 | 6 | 8 |
| 158 | 9 | 10 | 10 | 14 | 5 | 3 |
| 159 | 6 | 14 | 10 | 11 | 3 | 3 |
| 160 | 2 | 9 | 10 | 12 | 5 | 7 |
| 161 | 13 | 3 | 7 | 10 | 5 | 12 |
| 162 | 6 | 4 | 1 | 2 | 5 | 13 |
| 163 | 6 | 1 | 13 | 4 | 14 | 13 |
| 164 | 2 | 12 | 1 | 14 | 1 | 9 |
| 165 | 4 | 11 | 13 | 2 | 6 | 10 |
| 166 | 1 | 10 | 7 | 4 | 5 | 8 |
| 167 | 7 | 2 | 2 | 10 | 13 | 4 |
| 168 | 8 | 2 | 11 | 4 | 6 | 14 |
| 169 | 4 | 8 | 2 | 6 | 2 | 3 |
| 170 | 7 | 1 | 12 | 11 | 2 | 9 |
| 171 | 5 | 6 | 10 | 4 | 13 | 4 |
| 172 | 5 | 10 | 4 | 11 | 9 | 3 |
| 173 | 3 | 11 | 9 | 3 | 2 | 3 |
| 174 | 8 | 15 | 6 | 20 | 17 | 19 |
| 175 | 21 | 10 | 15 | 3 | 7 | 11 |
| 176 | 11 | 7 | 17 | 20 | 14 | 9 |
| 177 | 16 | 6 | 17 | 13 | 21 | 21 |
| 178 | 10 | 15 | 22 | 6 | 17 | 21 |
| 179 | 15 | 7 | 17 | 10 | 22 | 22 |
| 180 | 3 | 20 | 8 | 15 | 20 | 16 |
| 181 | 17 | 21 | 10 | 16 | 6 | 22 |
| 182 | 6 | 21 | 14 | 14 | 14 | 16 |
| 183 | 7 | 17 | 3 | 20 | 10 | 7 |
| 184 | 16 | 19 | 14 | 17 | 7 | 21 |
| 185 | 20 | 16 | 7 | 15 | 22 | 10 |
| 186 | 20 | 10 | 18 | 11 | 22 | 18 |
| 187 | 18 | 7 | 19 | 15 | 7 | 22 |
| 188 | 21 | 18 | 7 | 21 | 16 | 3 |
| 189 | 14 | 13 | 7 | 22 | 17 | 13 |
| 190 | 19 | 7 | 8 | 12 | 10 | 17 |
| 191 | 15 | 3 | 21 | 14 | 9 | 7 |
| 192 | 19 | 6 | 15 | 7 | 14 | 14 |
| 193 | 4 | 17 | 10 | 15 | 20 | 19 |
| 194 | 21 | 6 | 18 | 4 | 20 | 16 |
| 195 | 2 | 19 | 8 | 17 | 6 | 13 |
| 196 | 12 | 12 | 6 | 17 | 4 | 20 |
| 197 | 16 | 21 | 12 | 10 | 19 | 16 |
| 198 | 14 | 14 | 15 | 2 | 7 | 21 |
| 199 | 8 | 16 | 21 | 6 | 22 | 16 |
| 200 | 14 | 17 | 22 | 14 | 17 | 20 |
| 201 | 10 | 21 | 7 | 15 | 21 | 18 |
| 202 | 16 | 13 | 20 | 18 | 21 | 12 |
| 203 | 15 | 7 | 4 | 22 | 14 | 13 |
| 204 | 7 | 19 | 14 | 8 | 15 | 4 |
| 205 | 4 | 5 | 3 | 20 | 7 | 16 |
| 206 | 22 | 18 | 6 | 18 | 13 | 20 |
| 207 | 19 | 6 | 16 | 3 | 13 | 3 |
| 208 | 18 | 6 | 22 | 7 | 20 | 18 |
| 209 | 10 | 17 | 11 | 21 | 8 | 13 |
| 210 | 7 | 10 | 17 | 19 | 10 | 14 |

In Table IA, each of the numerals 1 to 22 (“numeric identifiers”) represents a 4mer (a sequence of 4 nucleotides) and the pattern of numeric identifiers 1 to 22 in the above list corresponds to the pattern of tetrameric oligonucleotide segments present in the tag, e.g., in the oligonucleotides of Table I, below. These oligonucleotides sequences have been found to be non-cross-hybridizing (See Janeczko, supra).

Each pattern is identified by a number in the left column, the “tag identifier,” which is associated with the pattern of numeric identifiers on that line. Each 4-mer is selected from the group of 4-mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY. Here W, X and Y represent nucleotide bases, A, G, C, etc., the assignment of bases being made according to rules described below. Given this numeric pattern, a 4-mer is assigned to a numeral. For example, 1=WXYY, 2=YWXY, etc. Once a given 4-mer has been assigned to a given numeral, it is not assigned for use in the position of a different numeral. It is possible, however, to assign a different 4-mer to the same numeral. That is, for example, the numeral 1 in one position could be assigned WXYY and another numeral 1, in a different position, could be assigned XXXW, but none of the other numerals 2 to 22 can then be assigned WXYY or XXXW. A different way of saying this is that each of 1 to 22 is assigned a 4-mer from the list of eighty-one 4-mers indicated so as to be different from all of the others of 1 to 22.

In the case of the specific oligonucleotides given in Table I, 1=WXYY, 2=YWXY, 3=XXXW, 4=YWYX, 5=WYXY, 6=YYWX, 7=YWXX, 8=WYXX, 9=XYYW, 10=XYWX, 11=YYXW, 12=WYYX, 13=XYXW, 14=WYYY, 15=WXYW, 16=WYXW, 17=WXXW, 18=WYYW, 19=XYYX, 20=YXYX, 21=YXXY and 22=XYXY.

Once the 4-mers are assigned to positions according to the above pattern, a particular set of oligonucleotides can be created by appropriate assignment of bases, A, T/U, G, C to W, X, Y. These assignments are made according to one of the following two sets of rules:

(i) Each of W, X and Y is a base in which:
(a) W=one of A, T/U, G, and C,
   X=one of A, T/U, G, and C,
   Y=one of A, T/U, G, and C,

and each of W, X and Y is selected so as to be different from all of the others of W, X and Y,
(b) an unselected said base of (i)(a) can be substituted any number of times for any one of W, X and Y;

### OR

(ii) Each of W, X and Y is a base in which:
(a) W=G or C,
   X=A or T/U,
   Y=A or T/U,
   and X≠Y, and
(b) a base not selected in (ii)(a) can be inserted into each sequence at one or more locations, the location of each insertion being the same in each sequence as that of every other sequence of the set;

In the case of the specific oligonucleotides given in Table I, W=G, X=A and Y=T.

In any case, given a set of oligonucleotides generated according to one of these sets of rules, it is possible to modify the members of a given set in relatively minor ways and thereby obtain a different set of sequences while more or less maintaining the cross-hybridization properties of the set subject to such modification. In particular, it is possible to insert up to 3 of A, T/U, G and C at any location of any sequence of the set of sequences. Alternatively, or additionally, up to 3 bases can be deleted from any sequence of the set of sequences.

A person skilled in the art would understand that given a set of oligonucleotides having a set of properties making it suitable for use as a family of tags (or tag complements), one can obtain another family with the same property by reversing the order of all of the members of the set. In other words, all the members can be taken to be read 5′ to 3′ or to be read 3′ to 5′.

A family of complements of the present invention is based on a given set of oligonucleotides defined as described above. Each complement of the family is based on a different oligonucleotide of the set and each complement contains at least 10 consecutive (i.e., contiguous) bases of the oligonucleotide on which it is based. For a given family of complements where one is seeking to reduce or minimize inter-sequence similarity that would result in cross-hybridization, each and every pair of complements meets particular homology requirements. Particularly, subject to limited exceptions, described below, any two complements within a set of complements are generally required to have a defined amount of dissimilarity.

In order to notionally understand these requirements for dissimilarity as they exist for a given pair of complements of a family, a phantom sequence is generated from the pair of complements. A “phantom” sequence is a single sequence that is generated from a pair of complements by selection, from each complement of the pair, of a string of bases wherein the bases of the string occur in the same order in both complements. An object of creating such a phantom sequence is to create a convenient and objective means of comparing the sequence identity of the two parent sequences from which the phantom sequence is created.

A phantom sequence may thus be generated from exemplary Sequence 1 and Sequence 2 as follows:

(SEQ ID NO: 1383)

| | |
|---|---|
| Sequence 1: | ATGTTTAGTGAAAAGTTAGTATTG |
| | * • |

(SEQ ID NO: 1384)

| | |
|---|---|
| Sequence 2: | ATGTTAGTGAATAGTATAGTATTG |
| | • ♦ |

(SEQ ID NO: 1385)

| | |
|---|---|
| Phantom Sequence: | ATGTTAGTGAAAGTTAGTATTG |

The phantom sequence generated from these two sequences is thus 22 bases in length. That is, one can see that there are 22 identical bases with identical sequence (the same order) in Sequence Nos. 1 and 2. There is a total of three insertions/deletions and mismatches present in the phantom sequence when compared with the sequences from which it was generated:

| | |
|---|---|
| ATGT-TAGTGAA-AGT-TAGTATTG | (SEQ ID NO: 1385) |

The dashed lines in this latter representation of the phantom sequence indicate the locations of the insertions/deletions and mismatches in the phantom sequence relative to the parent sequences from which it was derived. Thus, the “T” marked with an asterisk in Sequence 1, the “A” marked with a diamond in Sequence 2 and the “A-T” mismatch of Sequences 1 and 2 marked with two dots were deleted in generating the phantom sequence.

A person skilled in the art will appreciate that the term “insertion/deletion” is intended to cover the situations indicated by the asterisk and diamond. Whether the change is considered, strictly speaking, an insertion or deletion is merely one of vantage point. That is, one can see that the fourth base of Sequence 1 can be deleted therefrom to obtain the phantom sequence, or a “T” can be inserted after the third base of the phantom sequence to obtain Sequence 1.

One can thus see that if it were possible to create a phantom sequence by elimination of a single insertion/deletion from one of the parent sequences, that the two parent sequences would have identical homology over the length of the phantom sequence except for the presence of a single base in one of the two sequences being compared. Likewise, one can see that if it were possible to create a phantom sequence through deletion of a mismatched pair of bases, one base in each parent, that the two parent sequences would have identical homology over the length of the phantom sequence except for the presence of a single base in each of the sequences being compared. For this reason, the effect of an insertion/deletion is considered equivalent to the effect of a mismatched pair of bases when comparing the homology of two sequences.

Once a phantom sequence is generated, the compatibility of the pair of complements from which it was generated within a family of complements can be systematically evaluated.

According to one embodiment of the invention, a pair of complements is compatible for inclusion within a family of complements if any phantom sequence generated from the pair of complements has the following properties:
Any consecutive sequence of bases in the phantom sequence which is identical to a consecutive sequence of bases in each of the first and second complements from which it is generated is no more ((¾×L)−1) bases in length;
The phantom sequence, if greater than or equal to (⅚×L) in length, contains at least 3 insertions/deletions or mismatches when compared to t first and second complements from which it is generated; and
The phantom sequence is not greater than or equal to (( 11/12×L) in length.

Here, L₁ is the length of the first complement, L₂ is the length of the second complement, and L=L₁, or if L₁≠L₂, L is the greater of L₁ and L₂.

In particular preferred embodiments of the invention, all pairs of complements of a given set have the properties set out above. Under particular circumstances, it may be advantageous to have a limited number of complements that do not meet all of these requirements when compared to every other complement in a family.

In one case, for any first complement there are at most two second complements in the family which do not meet all of the three listed requirements. For two such complements, there would thus be a greater chance of cross-hybridization between their tag counterparts and the first complement. In another case, for any first complement there is at most one second complement which does not meet all of three listed requirements.

It is also possible, given this invention, to design a family of complements where a specific number or specific portion of the complements do not meet the three listed requirements. For example, a set could be designed where only one pair of complements within the set do not meet the requirements when compared to each other. There could be two pairs, three pairs, and any number of pairs up to and including all possible pairs. Alternatively, it may be advantageous to have a given proportion of pairs of complements that do not meet the requirements, say 10% of pairs, when compared with other sequences that do not meet one or more of the three requirements listed. This number could instead by 5%, 15%, 20%, 25%, 30%, 35%, or 40%.

The foregoing comparisons would generally be largely carried out using appropriate computer software. Although notionally described in terms of a phantom sequence for the sake of clarity and understanding, it will be understood that a competent computer programmer can carry out pair-wise comparisons of complements in any number of ways using logical steps that obtain equivalent results.

The symbols A, G, T/U; C take on their usual meaning in the art here. In the case of T and U, a person skilled in the art would understand that these are equivalent to each other with respect to the inter-strand hydrogen-bond (Watson-Crick) binding properties at work in the context of this invention. The two bases are thus interchangeable and hence the designation of T/U. Base analogues can be inserted in their respective places where desired.

In another broad embodiment, a family of 1168 sequences was determined using a computer algorithm to have desirable hybridization properties for use in nucleic acid detection assays. The sequence set of 1168 oligonucleotides have been partially characterized in hybridization assays, demonstrating the ability of family members to correctly hybridize to their complementary sequences with minimal cross hybridization. (See Janeczko, supra). These are the sequences having SEQ ID NOS: 211 to 1378 of Table II.

Variant families of sequences (seen as tags or tag complements) of a family of sequences taken from Table II are also part of the invention. For the purposes of discussion, a family or set of oligonucleotides will often be described as a family of tag complements, but it will be understood that such a set could just easily be a family of tags.

A family of complements is obtained from a set of oligonucleotides based on a family of oligonucleotides such as those of Table II. To simplify discussion, providing a family of complements based on the oligonucleotides of Table II will be described.

Firstly, the groups of sequences based on the oligonucleotides of Table II can be represented as shown in Table IIA.

TABLE IIA
Numeric sequences corresponding to nucleotide patterns of a set of oligonucleotides
Tag Identifier Numeric Patterns

| Tag | | | | | | | | | | | | | | | | | | | | | | | | |
|---|---|---|---|---|---|---|---|---|---|---|---|---|---|---|---|---|---|---|---|---|---|---|---|---|
| identifier | Numeric Pattern | | | | | | | | | | | | | | | | | | | | | | | |
| 211 | 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 |
| 212 | 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 |
| 213 | 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 |
| 214 | 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 |
| 215 | 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 |
| 216 | 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 |
| 217 | 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 |
| 218 | 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 |
| 219 | 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 |
| 220 | 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 |
| 221 | 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 |
| 222 | 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 |
| 223 | 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 |
| 224 | 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 |
| 225 | 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 |
| 226 | 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 |
| 227 | 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 |
| 228 | 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 |
| 229 | 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 |
| 230 | 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 |
| 231 | 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 |
| 232 | 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 |
| 233 | 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 |
| 234 | 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 |
| 235 | 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 |
| 236 | 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 |
| 237 | 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 |
| 238 | 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 |
| 239 | 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 |
| 240 | 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 |
| 241 | 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 |
| 242 | 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 |
| 243 | 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 |
| 244 | 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 |
| 245 | 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 |
| 246 | 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 |
| 247 | 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 |
| 248 | 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 |
| 249 | 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 |
| 250 | 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 |
| 251 | 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 |
| 252 | 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 |
| 253 | 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 |
| 254 | 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 |
| 255 | 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 |
| 256 | 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 |
| 257 | 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 |
| 258 | 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 |
| 259 | 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 |
| 260 | 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 |
| 261 | 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 |
| 262 | 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 |
| 263 | 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 |
| 264 | 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 |
| 265 | 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 |
| 266 | 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 |
| 267 | 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 |
| 268 | 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 |
| 269 | 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 |
| 270 | 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 |
| 271 | 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 |
| 272 | 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 |
| 273 | 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 |
| 274 | 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 |
| 275 | 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 |
| 276 | 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 |
| 277 | 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 |
| 278 | 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 |
| 279 | 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 |
| 280 | 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 |
| 281 | 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 |
| 282 | 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 |
| 283 | 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 |
| 284 | 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 |
| 285 | 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 |
| 286 | 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 |
| 287 | 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 |
| 288 | 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 |
| 289 | 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 |
| 290 | 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 |
| 291 | 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 |
| 292 | 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 |
| 293 | 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 |
| 294 | 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 |
| 295 | 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 |
| 296 | 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 |
| 297 | 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 |
| 298 | 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 |
| 299 | 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 |
| 300 | 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 |
| 301 | 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 |
| 302 | 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 |
| 303 | 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 |
| 304 | 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 |
| 305 | 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 |
| 306 | 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 |
| 307 | 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 |
| 308 | 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 |
| 309 | 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 |
| 310 | 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 |
| 311 | 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 |
| 312 | 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 |
| 313 | 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 |
| 314 | 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 |
| 315 | 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 |
| 316 | 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 |
| 317 | 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 |
| 318 | 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 |
| 319 | 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 |
| 320 | 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 |
| 321 | 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 |
| 322 | 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 |
| 323 | 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 |
| 324 | 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 |
| 325 | 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 |
| 326 | 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 |
| 327 | 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 |
| 328 | 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 |
| 329 | 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 |
| 330 | 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 |
| 331 | 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 |
| 332 | 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 |
| 333 | 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 |
| 334 | 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 |
| 335 | 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 |
| 336 | 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 |
| 337 | 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 |
| 338 | 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 |
| 339 | 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 |
| 340 | 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 |
| 341 | 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 |
| 342 | 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 |
| 343 | 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 |
| 344 | 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 |
| 345 | 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 |
| 346 | 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 |
| 347 | 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 |
| 348 | 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 |
| 349 | 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 |
| 350 | 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 |
| 351 | 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 |
| 352 | 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 |
| 353 | 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 |
| 354 | 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 |
| 355 | 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 |
| 356 | 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 |
| 357 | 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 |
| 358 | 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 |
| 359 | 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 |
| 360 | 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 |
| 361 | 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 |
| 362 | 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 |
| 363 | 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 |
| 364 | 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 |
| 365 | 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 |
| 366 | 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 |
| 367 | 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 |
| 368 | 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 |
| 369 | 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 |
| 370 | 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 |
| 371 | 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 |
| 372 | 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 |
| 373 | 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 |
| 374 | 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 |
| 375 | 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 |
| 376 | 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 |
| 377 | 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 |
| 378 | 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 |
| 379 | 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 |
| 380 | 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 |
| 381 | 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 |
| 382 | 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 |
| 383 | 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 |
| 384 | 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 |
| 385 | 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 |
| 386 | 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 |
| 387 | 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 |
| 388 | 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 |
| 389 | 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 |
| 390 | 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 |
| 391 | 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 |
| 392 | 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 |
| 393 | 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 |
| 394 | 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 |
| 395 | 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 |
| 396 | 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 |
| 397 | 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 |
| 398 | 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 |
| 399 | 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 |
| 400 | 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 |
| 401 | 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 |
| 402 | 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 |
| 403 | 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 |
| 404 | 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 |
| 405 | 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 |
| 406 | 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 |
| 407 | 3 | 2 | 2 | 2 | 1 | 2 | 2 | 3 | 2 | 1 | 1 | 1 | 3 | 1 | 2 | 3 | 2 | 1 | 1 | 3 | 2 | 3 | 1 | 1 |
| 408 | 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 |
| 409 | 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 |
| 410 | 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 |
| 411 | 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 |
| 412 | 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 |
| 413 | 2 | 3 | 2 | 3 | 2 | 1 | 2 | 1 | 1 | 3 | 1 | 2 | 1 | 2 | 2 | 2 | 3 | 2 | 1 | 1 | 3 | 1 | 1 | 3 |
| 414 | 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 |
| 415 | 3 | 2 | 3 | 2 | 2 | 1 | 2 | 2 | 1 | 3 | 2 | 2 | 2 | 1 | 1 | 3 | 2 | 2 | 1 | 3 | 1 | 3 | 2 | 1 |
| 416 | 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 |
| 417 | 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 |
| 418 | 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 |
| 419 | 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 |
| 420 | 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 |
| 421 | 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 |
| 422 | 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 |
| 423 | 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 |
| 424 | 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 |
| 425 | 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 |
| 426 | 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 |
| 427 | 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 |
| 428 | 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 |
| 429 | 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 |
| 430 | 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 |
| 431 | 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 |
| 432 | 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 |
| 433 | 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 |
| 434 | 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 |
| 435 | 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 |
| 436 | 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 |
| 437 | 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 |
| 438 | 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 |
| 439 | 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 |
| 440 | 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 |
| 441 | 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 |
| 442 | 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 |
| 443 | 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 |
| 444 | 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 |
| 445 | 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 |
| 446 | 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 |
| 447 | 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 |
| 448 | 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 |
| 449 | 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 |
| 450 | 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 |
| 451 | 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 |
| 452 | 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 |
| 453 | 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 |
| 454 | 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 |
| 455 | 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 |
| 456 | 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 |
| 457 | 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 |
| 458 | 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 |
| 459 | 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 |
| 460 | 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 |
| 461 | 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 |
| 462 | 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 |
| 463 | 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 |
| 464 | 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 |
| 465 | 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 |
| 466 | 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 |
| 467 | 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 |
| 468 | 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 |
| 469 | 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 |
| 470 | 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 |
| 471 | 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 |
| 472 | 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 |
| 473 | 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 |
| 474 | 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 |
| 475 | 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 |
| 476 | 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 |
| 477 | 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 |
| 478 | 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 |
| 479 | 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 |
| 480 | 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 |
| 481 | 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 |
| 482 | 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 |
| 483 | 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 |
| 484 | 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 |
| 485 | 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 |
| 486 | 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 |
| 487 | 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 |
| 488 | 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 |
| 489 | 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 |
| 490 | 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 |
| 491 | 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 |
| 492 | 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 |
| 493 | 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 |
| 494 | 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 |
| 495 | 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 |
| 496 | 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 |
| 497 | 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 |
| 498 | 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 |
| 499 | 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 |
| 500 | 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 |
| 501 | 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 |
| 502 | 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 |
| 503 | 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 |
| 504 | 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 |
| 505 | 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 |
| 506 | 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 |
| 507 | 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 |
| 508 | 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 |
| 509 | 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 |
| 510 | 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 |
| 511 | 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 |
| 512 | 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 |
| 513 | 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 |
| 514 | 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 |
| 515 | 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 |
| 516 | 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 |
| 517 | 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 |
| 518 | 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 |
| 519 | 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 |
| 520 | 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 |
| 521 | 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 |
| 522 | 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 |
| 523 | 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 |
| 524 | 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 |
| 525 | 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 |
| 526 | 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 |
| 527 | 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 |
| 528 | 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 |
| 529 | 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 |
| 530 | 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 |
| 531 | 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 |
| 532 | 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 |
| 533 | 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 |
| 534 | 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 |
| 535 | 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 |
| 536 | 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 |
| 537 | 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 |
| 538 | 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 |
| 539 | 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 |
| 540 | 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 |
| 541 | 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 |
| 542 | 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 |
| 543 | 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 |
| 544 | 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 |
| 545 | 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 |
| 546 | 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 |
| 547 | 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 |
| 548 | 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 |
| 549 | 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 |
| 550 | 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 |
| 551 | 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 |
| 552 | 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 |
| 553 | 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 |
| 554 | 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 |
| 555 | 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 |
| 556 | 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 |
| 557 | 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 |
| 558 | 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 |
| 559 | 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 |
| 560 | 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 |
| 561 | 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 |
| 562 | 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 |
| 563 | 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 |
| 564 | 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 |
| 565 | 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 |
| 566 | 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 |
| 567 | 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 |
| 568 | 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 |
| 569 | 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 |
| 570 | 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 |
| 571 | 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 |
| 572 | 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 |
| 573 | 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 |
| 574 | 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 |
| 575 | 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 |
| 576 | 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 |
| 577 | 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 |
| 578 | 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 |
| 579 | 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 |
| 580 | 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 |
| 581 | 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 |
| 582 | 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 |
| 583 | 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 |
| 584 | 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 |
| 585 | 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 |
| 586 | 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 |
| 587 | 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 |
| 588 | 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 |
| 589 | 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 |
| 590 | 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 |
| 591 | 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 |
| 592 | 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 |
| 593 | 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 |
| 594 | 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 |
| 595 | 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 |
| 596 | 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 |
| 597 | 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 |
| 598 | 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 |
| 599 | 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 |
| 600 | 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 |
| 601 | 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 |
| 602 | 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 |
| 603 | 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 |
| 604 | 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 |
| 605 | 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 |
| 606 | 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 |
| 607 | 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 |
| 608 | 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 |
| 609 | 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 |
| 610 | 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 |
| 611 | 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 |
| 612 | 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 |
| 613 | 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 |
| 614 | 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 |
| 615 | 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 |
| 616 | 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 |
| 617 | 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 |
| 618 | 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 |
| 619 | 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 |
| 620 | 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 |
| 621 | 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 |
| 622 | 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 |
| 623 | 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 |
| 624 | 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 |
| 625 | 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 |
| 626 | 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 |
| 627 | 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 |
| 628 | 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 |
| 629 | 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 |
| 630 | 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 |
| 631 | 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 |
| 632 | 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 |
| 633 | 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 |
| 634 | 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 |
| 635 | 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 |
| 636 | 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 |
| 637 | 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 |
| 638 | 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 |
| 639 | 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 |
| 640 | 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 |
| 641 | 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 |
| 642 | 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 |
| 643 | 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 |
| 644 | 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 |
| 645 | 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 |
| 646 | 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 |
| 647 | 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 |
| 648 | 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 |
| 649 | 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 |
| 650 | 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 |
| 651 | 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 |
| 652 | 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 |
| 653 | 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 |
| 654 | 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 |
| 655 | 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 |
| 656 | 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 |
| 657 | 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 |
| 658 | 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 |
| 659 | 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 |
| 660 | 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 |
| 661 | 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 |
| 662 | 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 |
| 663 | 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 |
| 664 | 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 |
| 665 | 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 |
| 666 | 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 |
| 667 | 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 |
| 668 | 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 |
| 669 | 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 |
| 670 | 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 |
| 671 | 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 |
| 672 | 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 |
| 673 | 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 |
| 674 | 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 |
| 675 | 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 |
| 676 | 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 |
| 677 | 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 |
| 678 | 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 |
| 679 | 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 |
| 680 | 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 |
| 681 | 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 |
| 682 | 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 |
| 683 | 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 |
| 684 | 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 |
| 685 | 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 |
| 686 | 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 |
| 687 | 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 |
| 688 | 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 |
| 689 | 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 |
| 690 | 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 |
| 691 | 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 |
| 692 | 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 |
| 693 | 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 |
| 694 | 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 |
| 695 | 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 |
| 696 | 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 |
| 697 | 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 |
| 698 | 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 |
| 699 | 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 |
| 700 | 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 |
| 701 | 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 |
| 702 | 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 |
| 703 | 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 |
| 704 | 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 |
| 705 | 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 |
| 706 | 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 |
| 707 | 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 |
| 708 | 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 |
| 709 | 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 |
| 710 | 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 |
| 711 | 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 |
| 712 | 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 |
| 713 | 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 |
| 714 | 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 |
| 715 | 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 |
| 716 | 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 |
| 717 | 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 |
| 718 | 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 |
| 719 | 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 |
| 720 | 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 |
| 721 | 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 |
| 722 | 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 |
| 723 | 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 |
| 724 | 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 |
| 725 | 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 |
| 726 | 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 |
| 727 | 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 |
| 728 | 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 |
| 729 | 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 |
| 730 | 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 |
| 731 | 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 |
| 732 | 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 |
| 733 | 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 |
| 734 | 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 |
| 735 | 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 |
| 736 | 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 |
| 737 | 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 |
| 738 | 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 |
| 739 | 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 |
| 740 | 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 |
| 741 | 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 |
| 742 | 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 |
| 743 | 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 |
| 744 | 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 |
| 745 | 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 |
| 746 | 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 |
| 747 | 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 |
| 748 | 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 |
| 749 | 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 |
| 750 | 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 |
| 751 | 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 |
| 752 | 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 |
| 753 | 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 |
| 754 | 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 |
| 755 | 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 |
| 756 | 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 |
| 757 | 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 |
| 758 | 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 |
| 759 | 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 |
| 760 | 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 |
| 761 | 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 |
| 762 | 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 |
| 763 | 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 |
| 764 | 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 |
| 765 | 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 |
| 766 | 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 |
| 767 | 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 |
| 768 | 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 |
| 769 | 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 |
| 770 | 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 |
| 771 | 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 |
| 772 | 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 |
| 773 | 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 |
| 774 | 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 |
| 775 | 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 |
| 776 | 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 |
| 777 | 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 |
| 778 | 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 |
| 779 | 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 |
| 780 | 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 |
| 781 | 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 |
| 782 | 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 |
| 783 | 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 |
| 784 | 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 |
| 785 | 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 |
| 786 | 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 |
| 787 | 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 |
| 788 | 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 |
| 789 | 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 |
| 790 | 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 |
| 791 | 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 |
| 792 | 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 |
| 793 | 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 |
| 794 | 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 |
| 795 | 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 |
| 796 | 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 |
| 797 | 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 |
| 798 | 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 |
| 799 | 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 |
| 800 | 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 |
| 801 | 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 |
| 802 | 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 |
| 803 | 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 |
| 804 | 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 |
| 805 | 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 |
| 806 | 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 |
| 807 | 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 |
| 808 | 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 |
| 809 | 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 |
| 810 | 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 |
| 811 | 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 |
| 812 | 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 |
| 813 | 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 |
| 814 | 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 |
| 815 | 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 |
| 816 | 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 |
| 817 | 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 |
| 818 | 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 |
| 819 | 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 |
| 820 | 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 |
| 821 | 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 |
| 822 | 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 |
| 823 | 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 |
| 824 | 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 |
| 825 | 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 |
| 826 | 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 |
| 827 | 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 |
| 828 | 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 |
| 829 | 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 |
| 830 | 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 |
| 831 | 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 |
| 832 | 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 |
| 833 | 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 |
| 834 | 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 |
| 835 | 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 |
| 836 | 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 |
| 837 | 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 |
| 838 | 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 |
| 839 | 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 |
| 840 | 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 |
| 841 | 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 |
| 842 | 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 |
| 843 | 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 |
| 844 | 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 |
| 845 | 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 |
| 846 | 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 |
| 847 | 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 |
| 848 | 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 |
| 849 | 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 |
| 850 | 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 |
| 851 | 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 |
| 852 | 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 |
| 853 | 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 |
| 854 | 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 |
| 855 | 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 |
| 856 | 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 |
| 857 | 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 |
| 858 | 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 |
| 859 | 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 |
| 860 | 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 |
| 861 | 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 |
| 862 | 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 |
| 863 | 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 |
| 864 | 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 |
| 865 | 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 |
| 866 | 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 |
| 867 | 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 |
| 868 | 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 |
| 869 | 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 |
| 870 | 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 |
| 871 | 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 |
| 872 | 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 |
| 873 | 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 |
| 874 | 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 |
| 875 | 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 |
| 876 | 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 |
| 877 | 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 |
| 878 | 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 |
| 879 | 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 |
| 880 | 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 |
| 881 | 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 |
| 882 | 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 |
| 883 | 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 |
| 884 | 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 |
| 885 | 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 |
| 886 | 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 |
| 887 | 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 |
| 888 | 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 |
| 889 | 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 |
| 890 | 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 |
| 891 | 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 |
| 892 | 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 |
| 893 | 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 |
| 894 | 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 |
| 895 | 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 |
| 896 | 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 |
| 897 | 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 |
| 898 | 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 |
| 899 | 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 |
| 900 | 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 |
| 901 | 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 |
| 902 | 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 |
| 903 | 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 |
| 904 | 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 |
| 905 | 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 |
| 906 | 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 |
| 907 | 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 |
| 908 | 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 |
| 909 | 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 |
| 910 | 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 |
| 911 | 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 |
| 912 | 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 |
| 913 | 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 |
| 914 | 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 |
| 915 | 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 |
| 916 | 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 |
| 917 | 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 |
| 918 | 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 |
| 919 | 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 |
| 920 | 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 |
| 921 | 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 |
| 922 | 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 |
| 923 | 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 |
| 924 | 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 |
| 925 | 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 |
| 926 | 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 |
| 927 | 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 |
| 928 | 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 |
| 929 | 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 |
| 930 | 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 |
| 931 | 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 |
| 932 | 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 |
| 933 | 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 |
| 934 | 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 |
| 935 | 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 |
| 936 | 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 |
| 937 | 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 |
| 938 | 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 |
| 939 | 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 |
| 940 | 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 |
| 941 | 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 |
| 942 | 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 |
| 943 | 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 |
| 944 | 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 |
| 945 | 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 |
| 946 | 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 |
| 947 | 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 |
| 948 | 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 |
| 949 | 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 |
| 950 | 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 |
| 951 | 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 |
| 952 | 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 |
| 953 | 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 |
| 954 | 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 |
| 955 | 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 |
| 956 | 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 |
| 957 | 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 |
| 958 | 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 |
| 959 | 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 |
| 960 | 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 |
| 961 | 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 |
| 962 | 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 |
| 963 | 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 |
| 964 | 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 |
| 965 | 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 |
| 966 | 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 |
| 967 | 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 |
| 968 | 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 |
| 969 | 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 |
| 970 | 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 |
| 971 | 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 |
| 972 | 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 |
| 973 | 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 |
| 974 | 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 |
| 975 | 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 |
| 976 | 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 |
| 977 | 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 |
| 978 | 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 |
| 979 | 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 |
| 980 | 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 |
| 981 | 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 |
| 982 | 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 |
| 983 | 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 |
| 984 | 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 |
| 985 | 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 |
| 986 | 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 |
| 987 | 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 |
| 988 | 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 |
| 989 | 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 |
| 990 | 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 |
| 991 | 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 |
| 992 | 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 |
| 993 | 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 |
| 994 | 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 |
| 995 | 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 |
| 996 | 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 |
| 997 | 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 |
| 998 | 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 |
| 999 | 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 |
| 1000 | 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 |
| 1001 | 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 |
| 1002 | 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 |
| 1003 | 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 |
| 1004 | 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 |
| 1005 | 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 |
| 1006 | 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 |
| 1007 | 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 |
| 1008 | 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 |
| 1009 | 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 |
| 1010 | 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 |
| 1011 | 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 |
| 1012 | 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 |
| 1013 | 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 |
| 1014 | 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 |
| 1015 | 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 |
| 1016 | 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 |
| 1017 | 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 |
| 1018 | 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 |
| 1019 | 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 |
| 1020 | 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 |
| 1021 | 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 |
| 1022 | 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 |
| 1023 | 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 |
| 1024 | 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 |
| 1025 | 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 |
| 1026 | 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 |
| 1027 | 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 |
| 1028 | 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 |
| 1029 | 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 |
| 1030 | 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 |
| 1031 | 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 |
| 1032 | 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 |
| 1033 | 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 |
| 1034 | 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 |
| 1035 | 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 |
| 1036 | 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 |
| 1037 | 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 |
| 1038 | 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 |
| 1039 | 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 |
| 1040 | 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 |
| 1041 | 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 |
| 1042 | 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 |
| 1043 | 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 |
| 1044 | 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 |
| 1045 | 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 |
| 1046 | 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 |
| 1047 | 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 |
| 1048 | 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 |
| 1049 | 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 |
| 1050 | 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 |
| 1051 | 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 |
| 1052 | 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 |
| 1053 | 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 |
| 1054 | 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 |
| 1055 | 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 |
| 1056 | 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 |
| 1057 | 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 |
| 1058 | 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 |
| 1059 | 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 |
| 1060 | 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 |
| 1061 | 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 |
| 1062 | 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 |
| 1063 | 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 |
| 1064 | 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 |
| 1065 | 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 |
| 1066 | 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 |
| 1067 | 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 |
| 1068 | 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 |
| 1069 | 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 |
| 1070 | 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 |
| 1071 | 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 |
| 1072 | 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 |
| 1073 | 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 |
| 1074 | 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 |
| 1075 | 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 |
| 1076 | 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 |
| 1077 | 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 |
| 1078 | 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 |
| 1079 | 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 |
| 1080 | 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 |
| 1081 | 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 |
| 1082 | 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 |
| 1083 | 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 |
| 1084 | 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 |
| 1085 | 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 |
| 1086 | 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 |
| 1087 | 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 |
| 1088 | 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 |
| 1089 | 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 |
| 1090 | 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 |
| 1091 | 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 |
| 1092 | 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 |
| 1093 | 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 |
| 1094 | 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 |
| 1095 | 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 |
| 1096 | 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 |
| 1097 | 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 |
| 1098 | 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 |
| 1099 | 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 |
| 1100 | 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 |
| 1101 | 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 |
| 1102 | 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 |
| 1103 | 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 |
| 1104 | 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 |
| 1105 | 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 |
| 1106 | 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 |
| 1107 | 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 |
| 1108 | 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 |
| 1109 | 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 |
| 1110 | 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 |
| 1111 | 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 |
| 1112 | 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 |
| 1113 | 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 |
| 1114 | 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 |
| 1115 | 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 |
| 1116 | 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 |
| 1117 | 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 |
| 1118 | 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 |
| 1119 | 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 |
| 1120 | 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 |
| 1121 | 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 |
| 1122 | 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 |
| 1123 | 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 |
| 1124 | 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 |
| 1125 | 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 |
| 1126 | 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 |
| 1127 | 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 |
| 1128 | 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 |
| 1129 | 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 |
| 1130 | 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 |
| 1131 | 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 |
| 1132 | 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 |
| 1133 | 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 |
| 1134 | 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 |
| 1135 | 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 |
| 1136 | 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 |
| 1137 | 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 |
| 1138 | 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 |
| 1139 | 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 |
| 1140 | 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 |
| 1141 | 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 |
| 1142 | 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 |
| 1143 | 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 |
| 1144 | 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 |
| 1145 | 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 |
| 1146 | 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 |
| 1147 | 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 |
| 1148 | 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 |
| 1149 | 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 |
| 1150 | 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 |
| 1151 | 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 |
| 1152 | 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 |
| 1153 | 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 |
| 1154 | 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 |
| 1155 | 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 |
| 1156 | 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 |
| 1157 | 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 |
| 1158 | 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 |
| 1159 | 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 |
| 1160 | 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 |
| 1161 | 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 |
| 1162 | 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 |
| 1163 | 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 |
| 1164 | 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 |
| 1165 | 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 |
| 1166 | 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 |
| 1167 | 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 |
| 1168 | 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 |
| 1169 | 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 |
| 1170 | 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 |
| 1171 | 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 |
| 1172 | 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 |
| 1173 | 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 |
| 1174 | 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 |
| 1175 | 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 |
| 1176 | 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 |
| 1177 | 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 |
| 1178 | 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 |
| 1179 | 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 |
| 1180 | 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 |
| 1181 | 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 |
| 1182 | 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 |
| 1183 | 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 |
| 1184 | 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 |
| 1185 | 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 |
| 1186 | 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 |
| 1187 | 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 |
| 1188 | 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 |
| 1189 | 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 |
| 1190 | 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 |
| 1191 | 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 |
| 1192 | 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 |
| 1193 | 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 |
| 1194 | 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 |
| 1195 | 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 |
| 1196 | 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 |
| 1197 | 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 |
| 1198 | 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 |
| 1199 | 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 |
| 1200 | 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 |
| 1201 | 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 |
| 1202 | 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 |
| 1203 | 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 |
| 1204 | 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 |
| 1205 | 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 |
| 1206 | 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 |
| 1207 | 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 |
| 1208 | 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 |
| 1209 | 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 |
| 1210 | 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 |
| 1211 | 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 |
| 1212 | 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 |
| 1213 | 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 |
| 1214 | 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 |
| 1215 | 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 |
| 1216 | 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 |
| 1217 | 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 |
| 1218 | 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 |
| 1219 | 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 |
| 1220 | 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 |
| 1221 | 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 |
| 1222 | 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 |
| 1223 | 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 |
| 1224 | 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 |
| 1225 | 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 |
| 1226 | 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 |
| 1227 | 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 |
| 1228 | 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 |
| 1229 | 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 |
| 1230 | 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 |
| 1231 | 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 |
| 1232 | 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 |
| 1233 | 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 |
| 1234 | 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 |
| 1235 | 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 |
| 1236 | 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 |
| 1237 | 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 |
| 1238 | 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 |
| 1239 | 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 |
| 1240 | 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 |
| 1241 | 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 |
| 1242 | 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 |
| 1243 | 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 |
| 1244 | 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 |
| 1245 | 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 |
| 1246 | 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 |
| 1247 | 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 |
| 1248 | 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 |
| 1249 | 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 |
| 1250 | 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 |
| 1251 | 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 |
| 1252 | 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 |
| 1253 | 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 |
| 1254 | 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 |
| 1255 | 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 |
| 1256 | 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 |
| 1257 | 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 |
| 1258 | 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 |
| 1259 | 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 |
| 1260 | 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 |
| 1261 | 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 |
| 1262 | 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 |
| 1263 | 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 |
| 1264 | 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 |
| 1265 | 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 |
| 1266 | 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 |
| 1267 | 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 |
| 1268 | 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 |
| 1269 | 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 |
| 1270 | 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 |
| 1271 | 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 |
| 1272 | 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 |
| 1273 | 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 |
| 1274 | 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 |
| 1275 | 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 |
| 1276 | 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 |
| 1277 | 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 |
| 1278 | 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 |
| 1279 | 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 |
| 1280 | 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 |
| 1281 | 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 |
| 1282 | 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 |
| 1283 | 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 |
| 1284 | 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 |
| 1285 | 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 |
| 1286 | 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 |
| 1287 | 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 |
| 1288 | 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 |
| 1289 | 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 |
| 1290 | 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 |
| 1291 | 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 |
| 1292 | 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 |
| 1293 | 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 |
| 1294 | 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 |
| 1295 | 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 |
| 1296 | 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 |
| 1297 | 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 |
| 1298 | 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 |
| 1299 | 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 |
| 1300 | 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 |
| 1301 | 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 |
| 1302 | 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 |
| 1303 | 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 |
| 1304 | 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 |
| 1305 | 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 |
| 1306 | 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 |
| 1307 | 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 |
| 1308 | 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 |
| 1309 | 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 |
| 1310 | 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 |
| 1311 | 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 |
| 1312 | 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 |
| 1313 | 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 |
| 1314 | 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 |
| 1315 | 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 |
| 1316 | 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 |
| 1317 | 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 |
| 1318 | 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 |
| 1319 | 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 |
| 1320 | 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 |
| 1321 | 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 |
| 1322 | 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 |
| 1323 | 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 |
| 1324 | 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 |
| 1325 | 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 |
| 1326 | 2 | 2 | 1 | 1 | 1 | 2 | 3 | 2 | 3 | 2 | 3 | 1 | 3 | 1 | 1 | 3 | 1 | 2 | 3 | 1 | 1 | 2 | T | 1 |
| 1327 | 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 |
| 1328 | 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 |
| 1329 | 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 |
| 1330 | 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 |
| 1331 | 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 |
| 1332 | 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 |
| 1333 | 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 |
| 1334 | 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 |
| 1335 | 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 |
| 1336 | 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 |
| 1337 | 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 |
| 1338 | 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 |
| 1339 | 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 |
| 1340 | 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 |
| 1341 | 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 |
| 1342 | 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 |
| 1343 | 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 |
| 1344 | 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 |
| 1345 | 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 |
| 1346 | 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 |
| 1347 | 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 |
| 1348 | 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 |
| 1349 | 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 |
| 1350 | 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 |
| 1351 | 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 |
| 1352 | 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 |
| 1353 | 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 |
| 1354 | 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 |
| 1355 | 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 |
| 1356 | 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 |
| 1357 | 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 |
| 1358 | 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 |
| 1359 | 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 |
| 1360 | 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 |
| 1361 | 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 |
| 1362 | 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 |
| 1363 | 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 |
| 1364 | 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 |
| 1365 | 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 |
| 1366 | 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 |
| 1367 | 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 |
| 1368 | 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 |
| 1369 | 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 |
| 1370 | 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 |
| 1371 | 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 |
| 1372 | 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 |
| 1373 | 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 |
| 1374 | 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 |
| 1375 | 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 |
| 1376 | 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 |
| 1377 | 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 |
| 1378 | 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 |

In Table IIA, each of the numerals 1 to 3 (“numeric identifiers”) represents a nucleotide base and the pattern of numeric identifiers (1, 2 or 3) in the above list corresponds to the pattern of nucleotide bases present in the tag, e.g., in the oligonucleotides of Table II, below. These oligonucleotides have been found to be non- or minimally cross-hybridizing (See Janeczko, supra).

Each pattern is identified by a number in the left column, the “tag identifier,” which is associated with the pattern of numeric identifiers on that line. Each nucleotide base is selected from the group of nucleotide bases consisting of A, C, G, and T/U. A particularly preferred embodiment of the invention, in which a specific base is assigned to each numeric identifier is shown in Table II, below.

In another broad aspect, the invention is a composition comprising INVADER assay probes comprising tags or tag complements, wherein each tag portion of the molecule comprises an oligonucleotide selected from a set of oligonucleotides based on a group of sequences as specified by numeric identifiers set out in Table IIA. In the sequences, each of 1 to 3 is a nucleotide base selected to be different from the others of 1 to 3 with the proviso that up to three nucleotide bases of each sequence can be substituted with any nucleotide base provided that:

for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein:
the score of an alignment is determined according to the equation (A×M)−(B×mm)−(C×(og+eg))−(D−eg)), wherein:
for each of (i) to (iv):
   (i) m=6, mm=6, og=0 and eg=6,
   (ii) m=6, mm=6, og=5 and eg=1,
   (iii) m=6, mm=2, og=5 and eg=1, and
   (iv) m=6, mm=6, og=6 and eg=0,
A is the total number of matched pairs of bases in the alignment;
B is the total number of internal mismatched pairs in the alignment;
C is the total number of internal gaps in the alignment; and.
D is the total number of internal indels in the alignment minus the total number of internal gaps in the alignment; and
wherein the maximum score is determined separately for each of (i), (ii), (iii) and (iv).

An explanation of the meaning of the parameters set out above is given in the section describing detailed embodiments.

In another broad aspect, the invention is a composition comprising INVADER assay probes comprising tags or tag complements, wherein each tag portion of the molecule comprises an oligonucleotide selected from a set of oligonucleotides based on a group of sequences as specified by numeric identifiers set out in Table IIA wherein each of 1 to 3 is a nucleotide base selected to be different from the others of 1 to 3 with the proviso that up to three nucleotide bases of each sequence can be substituted with any nucleotide base provided that:

for any pair of sequences of the set:
M1≦19, M2≦17, M3≦21, M4≦18, and M5≦20, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein
the score of an alignment is determined according to the equation (A×m)−(B×mm)−(C×(og+eg))−(D×eg)), wherein:
for each of (i) to (iv)
   (i) m=6, mm=6, og=0 and eg=6,
   (ii) m=6, mm=6, og=5 and eg=1,
   (iii) m=6, mm=2, og=S and eg=1, and
   (iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv).

In another broad aspect, the invention is a composition comprising INVADER assay probes comprising tags or tag complements, wherein each tag portion of the molecule comprises an oligonucleotide selected from a set of oligonucleotides based on a group of sequences as specified by numeric identifiers set out in Table IIA wherein each of 1 to 3 is a nucleotide base selected to be different from the others of 1 to 3 with the proviso that up to three nucleotide bases of each sequence can be substituted with any nucleotide base provided that:

for any pair of sequences of the set:
M1≦19, M2≦17, M3≦21, M4≦18, and M5≦20, where:
M1 is the maximum number of matches for any alignment in which there are n internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score, wherein:
the score of an alignment is determined according to the equation 3A-B-3C-D, wherein:
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv).

In preferred aspects, the invention provides a composition in which, for the group of 24-mer sequences in which 1=A, 2=T and 3=G, under a defined set of conditions in which the maximum degree of hybridization between a sequence and any complement of a different sequence of the group of 24-mer sequences does not exceed 30% of the degree of hybridization between said sequence and its complement, for all said oligonucleotides of the composition, the maximum degree of hybridization between an oligonucleotide and a complement of any other oligonucleotide of the composition does not exceed 50% of the degree of hybridization of the oligonucleotide and its complement.

More preferably, the maximum degree of hybridization between a sequence and any complement of a different sequence does not exceed 30% of the degree of hybridization between said sequence and its complement, the degree of hybridization between each sequence and its complement varies by a factor of between 1 and up to 10, more preferably between 1 and up to 9, more preferably between 1 and up to 8, more preferably between 1 and up to 7, more preferably between 1 and up to 6, and more preferably between 1 and up to 5.

It is also preferred that the maximum degree of hybridization between a sequence and any complement of a different sequence does not exceed 25%, more preferably does not exceed 20%, more preferably does not exceed 15%, more preferably does not exceed 10%, more preferably does not exceed 5%.

Even more preferably, the above-referenced defined set of conditions results in a level of hybridization that is the same as the level of hybridization obtained when hybridization conditions include 0.2 M NaCl, 0.1 M Tris, 0.08% Triton X-100, pH 8.0 at 37.degree. C.

In the composition, the defined set of conditions can include the group of 24-mer sequences being covalently linked to beads.

In a particular preferred aspect, for the group of 24-mers the maximum degree of hybridization between a sequence and any complement of a different sequence does not exceed 15% of the degree of hybridization between said sequence and its complement and the degree of hybridization between each sequence and its complement varies by a factor of between 1 and up to 9, and for all oligonucleotides of the set, the maximum degree of hybridization between an oligonucleotide and a complement of any other oligonucleotide of the set does not exceed 20% of the degree of hybridization of the oligonucleotide and its complement.

It is possible that each 1 is one of A, T/U, G and C; each 2 is one of A, T/U, G and C; and each 3 is one of A, T/U, G and C; and each of 1, 2 and 3 is selected so as to be different from all of the others of 1, 2 and 3. More preferably, 1 is A or T/U, 2 is A or T/U and 3 is G or C. Even more preferably, 1 is A, 2 is T/U, and 3 is G.

In certain preferred composition, each of the oligonucleotides is from twenty-two to twenty-six bases in length, or from twenty-three to twenty-five, and preferably, each oligonucleotide is of the same length as every other said oligonucleotide.

In a particularly preferred embodiment, each oligonucleotide is twenty-four bases in length.

It is preferred that no oligonucleotide contains more than four contiguous bases that are identical to each other.

It is also preferred that the number of G's in each oligonucleotide does not exceed L/4 where L is the number of bases in said sequence.

For reasons described below, the number of G's in each said oligonucleotide is preferred not to vary from the average number of G's in all of the oligonucleotides by more than one. Even more preferably, the number of G's in each said oligonucleotide is the same as-every other said oligonucleotide. In the embodiment disclosed below in which oligonucleotides were tested, the sequence of each was twenty-four bases in length and each oligonucleotide contained 6 G's.

It is also preferred that, for each nucleotide, there is at most six bases other than G between every pair of neighboring pairs of G's.

Also, it is preferred that, at the 5′-end of each oligonucleotide at least one of the first, second, third, fourth, fifth, sixth and seventh bases of the sequence of the oligonucleotide is a G. Similarly, it is preferred, at the 3′-end of each oligonucleotide that at least one of the first, second, third, fourth, fifth, sixth and seventh bases of the sequence of the oligonucleotide is a G.

It is possible to have sequence compositions that include one hundred and sixty said molecules, or that include one hundred and seventy said molecules, or that include one hundred and eighty said molecules, or that include one hundred and ninety said molecules, or that include two hundred said molecules, or that include two hundred and twenty said molecules, or that include two hundred and forty said molecules, or that include two hundred and sixty said molecules, or that include two hundred and eighty said molecules, or that include three hundred said molecules, or that include four hundred said molecules, or that include five hundred said molecules, or that include six hundred said molecules, or that include seven hundred said molecules, or that include eight hundred said molecules, or that include nine hundred said molecules, or that include one thousand said molecules.

It is possible, in certain applications, for each molecule to be linked to a solid phase support so as to be distinguishable from a mixture containing other of the molecules by hybridization to its complement. Such a molecule can be linked to a defined location on a solid phase support such that the defined location for each molecule is different than the defined location for different others of the molecules.

In certain embodiments, each solid phase support is a microparticle and each said molecule is covalently linked to a different microparticle than each other different said molecule.

In another broad aspect, the invention is a composition comprising INVADER assay probes comprising tags or tag complements, wherein the composition comprises a set of 150 molecules for use as tags or tag complements wherein each molecule includes an oligonucleotide having a sequence of at least sixteen nucleotide bases wherein for any pair of sequences of the set:
M1≦19/24×L₁, M2≦17/24×L₁, M3≦21/24×L₁, M4≦18/24×L₁, M5≦20/24×L₁, where L₁ is the length of the shortest sequence of the pair, where:
   M1 is the maximum number of matches for any alignment of the pair of sequences in which there are no internal indels;
   M2 is the maximum length of a block of matches for any alignment of the pair of sequences;
   M3 is the maximum number of matches for any alignment of the pair of sequences having a maximum score;
   M4 is the maximum sum of the lengths of the longest two blocks of matches for any alignment of the pair of sequences of maximum score; and
   M5 is the maximum sum of the lengths of all the blocks of matches having length of at least 3, for any alignment of the pair of sequences of maximum score, wherein:
   the score of an alignment is determined according to the equation (A×m)−(B×mm)−(C×(og+eg))−(D×eg)), wherein:
   for each of (i) to (iv):
      (i) m=6, mm=6, og=0 and eg=6,
      (ii) m=6, mm=6, og=5 and eg=1,
      (iii) m=6, mm=2, og=5 and eg=1, and
      (iv) m=6, mm=6, og=6 and eg=0,
   A is the total number of matched pairs of bases in the alignment;
   B is the total number of internal mismatched pairs in the alignment;
   C is the total number of internal gaps in the alignment; and
   D is the total number of internal indels in the alignment minus the total number of internal gaps in the alignment; and
   wherein the maximum score is determined separately for each of (i), (ii), (iii) and (iv).

In yet another broad aspect, the invention is a composition that includes a set of 150 molecules for use as tags or tag complements wherein each molecule has an oligonucleotide having a sequence of at least sixteen nucleotide bases wherein for any pair of sequences of the set:
M1≦19, M2≦17, M3≦21, M4≦18, and M5≦20, where:
   M1 is the maximum number of matches for any alignment of the pair of sequences in which there are no internal indels;
   M2 is the maximum length of a block of matches for any alignment of the pair of sequences;
   M3 is the maximum number of matches for any alignment of the pair of sequences having a maximum score;
   M4 is the maximum sum of the lengths of the longest two blocks of matches for any alignment of the pair of sequences of maximum score; and
   M5 is the maximum sum of the lengths of all the blocks of matches having a length of at least 3, for any alignment of the pair of sequences of maximum score, wherein:
the score of a said alignment is determined according to the equation 3A-B-3C-D, wherein:
   A is the total number of matched pairs of bases in the alignment;
   B is the total number of internal mismatched pairs in the alignment;
   C is the total number of internal gaps in the alignment; and
   D is the total number of internal indels in the alignment minus the total number of internal gaps in the alignment.

In certain embodiments of the invention, each sequence of a composition has up to fifty bases. More preferably, however, each sequence is between sixteen and forty bases in length, or between sixteen and thirty-five bases in length, or between eighteen and thirty bases in length, or between twenty and twenty-eight bases in length, or between twenty-one and twenty-seven bases in length, or between twenty-two and twenty-six bases in length.

Often, each sequence is of the same length as every other said sequence. In particular embodiments disclosed-herein, each sequence is twenty-four bases in length.

Again, it can be preferred that no sequence contains more than four contiguous bases that are identical to each other, etc., as described above.

In certain preferred embodiments, the composition is such that, under a defined set of conditions, the maximum degree of hybridization between an oligonucleotide and any complement of a different oligonucleotide of the composition does not exceed about 30% of the degree of hybridization between said oligonucleotide and its complement, more preferably 20%, more preferably 15%, more preferably 10%, more preferably 6%.

Preferably, the set of conditions results in a level of hybridization that is the same as the level of hybridization obtained when hybridization conditions include 0.2 M NaCl, 0.1 M Tris, 0.08% Triton X-100, pH 8.0 at 37.degree. C., and the oligonucleotides are covalently linked to microparticles. Of course it is possible that these specific conditions be used for determining the level of hybridization.

It is also preferred that under such a defined set of conditions, the degree of hybridization between each oligonucleotide and its complement varies by a factor of between 1 and up to 8, more preferably up to 7, more preferably up to 6, more preferably up to 5. In a particular disclosed embodiment, the observed variance in the degree of hybridization was a factor of only 5.3, i.e., the degree of hybridization between each oligonucleotide and its complement varied by a factor of between 1 and 5.6.

In certain preferred embodiments, under the defined set of conditions, the maximum degree of hybridization between a said oligonucleotide and any complement of a different oligonucleotide of the composition does not exceed about 15%, more preferably 10%, more preferably 6%.

In one preferred embodiment, the set of conditions results in a level of hybridization that is the same as the level of hybridization obtained when hybridization conditions include 0.2 M NaCl, 0.1 M Tris, 0.08% Triton X-100, pH 8.0 at 37° C., and the oligonucleotides are covalently linked to microparticles.

Also, under the defined set of conditions, it is preferred that the degree of hybridization between each oligonucleotide and its complement varies by a factor of between 1 and up to 8, more preferably up to 7, more preferably up to 6, more preferably up to 5.

Any composition of the invention can include one hundred and sixty of the oligonucleotide molecules, or one hundred and seventy of the oligonucleotide molecules, or one hundred and eighty of the oligonucleotide molecules, or one hundred and ninety of the oligonucleotide molecules, or two hundred of the oligonucleotide molecules, or two hundred and twenty of the oligonucleotide molecules, or two hundred and forty of the oligonucleotide molecules, or two hundred and sixty of the oligonucleotide molecules, or two hundred and eighty of the oligonucleotide molecules, or three hundred of the oligonucleotide molecules, or four hundred of the oligonucleotide molecules, or five hundred of the oligonucleotide molecules, or six hundred of the oligonucleotide molecules, or seven hundred of the oligonucleotide molecules, or eight hundred of the oligonucleotide molecules, or nine hundred of the oligonucleotide molecules, or one thousand or more of the oligonucleotide molecules.

A composition of the invention can comprise a family of 5′ tags, or it can comprise a family of 5′ tag complements.

An oligonucleotide molecule belonging to a family of molecules of the invention can have incorporated thereinto one more analogues of nucleotide bases, preference being given those that undergo normal Watson-Crick base pairing.

The invention includes kits for sorting and identifying polynucleotides. Such a kit can include one or more solid phase supports each having one or more spatially discrete regions, each such region having a uniform population of substantially identical tag complements covalently attached. The tag complements are made up of a set of oligonucleotides of the invention.

The one or more solid phase supports can be a planar substrate in which the one or more spatially discrete regions is a plurality of spatially addressable regions.

The tag complements can also be coupled to microparticles. See, e.g., U.S. Pat. No. 6,916,661, which is incorporated herein by reference. Microparticles preferably each have a diameter in the range of from 5 to 40 μm.

Such a kit preferably includes microparticles that are spectrophotometrically unique, and therefore distinguishable from each other according to conventional laboratory techniques. Of course for such kits to work, each type of microparticle would generally have only one tag complement associated with it, and usually there would be a different oligonucleotide tag complement associated with (attached to) each type of microparticle.

The invention provides a method for sorting complex mixtures of molecules, e.g., in multiplex INVADER assays, by the use of families of oligonucleotide sequence tags. The families of oligonucleotide sequence tags are designed so as to provide minimal cross hybridization during the sorting process. Thus any sequence within a family of sequences will not cross hybridize with any other sequence derived from that family under appropriate hybridization conditions known by those skilled in the art. The invention is particularly useful in highly parallel processing of analytes.

### EXAMPLES

### Example 1

### Family I Tags

### Families of Oligonucleotide Sequence Tags

The present invention includes a family of 24-mer polynucleotides, that have been demonstrated to be minimally cross-hybridizing with each other. This family of polynucleotides is thus useful as a family of tags, and their complements as tag complements.

The oligonucleotide sequences that belong to families of sequences that do not exhibit cross hybridization behavior can be derived by computer programs (described in U.S. Provisional Patent Application No. 60/181,563 filed Feb. 10, 2000). The programs use a method of generating a maximum number of minimally cross-hybridizing polynucleotide sequences that can be summarized as follows. First, a set of sequences of a given length are created based on a given number of block elements. Thus, if a family of polynucleotide sequences 24 nucleotides (24-mer) in length is desired from a set of 6 block elements, each element comprising 4 nucleotides, then a family of 24-mers is generated considering all positions of the 6 block elements. In this case, there will be 6⁶ (46,656) ways of assembling the 6 block elements to generate all possible polynucleotide sequences 24 nucleotides in length.

Constraints are imposed on the sequences and are expressed as a set of rules on the identities of the blocks such that homology between any two sequences will not exceed the degree of homology desired between these two sequences. All polynucleotide sequences generated that obey the rules are saved. Sequence comparisons are performed in order to generate an incidence matrix. The incidence matrix is presented as a simple graph and the sequences with the desired property of being minimally cross hybridizing are found from a clique of the simple graph, which may have multiple cliques. Once a clique containing a suitably large number of sequences is found, the sequences are experimentally tested to determine if it is a set of minimally cross hybridizing sequences. This method was used to obtain the 210 non cross-hybridizing tags of Table I (See Janeczko, supra).

The method includes a rational approach to the selection of groups of sequences that are used to describe the blocks. For example there are n⁴ different tetramers that can be obtained from n different nucleotides, non-standard bases or analogues thereof. In a more preferred embodiment there are 44 or 256 possible tetramers when natural nucleotides are used. More preferably 81 possible tetramers when only 3 bases are used A, T and G. Most preferably 32 different tetramers when all sequences have only one G.

Block sequences can be composed of a subset of natural bases most preferably A, T and G. Sequences derived from blocks that are deficient in one base possess useful characteristics, for example, in reducing potential secondary structure formation or reduced potential for cross hybridization with nucleic acids in nature. Sets of block sequences that are most preferable in constructing families of non cross-hybridizing tag sequences should contribute approximately equivalent stability to the formation of the correct duplex as all other block sequences of the set. This should provide tag sequences that behave isothermally. This can be achieved, for example, by maintaining a constant base composition for all block sequences such as one G and three A's or T's for each block sequence. Preferably, non-cross hybridizing sets of block sequences will be comprised from blocks of sequences that are isothermal. The block sequences should be different from each other by at least one mismatch. Guidance for selecting such sequences is provided by methods for selecting primer and or probe sequences that can be found in published techniques (Robertson et al., Methods Mol Biol; 98:121-54 (1998); Rychlik et al, Nucleic Acids Research, 17:8543-8551 (1989); Breslauer et al., Proc Natl Acad. Sci., 83:3746-3750 (1986)) and the like. Additional sets of sequences can be designed by extrapolating on the original family of non cross-hybridizing sequences by simple methods known to those skilled in the art.

A preferred family of 100 tags is shown as SEQ ID NOs:1173 to 1382 in Table I. Characterization of the family of 100 sequence tags was performed to determine the ability of these sequences to form specific duplex structures with their complementary sequences and to assess the potential for cross hybridization. (See Janeczko, supra). The results indicated that the family of sequences are non-cross hybridizing (tag) sequences.

The family of 100 non-cross-hybridizing sequences can be expanded by incorporating additional tetramer sequences that are used in constructing further 24-mer oligonucleotides. (See Janeczko, supra). An additional set of 73 tag sequences so obtained (SEQ ID NOs:1273 to 1345 of Table 1) is composed of sequences that, when compared to any of SEQ ID NOs: 1173-1382, of Table I have no greater similarity than the sequences of the original 100 sequence tags of Table I. The set of 173 24-mer oligonucleotides were expanded again to include those having SEQ ID NOs:1346 to 1382 as follows. The 4-mers WXYW, XYXW, WXXW, WYYW, XYYX, YXYX, YXXY and XYXY where W=G, X=A, and Y=U/T were used in combination with the fourteen 4-mers used in the generation of SEQ ID NOs:1173-1345 to generate potential 24-base oligonucleotides. Excluded from the set were those containing the sequence patterns GG, AAAA and TTTT. To be included in the set of additional 24-mers, a sequence also had to have at least one of the 4-mers containing two G's: WXYW (GATG), WYXW (GTAG), WXXW (GAAG), WYYW (GTTG) while also containing exactly six G's. Also required for a 24-mer to be included was that there be at most six bases between every neighboring pair of G's. Another way of putting this is that there are at most six non-G's between any two G's. Also, each G nearest the 5′-end of its oligonucleotide (the left-hand side as written in Table I) was required to occupy one of the first to seventh positions (counting the 5′-terminal position as the first position.) A set of candidate sequences was obtained by eliminating any new sequence that was found to have a maximum simple homology of 16/24 or more with any of the previous set of 173 oligonucleotides (Table 1, SEQ ID NOs:1173-1345). As above, an arbitrary 174^{th} sequence was chosen and candidate sequences eliminated by comparison therewith. In this case the permitted maximum degree of simple homology was 16/24. A second sequence was also eliminated if there were ten consecutive matches between the two (i.e., it was notionally possible to generate a phantom sequence containing a sequence of 10 bases that is identical to a sequence in each of the sequences being compared). A second sequence was also eliminated if it was possible to generate a phantom sequence 20 bases in length or greater.

A property of the polynucleotide sequences shown in Table I is that the maximum block homology between any two sequences is never greater than 662/3 percent. This is because the computer algorithm by which the sequences were initially generated was designed to prevent such an occurrence. It is within the capability of a person skilled in the art, given the family of sequences of Table I, to modify the sequences, or add other sequences while largely retaining the property of minimal-cross hybridization which the polynucleotides of Table I have been demonstrated to have.

TABLE I

| | |
|---|---|
| SEQ ID NO: | 5′ TAG |

| | |
|---|---|
| 1173 | GATTTGTATTGATTGAGATTAAAG |
| 1174 | TGATTGTAGTATGTATTGATAAAG |
| 1175 | GATTGTAAGATTTGATAAAGTGTA |
| 1176 | GATTTGAAGATTATTGGTAATGTA |
| 1177 | GATTGATTATTGTGATTTGAATTG |
| 1178 | GATTTGATTGTAAAAGATTGTTGA |
| 1179 | ATTGGTAAATTGGTAAATGAATTG |
| 1180 | ATTGGATTTGATAAAGGTAAATGA |
| 1181 | GTAAGTAATGAATGTAAAAGGATT |
| 1182 | GATTGATTGATTGATTGATTTGAT |
| 1183 | TGATGATTAAAGAAAGTGATTGAT |
| 1184 | AAAGGATTTGATTGATAAAGTGAT |
| 1185 | TGTAGATTTGTATGTATGTATGAT |
| 1186 | GATTTGATAAAGAAAGGATTGATT |
| 1187 | GATTAAAGTGATTGATGATTTGTA |
| 1188 | AAAGAAAGAAAGAAAGAAAGTGTA |
| 1189 | TGTAAAAGGATTGATTTGTATGTA |
| 1190 | AAAGTGTAGATTGATTAAAGAAAG |
| 1191 | AAAGTTGATTGATTGAAAAGGTAT |
| 1192 | TTGATTGAGATTGATTTTGAGTAT |
| 1193 | TGAATTGATGAATGAATGAAGTAT |
| 1194 | GTAATGAAGTATGTATGTAAGTAA |
| 1195 | TGATGATTTGAATGAAGATTGATT |
| 1196 | TGATAAAGTGATAAAGGATTAAAG |
| 1197 | TGATTTGAGTATTTGAGATTTTGA |
| 1198 | TGTAGTAAGATTGATTAAAGGTAA |
| 1199 | GTATAAAGGATTGATTTTGAAAAG |
| 1200 | GTATTTGAGTAAGTAATTGATTGA |
| 1201 | GTAAAAAGTTGAGTATTGAAAAAG |
| 1202 | GATTTGATAAAGGATTTGTATTGA |
| 1203 | GATTGTATTGAAGTATTGTAAAAG |
| 1204 | TGATGATTTTGATGAAAAAGTTGA |
| 1205 | TGATTTGAGATTAAAGAAAGGATT |
| 1206 | TGATTGAATTGAGTAAAAAGGATT |
| 1207 | AAAGTGTAAAAGGATTTGATGTAT |
| 1208 | AAAGGTATTTGAGATTTGATTGAA |
| 1209 | AAAGTTGAGATTTGAATGATTGAA |
| 1210 | TGTATTGAAAAGGTATGATTTGAA |
| 1211 | GTATTGTATTGAAAAGGTAATTGA |
| 1212 | TTGAGTAATGATAAAGTGAAGATT |
| 1213 | TGAAGATTTGAAGTAATTGAAAAG |
| 1214 | TGAAAAAGTGTAGATTTTGAGTAA |
| 1215 | TGTATGAATGAAGATTTGATTGTA |
| 1216 | AAAGTTGAGTATTGATTTGAAAAG |
| 1217 | GATTTGTAGATTTGTATTGAGATT |
| 1218 | AAAGAAAGGATTTGTAGTAAGATT |
| 1219 | GTAAAAAGAAAGGTATAAAGGTAA |
| 1220 | GATTAAAGTTGATTGAAAAGTGAA |
| 1221 | TGAAAAAGGTAATTGATGTATGAA |
| 1222 | AAAGGATTAAAGTGAAGTAATTGA |
| 1223 | ATGAATTGGTATGTATATGAATGA |
| 1224 | TGAAATGAATGAATGATGAAATTG |
| 1225 | ATTGATTGTGAATGAAATGAATTG |
| 1226 | ATTGAAAGATGAAAAGATGAAAAG |
| 1227 | ATTGTTGAAAAGTGTAATGATTGA |
| 1228 | ATGATGTAATGAAAAGATTGTGTA |
| 1229 | AAAGATTGAAAGATGATGTAATTG |
| 1230 | ATTGATGAGTATATTGTGTAGTAA |
| 1231 | AAAGATTGTGTAATTGATGATGAA |
| 1232 | AAAGGTATATTGTGTAATGAGTAA |
| 1233 | TGTAATGAGTATTGTAATTGAAAG |
| 1234 | GTATAAAGAAAGATTGGTAAATGA |
| 1235 | TTGAGTAATTGAATTGTGAAATGA |
| 1236 | TGTATTGAATGAATTGTTGATGTA |
| 1237 | TGTAATTGGTAAATGAGTAAAAAG |
| 1238 | TGAATGAAATTGATGAGTATAAAG |
| 1239 | GTAAGTAAATTGAAAGATTGATGA |
| 1240 | GTAAATGATGATATTGGTATATTG |
| 1241 | ATTGTTGATGATTGATTGAAATGA |
| 1242 | ATTGTGAAGTATAAAGATGATTGA |
| 1243 | ATGAAAAGTTGAGTAAATTGTGAT |
| 1244 | ATGAATTGAAAGTGATTGAAAAAG |
| 1245 | GTAAATTGATGAAAAGTTGATGAT |
| 1246 | AAAGTGATGTATATGAGTAAATTG |
| 1247 | GTAATGATAAAGATGATGATATTG |
| 1248 | TTGAAAAGATTGGTAATGATATGA |
| 1249 | AAAGTGAAAAAGATTGATTGATGA |
| 1250 | ATTGATGAGATTGATTATTGTGTA |
| 1251 | ATGAGATTATTGGATTTGTAGATT |
| 1252 | TGAAGATTATGAATTGGTAAGATT |
| 1253 | ATTGGATTATGAGATTATGATTGA |
| 1254 | ATTGTTGAATTGGATTAAAGATGA |
| 1255 | AAAGATGAGTAAGTAAATTGGATT |
| 1256 | AAAGGTAAGATTATTGATGAAAAG |
| 1257 | ATTGATGAGATTAAAGTTGAATTG |
| 1258 | GATTATTGGATTATGAAAAGGATT |
| 1259 | GATTTGTAATTGTTGAGTAAATGA |
| 1260 | AAAGAAAGATTGTTGAGATTATGA |
| 1261 | GTATAAAGGATTTTGAATTGATGA |
| 1262 | TTGAGATTGTAAATGAATTGTTGA |
| 1263 | GTATATTGATTGTGTAATGAAAAG |
| 1264 | TGATATGAATTGGATTATTGGTAT |
| 1265 | ATGAATGATGAATGATGATTATTG |
| 1266 | ATGAATTGATTGGATTGTAATGAT |
| 1267 | GATTGTAATTGAGTAAATTGATGA |
| 1268 | GATTATTGGATTAAAGGTAAATGA |
| 1269 | ATTGTTGAATTGATGAGATTTGAT |
| 1270 | GATTATGAGTAAATTGATTGTGAT |
| 1271 | GATTATTGTTGATGAATGATATTG |
| 1272 | TGTAAAAGATTGAAAGGTATGATT |
| 1273 | GTATTTAGATGAGTTTGTTAGATT |
| 1274 | TGAAGTTATGTAATAGAAAGTGAT |
| 1275 | GTATGTATTGTATGTAGTTAATTG |
| 1276 | TGATATAGATAGTTAGATAGATAG |
| 1277 | ATGATGATGTATTGTAGTTATGAA |
| 1278 | TTAGTGAATGTATTAGTTGATGTA |
| 1279 | GTTAGTTAGATTATTGTTAGTTAG |
| 1280 | GTTAATTGTGTAGTTTGTTATTGA |
| 1281 | GTTATGAAATAGTGATATTGTTAG |
| 1282 | ATTGTTAGAAAGTGTAGATTAAAG |
| 1283 | ATGAGTATGTTATTAGTGTATGTA |
| 1284 | TGTAATAGTGAAGTTAGATTGTAT |
| 1285 | ATTGATAGATGATTAGTTAGTTGA |
| 1286 | ATGAGTTTGTTTATGAGATTAAAG |
| 1287 | TGATGTTTGATTATGATGTAGTAT |
| 1288 | ATGAGTTAGTTATGAATTAGATGA |
| 1289 | ATTGTTAGTGATGTTAGTAATTAG |
| 1290 | TGATGTAAGTATTGATGTTAGTTT |
| 1291 | GATTGTAAATAGAAAGTGAAGTAA |
| 1292 | ATTGTGTATGAAGTATTGTATGAT |
| 1293 | ATAGTGATGTTATGAAGATTGTTA |
| 1294 | TTAGATGAATTGTGAAGTATTTAG |
| 1295 | GTAAGTTATGATTGATGTTATGAA |
| 1296 | GTATTGATGTTTAAAGTGTAATAG |
| 1297 | GATTGTAAGTAAGATTGTATATTG |
| 1298 | GTTTGTATTTAGATGAATAGAAAG |
| 1299 | GTTTGATTTGTAATAGTGATTGTA |
| 1300 | TGTATGTAGTATTTAGAAAGATGA |
| 1301 | ATGAATTGTGATAAAGAAAGTTAG |
| 1302 | TTAGTGTAGTAAGTTTAAAGTGTA |
| 1303 | GTATGATTGTTTGTAATTAGTGAT |
| 1304 | GTTTAAAGTTAGTTGAGTTAGTAT |
| 1305 | ATAGTGTATGTAGATTATGAGATT |
| 1306 | TTGAATGATTAGTTGAGTATGATT |
| 1307 | GTATGTAAGTTAGTATGATTTGAA |
| 1308 | TGTAGTATATTGTTGAATTGTGAT |
| 1309 | ATAGTGATTGTATGTATGATAAAG |
| 1310 | TTAGTGATTGATGTATATTGAAAG |
| 1311 | GTAAGATTATGAGTTATGATGTAA |
| 1312 | GTTATGAAATTGTTAGTGTAGATT |
| 1313 | GTTAGATTTGTAGTTTAAAGATAG |
| 1314 | TTAGTGATTGAAATGATGTAGATT |
| 1315 | AAAGTGTAGTTATTAGTTAGTTAG |
| 1316 | AAAGAAAGTGTATGATGTTATTAG |
| 1317 | GATTGTATATTGTGTATGATGATT |
| 1318 | TTGAGATTGTTATGATATGAGTAT |
| 1319 | ATGAGTATGATTGTTATGATGTTT |
| 1320 | TGATTTAGTGAAATTGTGTATTAG |
| 1321 | TGAATGTATGTAGTATGTTTGTTA |
| 1322 | GTTAGTATTGATGATTATGAGTTA |
| 1323 | GTATATTGTGATTTAGTTGAGATT |
| 1324 | GTTAGTTTAAAGTTGAGATTGTTT |
| 1325 | GTATATTGTTAGATGAGATTTGTA |
| 1326 | TGATGTATGTTAGTTTATGAATGA |
| 1327 | TGTAGTATGTAATGTAGTATTTGA |
| 1328 | ATGAGTTATGTATTGAGTTAGTAT |
| 1329 | TGTATGATGATTATAGTTGAGTAA |
| 1330 | ATTGATGAATGAGTTTGTATAAAG |
| 1331 | TTGAGTTTATGATTAGAAAGAAAG |
| 1332 | TGATATTGATGAGTTAGTATTGAA |
| 1333 | ATAGAAAGTGAAATGAGTATGTTA |
| 1334 | TTGATGTAGATTTGATGTATATAG |
| 1335 | TTGAGATTATAGTGTAGTTTATAG |
| 1336 | TGATGTTAGATTGTTTGATTATTG |
| 1337 | TGTATTAGATAGTGATTTGAATGA |
| 1338 | GATTATGATGAATGTAGTATGTAA |
| 1339 | TGAATGATTGATATGAATAGTGTA |
| 1340 | GTAATGATTTAGTGTATTGAGTTT |
| 1341 | TGTAGTAATGATTTGATGATAAAG |
| 1342 | TGAAGATTGTTATTAGTGATATTG |
| 1343 | GTATTTGAATGATGTAATAGTGTA |
| 1344 | GTATATGATGTATTAGATTGAAAG |
| 1345 | AAAGTTAGATTGAAAGTGATAAAG |
| 1346 | GTAAGATGTTGATATAGAAGATTA |
| 1347 | TAATATGAGATGAAAGTGAATTAG |
| 1348 | TTAGTGAAGAAGTATAGTTTATTG |
| 1349 | GTAGTTGAGAAGATAGTAATTAAT |
| 1350 | ATGAGATGATATTTGAGAAGTAAT |
| 1351 | GATGTGAAGAAGATGAATATATAT |
| 1352 | AAAGTATAGTAAGATGTATAGTAG |
| 1353 | GAAGTAATATGAGTAGTTGAATAT |
| 1354 | TTGATAATGTTTGTTTGTTTGTAG |
| 1355 | TGAAGAAGAAAGTATAATGATGAA |
| 1356 | GTAGATTAGTTTGAAGTGAATAAT |
| 1357 | TATAGTAGTGAAGATGATATATGA |
| 1358 | TATAATGAGTTGTTAGATATGTTG |
| 1359 | GTTGTGAAATTAGATGTGAAATAT |
| 1360 | TAATGTTGTGAATAATGTAGAAAG |
| 1361 | GTTTATAGTGAAATATGAAGATAG |
| 1362 | ATTATGAAGTAAGTTAATGAGAAG |
| 1363 | GATGAAAGTAATGTTTATTGTGAA |
| 1364 | ATTATTGAGATGTGAAGTTTGTTT |
| 1365 | TGTAGAAGATGAGATGTATAATTA |
| 1366 | TAATTTGAGTTGTGTATATAGTAG |
| 1367 | TGATATTAGTAAGAAGTTGAATAG |
| 1368 | GTTAGTTATTGAGAAGTGTATATA |
| 1369 | GTAGTAATGTTAATGAATTAGTAG |
| 1370 | GTTTGTTTGATGTGATTGAATAAT |
| 1371 | GTAAGTAGTAATTTGAATATGTAG |
| 1372 | GTTTGAAGATATGTTTGAAGTATA |
| 1373 | ATGATAATTGAAGATGTAATGTTG |
| 1374 | GTAGATAGTATAGTTGTAATGTTA |
| 1375 | GATGTGAATGTAATATGTTTATAG |
| 1376 | TGAAATTAGTTTGTAAGATGTGTA |
| 1377 | TGTAGTATAAAGTATATGAAGTAG |
| 1378 | ATATGTTGTTGAGTTGATAGTATA |
| 1379 | ATTATTGAGTAGAAAGATAGAAAG |
| 1380 | GTTGTTGAATATTGAATATAGTTG |
| 1381 | ATGAGAAGTTAGTAATGTAAATAG |
| 1382 | TGAAATGAGAAGATTAATGAGTTT |

There are 210 polynucleotide sequences given in Table I. Since all 210 of this family of polynucleotides can work with each other as a minimally cross-hybridizing set, then any plurality of polynucleotides that is a subset of the 210 can also act as a minimally cross-hybridizing set of polynucleotides. An application in which, for example, 30 molecules are to be sorted using a family of polynucleotide tags and tag complements could thus use any group of 30 sequences shown in Table I. This is not to say that some subsets may be found in practical sense to be more preferred than others. For example, it may be found that a particular subset is more tolerant of a wider variety of conditions under which hybridization is conducted before the degree of cross-hybridization becomes unacceptable.

It may be desirable to use polynucleotides that are shorter in length than the 24 bases of those in Table I. A family of subsequences (i.e., subframes of the sequences illustrated) based on those contained in Table I having as few as 10 bases per sequence could be chosen, so long as the subsequences are chosen to retain homological properties between any two of the sequences of the family important to their non cross-hybridization.

The selection of sequences using this approach would be amenable to a computerized process. Thus for example, a string of 10 contiguous bases of the first 24-mer of Table I could be selected: GATTTGTATTGATTGAGATTAAAG.

A string of contiguous bases from the second 24-mer could then be selected and compared for maximum homology against the first chosen sequence:

TGATTGTAGTATGTATTGATAAAG

Systematic pairwise comparison could then be carried out to determine if the maximum homology requirement of 662/3 percent is violated:

| | |
|---|---|
| Alignment | Matches |

| | |
|---|---|
| GATTTGTATT | 1 |
| ATTGATAAAG | |
| GATTTGTATT | 0 |
| ATTGATAAAG | |
| GATTTGTATT | 1 |
| ATTGATAAAG | |
| GATTTGTATT | 1 |
| ATTGATAAAG | |
| GATTTGTATT | 1 |
| ATTGATAAAG | |
| GATTTGTATT | 1 |
| ATTGATAAAG | |
| GATTTGTATT | 3 |
| ATTGATAAAG | |
| GATTTGTATT | 1 |
| ATTGATAAAG | |
| GATTTGTATT | 2 |
| ATTGATAAAG | |
| GATTTGTATT | 2 |
| ATTGATAAAG | |
| GATTTGTATT | 5 (*) |
| ATTGATAAAG | |
| GATTTGTATT | 3 |
| ATTGATAAAG | |
| GATTTGTATT | 3 |
| ATTGATAAAG | |
| GATTTGTATT | 2 |
| ATTGATAAAG | |
| GATTTGTATT | 1 |
| ATTGATAAAG | |
| GATTTGTATT | 1 |
| ATTGATAAAG | |
| GATTTGTATT | 3 |
| ATTGATAAAG | |
| GATTTGTATT | 1 |
| ATTGATAAAG | |
| GATTTGTATT | 0 |
| ATTGATAAAG | |

As can be seen, the maximum homology between the two selected subsequences is 50 percent (5 matches out of the total length of 10), and so these two sequences are compatible with each other.

A 10mer subsequence can be selected from the third 24-mer sequence of Table I, and pairwise compared to each of the first two 10mer sequences to determine its compatability therewith, etc. and in this way a family of 10mer sequences developed.

It is within the scope of this invention, to obtain families of sequences containing 11mer, 12mer, 13mer, 14-mer, 15mer, 16mer, 17mer, 18mer, 19mer, 20mer, 21 mer, 22mer and 23mer sequences by analogy to that shown for 10mer sequences.

It may be desirable to have a family of sequences in which there are sequences greater in length than the 24-mer sequences shown in Table I. It is within the capability of a person skilled in the art, given the family of sequences shown in Table I, to obtain such a family of sequences. One possible approach would be to insert into each sequence at one or more locations a nucleotide, non natural base or analogue such that the longer sequence should not have greater similarity than any two of the original non cross hybridizing sequences of Table I and the addition of extra bases to the tag sequences should not result in a major change in the thermodynamic properties of the tag sequences of that set for example the GC content must be maintained between 10%-40% with a variance from the average of 20%. This method of inserting bases could be used to obtain a family of sequences up to 40 bases long.

### Example 2

### Family II Tags

The present invention also provides INVADER assays making use of a family of 1168 24-mer polynucleotides that have been demonstrated to be minimally cross-hybridizing with each other. This family of polynucleotides is thus useful as a family of tags, and their complements as tag complements.

In order to be considered for inclusion into the family, a sequence had to satisfy a certain number of rules regarding its composition. For example, repetitive regions that present potential hybridization problems such as four or more of a similar base (e.g., AAAA or TTTT) or pairs of Gs were forbidden. Another rule is that each sequence contains exactly six Gs and no Cs, in order to have sequences that are more or less isothermal. Also required for a 24-mer to be included is that there must be at most six bases between every neighboring pair of Gs. Another way of putting this is that there are at most six non-Gs between any two consecutive Gs. Also, each G nearest the 5′-end (resp. 3′-end) of its oligonucleotide (the left-hand (resp. right-hand) side as written in Table II) was required to occupy one of the first to seventh positions (counting the 5′-terminal (resp. 3′-terminal) position as the first position.)

Depending on the application for which these families of sequences will be used, various rules are designed. A certain number of rules can specify constraints for sequence composition (such as the ones described in the previous paragraph). The other rules are used to judge whether two sequences are too similar. Based on these rules, a computer program can derive families of sequences that exhibit minimal or no cross-hybridization behavior. The exact method used by the computer program is not crucial since various computer programs can derive similar families based on these rules. Such a program is for example described in international patent application No. PCT/CA 01/00141 published under WO 01/59151 on Aug. 16, 2001. Other, programs can use different methods, such as the ones summarized below.

A first method of generating a maximum number of minimally cross-hybridizing polynucleotide sequences starts with any number of non-cross-hybridizing sequences, for example just one sequence, and increases the family as follows. A certain number of sequences is generated and compared to the sequences already in the family. The generated sequences that exhibit too much similarity with sequences already in the family are dropped. Among the “candidate sequences” that remain, one sequence is selected and added to the family. The other candidate sequences are then compared to the selected sequence, and the ones that show too much similarity are-dropped. A new sequence is selected from the remaining candidate sequences, if any, and added to the family, and soon until there are no candidate sequences left. At this stage, the process can be repeated (generating a certain number of sequences and comparing them to the sequences in the family, etc.) as often as desired. The family obtained at the end of this method contains only minimally cross-hybridizing sequences.

A second method of generating a maximum number of minimally cross-hybridizing polynucleotide sequences starts with a fixed-size family of polynucleotide sequences. The sequences of this family can be generated randomly or designed by some other method. Many sequences in this family may not be compatible with each other, because they show too much similarity and are not minimally cross-hybridizing. Therefore, some sequences need to be replaced by new ones, with less similarity. One way to achieve this consists of repeatedly replacing a sequence of the family by the best (that is, lowest similarity) sequence among a certain number of (for example, randomly generated) sequences that are not part of the family. This process can be repeated until the family of sequences shows minimal similarity, hence minimal cross-hybridizing, or until a set number of replacements has occurred. If, at the end of the process, some sequences do not obey the similarity rules that have been set, they can be taken out of the family, thus providing a somewhat smaller family that only contains minimally cross-hybridizing sequences. Some additional rules can be added to this method in order to make it more efficient, such as rules to determine which sequence will be replaced.

Such methods have been used to obtain the 1168 non-cross-hybridizing tags of Table II (see also U.S. Patent Publication 20050186573).

One embodiment of the invention is a composition comprising molecules for use as tags or tag complements on INVADER assay probes, wherein each molecule comprises an oligonucleotide selected from a set of oligonucleotides based on the group of sequences set out in Table IIA, wherein each of the numeric identifiers 1 to 3 (see the Table) is a nucleotide base selected to be different from the others of 1 to 3. According to this embodiment, several different families of specific sets of oligonucleotide sequences are described, depending upon the assignment of bases made to the numeric identifiers 1 to 3.

The sequences contained in Table II have a mathematical relationship to each other, described as follows.

Let S and T be two DNA sequences of lengths s and t respectively. While the term “alignment” of nucleotide sequences is widely used in the field of biotechnology, in the context of this invention the term has a specific meaning illustrated here. An alignment of S and T is a 2× matrix A (with p≧s and p≧t) such that the first (or second) row of A contains the characters of S (or T respectively) in order, interspersed with p-s (or p-t respectively) spaces. It assumed that no column of the alignment matrix contains two spaces, i.e., that any alignment in which a column contains two spaces is ignored and not considered here. The columns containing the same base in both rows are called matches, while the columns containing different bases are called mismatches. Each column of an alignment containing a space in its first row is called an insertion and each column containing a space in its second row is called a deletion while a column of the alignment containing a space in either row is called an indel. Insertions and deletions within a sequence are represented by the character ‘-’. A gap is a continuous sequence of spaces in one of the rows (that is neither immediately preceded nor immediately followed by another space in the same row), and the length of a gap is the number of spaces in that gap. An internal gap is one in which its first space is preceded by a base and its last space is followed by a base and an internal indel is an belonging to an internal gap. Finally, a block is a continuous sequence of matches (that is neither immediately preceded nor immediately followed by another match), and the length of a block is the number of matches in that block. In order to illustrate these definitions, two sequences S=TGATCGTAGCTACGCCGCG (of length s=19; SEQ ID NO:1169) and T=CGTACGATTGCAACGT (of length t=16, SEQ ID NO:1170) are considered. Exemplary alignment R1 of S and T (with p=23) is:

### Alignment R₁

| — | — | — | — | T | G | A | T | C | G | T | A | G | C | T | A | C | G | C | C | G | C | G |
|---|---|---|---|---|---|---|---|---|---|---|---|---|---|---|---|---|---|---|---|---|---|---|
| C | G | T | A | C | G | A | T | — | — | T | — | G | C | A | A | C | G | T | — | — | — | — |
| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |

Columns 1 to 4, 9, 10, 12 and 20 to 23 are indels, columns 6, 7, 8, 11, 13, 14, 16, 17 and 18 are matches, and columns 5, 15 and 19 are mismatches. Columns 9 and 10 form a gap of length 2, while columns 16 to 18 form a block of length 3. Columns 9, 10 and 12 are internal indels.

A score is assigned to the alignment A of two sequences by assigning weights to each of matches, mismatches and gaps as follows:

the reward for a match m,

the penalty for a mismatch mm,

the penalty for opening a gap og

the penalty for extending a gap eg.

Once these values are set, a score to each column of the alignment is assigned according to the following rules:

1. assign 0 to each column preceding the first match and to each column following the last match.

2. for each of the remaining columns, assign m if it is a match, mm if it is a mismatch, -og-eg if it is the first indel of a gap, -eg if it is an indel but not the first indel of a gap.

The score of the alignment A is the sum of the scores of its columns. An alignment is said to be of maximum score if no other alignment of the same two sequences has a higher score (with the same values of m, mm, og and eg). A person knowledgeable in the field will recognize this method of scoring an alignment as scoring a local (as opposed to global) alignment with affine gap penalties (that is, gap penalties that can distinguish between the first indel of a gap and the other indels). It will be appreciated that the total number of indels that open a gap is the same as the total number of gaps and that an internal indel is not one of those assigned a 0 in rule (1) above. It will also be noted that foregoing rule (1) assigns a 0 for non-internal mismatches. An internal mismatch is a mismatch that is preceded and followed (not necessarily immediately) by a match.

As an illustration, if the values of m, mm, og and eg are set to 3, 1, 2 and 1 respectively, alignment R₁ has a score of 19, determined as shown below:

### Scoring of Alignment R₁

| — | — | — | — | T | G | A | T | C | G | T | A | G | C | T | A | C | G | C | C | G | C | G |
|---|---|---|---|---|---|---|---|---|---|---|---|---|---|---|---|---|---|---|---|---|---|---|
| C | G | T | A | C | G | A | T | — | — | T | — | G | C | A | A | C | G | T | — | — | — | — |
| 0 | 0 | 0 | 0 | 0 | 3 | 3 | 3 | −3 | −1 | 3 | −3 | 3 | 3 | −1 | 3 | 3 | 3 | 0 | 0 | 0 | 0 | 0 |

Note that for two given sequences S and T, there are numerous alignments. There are often several alignments of maximum score.

Based on these alignments, five sequence similarity measures are defined as follows. For two sequences S and T, and weights {m, mm, og, eg}:
M1 is the maximum number of matches over all alignments free of internal indels;
M2 is the maximum length of a block over all alignments;
M3 is the maximum number of matches over all alignments of maximum score;
M4 is the maximum sum of the lengths of the longest two blocks over all alignments of maximum score;
M5 is the maximum sum of the lengths of all the blocks of length at least 3, over all alignments of maximum score.

Notice that, by definition, the following inequalities between these similarity measures are obtained: M4≦M3 and M5≦M3. Also, in order to determine M2 it is sufficient to determine the maximum length of a block over all alignments free of internal indels. For two given sequences, the values of M3 to M5 can vary depending on the values of the weights {m, mm, og, eg}, but not M1 and M2.

For weights {3, 1, 2, 1}, the illustrated alignment is not a maximum score alignment of the two example sequences. But for weights {6, 6, 0, 6} it is; hence this alignment shows that for these two example sequences, and weights {6, 6, 0, 6}, M2≧3, M3≧9, M4≧6 and M5≧6. In order to determine the exact values of M1 to M5, all the necessary alignments need to be considered. M1 and M2 can be found by looking at the s+t−1 alignments free of internal indels, where s and t are the lengths of the two sequences considered. Mathematical tools known as dynamic programming can be implemented on a computer and used to determine M3 to M5 in a very quick way. Using a computer program to do these calculations, it was determined that:

with the weights {6, 6, 0, 6}, M1=8, M2=4, M3=10, M4=6 and M5=6;

with the weights {3, 1, 2, 1}, M=8, M2=4, M3=10, M4=6 and M5=4.

According to the preferred embodiment of this invention, two sequences S and T each of length 24 are too similar if at least one of the following happens:

M1>16 or

M2>13 or

M3>20 or

M4>16 or

M5>19

when using either weights {6, 6, 0, 6}, or {6, 6, 5, 1}, or {6, 2, 5, 1}, or {6, 6, 6, 0}. In other words, the five similarity measures between S and T are determined for each of the above four sets of weights, and checked against these thresholds (for a total of 20 tests).

The above thresholds of 16, 13, 20, 16 and 19, and the above sets of weights, were used to obtain the sequences listed in Table I. Additional sequences can thus be added to those of Table I as long as the above alignment rules are obeyed for all sequences.

It is also possible to alter thresholds M1, M2, etc., while remaining within the scope of this invention. It is thus possible to substitute or add sequences to those of Table II, or more generally to those of Table IIA to obtain other sets of sequences that would also exhibit reasonably low cross-hybridization. More specifically, a set of 24-mer sequences in which there are no two sequences that are too similar, where too similar is defined as:

M1>19 or

M2>17 or

M3>21 or

M4>18 or

M5>20

when using either weights {6, 6, 0, 6}, or {6, 6, 5, 1}, or {6, 2, 5, 1}, or {6, 6, 6, 0}, would also exhibit low cross-hybridization. Reducing any of the threshold values provides sets of sequences with even lower cross-hybridization. Alternatively, ‘too similar’ can also be defined as:

M1>19 or

M2>17 or

M3>21 or

M4>18 or

M5>20

when using either weights {3, 1, 2, 1}. Alternatively, other combinations of weights will lead to sets of sequences with low cross-hybridization.

Notice that using weights {6, 6, 0, 6} is equivalent to using weights {1, 1, 0, 1}, or weights {2, 2, 0, 2}, . . . (that is, for any two sequences, the values of M1 to M5 are exactly the same whether weights {6, 6, 0, 6} or {1, 1, 0, 1} or {2, 2, 0, 2} or any other multiple of {1, 1, 0, 1} is used).

When dealing with sequences of length other than 24, or sequences of various lengths, the definition of similarity can be adjusted. Such adjustments are obvious to the persons skilled in the art. For example, when comparing a sequence of length L1 with a sequence of length L2 (with L1<L2), they can be considered as too similar when

M1>19/24×L1

M2>17/24×L1

M3>21/24×L1

M4>18/24×L1

M5>20/24×L1

when using either weights {6, 6, 0, 6}, or {6, 6, 5, 1}, or {6, 2, 5, 1} or {6, 6, 6, 0}.

Polynucleotide sequences can be composed of a subset of natural bases most preferably A, T and G. Sequences that are deficient in one base possess useful characteristics, for example, in reducing potential secondary structure formation or reduced potential for cross hybridization with nucleic acids in nature. Also, it is preferable to have tag sequences that behave isothermally. This can be achieved for example by maintaining a constant base composition for all sequences such as six Gs and eighteen As or Ts' for each sequence. Additional sets of sequences can be designed by extrapolating on the original family of non-cross-hybridizing sequences by simple methods known to those skilled in the art.

There are 1168 polynucleotide sequences given in Table II. This family of 1168 sequence tags have been shown to form specific duplex structures with their complementary sequences, and with low potential for cross-hybridization within the sequence set (see, e.g., U.S. Patent Publication 20050186573).

Since all 1168 of this family of polynucleotides can work with each other as a minimally cross-hybridizing set, then any plurality of polynucleotides that is a subset of the 1168 can also act as a minimally cross-hybridizing set of polynucleotides. An application in which, for example, 30 molecules are to be sorted using a family of polynucleotide tags and tag complements could thus use any group of 30 sequences shown in Table II. This is not to say that some subsets may be found in a practical sense to be more preferred than others. For example, it may be found that a particular subset is more tolerant of a wider variety of conditions under which hybridization is conducted before the degree of cross-hybridization becomes unacceptable.

It may be desirable to use polynucleotides that are shorter in length than the 24 bases of those in Table II. A family of subsequences (i.e., subframes of the sequences illustrated) based on those contained in Table II having as few as 10 bases per sequence could be chosen, so long as the subsequences are chosen to retain homological properties between any two of the sequences of the family important to their non cross-hybridization.

The selection of sequences using this approach is amenable to a computerized process. Thus for example, a string of 10 contiguous bases of the first 24-mer of Table II could be selected: AAATTGTGAAAGATTGTTTGTGT-A (SEQ ID NO:1).

The same string of contiguous bases from the second 24-mer could then be selected and compared for similarity against the first chosen sequence: GTTAGAGTTAATTGTATTTGATGA (SEQ ID NO:2 of Table II). A systematic pairwise comparison could then be carried out to determine if the similarity requirements are violated. If the pair of sequences does not violate any set property, a 10-mer subsequence can be selected from the third 24-mer sequence of Table II, and compared to each of the first two 10-mer sequences (in a pairwise fashion to determine its compatibility therewith, etc. In this way a family of 10-mer sequences may be developed.

It is within the scope of this invention, to obtain families of sequences containing 1mer, 12mer, 13mer, 14-mer, 15mer, 16mer, 17mer, 18mer, 19mer, 20mer, 21mer, 22mer and 23mer sequences by analogy to that shown for 10mer sequences. It may be desirable to have a family of sequences in which there are sequences greater in length than the 24-mer sequences shown in Table II. It is within the capability of a person skilled in the art, given the family of sequences shown in Table II, to obtain such a family of sequences. One approach would be to insert into each sequence at one or more locations a nucleotide, non-natural base or analogue such that the longer sequence should not have greater similarity than any two of the original non-cross-hybridizing sequences of Table II and the addition of extra bases to the tag sequences should not result in a major change in the thermodynamic properties of the tag sequences of that set for example the GC content must be maintained between 10%-40% with a variance from the average of 20%. This method of inserting bases could be used to obtain, for example, a family of sequences up to 40 bases long.

Given a particular family of sequences that can be used as a family of tags (or tag complements), e.g., those of Table II, a skilled person will readily recognize variant families that work equally as well.

Again taking the sequences of Table II for example, every T could be converted to an A and vice versa and no significant change in the cross-hybridization properties would be expected to be observed. This would also be true if every G were converted to a C.

TABLE II

| | |
|---|---|
| SEQ ID NO: | 5′ TAG |

| | |
|---|---|
| 1 | AAATTGTGAAAGATTGTTTGTGTA |
| 2 | GTTAGAGTTAATTGTATTTGATGA |
| 3 | ATGTTAAAGTAAGTGTTGAAATGT |
| 4 | TGATGTTAGAAGTATATTGTGAAT |
| 5 | TTTGTGTAGAATATGTGTTGTTAA |
| 6 | ATAAGTGTAAGTGAAATAAGAAGA |
| 7 | AAGAGTATTTGTTGTGAGTTAAAT |
| 8 | GTGTTTATGTTATATGTGAAGTTT |
| 9 | AAAGAGAATAGAATATGTGTAAGT |
| 10 | TATGAAAGAGTGAGATAATGTTTA |
| 11 | ATGAGAAATATGTTAGAATGTGAT |
| 12 | TTAGTTGTTGATGTTTAGTAGTTT |
| 13 | GTAAAGAGTATAAGTTTGATGATA |
| 14 | AAAGTAAGAATGATGTAATAAGTG |
| 15 | GTAGAAATAGTTTATTGATGATTG |
| 16 | TGTAAGTGAAATAGTGAGTTATTT |
| 17 | AAATAGATGATATAAGTGAGAATG |
| 18 | ATAAGTTATAAGTGTTATGTGAGT |
| 19 | TATAGATAAAGAGATGATTTGTTG |
| 20 | AGAGTTGAGAATGTATAGTATTAT |
| 21 | AAGTAGTTTGTAAGAATGATTGTA |
| 22 | TTATGAAATTGAGTGAAGATTGAT |
| 23 | GTATATGTAAATTGTTATGTTGAG |
| 24 | GAATTGTATAAAGTATTAGATGTG |
| 25 | TAGATGAGATTAAGTGTTATTTGA |
| 26 | GTTAAGTTTGTTTATGTATAGAAG |
| 27 | GAGTATTAGTAAAGTGATATGATA |
| 28 | GTGAATGATTTAGTAAATGATTGA |
| 29 | GATTGAAGTTATAGAAATGATTAG |
| 30 | AGTGATAAATGTTAGTTGAATTGT |
| 31 | TATATAGTAAATGTTTGTGTGTTG |
| 32 | TTAAGTGTTAGTTATTTGTTGTAG |
| 33 | GTAGTAATATGAAGTGAGAATATA |
| 34 | TAGTGTATAGAATGTAGATTTAGT |
| 35 | TTGTAGATTAGATGTGTTTGTAAA |
| 36 | TAGTATAGAGTAGAGATGATATTT |
| 37 | ATTGTGAAAGAAAGAGAAGAAATT |
| 38 | TGTGAGAATTAAGATTAAGAATGT |
| 39 | ATATTAGTTAAGAAAGAAGAGTTG |
| 40 | TTGTAGTTGAGAAATATGTAGTTT |
| 41 | TAGAGTTGTTAAAGAGTGTAAATA |
| 42 | GTTATGATGTGTATAAGTAATATG |
| 43 | TTTGTTAGAATGAGAAGATTTATG |
| 44 | AGTATAGTTTAAAGAAGTAGTAGA |
| 45 | GTGAGATATAGATTTAGAAAGTAA |
| 46 | TTGTTTATAGTGAAGTGAATAGTA |
| 47 | AAGTAAGTAGTAATAGTGTGTTAA |
| 48 | ATTTGTGAGTTATGAAAGATAAGA |
| 49 | GAAAGTAGAGAATAAAGATAAGAA |
| 50 | ATTTAAGATTGTTAAGAGTAGAAG |
| 51 | GTTTAAAGATTGTAAGAATGTGTA |
| 52 | TTTGTGAAGATGAAGTATTTGTAT |
| 53 | TGTGTTTAGAATTTAGTATGTGTA |
| 54 | GATAATGATTATAGAAAGTGTTTG |
| 55 | GTTATTTGTAAGTTAAGATAGTAG |
| 56 | AGTTTATTGAAAGAGTTTGAATAG |
| 57 | TTGTGTTTATTGTGTAGTTTAAAG |
| 58 | ATTGTGAGAAGATATGAAAGTTAT |
| 59 | TGAGAATGTAAAGAATGTTTATTG |
| 60 | ATGTGAAAGTTATGATGTTAATTG |
| 61 | GTTTAGTATTAGTTGTTAAGATTG |
| 62 | GATTGATATTTGAATGTTTGTTTG |
| 63 | TGAATTGAAAGTGTAATGTTGTAT |
| 64 | GATTGTATTGTTGAGAATAGAATA |
| 65 | AAATTTGAGATTTGTGATAGAGTA |
| 66 | GTAATTAGATTTGTTTGTTGTTGT |
| 67 | GTTTGTATTGTTAGTGAATATAGT |
| 68 | ATGTAGTAGTAGATGTTTATGAAT |
| 69 | TGTTTAAAGATGATTGAAGAAATG |
| 70 | TGTGATAATGATGTTATTTGTGTA |
| 71 | ATAGTTGTGAGAATTTGTAATTAG |
| 72 | ATAGATGTAAGAGAAATTGTGAAA |
| 73 | AGATTAAGAGAAGTTAATAGAGTA |
| 74 | GAAGTAAATTGTGAATGAAAGAAA |
| 75 | AATGTAAGAAAGAAGATTGTTGTA |
| 76 | TTTGATTTATGTGTTATGTTGAGT |
| 77 | GTATTGAGAAATTTGAAGAATGAA |
| 78 | GAATTGTATGAAATGAATTGTAAG |
| 79 | TATTGTAGAAGTAAAGTTAGAAGT |
| 80 | TTTATGTAATGATAAGTGTAGTTG |
| 81 | ATATAGTTGAAATTGTGATAGTGT |
| 82 | ATAAGAAATTAGAGAGTTGTAAAG |
| 83 | GAATTGTGAAATGTGATTGATATA |
| 84 | AAATAAGTAGTTTAATGAGAGAAG |
| 85 | GATTAAAGAAGTAAGTGAATGTTT |
| 86 | TATGTGTGTTGTTTAGTGTTATTA |
| 87 | GAGTTATATGTAGTTAGAGTTATA |
| 88 | GAAAGAAAGAAGTGTTAAGTTAAA |
| 89 | TAGTATTAGTAAGTATGTGATTGT |
| 90 | TTGTGTGATTGAATATTGTGAAAT |
| 91 | ATGTGAAAGAGTTAAGTGATTAAA |
| 92 | GATTGAATGATTGAGATATGTAAA |
| 93 | AAGATGATAGTTAAGTGTAAGTTA |
| 94 | TAGTTGTTATTGAGAATTTAGAAG |
| 95 | TTTATAGTGAATTATGAGTGAAAG |
| 96 | GATAGATTTAGAATGAATTAAGTG |
| 97 | TTTGAAGAAGAGATTTGAAATTGA |
| 98 | ATGAATAAGAGTTGATAAATGTGA |
| 99 | TGTTTATGTAGTGTAGATTGAATT |
| 100 | TTTAAGTGAGTTATAGAAGTAGTA |
| 101 | GATTTATGTGTTTGAAGTTAAGAT |
| 102 | TAGTTAGAGAAAGTGATAAAGTTA |
| 103 | GTAATGATAATGAAGTGTATATAG |
| 104 | AATGAAGTGTTAGTATAGATAGTA |
| 105 | TAAATTGAGTTTGTTTGATTGTAG |
| 106 | TAATGAAGAATAAGTATGAGTGTT |
| 107 | AAATGTAATAGTGTTGTTAGTTAG |
| 108 | AGAGTTAGTGAAATGTTGTTAAAT |
| 109 | GAAATAGAAATGTATTGTTTGTGA |
| 110 | AGTTATAAGTTTGTGAGAATTAAG |
| 111 | GAGTTTATAGTTAGAATATGTTGT |
| 112 | AGAGTTATTAGAAGAAGATTTAAG |
| 113 | GAGTTAATGAAATAAGTATTTGTG |
| 114 | ATGATGAATAGTTGAAGTATATAG |
| 115 | ATAGATATGAGATGAAAGTTAGTA |
| 116 | TATGTAAAGAAAGTGAAAGAAGAA |
| 117 | TGAATGTAGAAATGAATGTTGAAA |
| 118 | AATTGAATAGTGTGTGAGTTTAAT |
| 119 | AGATATTGTTTGATTAATGAAGAG |
| 120 | AAAGTTGTAAAGTTGAAGATAAAG |
| 121 | GTTAAGAGATTATGAGATGTATTA |
| 122 | AGAAGATATAAGAAGATTGAATTG |
| 123 | GTAGAAATTTGAATTGATGTGAAA |
| 124 | AAGAGTAGATTGATAAGTATATGA |
| 125 | TGATATAGTAGTGAAGAAATAAGT |
| 126 | AGATAATGATGAGAAATGAAGATA |
| 127 | ATGTGAAAGTATTTGTGATATAGT |
| 128 | AATAAGAGAATTGATATGAAGATG |
| 129 | TAAGTGTATTTAGTAGAATGAAGT |
| 130 | TATGTTAGATTTGTTGAGATTGAT |
| 131 | AGTTTGTATGAAGAGATAGTATTT |
| 132 | GAGAAATGTTATGTATTTAGTAGT |
| 133 | TATGTGAGAATGTGTTTGATTTAA |
| 134 | GTATGTTTGTTTATAGAATGTATG |
| 135 | GAGTATATAGAAGAAAGAAATTTG |
| 136 | ATGAGTGAAGTAAATGTAGTTATT |
| 137 | TTAAGAAGTGAGTTATTGTGATAT |
| 138 | ATGAAATGAGAATATTGTTGTTTG |
| 139 | GATTAATGATTATGTGAATTGATG |
| 140 | GAAATGTTAAAGATATGAAAGTAG |
| 141 | TATTGTTGATTTGATATTAGTGTG |
| 142 | TTTATGTTTGTGTATGTAAGTAGT |
| 143 | AATTGAAAGAATTGTGTGAATTGA |
| 144 | TGAGTTTGAATTTGTTTGAGTAAT |
| 145 | GATGTATAATGATGTGTGTAAATT |
| 146 | ATGTGAGAGAAGAATTTGTTTATT |
| 147 | GTGATAAAGTATTGTTGATAGAAA |
| 148 | GAAGTAGAATAGAAAGTTAATAGA |
| 149 | TTGTGTAGTTAAGAGTTGTTTAAT |
| 150 | TAGTAGTAAGTTGTTAGAATAGTT |
| 151 | AATTTGAAGTATAATGAATGTGTG |
| 152 | TAGAAATTGTAGTATTTGAGAGAA |
| 153 | TGTATATGTTAATGAGATGTTGTA |
| 154 | TATTTGATAAGAGAATGAAGAAGT |
| 155 | TTGAATAGTGTAATGAATATGATG |
| 156 | GTAGTTTGTGAATAGAATTAGTTT |
| 157 | AAAGATGATTGTAATTTGTGTGAA |
| 158 | GAAGATTGTTGAGTTAATAGATAA |
| 159 | AGATTATGTAGTGATGTAAATGTT |
| 160 | GAATTTAGATGTAGATATGAATGT |
| 161 | GATAGAAGTGTATTAAGTAAGTTA |
| 162 | TATGAATTATGAGAAGAATAGAGT |
| 163 | TTTGTTATGAAGTGATTTGTTTGT |
| 164 | GTAAAGATTGTGTTATATGAAATG |
| 165 | TTGTGATAGTAGTTAGATATTTGT |
| 166 | GAATTAAGATAAAGAAGAGAAGTA |
| 167 | GATTGTAGAATGAATTTGTAGTAT |
| 168 | AAATAAGAGAGAGAATGATTTAGT |
| 169 | AATTATGTGAATAGATTGTTGAAG |
| 170 | TTAAGATTTATGTGATAGTAGAGT |
| 171 | TTAAAGATAGTGTTTGTTGTGTTA |
| 172 | TATTGATTTATGAAGAGTATAGTG |
| 173 | AAATTTGATGAGTAGTTTAAGAGA |
| 174 | ATAAAGTTGTTTGATGTTTGAATG |
| 175 | GATTGTGATGAATAATGTTATTGA |
| 176 | GATGAAGAAATATGATATGAATAG |
| 177 | TTAAAGTTATTGAAGTGAAGTTGA |
| 178 | TTGTAAGAAATAGAGATTTGTGTT |
| 179 | GAGATTGAGTTTAAGTATTAGATT |
| 180 | AGTGATAATAGAATGATAAATGTG |
| 181 | GATAATAGTGAATTTGAGTTGTAT |
| 182 | AGATATTTGTAGTAGAAAGTATGT |
| 183 | GTTATGAATGTTGAATTTGAATGT |
| 184 | ATGAAAGATTTAGTTGTGAGATAT |
| 185 | AAATAGAGAAGTTATGATGTGATA |
| 186 | TTAGTGAGAAATGTTTAATGTGAT |
| 187 | TGAAGAATATGTGAAATTAGTTTG |
| 188 | GTTTGATAGTTTAATGAGTATTGA |
| 189 | GTTGTAAGTAATGATAAAGTATGA |
| 190 | TAAGAGTAGTAATTGTTGTTTAGA |
| 191 | TTTGAGAGAGTATGTATGATTATT |
| 192 | ATTGATTGTGAATTAGATAGAAGA |
| 193 | GATTAGTATTTAGTAGTAATAGAG |
| 194 | TATGTATTAGAGATATTGAAAGTG |
| 195 | TATGTGAAAGTAATGATAAATGAG |
| 196 | GTAATTAGTAATGATTTGAATGAG |
| 197 | GTTTATTGTAAAGATGTAAGTGAA |
| 198 | TAGTAGAATTGTTGTTAAAGAATG |
| 199 | TATTGTTAGTTATGTAGTGTGTAA |
| 200 | GAGTGAAAGTTATATGAAAGTATA |
| 201 | ATATAGAAGTTGATGAGTTTATGA |
| 202 | TTTAGAAGTAAGAATAAGTGAGTA |
| 203 | TGTGTATAAGATATTTGTAAGAAG |
| 204 | TAGAAGAGTTGTATTGTTATAAGT |
| 205 | GTGTTATTAGTTTAAGTTAGAGTA |
| 206 | AATATAGTGATGTGAAATTGAATG |
| 207 | TTAGAGAATAGAGTGATTATAGTT |
| 208 | GAAGTGAGTTAATGATTTGTAAAT |
| 209 | AATGTAAAGTAAAGAAAGTGATGA |
| 210 | GTTAGTTATGATGAATATTGTGTA |
| 211 | AAATGAGTTAGAGTAGAATTATGT |
| 212 | GATATAGAAGATTAGTTAGTGATA |
| 213 | ATAGTTTGTTGAGATTTATGAGTA |
| 214 | TAGAATAGTTAGTAGTAAGAGTAT |
| 215 | GAATTTGTATTGTGAAGTTTAGTA |
| 216 | GTAGTAAGAAGAGAATTAGATTAA |
| 217 | AATGTGTTATGTATGTAAATAGTG |
| 218 | GAATTAGTTAGAGTAAATTGTTTG |
| 219 | GAAATTGAAGATAGTAAGAAATGA |
| 220 | GTGTATTATGTGATTTATGATAGA |
| 221 | TATTATGAGAAAGTTGAATAGTAG |
| 222 | TATGTATTGTATTGAGTAGATGAA |
| 223 | GTGATTGAATAGTAGATTGTTTAA |
| 224 | AGTAAGTTGTTTGATTGAAATTTG |
| 225 | GAAGTTTGATTTAAGTTTAAGAAG |
| 226 | GAGAAGATAAATGATATTGTTATG |
| 227 | ATGATGAGTTGTTAATAGTTAGTT |
| 228 | TATGATATTTGAAGAGTGTTAAGA |
| 229 | GAGATGATTAAAGTGATTTATGAA |
| 230 | ATAGTTAAGAGTGATGAGAATAAA |
| 231 | TTTATTGTTAGATAAAGAGTTGAG |
| 232 | AGAATATTGATAGTTGAAGTTGAA |
| 233 | TAGTGTAAAGTGTAGATTGTAAAT |
| 234 | AGTAGTGATATGATTTGAATATTG |
| 235 | TGTATTGAATTAGAATAGTGAGAA |
| 236 | TGATATGAGATAGAAGTTTAATGT |
| 237 | GAAGAAGTAAGTATAAAGTAAATG |
| 238 | TTTAAGTGTGATAAGAAAGATAGA |
| 239 | TATTGTTGAATGTGTTTAAAGAGA |
| 240 | GAATAATGATGAGATGATTATTGA |
| 241 | TAGAGAAAGAGAGAATTGTATTAA |
| 242 | ATGTATAATGAGATATGTTTGTGA |
| 243 | AATAGATAAGATTGATTGTGTTTG |
| 244 | TTTGATGATAATAGAAGAGAATGA |
| 245 | AGATGAATAAGTTGTGAATGTTTA |
| 246 | AGATGAAAGAAAGTGTAGAATATT |
| 247 | TGTTAAATGTATGTAGTAATTGAG |
| 248 | TAGTAGTGTGAAGTTATTTGTTAT |
| 249 | AGTGAATGTTTGTAAAGAGTTTAA |
| 250 | GATAAATGAGAATTGAGTAATTGT |
| 251 | TGATGAGAAATTGTTTAAGTGTTT |
| 252 | AAATAAGTAGTGTGAGTAATAGTA |
| 253 | TATGAAATATGTGATAGTAAGAGA |
| 254 | ATTGTAAGAGTGATTATAGATGAT |
| 255 | AGAGTAAGAATGAAAGAGATAATA |
| 256 | TAAGTAAGTAGATGTTAAAGAGAT |
| 257 | AAATAGAAAGAATTGTAGAGTAGT |
| 258 | ATAGATTTAAGTGAAGAGAGTTAT |
| 259 | GAATGTTTGTAAATGTATAGATAG |
| 260 | AAATAGAATGAGTAGTGAAATATG |
| 261 | TTGAATTATGTAGAGAAAGTAAAG |
| 262 | TAGTAAATTGAGAGTAGTTGAATT |
| 263 | TGTAAAGTGTTTATAGTGTGTAAT |
| 264 | ATATGATTTGAGATGAGAATGTAA |
| 265 | AATATTGATATGTGTTGTGAAGTA |
| 266 | AGTGAGATTATGAGTATTGATTTA |
| 267 | TTGTATTTAGATAGTGAGATTATG |
| 268 | ATAGAAATGAAAGATAGATAGAAG |
| 269 | GATTGTATATGTAAAGTAGTTTAG |
| 270 | TATGAATGTTATTGTGTGTTGATT |
| 271 | GATATTAGTAGAGTAAGTATATTG |
| 272 | TGAGATGAATTTGTGTTATGATAT |
| 273 | TATGAATGAAGTAAAGAGATGTAA |
| 274 | GAGTGAATTTGTTGTAATTTGTTT |
| 275 | AGAAATTGTAGAGTTAATTGTGTA |
| 276 | GTGTTAATGAAAGTTGTGAATAAT |
| 277 | TGTGATTTGTTAAGAAGATTAATG |
| 278 | AGTAGTATTGTAAAGTATAAAGAG |
| 279 | TGATTGTTGTATAGTTATTGTGTA |
| 280 | GATTGTAGTTTAATGTTAAGAATG |
| 281 | ATGAAATAAGAAATTGAGTAGAGA |
| 282 | TATGATGATATTTGTTGTATGTGT |
| 283 | TTTAGAGTTTGATTAGTATGTTTG |
| 284 | AATAAGAGATTGTGATGAGAAATA |
| 285 | AATGAATAGAATAGAGAATGTAGA |
| 286 | GTAGTAGTAATTTGAATGTTTGAA |
| 287 | AGTGAGTAATTGATTGATTGTTAA |
| 288 | GAATAATGTTTAGTGTGTTTGAAA |
| 289 | ATATGAAAGTAGAGAAAGTGTTAT |
| 290 | TGAGTTATTGTATTTAGTTTGAAG |
| 291 | TAGTTGAGTTTAAAGTTGAAAGAA |
| 292 | TAAAGAGTGATGTAAATAGAAGTT |
| 293 | TGTAGTGTTTAGAGTAAGTTATTA |
| 294 | AGAGATTAATGTGTTGAAAGATTA |
| 295 | GTAATAAGTTGTGAAAGAAGATTA |
| 296 | GAGATGTTATAGATAATGAAAGAA |
| 297 | TTTAGTTGATTGTTGAATAGAGTA |
| 298 | ATTATTGAAAGTAGATGTTAGATG |
| 299 | TTTATGTGTGATTGAGTGTTTAAT |
| 300 | TATTTAGTTAGATAGATAGAGAGT |
| 301 | ATGTGTTTATGTGAAAGATTTGTA |
| 302 | ATAGTAATTAGAAGAGAAGAATGT |
| 303 | TATGAGTGATTAGAATTGTATTTG |
| 304 | TTAATGTATTGTTTAAAGAGTGTG |
| 305 | ATAGAGAATTAAGAATTGTTTGAG |
| 306 | GTTATAAGTAGAAATGTATAGAAG |
| 307 | AGTAATTAGTTTGAAATGTGTAGT |
| 308 | GAAAGATTATGATTGTAAAGTGAT |
| 309 | GTAAGATTAGAAGTTAATGAAGAA |
| 310 | GAGAATGTTGAATAAGAAGTAATT |
| 311 | TTAAGAGTGTTTGAATAGTGTTTA |
| 312 | ATAAAGAAAGAGTATGAGATTATG |
| 313 | AGTTATTGATTGAAGATGAGAAAT |
| 314 | GTTTGTGTTTGTATAAGTTGTTAA |
| 315 | TTGTATGTGAGTTTAGATTAATGA |
| 316 | TAGTTAAAGTATAGTTGTTTGAGT |
| 317 | AAATTTGTGTTGAGATTTGTATAG |
| 318 | TATTAGTGTTATGATAAAGAGAAG |
| 319 | TATAAGAAGTAATTTGAGAAGAGT |
| 320 | TAAGTTGAGATGTTTGTTTGATAA |
| 321 | GTGTAGATTTATGAATTGAGTAAT |
| 322 | TATAGAGAAGTGTTTAGTTGTATA |
| 323 | ATAAAGAAGAATAGTTGTTGTGTA |
| 324 | AGATTGAAATAGATTAGAAAGTTG |
| 325 | GTTGTTATAAGAAATAGTTTGTTG |
| 326 | AGAAATAGAGTAAGAGTGTTTAAA |
| 327 | AGAGATAGTAGTAAATAGTTATTG |
| 328 | AAATGATTGTGTAAGTTATGTATG |
| 329 | AAGAAGTAAGAGAGAAATTTGAAT |
| 330 | GTGTGTATTTAGTTGATAATTGAT |
| 331 | ATTGTTGTTGTTGAGAAATGTATT |
| 332 | AGATAAGTTAAAGTAAAGAGAATG |
| 333 | TAGTTGAAGTTAGTTTAAGTGTTA |
| 334 | AGTAAGAATGTAATATGATGATAG |
| 335 | ATGAGATTGAAAGATTTATGAATG |
| 336 | TGATTGAATTAGAGAGAATGTATA |
| 337 | AGTTAGTAAGAGAATATAGTGAAT |
| 338 | ATTAAGATTGTATAGTTAGTGATG |
| 339 | GAGATAAAGAATTGAAATAGAAGA |
| 340 | AGAGTAAATGTTAAGAAAGAAGTT |
| 341 | AAAGTTTGTTATGTGTGAAGAATT |
| 342 | ATTGTGTTTAAGAAATATGATGAG |
| 343 | TATTGAAATGAGATGTATGTAGTT |
| 344 | ATTTGTGTGATGTTTGAAATATGA |
| 345 | TAAGATAATAGTGAGAGAAATTGA |
| 346 | ATTTATGATTAGTGTAAGTGTTGT |
| 347 | GATTAAGAATAAAGTGTGAAGAAT |
| 348 | GTAATTGATGAAGAGTTAGTTTAT |
| 349 | TGTGTTATGTTATAAGAAGTGATA |
| 350 | AGAGAAATTGAATTTAGAAATGTG |
| 351 | TTATTGAATGTGAGAAAGTATTTG |
| 352 | TGTTAATGAGAAGATAATGATAGT |
| 353 | GAAAGTATTTGTTGATTATTGTTG |
| 354 | TAGTTTATGTAGTTAATTGTTGAG |
| 355 | GTTGAAAGATAGTTTGATATGTAT |
| 356 | TTAGAAGATAGATTATTGAGAAAG |
| 357 | AATAATGTTGTGAAATAGATGTGA |
| 358 | AGTAAGAAAGTTTAGTTTAGTTAG |
| 359 | TAGTTTAATGAGATGTTTGATATG |
| 360 | TTAAAGATGTTAAAGAATGAGTGA |
| 361 | AAAGTGTGTATATGTTAGAAAGTA |
| 362 | ATTAAGTTATGTGTTTATGTGTTG |
| 363 | TTTGAAGAAGTGTTTGTATTATGT |
| 364 | TGTTAAGAAGTTTAGTTAAAGTTG |
| 365 | TTTAAGTATAAGATTGTGTGAGAT |
| 366 | AGATATTTGATAGATAGAAGAAAG |
| 367 | ATTTAGAGTTGTAAGAAGATATTG |
| 368 | GAGAAATTGTAATTGTTAGAGTAT |
| 369 | GAAGTATATGTTAAGATGTAATAG |
| 370 | AATATTGAAGATGTAGTGAGTTAT |
| 371 | GAGTTTAGAAATGATAAAGAATTG |
| 372 | TAAGAAATGAGTTATATGTTGAGA |
| 373 | TTGATATAAGAAGTTGTGATAAGT |
| 374 | AAGTGTTTAATGTAAGAGAATGAA |
| 375 | GTTGTGAGAATTAGAAATAGTATA |
| 376 | TTTAGTTTGATGTGTTTATGAGAT |
| 377 | GTAATTGAAAGTATGAGTAGTAAT |
| 378 | TAGTTGAATAAGATTGAGAGAAAT |
| 379 | TTAAGTGAAGTGTTGTTTATTGAA |
| 380 | ATTGATTTGTTGAAATAAGTGTTG |
| 381 | TGAATTGTTGATAAGTTATGAAGA |
| 382 | GTTTGTTATTGAGTAAGTTGAATT |
| 383 | TGATTTAGTATGTATTAGAGTTGA |
| 384 | TAAATAGAGATGAGAATAAGAAAG |
| 385 | AGAATGTTATATGTAGAGAAATTG |
| 386 | ATTTATGTAGTTGAGAGTGATAAA |
| 387 | GTAAAGATAGTTTGAGTAATTTGA |
| 388 | GAAATAGTATAATGTTAAGTGAGA |
| 389 | ATTGTATATTGTGTTGAAGAAAGT |
| 390 | GAGTTAAGTGTAAATGAAATGTAA |
| 391 | ATAGATTGTGTGAAAGAAAGAATT |
| 392 | TTAATAGAAGTTTGTAGTATGATG |
| 393 | TTGTATGTGAGAATAAAGTTTAGT |
| 394 | GTGATTAGATATGATGATATGAAT |
| 395 | TGAAGAAGAATTTAGATTTGTAAG |
| 396 | TGTATGATTATTGATTAGTGTGTT |
| 397 | TGTGAAAGAGAATGATAGATATTT |
| 398 | AATTGAAATGAGTGTGTTTAAGAA |
| 399 | ATTATAGAGTTAGTTTAGAATGAG |
| 400 | AAAGATAGAAATTGAGTGTATGAT |
| 401 | GTAGTTTGTTAATGTTGTATAATG |
| 402 | AGAGATATTAGAATGTAAGAATAG |
| 403 | AGAAGTTTGAAATATGATAGAATG |
| 404 | TAGAATGTAAAGTTTAGTATAGAG |
| 405 | AGTAGATGTATGTTAATGTGAATA |
| 406 | TGAAAGTGAAATATGAAATGTTGT |
| 407 | ATAGTATATTGAGTTTGTATGAAG |
| 408 | GAAGAAATGTTTGTAGAATAAGTA |
| 409 | AATGAGTATTGAAGAAATGTATAG |
| 410 | GTGATAGAATTTGTGTTTAATGAA |
| 411 | TGTAGTATGAAGAATAATGAAATG |
| 412 | ATAGAAGTTAATGATAATTGTGTG |
| 413 | GTGATTGTAAGTAAGTAAAGATAA |
| 414 | TATGTAGTTTGTGTTATTTGAAGA |
| 415 | TGAGTAAGTTTGTATGTTTAAGTA |
| 416 | TAAATGTATGAGTGTGTAAAGAAA |
| 417 | GTAAGAGTATTGAAATTAGTAAGA |
| 418 | GTTGAGTGTAAAGATTATTGATAA |
| 419 | AGTATGAGTTATTAGATAAAGTGA |
| 420 | ATTTGTTATAGAGTTGTGTTGTAT |
| 421 | TAATTAGTAGTGTGTTGAAATTTG |
| 422 | TGTATTGAGATTGTTATTGTATTG |
| 423 | GTTATTAGAAGAGATAATTGAGTT |
| 424 | TTGAGTTGTGATTAAGTAGTATAT |
| 425 | GATAGTATAATGATTGAAGTAATG |
| 426 | GTGAAAGATATTTGAGAGATAAAT |
| 427 | AGTTATGATTTGAAGAAATTGTTG |
| 428 | GTAAGTATTTGAATTTGATGAGTT |
| 429 | TAATAGTGTTATAAGTGAAAGAGT |
| 430 | AAATGAATTGATGTGTATATGAAG |
| 431 | AGAAAGTGAGTTGTTAAGTATTTA |
| 432 | TTTATGTGTGAATTGTGTATATAG |
| 433 | GTAATATGATAGAAATGTAAAGAG |
| 434 | GAGAATTGTTTAAAGATAGTTGTA |
| 435 | GAATTTGTTAAGAATGAGTTTGAT |
| 436 | ATAGTGATGATTAAAGAGAATTTG |
| 437 | ATAGATGTTTAGTTGAGATTATTG |
| 438 | AAGAGTGTAAATAGAAAGTGATAT |
| 439 | TGTGTATTGATTGTTGAGATAAAT |
| 440 | TAGTATAGTGAGAAAGAGTTAAAT |
| 441 | AAAGATAAGAAAGAGATGATGTTT |
| 442 | GAAGTTATTGAAATAGAGAAGTAT |
| 443 | ATGTATGTATAGAAAGAGTAAATG |
| 444 | GATGTTTGTAAAGATTGAAATTGA |
| 445 | AATTTAGAGAGTATTTGTGTTGTA |
| 446 | AATTTGTTTGAAAGAAAGTAAGTG |
| 447 | AAAGAGTAGTGTTATTGTTAGATA |
| 448 | GTATGTTGTATATGTTGTTGATAT |
| 449 | GTAGAATTTGTTGAGTATTTGTAA |
| 450 | ATGAATTTAGTTAGTGTAAGAAAG |
| 451 | ATGATAAGAAATGTTGATGAAGTA |
| 452 | TTGATGATGAAGATAATGTAGATA |
| 453 | AGATGATATGATATAGATTAGATG |
| 454 | TTGAAAGTTAGAAAGATAGATGTT |
| 455 | GTTTAATGTTAGTTAGAAAGTAAG |
| 456 | GAGATTTAAGTTTGAAGTGAAATA |
| 457 | TTTGTTAGTAGTTGTTATAAGAGA |
| 458 | TATGAGAATAGTTTGTTAGTGAAT |
| 459 | TTGAAAGTTTAAAGAAGAGATAAG |
| 460 | AAGTGAGTTGAAATGAAATATGTT |
| 461 | GTTAGAAATGAAATGAGTAGTTAT |
| 462 | TAAGTATTGTATTTGTGTGTGTAT |
| 463 | TGTATTAGTAAAGAAGAGAGAATA |
| 464 | GAGAAGAGAAATAAGTTGAAATAA |
| 465 | GTAAAGTAGAAATAGAATTGAGTT |
| 466 | GTGTGTTATTTGTTTGTAAAGTAT |
| 467 | TTTGATGTATGAATATAGTATGAG |
| 468 | AAGATTGTGTGAATAGTTGAAATT |
| 469 | TATAAAGTTTGAAGATGAGTGATA |
| 470 | AGATAAAGAGATTTAAGATGTATG |
| 471 | GAAGAATTAAGTTGAGAATTAAGA |
| 472 | TAGAGAAATTTGATAAAGAAAGAG |
| 473 | AAAGTTTATGAAGTTATTGAGTAG |
| 474 | AAATAGTGTAAGTAAAGAGATGAT |
| 475 | TATGATGATTTAGTTATAAGAGTG |
| 476 | TAGATAAATGTTATGATGAGTAAG |
| 477 | AGATTGATTGTGATGATTTGTATA |
| 478 | TTAAGAAGAATTGTATATGAGAGT |
| 479 | GTAGAATGTTTAGAGTTGAATATA |
| 480 | GAGAAATAGTAAGAAGTAAATAGA |
| 481 | ATTGAAGTTGTTATGTGAAGATTT |
| 482 | TAAATGTTGTGTAGAGTAATTAGA |
| 483 | AAATAAGAGTTTGAGAAGTTGTTT |
| 484 | AGTTGTAATAAGAAGTGATTTAAG |
| 485 | GTTAGAATGTATATAGAGTTAGAT |
| 486 | TTGATATTGAAAGAGAAAGTTATG |
| 487 | TTAAAGAGAGAAATGTTTGATTAG |
| 488 | TGTGAATTTGAGTATTAGTAAGAA |
| 489 | TAATTTGAATGTGAAAGTTGTTAG |
| 490 | ATGTGTTTGAAAGATGATGATTTA |
| 491 | AAGTTATGTTGATATTGAGTGAAA |
| 492 | TAGATAAAGAAGATAGAGATTTAG |
| 493 | GATGAATGTAGATATATGTAATGA |
| 494 | GAAGAATAGTTTATGTAAATGATG |
| 495 | GTAGTATATAGTTAAAGATGAGTT |
| 496 | GTTATTTGTGTATGATTATGATTG |
| 497 | AGAGATTAGAAATTGAGAGAATTA |
| 498 | GTATGATAGAGTTTATAGTGATAA |
| 499 | GTTAGAAAGAATGAAATTGAAGTA |
| 500 | AAGAATGAGAATATAGAGATGAAT |
| 501 | AAAGAGAATAGTGTTTAAGAAGAT |
| 502 | GATGTGTTATTGATAGAAATTAGA |
| 503 | TAGAGTTATAGAGATATTGTATGA |
| 504 | GAGAGTTGAATAAGTTAAAGATAT |
| 505 | AGATATGAAATAGATTGTTAGAGA |
| 506 | GAGTGAATAGAAAGATATGTTAAT |
| 507 | AAAGAGATATTGAAGAGAATAAAG |
| 508 | GTTATAGAATAAGTTGTAAAGTGT |
| 509 | TGATAGTATGATAATGTGTTTATG |
| 510 | TTTGTTGTTAAGTATGTGATTTAG |
| 511 | TAAAGTGTTGTGTTAAAGATTAAG |
| 512 | TGTGTTTGATTGATTAATGTTATG |
| 513 | ATTAATGAATGAGTGTTGTAATGT |
| 514 | TAGATGTTTGTGAGTTTGATATTA |
| 515 | GAATGAATAGTAATAGATGATTTG |
| 516 | AATAGTGTGTTGTTATATGATTAG |
| 517 | TAGATTAGAAGATGTTGTGTATTA |
| 518 | AATGTGTGTGTTAAATGAATTTGT |
| 519 | GAATTAAGTATATGAGTGTAGAAA |
| 520 | TTATTGTGTGTAAGTAGTGTAAAT |
| 521 | GTAGTAAAGAGAATTGTTTAGTAT |
| 522 | AAGTTTGTAAGAAGTAGTTGAATA |
| 523 | AGTTATAGTATAGTAGTATAGAGA |
| 524 | GAAAGAAATGTGTATAGTTTAATG |
| 525 | TTGTGAGTAATGAATGATGTATTA |
| 526 | GTAGAGTTGTAAATAGAGAATAAA |
| 527 | ATTAATGTAGATTGTAAGAGATAG |
| 528 | TTAGTGTGTTTGTAGATAGAATTA |
| 529 | AGAGAGTTTGTGTATATGTATAAA |
| 530 | TTAAGTTTAGTGAGATTTGTTAAG |
| 531 | ATGAAGTTTATTGAATAGTAGTGA |
| 532 | ATATTTGTGTTGTATGTTTGTGAA |
| 533 | AAAGTGTTTATAGAAGATTTGATG |
| 534 | AAGAGATATGATTTGTTAGTTGTA |
| 535 | AAGAAGAAATGAGTGATAATGTAA |
| 536 | TAGTGTTTGATATGTTAAGAAGTT |
| 537 | GTAGAAAGTGATAGATTAGTAATA |
| 538 | GATAAATGTTAAGTTAGTATGATG |
| 539 | AGATTAGAAGAATTGTTTAGAATG |
| 540 | ATATTTGAGAAGTGTGAAATGAAT |
| 541 | TGAGTAAATAGTTTATGAGTAGTA |
| 542 | TTAGAGAGTAGATAAAGATTTGAT |
| 543 | ATTGTTTAAGTTGTTGATAAGATG |
| 544 | GTTGTAAAGTTAAAGTGTGAATTT |
| 545 | ATAGATTGTGTGTTTGTTATAGTA |
| 546 | GTAAGTTATTGAGAATGATAATAG |
| 547 | TAGATTAGTTGATAAGTGTGTAAT |
| 548 | AAATGTAAATGAAGAGTGTTTGTT |
| 549 | GATAGAAGAAATGTATATAGTGAT |
| 550 | TATAGAGTGTATGTTATGATAAAG |
| 551 | TATGAAGTGATAAGATGAAGAATT |
| 552 | TGTTGAGAATAGTAAGAGAATTTA |
| 553 | TAGATAATGTGAAGTAATAAGTGA |
| 554 | GTATTATGATGATAGTAGTAAGTA |
| 555 | AGATATGATTTAGTATTGAATGTG |
| 556 | AATTAAGTTTGTAGAGTGATTTGA |
| 557 | AAGAAATAGATGTAGTAAGATGTT |
| 558 | TTGAGAAGTTGTTGTAATAAGAAT |
| 559 | AGTGTGAAATAGTGAAAGTTTAAA |
| 560 | TTTATGTAGTAGATTTATGTGAAG |
| 561 | ATTAATGAGAAATTAGTGTGTTAG |
| 562 | ATGTTAATAGTGATAGTAAAGTGA |
| 563 | TATGTTGATAAATGATTATGAGTG |
| 564 | TTATTAGAGTTGTGTGTGATATAT |
| 565 | TGTTGTTATGATTGAGTTAGAATA |
| 566 | AATTTGAGTTAAGAAGAAGTGTAA |
| 567 | AAAGATAAAGTTAAGTGTTTGTAG |
| 568 | TGTTGAGATGATATTGTATAAGTT |
| 569 | TAAATAGTGAATGAGTTATAGAGT |
| 570 | ATAGATGTTATGATAGTTAGTTAG |
| 571 | GTTAAGTGAAGATATGTATTGTTA |
| 572 | TAAGAAAGTAAAGTTTGTAGATGT |
| 573 | AAGAGAAAGTTTGATTGAATAAAG |
| 574 | ATATTAGATGTGAGTTATATGTGT |
| 575 | AGTTTGAGTTTAGTATTGTGAATA |
| 576 | ATGTTAAATGAGAGATTGTGTATA |
| 577 | TAAATGTTGTGATTATTGTGAGAT |
| 578 | TAAGAATTGAAGTAAGAGTTATTG |
| 579 | AGAGATAGAATTAAGTTTGTTGAT |
| 580 | GAAGAATGTTAAGAAATATGTAAG |
| 581 | TATTTGTGATTAAGAAGTTGAGAA |
| 582 | AGTTAGAATTTGTGTAGTAGAATT |
| 583 | AAGTTTATTGTTGATGTTGTATTG |
| 584 | GAATGAGTTTAAGAGTTTATAGTA |
| 585 | AGTGAAGATTGTATGTAGTATAAA |
| 586 | AGTTGAAATGAGTATTAAGTAATG |
| 587 | ATGTGTTATTTGAGATGAGTAATT |
| 588 | AAATAGTGTTGTTGAAGTTGTTAT |
| 589 | GTAGAGAAAGATATATGTAGTTTA |
| 590 | GAGAGTATTTGATGAATGATTATA |
| 591 | GAGTATAAGTTTAGTGTATATTGA |
| 592 | ATAATGTGATTATTGATTGAGAGA |
| 593 | TTAGTTGTTATGTGAGAGTAATAA |
| 594 | AAATGAGTATATTGAATTGTGATG |
| 595 | AATTAGAAGTAAGTAGAGTTTAAG |
| 596 | TGTAAGTTTAAAGTAAGAAATGTG |
| 597 | GAAATGATAAGTTGATATAAGAAG |
| 598 | AATGAGTAGTTTGTATTTGAGTTT |
| 599 | AGTGAATGTAAGATTATGTATTTG |
| 600 | GTAATTGAATTGAAAGATAAGTGT |
| 601 | TATGTTTAAGTAGTGAAATAGAGT |
| 602 | GTATTGAAATTGAATTAGAAGTAG |
| 603 | AATATGTAATGTAGTTGAAAGTGA |
| 604 | TGAATATTGAGAATTATGAGAGTT |
| 605 | TAGTGTAAATGATGAAGAAAGTAT |
| 606 | GTATGTGTAAAGAAATTTGATGTA |
| 607 | AATTGTTTGAAAGTTTGTTGAGAA |
| 608 | AATTGTTTGAGTAGTATTAGTAGT |
| 609 | TAATTGAGTTTGAATAAGAGAGTT |
| 610 | TGTTGATTGTAAGTGTTTATTGTT |
| 611 | GAAATTTGTGAGTATGTATTTGAA |
| 612 | TAAGAATGAATGTGAAGTGAATAT |
| 613 | TAATGTGAAGTTTGTGAAAGATAT |
| 614 | TTGTATATGAAAGTAAGAAGAAGT |
| 615 | TAGAGAGAAGAAGAAATAAGAATA |
| 616 | ATTTGAAATGTTAATGAGAGAGAT |
| 617 | TTGTGTGTATATAGTATTAGAATG |
| 618 | ATTGTTAGTATTGATGTGAAGTTA |
| 619 | TGTTTGTATTTGAATGAAATGAAG |
| 620 | TGTTAGATTGTGTTAAATGTACTT |
| 621 | TATAGAGTATTGTATAGACAGAAA |
| 622 | AAATAGTAAGAATGTACTTGTTGA |
| 623 | TGAGTGTGATTTATCATTAAGTTA |
| 624 | ACAATTTGTTGTACTGTTATGATT |
| 625 | GATTGAAGAAAGAAATAGTTTGAA |
| 626 | GATAATAGAGAATAGTAGAGTTAA |
| 627 | GATTGAAATTTGTAGTTATAGTGA |
| 628 | GATTTAAGAAGATGAATAATGTAG |
| 629 | TTTGAGAGAAAGTAGAATAAGATA |
| 630 | GATTAAGAGTAAATGAGTATAAGA |
| 631 | TTTGATAGAATTGAAATTTGAGAG |
| 632 | TGAAGAAGAGTGTTATAAGATTTA |
| 633 | GTGAAATGATTTAGAGTAATAAGT |
| 634 | AAATAAGAATAGAGAGAGAAAGTT |
| 635 | GTTGTAAAGTAATAGAGAAATTAG |
| 636 | AGTGATTTAGATTATGTGATGATT |
| 637 | AGAGTATAGTTTAGATTTATGTAG |
| 638 | ATGATTAGATAGTGAAATTGTTAG |
| 639 | ATGAAATGTATTAGTTTAGAGTTG |
| 640 | ATATTGAGTGAGAGTTATTGTTAA |
| 641 | AGATGTGTATTGAATTAAGAAGTT |
| 642 | TAATGTGTTGATAGAATAGAGATA |
| 643 | AAATTAGTTGAAAGTATGAGAAAG |
| 644 | TTTAGAGTTGAAGAAATGTTAATG |
| 645 | GATTGTTGATTATTGATGAATTTG |
| 646 | TGTTGTTGTTGAATTGAAGAATTA |
| 647 | ATTAAGTAAGAATTGAGAGTTTGA |
| 648 | GTATGTTGTAATGTATTAAGAAAG |
| 649 | TAGTTGTGATTTATGTAATGATTG |
| 650 | TGATAATGAAAGTTTATAGAGAGA |
| 651 | GTAAGATTGTTTGTATGATAAGAT |
| 652 | TTGAATTAAGAGTAAGATGTTTAG |
| 653 | AAGTGTTTGTTTAGAGTAAAGATA |
| 654 | AGAGAGATAAAGTATAGAAGTTAA |
| 655 | ATTATGAATAGTTAGAAAGAGAGT |
| 656 | TTGTTGATATTAGAGAATGTGTTT |
| 657 | TTTATTGAGAGTTTGTTATTTGTG |
| 658 | AGTGTTAAGAAGTTGATTATTGAT |
| 659 | GAGAAATGATTGAATGTTGATAAT |
| 660 | GATAAGTATTAGTATGAGTGTAAT |
| 661 | TTTGATTTAAGAGTGTTGAATGTA |
| 662 | AAGTTAGTAAATAGAGTAGAAAGA |
| 663 | GTAAAGTATGAATATGTGAAATGT |
| 664 | TAATAAGTGTGTTGTGAATGTAAT |
| 665 | AAAGATTTAGAGTAGAAAGAGAAT |
| 666 | TTAGTTTGAGTTGAAATAGTAAAG |
| 667 | TAATAGTATGAGTAAGATTGAAAG |
| 668 | GAAGATTAGATTGATGTTAGTTAA |
| 669 | TAAAGAGAGAAGTTAGTAATAGAA |
| 670 | TAAGTATGAGAAATGATGTGTTAT |
| 671 | GAGTTTGTTTGTTAGTTATTGATA |
| 672 | AAGTAAAGAAATGTTAAGAGTAGT |
| 673 | ATGAGAATTGTTGTTGAAATGTAA |
| 674 | TTAGATTAGAGTAGTAGAAGAATA |
| 675 | TAGTGATGAAGAAGTTAGAAATTA |
| 676 | TAATGTAGTAATGTGATGATAAGT |
| 677 | TTGAGAAAGAATAAGTAGTGTAAA |
| 678 | TAATGAGTGAGATTATAGATTGTT |
| 679 | GTATAAGAAATGTGTGTTTGATTA |
| 680 | GTGAATGTGTTAATGAAGATATAT |
| 681 | GAAAGTTATTAGTAGTTAAAGATG |
| 682 | TAGAATTGTGTTTGATAAGTGATA |
| 683 | TGATTTAGATTGAGAGTTAAATGA |
| 684 | ATTATTGAGTTTGAATGTTGATAG |
| 685 | ATAGTAGTTATGTTTGATTTAGTG |
| 686 | ATAGAAGAAGAATAAAGTTAGAGA |
| 687 | GATGTTGAAAGTAATGAATTTGTA |
| 688 | GAGATTGATAGTAGAAATGATAAA |
| 689 | TGAGAGAATAAAGTATGAATTTGA |
| 690 | TATAAAGATGATGTGAATTAGTAG |
| 691 | TTATGTAAGAATGTTTGAGAGAAA |
| 692 | AGTAAATGATGAATGATATGATGA |
| 693 | GAAATTTGTGTTAAAGTTGAATGA |
| 694 | GATGAATGATTGTGTTTAAGTATA |
| 695 | GAAATAAGTGAGAGTTAATGAAAT |
| 696 | TGTTGAAATAGTTATTAGTTTGTG |
| 697 | TTTGAGAGTATATTGATATGAGAA |
| 698 | ATTGTGTGTAAAGTAAGATTTAAG |
| 699 | TATAGTTTGAAGTGTGATGTATTT |
| 700 | GTGAAGTTATAGTGTATAAAGAAT |
| 701 | GTATGTTGAATAGTAAATAGATTG |
| 702 | TTAGAAAGTGTGATTTGTGTATTT |
| 703 | TTTAGTAATATGTAAGAGATGTGA |
| 704 | AGTATGTATAGATGATGTTTGTTT |
| 705 | ATTTAAGTAAAGTGTAGAGATAAG |
| 706 | ATTTGTGTTGAATTGTAAAGTGAA |
| 707 | ATGTTATTAGATTGTGATGAATGA |
| 708 | TAGTAGTAGAATATGAAATTAGAG |
| 709 | TTTAATGAGAAGAGTTAGAGTATA |
| 710 | AAAGTTTAGTAGAGTGTATGTAAA |
| 711 | ATATATGATAGTAGAGTAGATTAG |
| 712 | TGAGAAGTTAATTGTATAGATTGA |
| 713 | TATAGAGATGTTATATGAAGTTGT |
| 714 | AAATTTGTTAAGTTGTTGTTGTTG |
| 715 | TTGTTGAAGATGAAAGTAGAATTA |
| 716 | AAGAGATAAGTAGTGTTTATGTTT |
| 717 | AATAAGAAGAAGTGAAAGATTGAT |
| 718 | TAAGTTAAAGTTGATGATTGATAG |
| 719 | ATATAAGATAAGAGTGTAAGTGAT |
| 720 | GTTAAATGTTGTTGTTTAAGTGAT |
| 721 | GAGTTAAGTTATTAGTTAAGAAGT |
| 722 | TATTAGAGTTTGAGAATAAGTAGT |
| 723 | TAATGTTGTTATGTGTTAGATGTT |
| 724 | GAAAGTTGATAGAATGTAATGTTT |
| 725 | TGATAGATGAATTGATTGATTAGT |
| 726 | ATGATAGAGTAAAGAATAAGTTGT |
| 727 | AGTAAGTGTTAGATAGTATTGAAT |
| 728 | ATGTAGATTAAAGTAGTGTATGTT |
| 729 | TTATTGATAATGAGAGAGTTAAAG |
| 730 | ATTTGTTATGATAAATGTGTAGTG |
| 731 | TTGAAGAAATAAGAGTAATAAGAG |
| 732 | TGTGTAATAAGTAGTAAGATTAGA |
| 733 | ATGAAAGTTAGAGTTTATGATAAG |
| 734 | ATTAGTTAAGAGAGTTTGTAGATT |
| 735 | TGTAGTATTGTATGATTAAAGTGT |
| 736 | AGTTGATAAAGAAGAAGAGTATAT |
| 737 | GTAATGAGATAAAGAGAGATAATT |
| 738 | TGTGTTGAAGATAAAGTTTATGAT |
| 739 | AAGAAGAGTAGTTAGAATTGATTA |
| 740 | GAATGAAGATGAAGTTTGTTAATA |
| 741 | AAATTGTTGAGATAAGATAGTGAT |
| 742 | TGATTGTTTAATGATGTGTGATTA |
| 743 | ATGAAGTATTGTTGAGTGATTTAA |
| 744 | GTGTAAATGTTTGAGATGTATATT |
| 745 | AATTGATGAGTTTAAAGAGTTGAT |
| 746 | TTTGTGTAATATGATTGAGAGTTT |
| 747 | GTAGTAGATGATTAAGAAGATAAA |
| 748 | TTTAATGTGAAATTTGTTGTGAGT |
| 749 | GTAAAGAATTAGATAAAGAGTGAT |
| 750 | AATAGTTAAGTTTAAGAGTTGTGT |
| 751 | GTGTGATGTTTATAGATTTGTTAT |
| 752 | GTATAGTGTGATTAGATTTGTAAA |
| 753 | GTTGTAAGAAAGATATGTAAGAAA |
| 754 | ATATTAGATTGTAAAGAGAGTGAA |
| 755 | GAGTGATATTGAAATTAGATTGTA |
| 756 | TAAGAAGTTAAAGAAGAGAGTTTA |
| 757 | GATGTTAGATAAAGTTTAAGTAGT |
| 758 | GTGATTGTATGAGAAATGTTAAAT |
| 759 | TGATTATTGTAAGAAAGATTGAGA |
| 760 | AAGAATTGTGTAAGTTTATGAGTA |
| 761 | TTGTATTTAGAAGATTTGTAGATG |
| 762 | TATATGTTTGTGTAAGAAGAAATG |
| 763 | GATAATGTGTGAATTTGTGAATAA |
| 764 | TTAGAAATGTGAGATTTAAGAGTT |
| 765 | AGTGTAGAATTTGTATTTAGTTGT |
| 766 | TAGTTAAGATAGAGTAAATGATAG |
| 767 | GAAGTGATATTGTAAATTGATAAG |
| 768 | GTAATTGTGTTAGATTTAAGAAGT |
| 769 | TGATATTTGTGAATTGATAGTATG |
| 770 | AAGTAAAGAGATATAGTTAAGTTG |
| 771 | ATTAGTTAAGTTATTTGTGAGTGA |
| 772 | AGATGAAGTAGTTTATGAATTAGA |
| 773 | TGAGTTAGTTAAGTGATAGTTAAA |
| 774 | TTATTGTAGATTTAGAGAAGATGA |
| 775 | TATTTGTGTTTGTTGATTAGATAG |
| 776 | GTATAATGTGTGTGAAAGTTATAA |
| 777 | TATATGTTGAGTATAAAGAGAGAA |
| 778 | TTAGTTAGTTTAAAGATTGTGAGT |
| 779 | TTTAGAATAAGTGATGTGATGAAA |
| 780 | AGAGTAATGTGTAAATAGTTAGAT |
| 781 | TGTGATAAAGAGAAATTAGTTGTT |
| 782 | GAATTTAGTGAATGTTTGAGATTA |
| 783 | TGTGATGTGTAAGTATATGAAATT |
| 784 | TTGTGAATGATTAATGAATAGAAG |
| 785 | AATGTTGTTTAGATTGAGAAAGTT |
| 786 | AGATTGTGTTAGTATTAGTATAAG |
| 787 | TTGATGTATTAGAAAGTTTATGTG |
| 788 | TATGATTGTGTGTTAGAGAATTTA |
| 789 | TAGTGTAGATATTTGATAGTTATG |
| 790 | AGTTTAATGTGTTTAGTTGTTATG |
| 791 | TGTGTAAAGTAGAAAGTAAAGATT |
| 792 | GTTATGATATAGTGAGTTGTTATT |
| 793 | TTTGATTGAATGTTAATAGTGTGT |
| 794 | AGAGTATTAGTAGTTATTGTAAGT |
| 795 | TAAGTAGAAAGAAGAAGATATTTG |
| 796 | AGAAAGAGAATTATGTAATGAAAG |
| 797 | TTAGATTTGTTAGTGTGATTTAAG |
| 798 | GATGATTAAGATATAGAGATAGTT |
| 799 | ATATTTGAGTGATTAAGAGTAATG |
| 800 | TGTATTGTGAGTTAAGTATAAGTT |
| 801 | AATTTAGTAGAAAGTGTTGTGTTT |
| 802 | GTTAGAAGATTAAGTTGAATAATG |
| 803 | TAAAGTATGTGAGATGATTTATGT |
| 804 | TGAAATGATTAAAGATGAAGATGA |
| 805 | TTATTAGATGTTGAGTGTTTGTTT |
| 806 | TAGTGTTTAAAGAGTAGTATATGA |
| 807 | AGTTATAAGTAAATGATGTTGATG |
| 808 | TTAAGAGAGAAATAAGTGTATTGT |
| 809 | GATATTGAAATGTGTAAATGATGA |
| 810 | ATGATGAATTAAGAAAGAAAGAGA |
| 811 | GAATAGTTTGATTTGTGTTTGTTA |
| 812 | AGTTGTTTAGATTTGATTTGTAAG |
| 813 | GTATGAGATTTGATATAAGATTAG |
| 814 | TTTATAGTGAGTATAGTGATGATT |
| 815 | TATATGTGAAGATATAAGTGTTTG |
| 816 | ATTGATAGATGATAGTAATTGAGT |
| 817 | TGATAGATGTGAAGAATTTGATTT |
| 818 | GAAGATATTGAAAGAATTTGATGT |
| 819 | GATGTTTAGTGTAGATATAGATTT |
| 820 | GAATATTGAGTTATAAGTAGTAGT |
| 821 | AGTGAGTAAGTAATAGAAAGATTT |
| 822 | GTAGAATAAGTAATTTGTGAGATA |
| 823 | GAGTTATTTGAGATTTAGATGTTT |
| 824 | GAAATGATGATTGAATTTAGAGAT |
| 825 | AAATAGTGTGAGAATAGTTAAGTA |
| 826 | ATGTGTTAAGTTGTAGAAGAATAA |
| 827 | ATAATGAGTTAATAGTGTAAGAAG |
| 828 | ATAAGAGATGTTTAAGTTAGAAAG |
| 829 | TGTTAGTGTTAGAAATATGAAAGA |
| 830 | TTTAGAAGATTGTTAGATAAGTTG |
| 831 | GTGTAATGTATAAGATAGTTAAGT |
| 832 | TATTAGAGAGAAATTGTAGAGATT |
| 833 | TAGTGAGATAAAGTAAAGTTTATG |
| 834 | TTGTGAAAGTTAAGTAAGTTAGTT |
| 835 | AAAGTGTAAGTTGAAGAATATTGA |
| 836 | GAATAGAGTGTTATTTGAAATAGA |
| 837 | TATAAGAGAGAGATAAGTAATAAG |
| 838 | TGAGTGAAATTGATAGAGTAAATT |
| 839 | GATGAATAAGTTTAAGTGAGAAAT |
| 840 | GTGTGATATGTTTATTGATTAAGT |
| 841 | TAAAGTGAGTGTAAATGATAATGA |
| 842 | GTAGAGTTTGATTTGAAAGAATAT |
| 843 | GAATATTGTTATGTTTGTTATGAG |
| 844 | GTGTAATAAGATGTATTGTTGTTT |
| 845 | TAAATTGATTGTGAGTTGAAGAAT |
| 846 | TGAGATAGTTATAGTTAAGTTTAG |
| 847 | AGTTTGTTAAGATTATGTAGAAAG |
| 848 | GAATGTGTAGAATAAGAGATTAAA |
| 849 | GTATTATGAAAGAAGTTGTTGTTT |
| 850 | GTGTTATAGAAGTTAAATGTTAAG |
| 851 | TTAAGAGTAGTGAATATGATAGTA |
| 852 | AATGTTATAAGATGAGAGTTTAGT |
| 853 | ATATAAGATTTGATGTAGTGTAGT |
| 854 | TATGTTTGTTGTTGTTAAGTTTGA |
| 855 | GATAGTTTAGTATAGAAGATAAAG |
| 856 | GTTGAATATAGAGATAGTAAATAG |
| 857 | AGAGAAGATTTAGTAAGAATGATA |
| 858 | TGAATGAGAAAGATATTGAGTATT |
| 859 | TGAAGATTATAGTAGTTGTATAGA |
| 860 | GATTAGTAGTATTGAAGATTATGT |
| 861 | TGAAATGTGTATTTGTATGTTTAG |
| 862 | ATTAAAGTTGATATGAAAGAAGTG |
| 863 | AATGTAGAGATTGTAGTGAATATT |
| 864 | TTATTTGTTGAGTGTAAATGTGAT |
| 865 | ATGTAATTGTGAATAATGTATGTG |
| 866 | GATTTGTATAGAGATTAGTAAGTA |
| 867 | AATATTGTTGTTTAGAGAAAGAAG |
| 868 | ATGATGATGTATTTGTAAAGAGTA |
| 869 | AATGTATTTGTGTGATTGTGTAAA |
| 870 | AGTGTTATGAAGAATAGTAAGAAT |
| 871 | GTTATGTAGAGATGAAAGAAATTA |
| 872 | GTTTGTATTAGATAAATGAGTTGT |
| 873 | TGATTTATGAGATTAAGAGAAAGA |
| 874 | TTTGTGTGTTATTGTAATTGAGAT |
| 875 | GATGTGTGATATGATTAAAGAAAT |
| 876 | AGATTATAGATTTGTAGAGAAAGT |
| 877 | GAAGAGTATGTAATAGTATTGTAT |
| 878 | TTTGTAATGTTGTTGAGTTTAAGA |
| 879 | AGTAAATAGTAGTATGAATAAGAG |
| 880 | GAATGTTGAATTGAAATATGAGTT |
| 881 | AGTAGTTAATTGATAGTAAGTTTG |
| 882 | AGTGTAAAGAAATGAATGAATAAG |
| 883 | TGTTAGATATTTGTGAAATGTGAA |
| 884 | TGTATGTTGAGTTTGAATTGTTAT |
| 885 | TGAGTGAATTAGTTATGTTGTTAT |
| 886 | GAAGAAAGAAATGAGAAAGATTAT |
| 887 | TTAAGTAAGTTGTGTTGATATTAG |
| 888 | ATGATGTGTTTGATTTGAATTGAA |
| 889 | AAGTAAGTGAAATTGTTGTTTGAA |
| 890 | ATGAAGTGTAAAGTTTGAAAGAAA |
| 891 | AGAGAGTAAGATAATTGTATAGTA |
| 892 | TTTATGAGATAGATGAAATAAGTG |
| 893 | AGAAATTAGTAGTAATGATTTGTG |
| 894 | GATTTGAGATTGAATGAGAATATA |
| 895 | GATTAGAAAGATGAATAAAGATGA |
| 896 | TAGATAGAAAGTATATGTTGTAGT |
| 897 | GAAGATAGTAAAGTAAAGTAAGTT |
| 898 | AAATGTGTGTTTAGTAGTTGTAAA |
| 899 | TTGTTGAAGTAAGAGATGAATAAA |
| 900 | TATTTGAGAGAAAGAAAGAGTTTA |
| 901 | TATTTAGTGATGAATTTGTGATGT |
| 902 | TTATAGTGATGATGATAAGTTGAT |
| 903 | TAAAGATAATTGTAGAAAGTAGTG |
| 904 | GTTTAGTATTGATATTGTGTGTAA |
| 905 | GTGTTGTGAATAAGATTGAAATAT |
| 906 | AAAGAAAGTATAAAGTGAGATAGA |
| 907 | TATTTGTAAGAAGTGTAGATATTG |
| 908 | TAGAAGATGAAATTGTGATTTGTT |
| 909 | ATAATAGTAAGTGAATGATGAGAT |
| 910 | AATGTGAATAAGATAAAGTGTGTA |
| 911 | ATTGAAGATAAAGATGTTGTTTAG |
| 912 | TGAAATAGAAGTGAGATTATAGTA |
| 913 | AGTTATTGTGAAAGAGTTTATGAT |
| 914 | AAATAGTAGTGATAGAGAAGATTT |
| 915 | AGTGTATGAAGTGTAATAAGATTA |
| 916 | TGATTAAGATTGTGTAGTGTTATA |
| 917 | AGTTTATGATATTTGTAGATGAGT |
| 918 | TATGTGTATGAAGATTATAGTTAG |
| 919 | GAAATTGTTGTATAGAGTGATATA |
| 920 | TAGAAATAGTTTAAGTATAGTGTG |
| 921 | TGATTTAGATGTTTATTGTGAGAA |
| 922 | AAGTTGATATTTGTTGTTAGATGA |
| 923 | TGATGTGATAATGAGAATAAAGAA |
| 924 | AAAGTTTAGTTTGTATTAGTAGAG |
| 925 | AGTTTGATGTGATAGTAAATAGAA |
| 926 | AAGTGTTATTGAATGTGATGTTAT |
| 927 | AAATTGAAGTGTGATAATGTTTGT |
| 928 | GTTTAGTGATTAAAGATAGATTAG |
| 929 | ATAAGTGTATAAGAGAAGTGTTAA |
| 930 | ATGAATTTGTTTGTGATGAAGTTA |
| 931 | AAAGAATTGAGAAATGAAAGTTAG |
| 932 | AGTGTAAGAGTATAAAGTATTTGA |
| 933 | GAATTAAGATTGTTATATGTGAGT |
| 934 | TATGAAAGTGTTGTTTAAGTAAGA |
| 935 | TAAAGTAAATGTTATGTGAGAGAA |
| 936 | AAAGATATTGATTGAGATAGAGTT |
| 937 | AAGTGATATGAATATGTGAGAAAT |
| 938 | AAATAGAGTTTGTTAATGTAAGTG |
| 939 | GATTTAGATGAGTTAAGAATTTAG |
| 940 | TTGTAAATGAGTGTGAATATTGTA |
| 941 | AGTAGTGTATTTGAGATAATAGAA |
| 942 | TGAGTTAAAGAGTTGTTGATATTT |
| 943 | AAAGAGTGTATTAGAAATAGTTTG |
| 944 | GTTTAGTTATTTGATGAGATAATG |
| 945 | AAGTGTAAATGAATAAAGAGTTGT |
| 946 | AATAAAGTGAGTAGAAGTGTAATT |
| 947 | TATTGAGTTTGTGTAAAGAAGATA |
| 948 | TTTATAGTTGTTGTGTTGAAAGTT |
| 949 | ATGAAATATGATTGTGTTTGTTGT |
| 950 | AAAGAGATGTAAAGTGAGTTATTA |
| 951 | TTGAAGAAAGTTAGATGATGAATT |
| 952 | ATGTTATTTGTTTAGTTTGTGTGA |
| 953 | AAATATGAATTTGAAGAGAAGTGA |
| 954 | GATTAGATATAGAATATTGAAGAG |
| 955 | TTAGAATAAGAGAAATGTATGTGT |
| 956 | TTTATGAAAGAGAAGTGTATTATG |
| 957 | GTAAGTATTAAGTGTGATTTAGTA |
| 958 | ATAAAGAGAAGTAAAGAGTAAAGT |
| 959 | ATTGTTAATTGAAGTGTATGAAAG |
| 960 | TATATAGTTGAGTTGAGTAAGATT |
| 961 | TAGATGAGATATATGAAAGATAGT |
| 962 | ATAAGAAGATGATTTGTGTAAATG |
| 963 | TTAGTAATAAGAAAGATGAAGAGA |
| 964 | GATTTGTGAGTAAAGTAAATAGAA |
| 965 | AAATAGATGTAGAATTTGTGTGTT |
| 966 | GAAATTAGTGTTTGTGTGTATTAT |
| 967 | ATTTGAGTATGATAGAAGATTGTT |
| 968 | ATAGAGTTGAAGTATGTAAAGTTT |
| 969 | TAATTTGTGAATGTTGTTATTGTG |
| 970 | TTAGTTTATGAGAGTGAGATTTAA |
| 971 | GTTGTTAGAGTGTTTATGAAATTT |
| 972 | TTTATTGTGATGTGAAATAAGAGA |
| 973 | GTAAGTAATATGATAGTGATTAAG |
| 974 | TGAGATGATGTATATGTAGTAATA |
| 975 | AATTGAGAAAGAGATAAATGATAG |
| 976 | TTTGAAGTGATGTTAGAATGTTTA |
| 977 | AGTTGTTGTGTAATTGTTAGTAAA |
| 978 | ATAGTGAGAAGTGATAAGATATTT |
| 979 | GTGTGATAAGTAATTGAGTTAAAT |
| 980 | TAGTTATTGTTTGTGAATTTGAGA |
| 981 | ATAGTTGAATAGTAATTTGAAGAG |
| 982 | ATGTTTGTGTTTGAATAGAGAATA |
| 983 | TGATAAAGATATGAGAGATTGTAA |
| 984 | TAAAGATGAGATGTTGTTAAAGTT |
| 985 | AAGTGAAATTTGTAAGAATTAGTG |
| 986 | GAAATGAGAGTTATTGATAGTTTA |
| 987 | TTTGTAAATGAGATATAGTGTTAG |
| 988 | GTTAATTGTGATATTTGATTAGTG |
| 989 | AGAGTGTTGATAAAGATGTTTATA |
| 990 | AATTGTGAGAAATTGATAAGAAGA |
| 991 | TTAAAGAGAATTGAGAAGAGAAAT |
| 992 | TTGTTAGAAGAATTGAATGTATGT |
| 993 | AGTTAAGATATGTGTGATGTTTAA |
| 994 | TGAGTTATGTTGTAATAGAAATTG |
| 995 | TTAGATAAGTTTAGAGATTGAGAA |
| 996 | ATGAGTAATAAGAGTATTTGAAGT |
| 997 | TGTTTAAGTGTAATGATTTGTTAG |
| 998 | TTGAAGAAGATTGTTATTGTTGAA |
| 999 | TATAGAAAGATTAAAGAGTGAATG |
| 1000 | TAAATTGTTAGAAATTTGAGTGTG |
| 1001 | ATTGTTAGTGTGTTATTGATTATG |
| 1002 | GAGAATTATGTGTGAATATAGAAA |
| 1003 | TTGATTGATAAAGTAAAGAGTGTA |
| 1004 | GTGTGTAAATTGAATATGTTAATG |
| 1005 | AAAGTAAAGAAAGAAGTTTGAAAG |
| 1006 | TTTAGTTGAAGAATAGAAAGAAAG |
| 1007 | GTGTAATAAGAGTGAATAGTAATT |
| 1008 | TATTGAAATAAGAGAGATTTGTGA |
| 1009 | ATGAGAAAGAAGAAGTTAAGATTT |
| 1010 | AAGAGTGAGTATATTGTTAAAGAA |
| 1011 | TTTGTAAAGTGATGATGTAAGATA |
| 1012 | GATGTTATGTGATGAAATATGTAT |
| 1013 | GTAGAATAAAGTGTTAAAGTGTTA |
| 1014 | AAAGAGTATGTGTGTATGATATTT |
| 1015 | AAAGATAAGAGTTAGTAAATTGTG |
| 1016 | AAGAATTAGAGAATAAGTGTGATA |
| 1017 | GATAAGAAAGTGAAATGTAAATTG |
| 1018 | GATGAAAGATGTTTAAAGTTTGTT |
| 1019 | AGTGTAAGTAATAAGTTTGAGAAA |
| 1020 | GTTGAGAATTAGAATTTGATAAAG |
| 1021 | TTAAGAAATTTGTATGTGTTGTTG |
| 1022 | AGAAGATTTAGATGAAATGAGTTT |
| 1023 | TAAGTTTGAGATAAAGATGATATG |
| 1024 | TGAGATAGTTTGTAATATGTTTGT |
| 1025 | AGTTTGAAATTGTAAGTTTGATGA |
| 1026 | TAGAATTGATTAATGATGAGTAGT |
| 1027 | AGAGATTTGTAATAAGTATTGAAG |
| 1028 | ATAATGATGTAATGTAAGTAGTGT |
| 1029 | TGAAATTTGATGAGAGATATGTTA |
| 1030 | TGTGTAAAGTATAGTTTATGTTAG |
| 1031 | TGAATAAGTGAAATAGAATGAATG |
| 1032 | AAAGAAAGATTGTAATAAGTAGAG |
| 1033 | AATGAAATAGTGTTAAATGAGTGT |
| 1034 | GTAGATAAAGATGTGAATTATGAT |
| 1035 | GATAGTATATGTGTGTATTTGTTT |
| 1036 | ATGTTTGTAGAAATGTTTGAAGAT |
| 1037 | AAATTTGTAGAGAGAAATTTGTTG |
| 1038 | TAGAATAAGATTAGTAAGTGTAGA |
| 1039 | TGATTTAGAGAAATATGAGTAGAA |
| 1040 | AATAGAGTATGTTGTTTATGAGAA |
| 1041 | GATGATGAAGAGTTTATTGTAAAT |
| 1042 | AAGTAAAGAAGAAGAAATGTGTTA |
| 1043 | TTGAAGAATTAAGTGTTTAGTGTA |
| 1044 | AGAAAGAATGTTGATTTATGATGT |
| 1045 | GATTAAAGAGATGTTGATTGAAAT |
| 1046 | AATGATAATTGTTGAGAGAGTAAT |
| 1047 | GTTTGTTGAAAGTGTAAAGTATAT |
| 1048 | TGAGTTATATGAGAAAGTGTAATT |
| 1049 | TTGTGAGAAAGAAGTATATAGAAT |
| 1050 | GTAAGTTTAGAGTTATAGAGTTTA |
| 1051 | GATAGATAGATAAGTTAATTGAAG |
| 1052 | AGAGATGATTGTTTATGTATTATG |
| 1053 | AAAGTTAAGAAATTGTAGTGATAG |
| 1054 | TTTGATATTGTTTGTGAGTGTATA |
| 1055 | ATTTGTAGAAAGTTGTTATGAGTT |
| 1056 | GATTTGAGTAAGTTTATAGATGAA |
| 1057 | AAGATAAAGTGAGTTGATTTAGAT |
| 1058 | GATATTGTAAGATATGTTGTAAAG |
| 1059 | GTAAGAGTGTATTGTAAGTTAATT |
| 1060 | GTGTGATTAGTAATGAAGTATTTA |
| 1061 | GTAAGAAAGATTAAGTGTTAGTAA |
| 1062 | AGTAGAAAGTTGAAATTGATTATG |
| 1063 | TAAGAGAAGTTGAGTAATGTATTT |
| 1064 | GTTAAGAAATAGTAGATAAGTGAA |
| 1065 | TAAGTAAATTGAAAGTGTATAGTG |
| 1066 | AAGATGTATGTTTATTGTTGTGTA |
| 1067 | ATTTAGAATATAGTGAAGAGATAG |
| 1068 | GTTATGAAAGAGTATGTGTTAAAT |
| 1069 | TATTATGTGAAGAAGAATGATTAG |
| 1070 | TAATAAGTTGAAGAGAATTGTTGT |
| 1071 | TGATGTTTGATGTAATTGTTAAAG |
| 1072 | GTGAAAGATTTGAGTTTGTATAAT |
| 1073 | AGAGAATATAGATTGAGATTTGTT |
| 1074 | TTTGAGATGTGATGATAAAGTTAA |
| 1075 | GTTGTAAATTGTAGTAAAGAAGTA |
| 1076 | GTGTTATGATGTTGTTTGTATTAT |
| 1077 | ATTATTGTGTAGATGTATTAAGAG |
| 1078 | GTTAGAAAGATTTAGAAGTTAGTT |
| 1079 | TTGTGTATTAAGAGAGTGAAATAT |
| 1080 | GTTTAAGATAGAAAGAGTGATTTA |
| 1081 | AATGAGAAATAGATAGTTATTGTG |
| 1082 | TGAATTGAATAAGAATTTGTTGTG |
| 1083 | AATAAGATTGAATTAGTGAGTAAG |
| 1084 | AATGTTTGAGAGATTTAGTAAAGA |
| 1085 | AGTTTAGAATAGAAATGTGTTTGA |
| 1086 | TATAAGTAAGTGTTAAGATTTGAG |
| 1087 | GTAGTGAATAAGTTAGTGTTAATA |
| 1088 | AAGTGTGTTAAAGTAAATGTAGAT |
| 1089 | AGAGATGTTTATGTTGTGAATTAA |
| 1090 | AGTTGAATATTGATGATAAGAAGA |
| 1091 | TGAATGTGAGATGTTTAGAATAAT |
| 1092 | AATAATGATGTAAGTTTGAGTTTG |
| 1093 | AAAGAGTGAATAGAAATAAGAGAA |
| 1094 | AATAAAGTTATTGAGAGAGTTTAG |
| 1095 | AGTAGTGTTGTAGTTTAGTATATA |
| 1096 | GTAAGAATGTATTAGATATTTGTG |
| 1097 | GATAAATGTTTGATAAAGTAGTTG |
| 1098 | ATAGTATGTATGTGTGAAGATTTA |
| 1099 | ATGAATGTAGAGTGATTAGTTTAA |
| 1100 | GTAGTATTTAGTGATGTAAGAATA |
| 1101 | AGAATTGTATTGAAGAAGAATATG |
| 1102 | TTTATAGAATTGAGAGAAGTTAAG |
| 1103 | AAAGTAGTAGAGATTTGAGAATTA |
| 1104 | TTTAAAGAAAGTATTGTAAGAGTG |
| 1105 | AAATTGAGAAAGTGAATGAAGTTT |
| 1106 | AAGAAATAAGTATGATAGTAGTAG |
| 1107 | ATTTGAATTGTATTGTAGTTTGTG |
| 1108 | AAGAGAATAATGTAGAGATATAAG |
| 1109 | TGTGTAATAGTTGTTAATGAGTAA |
| 1110 | TATAGTTGTAGTTTAGATGAATGT |
| 1111 | ATTGTGTTAGAATGATGTTAATAG |
| 1112 | GTTTGTATAGTATTTGATTGATGT |
| 1113 | AGAGTAAAGTATGAGTTATGAATA |
| 1114 | GAAAGTTTAAGTGATGTATATTGT |
| 1115 | TTAAATGATAAAGAGTAGTGAAGT |
| 1116 | TTAAATGTGTGAGAAGATGAATAA |
| 1117 | ATTTGTATAAAGTGAAGAAGAGAA |
| 1118 | TGATTAGTATTTGTGAAGAGATTT |
| 1119 | TTTGAATGAAATTGATGATAGATG |
| 1120 | AGAGTAAGATTAAGAATAAGAAAG |
| 1121 | ATTGAATTGAGAAGTGAAGTAAAT |
| 1122 | TTTAGAGAAGTATTGTTTGAAAGA |
| 1123 | TAAAGTGAAAGATTTGAAATGATG |
| 1124 | GAAAGTTAGAGAAATGTAGAAATT |
| 1125 | GTGAATAATGAAGAAGTTATGTTA |
| 1126 | TTGTGAATAAAGTAGATGTGTTAT |
| 1127 | TTATATGATATGAGTTTGTGTTGA |
| 1128 | TTGATTTGTGTGAGTATTAGTTAT |
| 1129 | AAAGTGATTAAGTTAGTTTGAGAT |
| 1130 | TTGTATTTGTATAATGTTGAAGAG |
| 1131 | GTTTGAAATTAGTGTGAGAAATAT |
| 1132 | AATGTTGAGATTGATAATGTTGAA |
| 1133 | TAGTAGTAGTATTGTTGTAATAAG |
| 1134 | GTTGTAATTTGAGTGTTAGTTATT |
| 1135 | TGAATATGATAGTTAGTAATTGTG |
| 1136 | TGATAGTATGTTTGTGATTAAAGA |
| 1137 | GATGTATAAAGAGTATGTTATAAG |
| 1138 | AGTGAGATTTAGAAGATGTTATTA |
| 1139 | ATGAGAATTTGTTAAAGAGAAAGT |
| 1140 | AAAGAATTAGTATGATAGATGAGA |
| 1141 | TAGAGTTGTATAGTTTATAGTTGA |
| 1142 | GTAGAATGATTGTTTAGAAGATTT |
| 1143 | GTTTATGTTTGAGAAGAGTTATTT |
| 1144 | TAGAAGTTTGAAAGTTATTGATTG |
| 1145 | GATGAAGAGTATTTGTTATATGTA |
| 1146 | GATGAATATAGTAAGTATTGAGTA |
| 1147 | TAGTGATGAAATTTGAGATAGATA |
| 1148 | GAAAGAAATTGAAGAGTTTGATAT |
| 1149 | ATTTGAGTATTTGTGTATTGAATG |
| 1150 | ATGAGTTGAAATTTGAAGTATTGT |
| 1151 | TTAATAGTGAGAGAGTATATGTAA |
| 1152 | ATTAAGAGAGTGAGTAAATGTAAA |
| 1153 | AAGAATAGATGAGATTAGAAATAG |
| 1154 | AGTTTAAAGAGTTAGAATTGAAAG |
| 1155 | GTAAGATTTGTTGAATAAAGAAGA |
| 1156 | AGAGAAAGAAGTTAAAGTGATATT |
| 1157 | TAATAGAGAAGAGATGTATGAATA |
| 1158 | TTATTAGTGATAAGTGAAGTTTAG |
| 1159 | ATAATGTAAAGATGAGTTTATGAG |
| 1160 | TTGATTTGAGAGTTGATAAGATTT |
| 1161 | ATGATTATTGTGTGTAGAATTAGA |
| 1162 | TATAAAGATATAGTAGATGATGTG |
| 1163 | TTTAGTTGAGATGAAGTTATTAGA |
| 1164 | ATTGAATTGATATAGTGTAAAGTG |
| 1165 | GAAGAAAGATTATTGTATTGAGTT |
| 1166 | ATTGAGTGTAGTGATTTAGAAATA |
| 1167 | AATAAAGTGTTTAAGAGTAGAGTA |
| 1168 | GTAGAGATAATTGATGTGTAATTT |

Also, all of the sequences of a family could be taken to be constructed in the 5′-3′ direction, as is the convention, or all of the constructions of sequences could be in the opposition direction (3′-5′).

There are additional modifications that may be carried out. For example, C has not been used in the family of sequences. Substitution of C in place of one or more G's of a particular sequence would yield a sequence that is at least as low in homology with every other sequence of the family as was the particular sequence chosen for modification. It is thus possible to substitute C in place of one or more G's in any of the sequences shown in Table II. Analogously, substituting of C in place of one or more A's is possible, or substituting C in place of one or T's is possible.

It is preferred that the sequences of a given family are of the same, or roughly the same length. Preferably, all the sequences of a family of sequences of this invention have a length that is within five bases of the base-length of the average of the family. More preferably, all sequences are within four bases of the average base-length. Even more preferably, all or almost all sequences are within three bases of the average base-length of the family. Better still, all or almost all sequences have a length that is within two of the base-length of the average of the family, and even better still, within one of the base-length of the average of the family.

It is also possible for a person skilled in the art to derive sets of sequences from the family of sequences described in this specification and remove sequences that would be expected to have undesirable hybridization properties.

Given a particular family of sequences that can be used as a family of tags (or tag complements), e.g., those of Table I or Table II, or the combined sequences of these two tables, a skilled person will readily recognize variant families that work equally as well.

Again taking the sequences of Table I for example, every T could be converted to an A and vice versa and no significant change in the cross-hybridization properties would be expected to be observed. This would also be true if every G were converted to a C.

Also, all of the sequences of a family could be taken to be constructed in the 5′-3′ direction, as is the convention, or all of the constructions of sequences could be in the opposition direction (3′-5′).

There are additional modifications that can be carried out. For example, C has not been used in the family of sequences. Substitution of C in place of one or more T's of a particular sequence would yield a sequence that is at least as low in homology with every other sequence of the family as the particular sequence chosen to be modified was. It is thus possible to substitute C in place of one or more T's in any of the sequences shown in Table I. Analogously, substituting of C in place of one or more A's is possible, or substituting C in place of one or T's is possible.

It is preferred that the sequences of a given family are of the same, or roughly the same length. Preferably, all the sequences of a family of sequences of this invention have a length that is within five bases of the base-length of the average of the family. More preferably, all sequences are within four bases of the average base-length. Even more preferably, all or almost all sequences are within three bases of the average base-length of the family. Better still, all or almost all sequences have a length that is within two of the base-length of the average of the family.

It is also possible for a person skilled in the art to derive sets of sequences from the family of sequences that is the subject of this patent and remove sequences that would be expected to have undesirable hybridization properties.

### Methods for Synthesis of Oligonucleotide Families

Preferably oligonucleotide sequences of the invention are synthesized directly by standard phosphoramidite synthesis approaches and the like (Caruthers et al, Methods in Enzymology; 154, 287-313: 1987; Lipshutz et al, Nature Genet.; 21, 20-24: 1999; Fodor et al, Science; 251, 763-773: 1991). Alternative chemistries involving non natural bases such as peptide nucleic acids or modified nucleosides that offer advantages in duplex stability may also be used (Hacia et al; Nucleic Acids Res; 27: 4034-4039, 1999; Nguyen et al, Nucleic Acids Res.; 27, 1492-1498: 1999; Weiler et al, Nucleic Acids Res.; 25, 2792-2799:1997). It is also possible to synthesize the oligonucleotide sequences of this invention with alternate nucleotide backbones such as phosphorothioate or phosphoroamidate nucleotides. Methods involving synthesis through the addition of blocks of sequence in a step wise manner may also be employed (Lyttle et al, Biotechniques, 19: 274-280 (1995). Synthesis may be carried out directly on the substrate to be used as a solid phase support for the application or the oligonucleotide can be cleaved from the support for use in solution or coupling to a second support.

### Solid Phase Supports

There are several different solid phase supports that can be used with the invention. They include but are not limited to slides, plates, chips, membranes, beads, microparticles and the like. The solid phase supports can also vary in the materials that they are composed of including plastic, glass, silicon, nylon, polystyrene, silica gel, latex and the like. The surface of the support is coated with the complementary sequence of the same.

In some embodiments, the family of tag complement sequences are derivatized to allow binding to a solid support. Many methods of derivatizing a nucleic acid for binding to a solid support are known in the art (Hermanson G., Bioconjugate Techniques; Acad. Press: 1996). The sequence tag may be bound to a solid support through covalent or non-covalent bonds (Iannone et al, Cytometry; 39: 131-140, 2000; Matson et al, Anal. Biochem.; 224: 110-106, 1995; Proudnikov et al, Anal Biochem; 259: 34-41, 1998; Zammatteo et al, Analytical Biochemistry; 280:143-150, 2000). The sequence tag can be conveniently derivatized for binding to a solid support by incorporating modified nucleic acids in the terminal 5′ or 3′ locations.

A variety of moieties useful for binding to a solid support (e.g., biotin, antibodies, and the like), and methods for attaching them to nucleic acids, are known in the art. For example, an amine-modified nucleic acid base (available from, eg., Glen Research) may be attached to a solid support (for example, Covalink-NH, a polystyrene surface grafted with secondary amino groups, available from Nunc) through a bifunctional crosslinker (e.g., bis(sulfosuccinimidyl suberate), available from Pierce). Additional spacing moieties can be added to reduce steric hindrance between the capture moiety and the surface of the solid support.

### Attaching Tags to Analytes for Sorting

A family of oligonucleotide tag sequences can be conjugated to a population of analytes most preferably polynucleotide sequences in several different ways including but not limited to direct chemical synthesis, chemical coupling, ligation, amplification, and the like. Sequence tags that have been synthesized with primer sequences can be used for enzymatic extension of the primer on the target for example in PCR amplification.

### Kits Using Families of Tag Sequences

The families of INVADER assay probes comprising non cross-hybridizing 5′ tag sequences may be provided in kits for use in, for example, genetic analysis. Such kits include one or more probes comprising non-cross hybridizing sequences. Reagents may include enzymes, nucleotides, fluorescent labels and the like that would be required for specific applications. Instructions for correct use of the kit for a given application may be provided.

Filed herewith on compact disk, and expressly incorporated herein by reference, is a Sequence Listing provided as a file entitled “10956.txt,” 427 kb in size, created Jun. 7, 2006.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

What is claimed is:

1. A composition comprising a cleavage structure, said cleavage structure comprising:
a) a target nucleic acid having a first region and a second region, wherein said second region is located adjacent to and downstream of said first region;
b) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and is selected from the group consisting of tag identifiers 1-210 of Table I
wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is a base in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y=A or T/U,
and X≠Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, TIU, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−1) bases in length;
(ii) the phantom sequence, if greater than or equal to (⅚×L) in length, contains at least three insertions/deletions or mismatches when compared to the first and second sequences from which it is generated; and
(iii) the phantom sequence is not greater than or equal to ( 11/12)×L) in length;
where L=L₁, or if L₁≠L₂, where L is the greater of L₁ and L₂; and
wherein any base present may be substituted by an analogue thereof; and
c) a second nucleic acid molecule comprising a 3′ portion and a 5′ portion, wherein said 5′ portion is completely complementary to said second region of said target nucleic acid.

2. The composition of claim 1, further comprising a 5′ nuclease.

3. The composition of claim 2, wherein said 5′ nuclease is a FEN-1 nuclease.

4. The composition of claim 1, wherein said tag identifiers 1-210 are selected from the group consisting of SEQ ID NOS: 1173-1382.

5. A method for detecting the presence of a target nucleic acid molecule in a sample, comprising:
a) incubating a sample with a thermostable 5′ nuclease under conditions wherein a cleavage structure is formed, said cleavage structure comprising:
i) a target nucleic acid having a first region and a second region, wherein said second region is located adjacent to and downstream of said first region;
ii) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and is selected from the group consisting of tag identifiers 1-210
wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is a base in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y=A or T/U,
and X≠Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−1) bases in length;
(ii) the phantom sequence, if greater than or equal to (⅚×L) in length, contains at least three insertions/deletions or mismatches when compared to the first and second sequences from which it is generated; and
(iii) the phantom sequence is not greater than or equal to ( 11/12)×L) in length;
where L=L₁, or if L₁≠L₂, where L is the greater of L₁ and L₂; and
wherein any base present may be substituted by an analogue thereof; and
iii) a second nucleic acid molecule comprising a 3′ portion and a 5′ portion, wherein said 5′ portion is completely complementary to said second region of said target nucleic acid;
wherein said thermostable 5′ nuclease lacks synthesis activity, and wherein at least a portion of said first nucleic acid molecule is annealed to first region of said target nucleic acid, and wherein at least a portion of said second nucleic acid molecule is annealed to said second region of said target nucleic acid;
b) cleaving said cleavage structure with said thermostable 5′ nuclease so as to generate non-target cleavage product; and
c) detecting the cleavage of said cleavage structure.

6. The method of claim 5, wherein said non-target cleavage product comprises the 5′ portion of said first nucleic acid molecule, and wherein said detecting the cleavage of said cleavage structure comprises detecting annealing of said non-target cleavage product to a third nucleic acid molecule, wherein said third nucleic acid molecule comprises a nucleic acid sequence complementary to the sequence of the tag identifier selected in step (a) (iv).

7. The method of claim 5, wherein said detecting the cleavage of said cleavage structure comprises detection of fluorescence.

8. The method of claim 5, wherein said detecting the cleavage of said cleavage structure comprises detection of fluorescence energy transfer.

9. The method of claim 5, wherein said target nucleic acid comprises DNA.

10. The method of claim 5, wherein said 3′ portion of said second nucleic acid molecule comprises a 3′ terminal nucleotide not complementary to said target nucleic acid.

11. The method of claim 5, wherein said tag identifiers 1-210 are selected from the group consisting of SEQ ID NOS: 1173-1382.

12. A composition comprising a cleavage structure, said cleavage structure comprising:
i) a target nucleic acid having a first region and a second region, wherein said second region is located adjacent to and downstream of said first region;
ii) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and that is selected from the group consisting of tag identifiers 211-1378, wherein
each of 1 to 3 is a nucleotide base selected to be different from the others of 1 to 3, with the proviso that up to three nucleotide bases of each sequence can be substituted with any nucleotide base provided that for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein
the score of an alignment is determined according to the equation (A×m)−(B×mm)−(C×(o−g+eg))−(D×eg)), wherein:
for each of (i) to (iv):
(i) m=6, mm=6, og=0 and eg=6,
(ii) m=6, mm=6, og=5 and eg=1,
(iii) m=6, mm=2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv); and
iii) a second nucleic acid molecule comprising a 3′ portion and a 5′ portion, wherein said 5′ portion is completely complementary to said second region of said target nucleic acid.

13. The composition of claim 12, further comprising a 5′ nuclease.

14. The composition of claim 13, wherein said 5′ nuclease is a FEN-1 nuclease.

15. The composition of claim 12, wherein said tag identifiers 211-1378 are selected from the group consisting of SEQ ID NOS: 1-1172.

16. A method for detecting the presence of a target nucleic acid molecule in a sample, comprising:
a) incubating a sample with a thermostable 5′ nuclease under conditions wherein a cleavage structure is formed, said cleavage structure comprising:
i) a target nucleic acid having a first region and a second region, wherein said second region is located adjacent to and downstream of said first region;
ii) a first nucleic acid molecule comprising a 3′ portion and a 5′ portion, wherein at least a portion of said 3′ portion of said first nucleic acid molecule is completely complementary to said first region of said target nucleic acid, and wherein said 5′ portion contains a tag identifier that is not base-paired to said target nucleic acid and that is selected from the group consisting of tag identifiers 211-1378, wherein
each of 1 to 3 is a nucleotide base selected to be different from the others of 1 to 3, with the proviso that up to three nucleotide bases of each sequence can be substituted with any nucleotide base provided that for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein
the score of an alignment is determined according to the equation (A×m)−(B×mm)−(C×(o−g+eg))−(D×eg)), wherein:
for each of (i) to (iv):
(i) m=6, mm=6, og=0 and eg=6,
(ii) m=6, mm=6, og=5 and eg=1,
(iii) m=6, mm=2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv); and
iii) a second nucleic acid molecule comprising a 3′ portion and a 5′ portion, wherein said 5′ portion is completely complementary to said second region of said target nucleic acid;
wherein said thermostable 5′ nuclease lacks synthesis activity, and wherein at least a portion of said first nucleic acid molecule is annealed to first region of said target nucleic acid, and wherein at least a portion of said second nucleic acid molecule is annealed to said second region of said target nucleic acid;
b) cleaving said cleavage structure with said thermostable 5′ nuclease so as to generate non-target cleavage product; and
c) detecting the cleavage of said cleavage structure.

17. The method of claim 16, wherein said non-target cleavage product comprises the 5′ portion of said first nucleic acid molecule, and wherein said detecting the cleavage of said cleavage structure comprises detecting annealing of said non-target cleavage product to a third nucleic acid molecule, wherein said third nucleic acid molecule comprises a nucleic acid sequence complementary to the sequence of the tag identifier selected in step (a) (iv).

18. The method of claim 16, wherein said detecting the cleavage of said cleavage structure comprises detection of fluorescence.

19. The method of claim 16, wherein said detecting the cleavage of said cleavage structure comprises detection of fluorescence energy transfer.

20. The method of claim 16, wherein said target nucleic acid comprises DNA.

21. The method of claim 16, wherein said 3′ portion of said second nucleic acid molecule comprises a 3′ terminal nucleotide not complementary to said target nucleic acid.

22. The method of claim 16, wherein said tag identifiers 211-1378 are selected from the group consisting of SEQ ID NOS: 1-1172.
